(19) United States
(12) Patent Application Publication
(10) Pub. No.: US 2025/0082756 A1
(43) Pub. Date: Mar. 13, 2025

(54) MUC16 CHIMERIC ANTIGEN RECEPTORS

(71) Applicant: Regeneron Pharmaceuticals, Inc., Tarrytown, NY (US)

(72) Inventors: Tina Albershardt, Olympia, WA (US); Geoffrey Blackburn Parsons, Jamaica Plain, MA (US); David Dilillo, New York, NY (US)

(21) Appl. No.: 18/727,387

(22) PCT Filed: Jan. 10, 2023
(86) PCT No.: PCT/US2023/010518

§ 371 Date: Jul. 9, 2024

Related U.S. Application Data
(60) Provisional application No. 63/298,141, filed on Jan. 10, 2022.

(51) Int. Cl. A61K 39/00; A61P 35/00; C07K 14/725; C07K 16/30

(52) U.S. Cl. CPC A61K 40/4257; A61K 40/11; A61K 40/31; A61P 35/00; C07K 14/7051; C07K 16/3092; C07K 2317/24; C07K 2317/622; C07K 2317/92

(57) ABSTRACT

The present disclosure provides chimeric antigen receptors (CARs) targeting MUC16, genetically modified immune effector cells, and use of these compositions to treat cancer.

### RELATED APPLICATIONS

The instant application claims priority to U.S. Provisional Application No. 63/298,141, filed Jan. 10, 2022. The entire contents of the foregoing application are expressly incorporated by reference herein.

### TECHNICAL FIELD

The present invention relates to chimeric antigen receptors (CARs) comprising anti-MUC16 (mucin 16, cell surface associated) antibodies or antigen binding fragments thereof, immune effector cells genetically modified to express these CARs, and use of these compositions to effectively treat solid tumors.

### BACKGROUND

Mucin 16 (MUC16), also known as cancer antigen 125 (CA-125) is a single transmembrane domain overexpressed in solid tumors, such as ovarian cancer. Expression of MUC16 on cancer cells has been shown to protect tumor cells from the immune system (Felder, M. et al. 2014, Molecular Cancer, 13:129). Although antibodies targeting MUC16 have been investigated (e.g., oregovomab and abgovomab), they have had limited clinical success. Accordingly, additional therapies targeting this antigen are needed.

### SUMMARY OF THE DISCLOSURE

The present disclosure is based, at least in part, on the discovery of chimeric antigen receptors (CARs) specific for a MUC16 polypeptide. Specifically, the CARs described herein are specific for a fragment or portion of MUC16 present on the surface of a cell after proteolytic cleavage of the full-length MUC16 polypeptide. As demonstrated herein, the disclosed anti-MUC16 CARs comprise surprising and unexpected properties. As discussed in more detail in the Examples, the CARs of the disclosure exhibit strong antigen-dependent activity as evaluated by IFN-gamma release, high transduction efficiencies, and high expression (see, e.g., Example 2). In addition, the anti-MUC16 CARs disclosed herein exhibit antigen dependent cytotoxicity in dose-dependent manners, e.g., at least 60%, at least 65%, or at least 70% cytotoxicity (Example 3). Moreover, certain anti-MUC16 CARs disclosed herein exhibited increased expression of markers, such as CD62L and CD45RA (Example 4), pointing to a greater naïve-like phenotype with potential for greater in vivo persistence. Finally, certain anti-MUC16 CARs tested herein surprisingly demonstrated superior anti-tumor activity in vivo (Examples 6 and 7), including reduction of the number of cancer cells expressing MUC16 in vivo.

Accordingly, in some aspects, the present disclosure provides a chimeric antigen receptor (CAR) comprising: an extracellular domain comprising an anti-MUC16 antibody or antigen binding fragment thereof that binds one or more epitopes of a human MUC16 polypeptide; a transmembrane domain, one or more intracellular co-stimulatory signaling domains, and a primary signaling domain. In some aspects, the MUC16 antibody or antigen binding fragment thereof binds a membrane proximal fragment of MUC16 remaining on the cell surface after proteolytic cleavage of a full-length MUC16 polypeptide. In one aspect, the fragment of MUC16 remaining on the cell surface after proteolytic cleavage comprises SEQ ID NO: 151. In some aspects, the full-length MUC16 polypeptide comprises 16 sea urchin sperm, enterokinase and agrin (SEA) domains, numbered 1-16 from N-terminus to C-terminus, and wherein the fragment of MUC16 comprises SEA domains 12-16.

In any of the foregoing or related aspects, the anti-MUC16 antibody or antigen binding fragment that binds the human MUC16 polypeptide is selected from the group consisting of: Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a Camel Ig, a VHH, Ig NAR, a minibody, a diabody, a triabody, a tetrabody, a disulfide stabilized Fv protein (“dsFv”), and a single-domain antibody (sdAb, Nanobody) or fragment thereof. In some aspects, the anti-MUC16 antibody or antigen binding fragment thereof that binds the human MUC16 polypeptide is an scFv.

In any of the foregoing or related aspects, the anti-MUC16 antibody or antigen binding fragment thereof comprises CDRL1, CDRL2, and CDRL3 regions within a variable light chain amino acid sequence as set forth in SEQ ID NO: 7. In some aspects, the anti-MUC16 antibody or antigen binding fragment thereof comprises CDRL1, CDRL2, and CDRL3 regions within a variable light chain amino acid sequence as set forth in any one of SEQ ID NOs: 47, 59, 71, 83, 95, 107, 119, 133, or 145. In some aspects, the anti-MUC16 antibody or antigen binding fragment thereof comprises CDRH1, CDRH2, and CDRH3 regions within a variable heavy chain amino acid sequence as set forth in SEQ ID NO: 8. In some aspects, the anti-MUC16 antibody or antigen binding fragment thereof comprises CDRH1, CDRH2, and CDRH3 regions within a variable heavy chain amino acid sequence as set forth in any one of SEQ ID NOs: 48, 60, 72, 84, 96, 108, 120, 134, or 146. In some aspects, the anti-MUC16 antibody or antigen binding fragment thereof comprises one or more CDRs as set forth in any one of SEQ ID NOs: 1-3. In some aspects, the anti-MUC16 antibody or antigen binding fragment thereof comprises one or more CDRs as set forth in any one of (a) SEQ ID NOs: 41-43, (b) SEQ ID NOs: 53-55, (c) SEQ ID NOs: 65-67, (d) SEQ ID NOs: 77-79, (e) SEQ ID NOs: 89-91, (f) SEQ ID NOs: 101-103, (g) SEQ ID NOs: 113-115, (h) SEQ ID NOs: 127-129, or (i) SEQ ID NOs: 139-141. In some aspects, the anti-MUC16 antibody or antigen binding fragment thereof comprises one or more CDRs as set forth in any one of SEQ ID NOs: 4-6. In some aspects, the anti-MUC16 antibody or antigen binding fragment thereof comprises a variable light chain amino acid sequence as set forth in SEQ ID NO: 7. In some aspects, the anti-MUC16 antibody or antigen binding fragment thereof comprises a variable heavy chain amino acid sequence as set forth in SEQ ID NO: 8. In some aspects, the anti-MUC16 antibody or antigen binding fragment thereof comprises one or more CDRs as set forth in any one of (a) SEQ ID NOs: 44-46, (b) SEQ ID NOs: 56-58, (c) SEQ ID NOs: 68-70, (d) SEQ ID NOs: 80-82, (e) SEQ ID NOs: 92-94, (f) SEQ ID NOs: 104-106, (g) SEQ ID NOs: 116-118, (h) SEQ ID NOs: 130-132, or (i) SEQ ID NOs: 142-144.

In any of the foregoing or related aspects, the anti-MUC16 antibody or antigen binding fragment thereof comprises a variable light chain amino acid sequence having at least 90%, 91%, 92%, 93%, 94%, 95%, 96%, 97%, 98%, 99% identity to, or comprises SEQ ID NO: 7. In some aspects, the anti-MUC16 antibody or antigen binding fragment thereof comprises a variable light chain amino acid sequence having at least 90%, 91%, 92%, 93%, 94%, 95%, 96%, 97%, 98%, 99% identity to, or comprises any one of SEQ ID NOs: 47, 59, 71, 83, 95, 107, 119, 133, or 145. In some aspects, the anti-MUC16 antibody or antigen binding fragment thereof comprises a variable heavy chain amino acid sequence having at least 90%, 91%, 92%, 93%, 94%, 95%, 96%, 97%, 98%, 99% identity to, or comprises SEQ ID NO: 8. In some aspects, the anti-MUC16 antibody or antigen binding fragment thereof comprises a variable heavy chain amino acid sequence having at least 90%, 91%, 92%, 93%, 94%, 95%, 96%, 97%, 98%, 99% identity to, or comprises any one of SEQ ID NOs: 48, 60, 72, 84, 96, 108, 120, 134, or 146.

In any of the foregoing or related aspects, the transmembrane domain is from a polypeptide selected from the group consisting of: alpha, beta or zeta chain of the T-cell receptor, CD3ε, CD3ζ, CD4, CD5, CD8a, CD9, CD16, CD22, CD27, CD28, CD33, CD37, CD45, CD64, CD80, CD86, CD134, CD137, CD152, CD154, and PD1. In some aspects, the transmembrane domain is from a polypeptide selected from the group consisting of: CD8α, CD4, CD45, PD1, and CD154. In some aspects, the transmembrane domain is from CD8α. In some aspects, the transmembrane domain comprises a sequence having at least 90%, 91%, 92%, 93%, 94%, 95%, 96%, 97%, 98%, or 99% identity to, or comprises the sequence of SEQ ID NO: 154. In one embodiment, the transmembrane domain comprises a sequence having at least 95% identity to SEQ ID NO: 154. In one embodiment, the transmembrane domain comprises SEQ ID NO: 154.

In any of the foregoing or related aspects, the one or more co-stimulatory signaling domains are from a co-stimulatory molecule selected from the group consisting of: CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70. In some aspects, the one or more co-stimulatory signaling domains are from a co-stimulatory molecule selected from the group consisting of: CD28, CD134, and CD137 (4-1BB). In some aspects, the one or more co-stimulatory signaling domains is from CD137 (4-1BB). In some aspects, the co-stimulatory signaling domain comprises a sequence having at least 90%, 91%, 92%, 93%, 94%, 95%, 96%, 97%, 98%, or 99% identity to, or comprises the sequence of SEQ ID NO: 155. In one embodiment, the co-stimulatory signaling domain comprises a sequence having at least 95% identity to SEQ ID NO: 155. In one embodiment, the co-stimulatory signaling domain comprises SEQ ID NO: 155.

In any of the foregoing or related aspects, the primary signaling domain is from CD3ζ. In some aspects, the primary signaling domain comprises a sequence having at least 90%, 91%, 92%, 93%, 94%, 95%, 96%, 97%, 98%, or 99% identity to, or comprises the sequence of SEQ ID NO: 156. In one embodiment, the primary signaling domain comprises a sequence having at least 95% identity to SEQ ID NO: 156. In one embodiment, the primary signaling domain comprises SEQ ID NO: 156.

In any of the foregoing or related aspects, the CAR comprises a hinge region polypeptide. In some aspects, the hinge region polypeptide comprises a hinge region of CD8α. In some aspects, the hinge region polypeptide comprises a sequence having at least 90%, 91%, 92%, 93%, 94%, 95%, 96%, 97%, 98%, or 99% identity to, or comprises the sequence of SEQ ID NO: 153. In one embodiment, the hinge region polypeptide comprises a sequence having at least 95% identity to SEQ ID NO: 153. In one embodiment, the hinge region polypeptide comprises SEQ ID NO: 153.

In any of the foregoing or related aspects, the CAR comprises a signal peptide. In some aspects, the signal peptide comprises an IgG1 heavy chain signal polypeptide, granulocyte-macrophage colony stimulating factor receptor 2 (GM-CSFR2) signal polypeptide, Igx signal polypeptide, or a CD8a signal polypeptide. In some aspects, the signal polypeptide comprises a CD8a signal polypeptide. In some aspects, the signal polypeptide comprises a sequence having at least 90%, 91%, 92%, 93%, 94%, 95%, 96%, 97%, 98%, or 99% identity to, or comprises the sequence of SEQ ID NO: 152. In one embodiment, the signal polypeptide comprises a sequence having at least 95% identity to SEQ ID NO: 152. In one embodiment, the signal polypeptide comprises SEQ ID NO: 152.

In any of the foregoing or related aspects, the CAR further comprises a first polypeptide linker between the variable heavy chain and variable light chain domains. In some aspects, the polypeptide linker between the variable heavy chain and variable light chain domains comprises an amino acid sequence as set forth in any one of SEQ ID NOs: 14-25. In particular embodiments, the polypeptide linker between the variable heavy chain and variable light chain domains comprises a 3xG4S amino acid linker as set forth in SEQ ID NO: 24.

In any of the foregoing or related aspects, the CAR further comprises a second polypeptide linker between the transmembrane domain and one or more intracellular co-stimulatory signaling domains. In some aspects, the polypeptide linker between the transmembrane domain and one or more intracellular co-stimulatory signaling domains comprises the sequence of LYC.

In any of the foregoing or related aspects, the anti-MUC16 antibody or antigen binding fragment thereof comprises CDRL1, CDRL2, and CDRL3 regions within a variable light chain amino acid sequence as set forth in SEQ ID NO: 7 and CDRH1, CDRH2 and CDRH3 regions within a variable heavy chain amino acid sequence as set forth in SEQ ID NO: 8; the transmembrane domain is from a polypeptide selected from the group consisting of: CD8α, CD4, CD45, PD1, and CD154; the one or more co-stimulatory signaling domains are from a co-stimulatory molecule selected from the group consisting of: CD28, CD134, and CD137 (4-1BB); and the primary signaling domain is from CD3ζ.

In any of the foregoing or related aspects, the anti-MUC16 antibody or antigen binding fragment thereof comprises CDRL1, CDRL2, and CDRL3 regions within a variable light chain amino acid sequence as set forth in SEQ ID NO: 7 and CDRH1, CDRH2 and CDRH3 regions within a variable heavy chain amino acid sequence as set forth in SEQ ID NO: 8; the transmembrane domain is from CD8α; the one or more co-stimulatory signaling domains is from CD137 (4-1BB); and the primary signaling domain is from CD3ζ.

In any of the foregoing or related aspects, the CAR comprises an amino acid sequence having at least 90%, 91%, 92%, 93%, 94%, 95%, 96%, 97%, 98%, or 99% identity to, or comprising SEQ ID NO: 9 or SEQ ID NO: 11. In some aspects, the CAR comprises the amino acid sequence of SEQ ID NO: 9. In some aspects, the CAR comprises the amino acid sequence of SEQ ID NO: 11. In some aspects, the CAR comprises an amino acid sequence having at least 90%, 91%, 92%, 93%, 94%, 95%, 96%, 97%, 98%, or 99% identity to, or comprising any one of SEQ ID NOs: 49, 50, 51, 52, 61, 62, 63, 64, 73, 74, 75, 76, 85, 86, 87, 88, 97, 98, 99, 100, 109, 110, 111, 112, 121, 122, 123, 124, 125, 126, 135, 136, 137, 138, 147, 148, 149 or 150.

In some aspects, the disclosure provides a CAR that competes for binding to one or more epitopes of a human MUC16 polypeptide with a CAR disclosed herein.

In some aspects, the disclosure provides a polynucleotide encoding a CAR described herein. In some aspects, the polynucleotide sequence encoding the CAR is set forth in SEQ ID NO: 10 or SEQ ID NO: 12. In some aspects, the disclosure provides a polynucleotide comprising SEQ ID NO: 10. In some aspects, the disclosure provides a polynucleotide comprising SEQ ID NO: 12. In some aspects, the polynucleotide sequence encodes a polypeptide having at least 90%, 91%, 92%, 93%, 94%, 95%, 96%, 97%, 98%, or 99% identity to, or comprising any one of SEQ ID NOs: 9, 11, 49, 50, 51, 52, 61, 62, 63, 64, 73, 74, 75, 76, 85, 86, 87, 88, 97, 98, 99, 100, 109, 110, 111, 112, 121, 122, 123, 124, 125, 126, 135, 136, 137, 138, 147, 148, 149 or 150.

In some aspects, the disclosure provides a vector comprising a polynucleotide described herein. In some aspects, the vector is an expression vector. In some aspects, the vector is an episomal vector. In some aspects, the vector is a viral vector. In some aspects, the vector is a retroviral vector. In some aspects, the vector is a lentiviral vector. In some aspects, the lentiviral vector is selected from the group consisting of: human immunodeficiency virus 1 (HIV-1); human immunodeficiency virus 2 (HIV-2), visna-maedi virus (VMV) virus; caprine arthritis-encephalitis virus (CAEV); equine infectious anemia virus (EIAV); feline immunodeficiency virus (FIV); bovine immune deficiency virus (BIV); and simian immunodeficiency virus (SIV). In some aspects, the vector is an adeno-associated viral vector.

In any of the foregoing or related aspects, the vector comprises a left (5′) retroviral LTR, a Psi (Ψ) packaging signal, a central polypurine tract/DNA flap (cPPT/FLAP), a retroviral export element; a promoter operably linked to a polynucleotide described herein; and a right (3′) retroviral LTR. In some aspects, the vector comprises a heterologous polyadenylation sequence. In some aspects, the promoter of the 5′ LTR is replaced with a heterologous promoter. In some aspects, the heterologous promoter is a cytomegalovirus (CMV) promoter, a Rous Sarcoma Virus (RSV)promoter, or an Simian Virus 40 (SV40) promoter. In some aspects, the 5′ LTR or 3′ LTR is a lentivirus LTR. In some aspects, the 3′ LTR comprises one or more modifications. In some aspects, the 3′ LTR comprises one or more deletions. In some aspects, the 3′ LTR is a self-inactivating (SIN) LTR. In some aspects, the promoter operably linked to a polynucleotide described herein is selected from the group consisting of: a cytomegalovirus immediate early gene promoter (CMV), an elongation factor 1 alpha promoter (EF1-α), a phosphoglycerate kinase-1 promoter (PGK), a ubiquitin-C promoter (UBQ-C), a cytomegalovirus enhancer/chicken beta-actin promoter (CAG), polyoma enhancer/herpes simplex thymidine kinase promoter (MC1), a beta actin promoter (β-ACT), a simian virus 40 promoter (SV40), and a myeloproliferative sarcoma virus enhancer, negative control region deleted, dl587rev primer-binding site substituted (MND) promoter. In some aspects, the promoter operably linked to a polynucleotide described herein is an MND promoter.

In some aspects, the disclosure provides a cell comprising a vector described herein. In some aspects, the disclosure provides a cell comprising a CAR described herein. In some aspects, the cell is an immune effector cell. In some aspects, the immune effector cell is a cytotoxic T lymphocytes (CTLs), a tumor infiltrating lymphocytes (TILs), or a helper T cell. In some aspects, the cell is a T cell. In some aspects, the cell is an αβ T cell, a γδ T cell, a natural killer (NK) cell, or a natural killer T (NKT) cell.

In some aspects, the disclosure provides a composition comprising a cell described herein. In some aspects, the disclosure provides a pharmaceutical composition comprising a cell described herein and a physiologically acceptable excipient.

In some aspects, the disclosure provides a method of generating an immune effector cell comprising a CAR described herein comprising introducing into an immune effector cell a polynucleotide or vector described herein.

In some aspects, the disclosure provides a method of treating a cancer in a subject in need thereof, comprising administering to the subject a therapeutically effective amount of a composition described herein. In some aspects, the cancer is a solid cancer, optionally wherein the solid cancer expresses MUC16. In some aspects, the solid cancer is selected from the group consisting of sarcoma, prostate cancer, uterine cancer, thyroid cancer, testicular cancer, renal cancer, pancreatic cancer, ovarian cancer, cervical, mesothelioma, esophageal cancer, lung cancer, non-small-cell lung cancer (NSCLC), small cell lung cancer (SCLC), melanoma, hepatocellular carcinoma, head and neck cancer, gastric cancer, endometrial cancer, fallopian tube cancer, colorectal cancer, cholangiocarcinoma, breast cancer, and bladder cancer. In some aspects, the solid cancer is selected from the group consisting of ovarian, endometrial cancer, cervical cancer, mesothelioma, NSCLC, and SCLC. In some aspects, the solid cancer is ovarian cancer.

In any of the foregoing or related aspects, the composition is administered in combination with an additional therapeutic agent.

In some aspects, the disclosure provides a method of treating a cancer in a subject who has received or is receiving a therapeutic agent, comprising administering to the subject a therapeutically effective amount of a composition described herein.

In any of the foregoing or related aspects, the composition and therapeutic agent are administered sequentially or simultaneously.

In any of the foregoing or related aspects, the therapeutic agent is an immune checkpoint inhibitor, oncolytic virus, or co-stimulatory antibody. In some aspects, the immune checkpoint inhibitor binds a checkpoint protein or corresponding ligand selected from the group consisting of: programmed cell death protein 1 (PD-1; PDCD1), lymphocyte activation gene 3 protein (LAG-3), T cell immunoglobulin domain and mucin domain protein 3 (TIM-3), cytotoxic T lymphocyte antigen-4 (CTLA-4), band T lymphocyte attenuator (BTLA), glucocorticoid-induced tumor necrosis factor receptor (GITR), T cell immunoglobulin and immunoreceptor tyrosine-based inhibitory motif domain (TIGIT), V-domain Ig suppressor of T cell activation (VISTA), killer cell immunoglobulin-like receptor (KIR), ICOS, ICOSL, OX40, B7-H3, B7-H4, CD47, 4-1BB, CD27, and CD70. In some aspects, the immune checkpoint inhibitor is a PD-1 inhibitor or PD-L1 inhibitor. In some aspects, the PD-1 inhibitor or PD-L1 inhibitor is an anti-PD-1 or anti-PD-L1 antibody or antigen-binding fragment thereof. In some aspects, the PD-1 inhibitor is selected from the group consisting of: nivolumab, pembrolizumab, atezolizumab, and cemiplimab.

In any of the foregoing or related aspects, the co-stimulatory antibody is a bi-specific antibody. In some aspects, the bi-specific antibody binds a tumor associated antigen (TAA) and CD3. In some aspects, the bi-specific antibody binds a TAA and CD28. In some aspects, the TAA is selected from the group consisting of AFP, ALK, BAGE proteins, β-catenin, brc-abl, BRCA1, BORIS, CA9, carbonic anhydrase IX, caspase-8, CCR5, CD40, CDK4, CEA, CTLA4, cyclin-B1, CYP1B1, ErbB3, ErbB4, ETV6-AML, Fra-1, FOLR1, GAGE proteins (e.g., GAGE-1, -2), GD2, GloboH, glypican-3, GM3, gp100, Her2, HLA/B-raf, HLA/k-ras, HLA/MAGE-A3, hTERT, LMP2, MART-1, ML-IAP, Muc1, Muc2, Muc3, Muc4, Muc5, Muc16, MUM1, NA17, NY-BR1, NY-BR62, NY-BR85, OX40, p15, p53, PAP, PAX3, PAX5, PCTA-1, PRLR, RAGE proteins, Ras, RGS5, Rho, SART-1, SART-3, Steap-1, Steap-2, survivin, TGF-β, TMPRSS2, Tn, TRP-1, TRP-2, tyrosinase, uroplakin-3, alpha folate receptor (FRα), αvβ6 integrin, B cell maturation antigen (BCMA), B7-H3 (CD276), B7-H6, carbonic anhydrase IX (CAIX), CCR1, CD16, CD19, CD20, CD22, CD30, CD33, CD37, CD38, CD44, CD44v6, CD44v7/8, CD70, CD79a, CD79b, CD123, CD133, CD135 (also known as fmc like tyrosine kinase 3; FLT3), CD138, CD171, carcinoembryonic antigen (CEA), Claudin-6 (CLDN6), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), IL-11Rα, IL-13Rα2, Kappa, cancer/testis antigen 2 (LAGE-1A), Lambda, Lewis-Y (LeY), L1 cell adhesion molecule (L1-CAM), Leukocyte immunoglobulin-like receptor subfamily B member 2 (LILRB2); melanoma antigen gene (MAGE)-A1, MAGE-A3, MAGE-A4, MAGE-A6, MAGEA10, melanoma antigen recognized by T cells 1 (MelanA or MART1), Mesothelin (MSLN), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), tumor associated glycoprotein 72 (TAG72), T-cell immunoglobulin and mucin-domain containing-3 (TIM-3), tumor endothelial marker 1 (TEM1/CD248), tumor endothelial marker 7-related (TEM7R), trophoblast glycoprotein (TPBG), NKG2D ligands, vascular endothelial growth factor receptor 2 (VEGFR2), and Wilms tumor 1 (WT-1). In some aspects, the TAA is MSLN. In some aspects, the TAA is alpha folate receptor (FRα). In some aspects, the TAA is MUC16.

In any of the foregoing or related aspects, the therapeutic agent is a vascular endothelial growth factor (VEGF) inhibitor. In one embodiment, the VEGF inhibitor is bevacizumab. In one embodiment, the therapeutic agent is a cytokine. In one embodiment, the cytokine is IL-2 or masked IL-2. In one embodiment, the therapeutic agent is an oncolytic virus. In some embodiments, the oncolytic virus is selected from the group consisting of VSV (Voyager V1), HSV, adenovirus, maraba virus, measles virus, NDV, picornavirus, reovirus, or vaccinia virus. In one embodiment, the oncolytic virus is Voyager V1.

### BRIEF DESCRIPTION OF THE FIGURES

FIG. 1 is a schematic of the MUC16 polypeptide.

FIG. 2 is a graph showing vector copy number (VCN) from healthy donor T cells transduced with each of the shown MUC16 CAR vectors.

FIG. 3 is a graph showing CAR expression on healthy donor T cells transduced with each of the shown MUC16 CAR vectors.

FIG. 4A is a graph showing IFNγ release from MUC16 CAR T cells.

FIG. 4B is a graph showing IFNγ release from MUC16 CAR T cells co-cultured in the presence of OVCAR3 cells which express MUC16.

FIG. 4C is a graph showing IFNγ release from MUC16 CAR T cells co-cultured in the presence of Jurkat cells which do not express MUC16.

FIG. 4D is a graph showing IFNγ release from MUC16 CAR T cells co-cultured in the presence of PANC-1 cells which do not express MUC16.

FIG. 5 shows IFNγ release from MUC16 CAR T cells co-cultured in the presence of antigen negative tumor cell lines (Jurkat, PANC-1, HUH7, K562, A549, and RD)

FIG. 6A shows MUC16 ectodomain expression in RD cells stabling expressing high, medium, or low levels of the MUC16 ectodomain.

FIG. 6B shows IFNγ release from MUC16 CAR T cells co-cultured with RD cells stabling expressing high, medium, or low levels of the MUC16 ectodomain.

FIG. 7A shows MUC16 nub mRNA copy number in K562 tumor cells electroporated with titrated amounts of MUC16 nub mRNA.

FIG. 7B shows IFNγ release from MUC16 CAR T cells co-cultured with K562 tumor cells expressing titrated amounts of electoroporated MUC16 nub mRNA.

FIG. 8A shows cytotoxicity of MUC16 CAR T cells co-cultured with RD cells stabling expressing high, medium, or low levels of the MUC16 ectodomain, at 10:1, 5:1, and 2.5:1 effector to T cells (E:T) ratios.

FIG. 8B shows cytotoxicity of MUC16 CAR T cells co-cultured with RD cells stabling expressing high, medium, or low levels of the MUC16 ectodomain, at a 2.5:1 effector to T cells (E:T) ratio.

FIG. 9 shows analysis of phenotypic markers via flow cytometry of MUC16 CAR T cells.

FIGS. 10A and 10B provide graphs showing production of IFNγ by anti-MUC16 CAR expressing cells cultured in the presence of MUC16-nub ectodomain (FIG. 10A) or CA125 (FIG. 10B).

FIG. 11 is a graph showing reduction of MUC16 and luciferase expressing OVCAR3.FP tumor cells, as shown by reduction in luciferase expression. 14 days after mice were inoculated with OVCAR3.FP cells, mice treated with anti-MUC16 CAR T cells showed a reduction in luciferase. Mice were re-challenged with OVCAR3.FP tumor cells 28 days after anti-MUC16 CAR T cells were administered, and tumor cells were reduced indicating persistence of anti-MUC16 CAR T cells. Controls were untransduced cells (UTD), untreated mice (OVCAR3.FP), and cells transduced with vehicle only (Vehicle).

FIGS. 12A, 12B, and 12C show anti-tumor activity of MUC16 CAR T cells transduced with the indicated anti-MUC16 CAR constructs in NGS mice inoculated with OVCAR3 tumor cells.

FIGS. 13A, 13B, and 13C show anti-tumor activity of MUC16 CAR T cells transduced with the indicated anti-MUC16 CAR constructs in an OVCAR3.FP IP tumor model.

### BRIEF DESCRIPTION OF THE SEQUENCE IDENTIFIERS

SEQ ID NOs: 1-3 set forth amino acid sequences of exemplary light chain CDRs of an anti-MUC16 scFv for an anti-MUC16 CAR contemplated herein.

SEQ ID NOs: 4-6 set forth amino acid sequences of exemplary heavy chain CDRs of an anti-MUC16 scFv for an anti-MUC16 CAR contemplated herein.

SEQ ID NO: 7 sets forth an amino acid sequence of an exemplary light chain variable region of an anti-MUC16 scFv for an anti-MUC16 CAR contemplated herein.

SEQ ID NO: 8 sets forth an amino acid sequence of an exemplary heavy chain variable region of an anti-MUC16 scFv for an anti-MUC16 CAR contemplated herein.

SEQ ID NO: 9 sets forth an amino acid sequence of an exemplary anti-MUC16 CAR lacking a signal peptide contemplated herein.

SEQ ID NO: 10 sets forth a nucleotide sequence of an exemplary anti-MUC16 CAR lacking a signal peptide contemplated herein.

SEQ ID NO: 11 sets forth an amino acid sequence of an exemplary anti-MUC16 CAR contemplated herein.

SEQ ID NO: 12 sets forth a nucleotide sequence of an exemplary anti-MUC16 CAR contemplated herein.

SEQ ID NO: 13 sets forth the amino acid sequence of human MUC16.

SEQ ID NOs: 14-25 set forth amino acid sequences of exemplary linkers suitable for use in anti-MUC16 CARs contemplated herein.

SEQ ID NOs: 26-35 set forth amino acid sequences of exemplary 2A cleaving sites.

SEQ ID NOs: 36-38 set forth amino acid sequences of exemplary cleavage sites.

SEQ ID NO: 39 sets forth the amino acid sequence of a human MUC16 fragment after proteolytic cleavage comprising a myc-myc-his tag.

SEQ ID NO: 40 sets forth the amino acid sequence of human MUC16 lacking the nub ectodomain antigen (CA125).

SEQ ID NOs: 41-43 set forth amino acid sequences of exemplary light chain CDRs of an anti-MUC16 scFv for an anti-MUC16 CAR contemplated herein.

SEQ ID NOs: 44-46 set forth amino acid sequences of exemplary heavy chain CDRs of an anti-MUC16 scFv for an anti-MUC16 CAR contemplated herein.

SEQ ID NO: 47 sets forth an amino acid sequence of an exemplary light chain variable region of an anti-MUC16 scFv for an anti-MUC16 CAR contemplated herein.

SEQ ID NO: 48 sets forth an amino acid sequence of an exemplary heavy chain variable region of an anti-MUC16 scFv for an anti-MUC16 CAR contemplated herein.

SEQ ID NO: 49 sets forth an amino acid sequence of an exemplary anti-MUC16 CAR lacking a signal peptide contemplated herein.

SEQ ID NO: 50 sets forth an amino acid sequence of an exemplary anti-MUC16 CAR including a signal peptide contemplated herein.

SEQ ID NO: 51 sets forth an amino acid sequence of an exemplary anti-MUC16 CAR lacking a signal peptide contemplated herein.

SEQ ID NO: 52 sets forth an amino acid sequence of an exemplary anti-MUC16 CAR including a signal peptide contemplated herein.

SEQ ID NOs: 53-55 set forth amino acid sequences of exemplary light chain CDRs of an anti-MUC16 scFv for an anti-MUC16 CAR contemplated herein.

SEQ ID NOs: 56-58 set forth amino acid sequences of exemplary heavy chain CDRs of an anti-MUC16 scFv for an anti-MUC16 CAR contemplated herein.

SEQ ID NO: 59 sets forth an amino acid sequence of an exemplary light chain variable region of an anti-MUC16 scFv for an anti-MUC16 CAR contemplated herein.

SEQ ID NO: 60 sets forth an amino acid sequence of an exemplary heavy chain variable region of an anti-MUC16 scFv for an anti-MUC16 CAR contemplated herein.

SEQ ID NO: 61 sets forth an amino acid sequence of an exemplary anti-MUC16 CAR lacking a signal peptide contemplated herein.

SEQ ID NO: 62 sets forth an amino acid sequence of an exemplary anti-MUC16 CAR including a signal peptide contemplated herein.

SEQ ID NO: 63 sets forth an amino acid sequence of an exemplary anti-MUC16 CAR lacking a signal peptide contemplated herein.

SEQ ID NO: 64 sets forth an amino acid sequence of an exemplary anti-MUC16 CAR including a signal peptide contemplated herein.

SEQ ID NOs: 65-67 set forth amino acid sequences of exemplary light chain CDRs of an anti-MUC16 scFv for an anti-MUC16 CAR contemplated herein.

SEQ ID NOs: 68-70 set forth amino acid sequences of exemplary heavy chain CDRs of an anti-MUC16 scFv for an anti-MUC16 CAR contemplated herein.

SEQ ID NO: 71 sets forth an amino acid sequence of an exemplary light chain variable region of an anti-MUC16 scFv for an anti-MUC16 CAR contemplated herein.

SEQ ID NO: 72 sets forth an amino acid sequence of an exemplary heavy chain variable region of an anti-MUC16 scFv for an anti-MUC16 CAR contemplated herein.

SEQ ID NO: 73 sets forth an amino acid sequence of an exemplary anti-MUC16 CAR lacking a signal peptide contemplated herein.

SEQ ID NO: 74 sets forth an amino acid sequence of an exemplary anti-MUC16 CAR including a signal peptide contemplated herein.

SEQ ID NO: 75 sets forth an amino acid sequence of an exemplary anti-MUC16 CAR lacking a signal peptide contemplated herein.

SEQ ID NO: 76 sets forth an amino acid sequence of an exemplary anti-MUC16 CAR including a signal peptide contemplated herein.

SEQ ID NOs: 77-79 set forth amino acid sequences of exemplary light chain CDRs of an anti-MUC16 scFv for an anti-MUC16 CAR contemplated herein.

SEQ ID NOs: 80-82 set forth amino acid sequences of exemplary heavy chain CDRs of an anti-MUC16 scFv for an anti-MUC16 CAR contemplated herein.

SEQ ID NO: 83 sets forth an amino acid sequence of an exemplary light chain variable region of an anti-MUC16 scFv for an anti-MUC16 CAR contemplated herein.

SEQ ID NO: 84 sets forth an amino acid sequence of an exemplary heavy chain variable region of an anti-MUC16 scFv for an anti-MUC16 CAR contemplated herein.

SEQ ID NO: 85 sets forth an amino acid sequence of an exemplary anti-MUC16 CAR lacking a signal peptide contemplated herein.

SEQ ID NO: 86 sets forth an amino acid sequence of an exemplary anti-MUC16 CAR including a signal peptide contemplated herein.

SEQ ID NO: 87 sets forth an amino acid sequence of an exemplary anti-MUC16 CAR lacking a signal peptide contemplated herein.

SEQ ID NO: 88 sets forth an amino acid sequence of an exemplary anti-MUC16 CAR including a signal peptide contemplated herein.

SEQ ID NOs: 89-91 set forth amino acid sequences of exemplary light chain CDRs of an anti-MUC16 scFv for an anti-MUC16 CAR contemplated herein.

SEQ ID NOs: 92-94 set forth amino acid sequences of exemplary heavy chain CDRs of an anti-MUC16 scFv for an anti-MUC16 CAR contemplated herein.

SEQ ID NO: 95 sets forth an amino acid sequence of an exemplary light chain variable region of an anti-MUC16 scFv for an anti-MUC16 CAR contemplated herein.

SEQ ID NO: 96 sets forth an amino acid sequence of an exemplary heavy chain variable region of an anti-MUC16 scFv for an anti-MUC16 CAR contemplated herein.

SEQ ID NO: 97 sets forth an amino acid sequence of an exemplary anti-MUC16 CAR lacking a signal peptide contemplated herein.

SEQ ID NO: 98 sets forth an amino acid sequence of an exemplary anti-MUC16 CAR including a signal peptide contemplated herein.

SEQ ID NO: 99 sets forth an amino acid sequence of an exemplary anti-MUC16 CAR lacking a signal peptide contemplated herein.

SEQ ID NO: 100 sets forth an amino acid sequence of an exemplary anti-MUC16 CAR including a signal peptide contemplated herein.

SEQ ID NOs: 101-103 set forth amino acid sequences of exemplary light chain CDRs of an anti-MUC16 scFv for an anti-MUC16 CAR contemplated herein.

SEQ ID NOs: 104-106 set forth amino acid sequences of exemplary heavy chain CDRs of an anti-MUC16 scFv for an anti-MUC16 CAR contemplated herein.

SEQ ID NO: 107 sets forth an amino acid sequence of an exemplary light chain variable region of an anti-MUC16 scFv for an anti-MUC16 CAR contemplated herein.

SEQ ID NO: 108 sets forth an amino acid sequence of an exemplary heavy chain variable region of an anti-MUC16 scFv for an anti-MUC16 CAR contemplated herein.

SEQ ID NO: 109 sets forth an amino acid sequence of an exemplary anti-MUC16 CAR lacking a signal peptide contemplated herein.

SEQ ID NO: 110 sets forth an amino acid sequence of an exemplary anti-MUC16 CAR including a signal peptide contemplated herein.

SEQ ID NO: 111 sets forth an amino acid sequence of an exemplary anti-MUC16 CAR lacking a signal peptide contemplated herein.

SEQ ID NO: 112 sets forth an amino acid sequence of an exemplary anti-MUC16 CAR including a signal peptide contemplated herein.

SEQ ID NOs: 113-115 set forth amino acid sequences of exemplary light chain CDRs of an anti-MUC16 scFv for an anti-MUC16 CAR contemplated herein.

SEQ ID NOs: 116-118 set forth amino acid sequences of exemplary heavy chain CDRs of an anti-MUC16 scFv for an anti-MUC16 CAR contemplated herein.

SEQ ID NO: 119 sets forth an amino acid sequence of an exemplary light chain variable region of an anti-MUC16 scFv for an anti-MUC16 CAR contemplated herein.

SEQ ID NO: 120 sets forth an amino acid sequence of an exemplary heavy chain variable region of an anti-MUC16 scFv for an anti-MUC16 CAR contemplated herein.

SEQ ID NO: 121 sets forth an amino acid sequence of an exemplary anti-MUC16 CAR lacking a signal peptide contemplated herein.

SEQ ID NO: 122 sets forth an amino acid sequence of an exemplary anti-MUC16 CAR including a signal peptide contemplated herein.

SEQ ID NO: 123 sets forth an amino acid sequence of an exemplary anti-MUC16 CAR lacking a signal peptide contemplated herein.

SEQ ID NO: 124 sets forth an amino acid sequence of an exemplary anti-MUC16 CAR including a signal peptide contemplated herein.

SEQ ID NO: 125 sets forth an amino acid sequence of an exemplary anti-MUC16 CAR lacking a signal peptide contemplated herein.

SEQ ID NO: 126 sets forth an amino acid sequence of an exemplary anti-MUC16 CAR including a signal peptide contemplated herein.

SEQ ID NOs: 127-129 set forth amino acid sequences of exemplary light chain CDRs of an anti-MUC16 scFv for an anti-MUC16 CAR contemplated herein.

SEQ ID NOs: 130-132 set forth amino acid sequences of exemplary heavy chain CDRs of an anti-MUC16 scFv for an anti-MUC16 CAR contemplated herein.

SEQ ID NO: 133 sets forth an amino acid sequence of an exemplary light chain variable region of an anti-MUC16 scFv for an anti-MUC16 CAR contemplated herein.

SEQ ID NO: 134 sets forth an amino acid sequence of an exemplary heavy chain variable region of an anti-MUC16 scFv for an anti-MUC16 CAR contemplated herein.

SEQ ID NO: 135 sets forth an amino acid sequence of an exemplary anti-MUC16 CAR lacking a signal peptide contemplated herein.

SEQ ID NO: 136 sets forth an amino acid sequence of an exemplary anti-MUC16 CAR including a signal peptide contemplated herein.

SEQ ID NO: 137 sets forth an amino acid sequence of an exemplary anti-MUC16 CAR lacking a signal peptide contemplated herein.

SEQ ID NO: 138 sets forth an amino acid sequence of an exemplary anti-MUC16 CAR including a signal peptide contemplated herein.

SEQ ID NOs: 139-141 set forth amino acid sequences of exemplary light chain CDRs of an anti-MUC16 scFv for an anti-MUC16 CAR contemplated herein.

SEQ ID NOs: 142-144 set forth amino acid sequences of exemplary heavy chain CDRs of an anti-MUC16 scFv for an anti-MUC16 CAR contemplated herein.

SEQ ID NO: 145 sets forth an amino acid sequence of an exemplary light chain variable region of an anti-MUC16 scFv for an anti-MUC16 CAR contemplated herein.

SEQ ID NO: 146 sets forth an amino acid sequence of an exemplary heavy chain variable region of an anti-MUC16 scFv for an anti-MUC16 CAR contemplated herein.

SEQ ID NO: 147 sets forth an amino acid sequence of an exemplary anti-MUC16 CAR lacking a signal peptide contemplated herein.

SEQ ID NO: 148 sets forth an amino acid sequence of an exemplary anti-MUC16 CAR including a signal peptide contemplated herein.

SEQ ID NO: 149 sets forth an amino acid sequence of an exemplary anti-MUC16 CAR lacking a signal peptide contemplated herein.

SEQ ID NO: 150 sets forth an amino acid sequence of an exemplary anti-MUC16 CAR including a signal peptide contemplated herein.

SEQ ID NO: 151 sets forth the amino acid sequence of a human MUC16 fragment after proteolytic cleavage lacking a myc-myc-his tag.

SEQ ID NO: 152 sets forth the amino acid sequence of an exemplary signal peptide contemplated herein.

SEQ ID NO: 153 sets forth the amino acid sequence of an exemplary hinge region peptide contemplated herein.

SEQ ID NO: 154 sets forth the amino acid sequence of an exemplary transmembrane region peptide contemplated herein.

SEQ ID NO: 155 sets forth the amino acid sequence of an exemplary co-stimulatory peptide contemplated herein.

SEQ ID NO: 156 sets forth the amino acid sequence of an exemplary signaling region peptide contemplated herein.

### DETAILED DESCRIPTION

### A. Overview

In some embodiments, the present disclosure provides chimeric antigen receptors (CARs) comprising a binding domain for MUC16. MUC16, also known as cancer antigen 125, carcinoma antigen 125, carbohydrate antigen 125, or CA-125, is a highly glycosylated integral membrane glycoprotein. MUC16 is overexpressed in cancers, including ovarian cancer, breast cancer, pancreatic cancer, non-small-cell lung cancer, intrahepatic cholangiocarcinoma-mass forming type, adenocarcinoma of the uterine cervix, and adenocarcinoma of the gastric tract, and in diseases and conditions including inflammatory bowel disease, liver cirrhosis, cardiac failure, peritoneal infection, and abdominal surgery. (Haridas, D. et al., 2014, FASEB J., 28:4183-4199), and expression of MUC16 on cancer cells has been shown to protect the cancer cells from the immune system (Felder, M. et al., 2014, Molecular Cancer, 13:129). In some embodiments, immune effector cells expressing CARs comprising a binding domain for MUC16 surprisingly show increased immune response and cytotoxicity compared to untransduced effector cells when co-cultured with cancer cells.

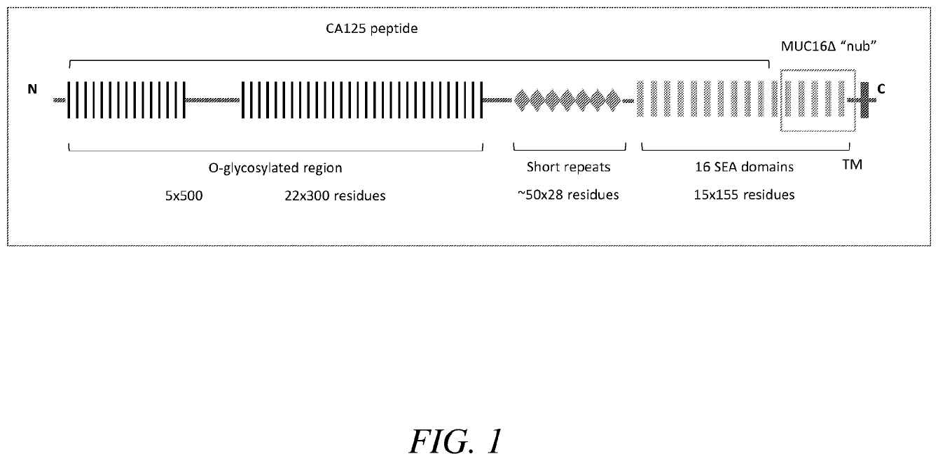

MUC16 comprises a single transmembrane domain consisting of three major domains: an extracellular N-terminal domain, a large tandem repeat domain interspersed with sea urchin sperm, enterokinase, and agrin (SEA) domains, and a carboxyl terminal domain that comprises a segment of the transmembrane region and a short cytoplasmic tail. FIG. 1 provides a schematic showing the domains of MUC16. Upon proteolytic cleavage of MUC16, the majority of the extracellular portion is shed into the blood stream. A fragment of MUC16 remains on the surface of the cell. In some embodiments, MUC16 comprises 16 SEA domains at the C-terminal end of the polypeptide, numbered 1 to 16 from the N-terminus to C-terminus. In some embodiments, the MUC16 fragment remaining on the surface of the cell comprises SEA domains 12-16. In some embodiments, the CARs described herein selectively bind SEA domains 12-16 of MUC16. In some embodiments, the CARs described herein do not bind SEA domains 1-11 of MUC16.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniquesfor the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid The Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and CC Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

In some embodiments, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant to include any elements listed after the phrase and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

### C. Chimeric Antigen Receptors

In some embodiments, improved genetically engineered receptors that redirect cytotoxicity of immune effector cells toward MUC16 expressing cells are provided. These genetically engineered receptors are referred to herein as chimeric antigen receptors (CARs). CARs are molecules that combine antibody-based specificity for a desired antigen (e.g., MUC16) with a T cell receptor-activating intracellular domain to generate a chimeric protein that exhibits a specific anti-MUC16 cellular immune activity. As used herein, the term, “chimeric,” describes being composed of parts of different proteins or DNAs from different origins.

In some embodiments, CARs contemplated herein comprise an extracellular domain (also referred to as a binding domain or antigen-specific binding domain) that binds to MUC16, a transmembrane domain, and an intracellular signaling domain. Engagement of the anti-MUC16 antigen binding domain of the CAR with MUC16 on the surface of a target cell results in clustering of the CAR and delivers an activation stimulus to the CAR-containing cell. The main characteristic of CARs is their ability to redirect immune effector cell specificity, thereby triggering proliferation, cytokine production, phagocytosis or production of molecules that can mediate cell death of the target antigen expressing cell in a major histocompatibility (MHC) independent manner, exploiting the cell specific targeting abilities of monoclonal antibodies, soluble ligands or cell specific co-receptors.

In some embodiments, a CAR comprises an extracellular binding domain that comprises a human anti-MUC16-specific binding domain; a transmembrane domain; one or more intracellular co-stimulatory signaling domains; and a primary signaling domain.

In some embodiments, a CAR comprises an extracellular binding domain that comprises a human anti-MUC16 antibody or antigen binding fragment thereof; one or more hinge domains or spacer domains; a transmembrane domain; one or more intracellular co-stimulatory signaling domains; and a primary signaling domain.

### 1. Binding Domain

In some embodiments, CARs contemplated herein comprise an extracellular binding domain that comprises an anti-MUC16 antibody or antigen binding fragment thereof that specifically binds to a human MUC16 polypeptide expressed on a target cell (e.g., cancer cell). The term “MUC16,” as used herein, refers to a human MUC16 protein, unless specified as being from a non-human species (e.g., “mouse MUC16,” “monkey MUC16,” etc.). MUC16 comprises three major domains: an extracellular N-terminal domain, a large tandem repeat domain interspersed with sea urchin sperm, enterokinase, agrin (SEA) domains and a carboxyl terminal domain that comprises a segment of the transmembrane region and a short cytoplasmic tail. The human MUC16 protein has the amino acid sequence shown in SEQ ID NO:13, and/or having the amino acid sequence as set forth in NCBI accession No. NP_078966. The human MUC16 membrane proximal domain (P13810-P14451) (MUC16 “nub”) having a myc-myc-his tag is shown as SEQ ID NO: 39. The human MUC16 membrane proximal domain (P13810-P14451) (MUC16 “nub”) lacking a myc-myc-his tag is shown as SEQ ID NO: 151. The term “CA125,” as used herein, refers to the human MUC16 polypeptide lacking the MUC16 membrane proximal domain (MUC16 “nub”) as shown as SEQ ID NO: 40.

The terms “MUC16-nub”, “nub”, “MUC16 ectodomain”, “MUC16-nub ectodomain”, and “ectodomain” as used herein are interchangeable and refer to a membrane proximal MUC16 polypeptide domain or fragment remaining on the cell surface after cleavage. In some embodiments, the MUC16-nub ectodomain comprises SEA domains 12-16.

As used herein, the terms, “binding domain,” “extracellular domain,” “extracellular binding domain,” “antigen-specific binding domain,” and “extracellular antigen specific binding domain,” are used interchangeably and provide a CAR with the ability to specifically bind to the target antigen of interest, e.g., MUC16. The binding domain may be derived either from a natural, synthetic, semi-synthetic, or recombinant source.

The terms “specific binding affinity” or “specifically binds” or “specifically bound” or “specific binding” or “specifically targets” as used herein, describe binding of an anti-MUC16 antibody or antigen binding fragment thereof (or a CAR comprising the same) to MUC16 at greater binding affinity than background binding. A binding domain (or a CAR comprising a binding domain or a fusion protein containing a binding domain) “specifically binds” to a MUC16 if it binds to or associates with MUC16 with an affinity or Kₐ (i.e., an equilibrium association constant of a particular binding interaction with units of 1/M) of, for example, greater than or equal to about 10⁵ M⁻¹. In certain embodiments, a binding domain (or a fusion protein thereof) binds to a target with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10³ M⁻¹. “High affinity” binding domains (or single chain fusion proteins thereof) refers to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater.

Alternatively, affinity may be defined as an equilibrium dissociation constant (K_{d}) of a particular binding interaction with units of M (e.g., 10⁻⁵ M to 10⁻¹³ μM, or less). Affinities of binding domain polypeptides and CAR proteins according to the present disclosure can be readily determined using conventional techniques, e.g., by competitive ELISA (enzyme-linked immunosorbent assay), or by binding association, or displacement assays using labeled ligands, or using a surface-plasmon resonance device such as the Biacore T100, which is available from Biacore, Inc., Piscataway, NJ, or optical biosensor technology such as the EPIC system or EnSpire that are available from Corning and Perkin Elmer respectively (see also, e.g., Scatchard et al. (1949) Ann. N.Y. Acad. Sci. 51:660; and U.S. Pat. Nos. 5,283,173; 5,468,614, or the equivalent).

In some embodiments, the affinity of specific binding is about 2 times greater than background binding, about 5 times greater than background binding, about 10 times greater than background binding, about 20 times greater than background binding, about 50 times greater than background binding, about 100 times greater than background binding, or about 1000 times greater than background binding or more.

In some embodiments, the extracellular binding domain of a CAR comprises an antibody or antigen binding fragment thereof. An “antibody” refers to a binding agent that is a polypeptide comprising at least a light chain or heavy chain immunoglobulin variable region which specifically recognizes and binds an epitope of an antigen, such as a peptide, lipid, polysaccharide, or nucleic acid containing an antigenic determinant, such as those recognized by an immune cell.

An “antigen (Ag)” refers to a compound, composition, or substance that can stimulate the production of antibodies or a T cell response in an animal, including compositions (such as one that includes a cancer-specific protein) that are injected or absorbed into an animal. An antigen reacts with the products of specific humoral or cellular immunity, including those induced by heterologous antigens, such as the disclosed antigens. In some embodiments, the target antigen is an epitope of a MUC16 polypeptide.

An “epitope” or “antigenic determinant” refers to the region of an antigen to which a binding agent binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation.

Antibodies include antigen binding fragments thereof, such as Camel Ig, Ig NAR, Fab fragments, Fab′ fragments, F(ab)′2 fragments, F(ab)′3 fragments, Fv, single chain Fv proteins (“scFv”), bis-scFv, (scFv)2, minibodies, diabodies, triabodies, tetrabodies, disulfide stabilized Fv proteins (“dsFv”), and single-domain antibody (sdAb, Nanobody) and portions of full length antibodies responsible for antigen binding. The term also includes genetically engineered forms such as chimeric antibodies (for example, humanized murine antibodies), heteroconjugate antibodies (such as, bispecific antibodies) and antigen binding fragments thereof. See also, Pierce Catalog and Handbook, 1994-1995 (Pierce Chemical Co., Rockford, IL); Kuby, J., Immunology, 3rd Ed., W. H. Freeman & Co., New York, 1997.

As used herein, the expression “bispecific antibody” means an antibody comprising at least a first antigen-binding domain and a second antigen-binding domain. Each antigen-binding domain within the bispecific antibody comprises at least one CDR that alone, or in combination with one or more additional CDRs and/or FRs, specifically binds to a particular antigen.

As would be understood by the skilled person and as described elsewhere herein, a complete antibody comprises two heavy chains and two light chains. Each heavy chain consists of a variable region and a first, second, and third constant region, while each light chain consists of a variable region and a constant region. Mammalian heavy chains are classified as α, δ, ε, γ, and μ. Mammalian light chains are classified as λ or κ. Immunoglobulins comprising the α, δ, ε, γ, and μ heavy chains are classified as immunoglobulin (Ig)A, IgD, IgE, IgG, and IgM. The complete antibody forms a “Y” shape. The stem of the Y consists of the second and third constant regions (and for IgE and IgM, the fourth constant region) of two heavy chains bound together and disulfide bonds (inter-chain) are formed in the hinge. Heavy chains γ, α and δ have a constant region composed of three tandem (in a line) Ig domains, and a hinge region for added flexibility; heavy chains μ and ε have a constant region composed of four immunoglobulin domains. The second and third constant regions are referred to as “CH2 domain” and “CH3 domain”, respectively. Each arm of the Y includes the variable region and first constant region of a single heavy chain bound to the variable and constant regions of a single light chain. The variable regions of the light and heavy chains are responsible for antigen binding.

Light and heavy chain variable regions contain a “framework” region (FR) interrupted by three hypervariable regions, also called “complementarity-determining regions” or “CDRs.” The CDRs can be defined or identified by conventional methods, such as by sequence according to Kabat et al (Wu, T T and Kabat, E. A., J Exp Med. 132(2):211-50, (1970); Borden, P. and Kabat E. A., PNAS, 84: 2440-2443 (1987); (see, Kabat et al., Sequences of Proteins of Immunological Interest, U.S. Department of Health and Human Services, 1991, which is hereby incorporated by reference), or by structure according to Chothia et al (Chothia, C. and Lesk, A. M., J Mol. Biol., 196(4): 901-917 (1987), Chothia, C. et al, Nature, 342: 877-883 (1989)).

The sequences of the framework regions of different light or heavy chains are relatively conserved within a species, such as humans. The framework region of an antibody, that is the combined framework regions of the constituent light and heavy chains, serves to position and align the CDRs in three-dimensional space. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, the CDRs located in the variable domain of the heavy chain of the antibody are referred to as CDRH1, CDRH2, and CDRH3, whereas the CDRs located in the variable domain of the light chain of the antibody are referred to as CDRL1, CDRL2, and CDRL3. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). Illustrative examples of light chain CDRs that are suitable for constructing anti-MUC16 CARs contemplated herein include, but are not limited to the CDR sequences set forth in SEQ ID NOs: 1-3. Illustrative examples of heavy chain CDRs that are suitable for constructing anti-MUC16 CARs contemplated herein include, but are not limited to the CDR sequences set forth in SEQ ID NOs: 4-6.

Illustrative examples of rules for predicting light chain CDRs include: CDR-L1 starts at about residue 24, is preceded by a Cys, is about 10-17 residues, and is followed by a Trp (typically Trp-Tyr-Gln, but also, Trp-Leu-Gln, Trp-Phe-Gln, Trp-Tyr-Leu); CDR-L2 starts about 16 residues after the end of CDR-L1, is generally preceded by Ile-Tyr, but also, Val-Tyr, Ile-Lys, Ile-Phe, and is 7 residues; and CDR-L3 starts about 33 residues after the end of CDR-L2, is preceded by a Cys, is 7-11 residues, and is followed by Phe-Gly-XXX-Gly (XXX is any amino acid).

Illustrative examples of rules for predicting heavy chain CDRs include: CDR-H1 starts at about residue 26, is preceded by Cys-XXX-XXX-XXX, is 10-12 residues and is followed by a Trp (typically Trp-Val, but also, Trp-Ile, Trp-Ala); CDR-H2 starts about 15 residues after the end of CDR-H1, is generally preceded by Leu-Glu-Trp-Ile-Gly, or a number of variations, is 16-19 residues, and is followed by Lys/Arg-Leu/Ile/Val/Phe/Thr/Ala-Thr/Ser/Ile/Ala; and CDR-H3 starts about 33 residues after the end of CDR-H2, is preceded by Cys-XXX-XXX (typically Cys-Ala-Arg), is 3 to 25 residues, and is followed by Trp-Gly-XXX-Gly.

In some embodiments, light chain CDRs and the heavy chain CDRs are determined according to the Kabat method. In some embodiments, light chain CDRs and the heavy chain CDR2 and CDR3 are determined according to the Kabat method, and heavy chain CDR1 is determined according to the AbM method, which is a comprise between the Kabat and Clothia methods, see e.g., Whitelegg N & Rees A R, Protein Eng. 2000 December; 13(12):819-24 and Methods Mol Biol. 2004; 248:51-91. Programs for predicting CDRs are publicly available, e.g., AbYsis (www.bioinf.org.uk/abysis/).

References to “V_{H}” or “VH” refer to the variable region of an immunoglobulin heavy chain, including that of an antibody, Fv, scFv, dsFv, Fab, or other antibody fragment as disclosed herein. References to “V_{L}” or “VL” refer to the variable region of an immunoglobulin light chain, including that of an antibody, Fv, scFv, dsFv, Fab, or other antibody fragment as disclosed herein.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” has framework residues from one species, such as human, and CDRs (which generally confer antigen binding) from another species, such as a mouse. In particular preferred embodiments, a CAR contemplated herein comprises antigen-specific binding domain that is a chimeric antibody or antigen binding fragment thereof.

A “humanized” antibody is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.”

In particular embodiments, an anti-MUC16 antibody or antigen binding fragment thereof, includes but is not limited to a Camel Ig (a camelid antibody (VHH)), Ig NAR, Fab fragments, Fab′ fragments, F(ab)′2 fragments, F(ab)′3 fragments, Fv, single chain Fv antibody (“scFv”), bis-scFv, (scFv)2, minibody, diabody, triabody, tetrabody, disulfide stabilized Fv protein (“dsFv”), and single-domain antibody (sdAb, Nanobody).

“Camel Ig” or “camelid VHH” as used herein refers to the smallest known antigen-binding unit of a heavy chain antibody (Koch-Nolte, et al, FASEB J., 21: 3490-3498 (2007)). A “heavy chain antibody” or a “camelid antibody” refers to an antibody that contains two VH domains and no light chains (Riechmann L. et al, J. Immunol. Methods 231:25-38 (1999); WO94/04678; WO94/25591; U.S. Pat. No. 6,005,079).

“IgNAR” of “immunoglobulin new antigen receptor” refers to class of antibodies from the shark immune repertoire that consist of homodimers of one variable new antigen receptor (VNAR) domain and five constant new antigen receptor (CNAR) domains. IgNARs represent some of the smallest known immunoglobulin-based protein scaffolds and are highly stable and possess efficient binding characteristics. The inherent stability can be attributed to both (i) the underlying Ig scaffold, which presents a considerable number of charged and hydrophilic surface exposed residues compared to the conventional antibody VH and VL domains found in murine antibodies; and (ii) stabilizing structural features in the complementary determining region (CDR) loops including inter-loop disulphide bridges, and patterns of intra-loop hydrogen bonds.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv (scFv)2 species. It is in this configuration that the three hypervariable regions (HVRs) of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al, PNAS USA 90: 6444-6448 (1993). Triabodies and tetrabodies are also described in Hudson et al., Nat. Med. 9:129-134 (2003).

“Single domain antibody” or “sdAb” or “nanobody” refers to an antibody fragment that consists of the variable region of an antibody heavy chain (VH domain) or the variable region of an antibody light chain (VL domain) (Holt, L., et al, Trends in Biotechnology, 21(11): 484-490).

“Single-chain Fv” or “scFv” antibody fragments comprise the VH and VL domains of antibody, wherein these domains are present in a single polypeptide chain and in either orientation (e.g., VL-VH or VH-VL). Generally, the scFv polypeptide further comprises a polypeptide linker between the VH and VL domains which enables the scFv to form the desired structure for antigen binding. For a review of scFv, see, e.g., Pluckthun, in The Pharmacology of Monoclonal Antibodies, vol. 113, Rosenburg and Moore eds., (Springer-Verlag, New York, 1994), pp. 269-315.

In some embodiments, the antigen-specific binding domain is a scFv. Single chain antibodies may be cloned form the V region genes of a hybridoma specific for a desired target. The production of such hybridomas has become routine. A technique which can be used for cloning the variable region heavy chain (V_{H}) and variable region light chain (V_{L}) has been described, for example, in Orlandi et al., PNAS, 1989; 86: 3833-3837.

In some embodiments, the antigen-specific binding domain is a scFv that binds a human MUC16 polypeptide. An illustrative example of a variable heavy chain that is suitable for constructing anti-MUC16 CARs contemplated herein is the amino acid sequence set forth in SEQ ID NO: 8. An illustrative example of a variable light chain that is suitable for constructing anti-MUC16 CARs contemplated herein is the amino acid sequence set forth in SEQ ID NO: 7.

In some embodiments, the antigen-specific binding domain of an anti-MUC16 CAR comprises a variable heavy chain comprising the amino acid sequence of SEQ ID NO: 8. In some embodiments, the antigen-specific binding domain of an anti-MUC16 CAR comprises a variable heavy chain comprising an amino acid sequence having at least 90%, 91%, 92%, 93%, 94%, 95%, 96%, 97%, 98%, or 99% identity to the amino acid sequence of SEQ ID NO: 8. In some embodiments, the antigen-specific binding domain of an anti-MUC16 CAR comprises a variable light chain comprising the amino acid sequence of SEQ ID NO: 7. In some embodiments, the antigen-specific binding domain of an anti-MUC16 CAR comprises a variable light chain comprising an amino acid sequence having at least 90%, 91%, 92%, 93%, 94%, 95%, 96%, 97%, 98%, or 99% identity to the amino acid sequence of SEQ ID NO: 7. In some embodiments, the antigen-specific binding domain of an anti-MUC16 CAR comprises a variable heavy chain comprising the amino acid sequence of SEQ ID NO: 8 and a variable light chain comprising the amino acid sequence of SEQ ID NO: 7.

In some embodiments, the antigen-specific binding domain of an anti-MUC16 CAR comprises a variable heavy chain comprising the amino acid sequence of SEQ ID NO:48. In some embodiments, the antigen-specific binding domain of an anti-MUC16 CAR comprises a variable heavy chain comprising an amino acid sequence having at least 90%, 91%, 92%, 93%, 94%, 95%, 96%, 97%, 98%, or 99% identity to the amino acid sequence of SEQ ID NO: 48.In some embodiments, the antigen-specific binding domain of an anti-MUC16 CAR comprises a variable light chain comprising the amino acid sequence of SEQ ID NO: 47. In some embodiments, the antigen-specific binding domain of an anti-MUC16 CAR comprises a variable light chain comprising an amino acid sequence having at least 90%, 91%, 92%, 93%, 94%, 95%, 96%, 97%, 98%, or 99% identity to the amino acid sequence of SEQ ID NO: 47. In some embodiments, the antigen-specific binding domain of an anti-MUC16 CAR comprises a variable heavy chain comprising the amino acid sequence of SEQ ID NO: 48 and a variable light chain comprising the amino acid sequence of SEQ ID NO: 47.

In some embodiments, the antigen-specific binding domain of an anti-MUC16 CAR comprises a variable heavy chain comprising the amino acid sequence of SEQ ID NO: 60. In some embodiments, the antigen-specific binding domain of an anti-MUC16 CAR comprises a variable heavy chain comprising an amino acid sequence having at least 90%, 91%, 92%, 93%, 94%, 95%, 96%, 97%, 98%, or 99% identity to the amino acid sequence of SEQ ID NO: 60. In some embodiments, the antigen-specific binding domain of an anti-MUC16 CAR comprises a variable light chain comprising the amino acid sequence of SEQ ID NO: 59. In some embodiments, the antigen-specific binding domain of an anti-MUC16 CAR comprises a variable light chain comprising an amino acid sequence having at least 90%, 91%, 92%, 93%, 94%, 95%, 96%, 97%, 98%, or 99% identity to the amino acid sequence of SEQ ID NO: 59. In some embodiments, the antigen-specific binding domain of an anti-MUC16 CAR comprises a variable heavy chain comprising the amino acid sequence of SEQ ID NO: 60 and a variable light chain comprising the amino acid sequence of SEQ ID NO: 59.

In some embodiments, the antigen-specific binding domain of an anti-MUC16 CAR comprises a variable heavy chain comprising the amino acid sequence of SEQ ID NO: 72. In some embodiments, the antigen-specific binding domain of an anti-MUC16 CAR comprises a variable heavy chain comprising an amino acid sequence having at least 90%, 91%, 92%, 93%, 94%, 95%, 96%, 97%, 98%, or 99% identity to the amino acid sequence of SEQ ID NO: 72. In some embodiments, the antigen-specific binding domain of an anti-MUC16 CAR comprises a variable light chain comprising the amino acid sequence of SEQ ID NO: 71. In some embodiments, the antigen-specific binding domain of an anti-MUC16 CAR comprises a variable light chain comprising an amino acid sequence having at least 90%, 91%, 92%, 93%, 94%, 95%, 96%, 97%, 98%, or 99% identity to the amino acid sequence of SEQ ID NO: 71. In some embodiments, the antigen-specific binding domain of an anti-MUC16 CAR comprises a variable heavy chain comprising the amino acid sequence of SEQ ID NO: 72 and a variable light chain comprising the amino acid sequence of SEQ ID NO: 71.

In some embodiments, the antigen-specific binding domain of an anti-MUC16 CAR comprises a variable heavy chain comprising the amino acid sequence of SEQ ID NO: 84.

In some embodiments, the antigen-specific binding domain of an anti-MUC16 CAR comprises a variable heavy chain comprising an amino acid sequence having at least 90%, 91%, 92%, 93%, 94%, 95%, 96%, 97%, 98%, or 99% identity to the amino acid sequence of SEQ ID NO: 84. In some embodiments, the antigen-specific binding domain of an anti-MUC16 CAR comprises a variable light chain comprising the amino acid sequence of SEQ ID NO: 83. In some embodiments, the antigen-specific binding domain of an anti-MUC16 CAR comprises a variable light chain comprising an amino acid sequence having at least 90%, 91%, 92%, 93%, 94%, 95%, 96%, 97%, 98%, or 99% identity to the amino acid sequence of SEQ ID NO: 83. In some embodiments, the antigen-specific binding domain of an anti-MUC16 CAR comprises a variable heavy chain comprising the amino acid sequence of SEQ ID NO: 84 and a variable light chain comprising the amino acid sequence of SEQ ID NO: 83.

In some embodiments, the antigen-specific binding domain of an anti-MUC16 CAR comprises a variable heavy chain comprising the amino acid sequence of SEQ ID NO: 96. In some embodiments, the antigen-specific binding domain of an anti-MUC16 CAR comprises a variable heavy chain comprising an amino acid sequence having at least 90%, 91%, 92%, 93%, 94%, 95%, 96%, 97%, 98%, or 99% identity to the amino acid sequence of SEQ ID NO: 96. In some embodiments, the antigen-specific binding domain of an anti-MUC16 CAR comprises a variable light chain comprising the amino acid sequence of SEQ ID NO: 95. In some embodiments, the antigen-specific binding domain of an anti-MUC16 CAR comprises a variable light chain comprising an amino acid sequence having at least 90%, 91%, 92%, 93%, 94%, 95%, 96%, 97%, 98%, or 99% identity to the amino acid sequence of SEQ ID NO: 95. In some embodiments, the antigen-specific binding domain of an anti-MUC16 CAR comprises a variable heavy chain comprising the amino acid sequence of SEQ ID NO: 96 and a variable light chain comprising the amino acid sequence of SEQ ID NO: 95.

In some embodiments, the antigen-specific binding domain of an anti-MUC16 CAR comprises a variable heavy chain comprising the amino acid sequence of SEQ ID NO: 108. In some embodiments, the antigen-specific binding domain of an anti-MUC16 CAR comprises a variable heavy chain comprising an amino acid sequence having at least 90%, 91%, 92%, 93%, 94%, 95%, 96%, 97%, 98%, or 99% identity to the amino acid sequence of SEQ ID NO: 108. In some embodiments, the antigen-specific binding domain of an anti-MUC16 CAR comprises a variable light chain comprising the amino acid sequence of SEQ ID NO: 107. In some embodiments, the antigen-specific binding domain of an anti-MUC16 CAR comprises a variable light chain comprising an amino acid sequence having at least 90%, 91%, 92%, 93%, 94%, 95%, 96%, 97%, 98%, or 99% identity to the amino acid sequence of SEQ ID NO: 107. In some embodiments, the antigen-specific binding domain of an anti-MUC16 CAR comprises a variable heavy chain comprising the amino acid sequence of SEQ ID NO: 108 and a variable light chain comprising the amino acid sequence of SEQ ID NO: 107.

In some embodiments, the antigen-specific binding domain of an anti-MUC16 CAR comprises a variable heavy chain comprising the amino acid sequence of SEQ ID NO: 120. In some embodiments, the antigen-specific binding domain of an anti-MUC16 CAR comprises a variable heavy chain comprising an amino acid sequence having at least 90%, 91%, 92%, 93%, 94%, 95%, 96%, 97%, 98%, or 99% identity to the amino acid sequence of SEQ ID NO: 120. In some embodiments, the antigen-specific binding domain of an anti-MUC16 CAR comprises a variable light chain comprising the amino acid sequence of SEQ ID NO: 119. In some embodiments, the antigen-specific binding domain of an anti-MUC16 CAR comprises a variable light chain comprising an amino acid sequence having at least 90%, 91%, 92%, 93%, 94%, 95%, 96%, 97%, 98%, or 99% identity to the amino acid sequence of SEQ ID NO: 119. In some embodiments, the antigen-specific binding domain of an anti-MUC16 CAR comprises a variable heavy chain comprising the amino acid sequence of SEQ ID NO: 120 and a variable light chain comprising the amino acid sequence of SEQ ID NO: 119.

In some embodiments, the antigen-specific binding domain of an anti-MUC16 CAR comprises a variable heavy chain comprising the amino acid sequence of SEQ ID NO: 134. In some embodiments, the antigen-specific binding domain of an anti-MUC16 CAR comprises a variable heavy chain comprising an amino acid sequence having at least 90%, 91%, 92%, 93%, 94%, 95%, 96%, 97%, 98%, or 99% identity to the amino acid sequence of SEQ ID NO: 134. In some embodiments, the antigen-specific binding domain of an anti-MUC16 CAR comprises a variable light chain comprising the amino acid sequence of SEQ ID NO: 133. In some embodiments, the antigen-specific binding domain of an anti-MUC16 CAR comprises a variable light chain comprising an amino acid sequence having at least 90%, 91%, 92%, 93%, 94%, 95%, 96%, 97%, 98%, or 99% identity to the amino acid sequence of SEQ ID NO: 133. In some embodiments, the antigen-specific binding domain of an anti-MUC16 CAR comprises a variable heavy chain comprising the amino acid sequence of SEQ ID NO: 134 and a variable light chain comprising the amino acid sequence of SEQ ID NO: 133.

In some embodiments, the antigen-specific binding domain of an anti-MUC16 CAR comprises a variable heavy chain comprising the amino acid sequence of SEQ ID NO: 146. In some embodiments, the antigen-specific binding domain of an anti-MUC16 CAR comprises a variable heavy chain comprising an amino acid sequence having at least 90%, 91%, 92%, 93%, 94%, 95%, 96%, 97%, 98%, or 99% identity to the amino acid sequence of SEQ ID NO: 146. In some embodiments, the antigen-specific binding domain of an anti-MUC16 CAR comprises a variable light chain comprising the amino acid sequence of SEQ ID NO: 145. In some embodiments, the antigen-specific binding domain of an anti-MUC16 CAR comprises a variable light chain comprising an amino acid sequence having at least 90%, 91%, 92%, 93%, 94%, 95%, 96%, 97%, 98%, or 99% identity to the amino acid sequence of SEQ ID NO: 145. In some embodiments, the antigen-specific binding domain of an anti-MUC16 CAR comprises a variable heavy chain comprising the amino acid sequence of SEQ ID NO: 146 and a variable light chain comprising the amino acid sequence of SEQ ID NO: 145.

MUC16-specific binding domains provided herein also comprise one, two, three, four, five, or six CDRs. Such CDRs may be nonhuman CDRs or altered nonhuman CDRs selected from CDRL1, CDRL2 and CDRL3 of the light chain and CDRH1, CDRH2 and CDRH3 of the heavy chain. In some embodiments, a MUC16-specific binding domain comprises (a) a light chain variable region that comprises a light chain CDRL1, a light chain CDRL2, and a light chain CDRL3, and (b) a heavy chain variable region that comprises a heavy chain CDRH1, a heavy chain CDRH2, and a heavy chain CDRH3.

In some embodiments, the antigen-specific binding domain of an anti-MUC16 CAR comprises the light chain CDRs of SEQ ID Nos: 1-3. In some embodiments, the antigen-specific binding domain of an anti-MUC16 CAR comprises the heavy chain CDRs of SEQ ID Nos: 4-6. In some embodiments, the antigen-specific binding domain of an anti-MUC16 CAR comprises the light chain CDRs of SEQ ID Nos: 1-3 and the heavy chain CDRs of SEQ ID Nos: 4-6.

In some embodiments, the antigen-specific binding domain of an anti-MUC16 CAR comprises the light chain CDRs of SEQ ID Nos: 41-43. In some embodiments, the antigen-specific binding domain of an anti-MUC16 CAR comprises the heavy chain CDRs of SEQ ID Nos: 44-46. In some embodiments, the antigen-specific binding domain of an anti-MUC16 CAR comprises the light chain CDRs of SEQ ID Nos: 41-43 and the heavy chain CDRs of SEQ ID Nos: 44-46.

In some embodiments, the antigen-specific binding domain of an anti-MUC16 CAR comprises the light chain CDRs of SEQ ID Nos: 53-55. In some embodiments, the antigen-specific binding domain of an anti-MUC16 CAR comprises the heavy chain CDRs of SEQ ID Nos: 56-58. In some embodiments, the antigen-specific binding domain of an anti-MUC16 CAR comprises the light chain CDRs of SEQ ID Nos: 53-55 and the heavy chain CDRs of SEQ ID Nos: 56-58.

In some embodiments, the antigen-specific binding domain of an anti-MUC16 CAR comprises the light chain CDRs of SEQ ID Nos: 65-67. In some embodiments, the antigen-specific binding domain of an anti-MUC16 CAR comprises the heavy chain CDRs of SEQ ID Nos: 68-70. In some embodiments, the antigen-specific binding domain of an anti-MUC16 CAR comprises the light chain CDRs of SEQ ID Nos: 65-67 and the heavy chain CDRs of SEQ ID Nos: 68-70.

In some embodiments, the antigen-specific binding domain of an anti-MUC16 CAR comprises the light chain CDRs of SEQ ID Nos: 77-79. In some embodiments, the antigen-specific binding domain of an anti-MUC16 CAR comprises the heavy chain CDRs of SEQ ID Nos: 80-82. In some embodiments, the antigen-specific binding domain of an anti-MUC16 CAR comprises the light chain CDRs of SEQ ID Nos: 77-79 and the heavy chain CDRs of SEQ ID Nos: 80-82.

In some embodiments, the antigen-specific binding domain of an anti-MUC16 CAR comprises the light chain CDRs of SEQ ID Nos: 89-91. In some embodiments, the antigen-specific binding domain of an anti-MUC16 CAR comprises the heavy chain CDRs of SEQ ID Nos: 92-94. In some embodiments, the antigen-specific binding domain of an anti-MUC16 CAR comprises the light chain CDRs of SEQ ID Nos: 89-91 and the heavy chain CDRs of SEQ ID Nos: 92-94.

In some embodiments, the antigen-specific binding domain of an anti-MUC16 CAR comprises the light chain CDRs of SEQ ID Nos: 101-103. In some embodiments, the antigen-specific binding domain of an anti-MUC16 CAR comprises the heavy chain CDRs of SEQ ID Nos: 104-106. In some embodiments, the antigen-specific binding domain of an anti-MUC16 CAR comprises the light chain CDRs of SEQ ID Nos: 101-103 and the heavy chain CDRs of SEQ ID Nos: 104-106.

In some embodiments, the antigen-specific binding domain of an anti-MUC16 CAR comprises the light chain CDRs of SEQ ID Nos: 113-115. In some embodiments, the antigen-specific binding domain of an anti-MUC16 CAR comprises the heavy chain CDRs of SEQ ID Nos: 116-118. In some embodiments, the antigen-specific binding domain of an anti-MUC16 CAR comprises the light chain CDRs of SEQ ID Nos: 113-115 and the heavy chain CDRs of SEQ ID Nos: 116-118.

In some embodiments, the antigen-specific binding domain of an anti-MUC16 CAR comprises the light chain CDRs of SEQ ID Nos: 127-129. In some embodiments, the antigen-specific binding domain of an anti-MUC16 CAR comprises the heavy chain CDRs of SEQ ID Nos: 130-132. In some embodiments, the antigen-specific binding domain of an anti-MUC16 CAR comprises the light chain CDRs of SEQ ID Nos: 127-129 and the heavy chain CDRs of SEQ ID Nos: 130-132.

In some embodiments, the antigen-specific binding domain of an anti-MUC16 CAR comprises the light chain CDRs of SEQ ID Nos: 139-141. In some embodiments, the antigen-specific binding domain of an anti-MUC16 CAR comprises the heavy chain CDRs of SEQ ID Nos: 142-144. In some embodiments, the antigen-specific binding domain of an anti-MUC16 CAR comprises the light chain CDRs of SEQ ID Nos: 139-141 and the heavy chain CDRs of SEQ ID Nos: 142-144.

Additional anti-MUC16 antibodies, and antigen-binding fragments thereof, are known in the art. See, for example, WO2018/058003, published Mar. 29, 2018, the entire contents of each which are expressly incorporated herein by reference in their entirety.

In some embodiments, the antigen-specific binding domain of an anti-MUC16 CAR binds human MUC16 within one or more five membrane-proximal SEA domains of human MUC16. In some embodiments, the antigen-specific binding domain of an anti-MUC16 CAR binds SEA domains 12-16, numbered N-terminus to C-terminus, of human MUC16. In some embodiments, the antigen-specific binding domain of an anti-MUC16 CAR binds SEA domains of human MUC16 as set forth in residues 13791-14451 of SEQ ID NO: 13. In some embodiments, the antigen-specific binding domain of an anti-MUC16 CAR binds within residues 13810-14451 of SEQ ID NO: 13. In some embodiments, the antigen-specific binding domain of an anti-MUC16 CAR does not bind SEA domains 1-11, numbered N-terminus to C-terminus, of human MUC16. In some embodiments, the antigen-specific binding domain of an anti-MUC16 CAR does not bind within residues 1-13790 of SEQ ID NO: 13. In some embodiments, the antigen-specific binding domain of an anti-MUC16 CAR does not bind within residues 1-13809 of SEQ ID NO: 13. In some embodiments, the antigen-specific binding domain of an anti-MUC16 CAR binds within SEQ ID NO: 39. In some embodiments, the antigen-specific binding domain of an anti-MUC16 CAR binds within SEQ ID NO: 151. In some embodiments, the antigen-specific binding domain of an anti-MUC16 CAR does not bind within SEQ ID NO: 40.

### 2. Linkers

In some embodiments, the CARs contemplated herein may comprise linker residues between the various domains, e.g., added for appropriate spacing and conformation of the molecule. In some embodiments the linker is a variable region linking sequence. A “variable region linking sequence,” is an amino acid sequence that connects the V_{H} and V_{L} domains and provides a spacer function compatible with interaction of the two sub-binding domains so that the resulting polypeptide retains a specific binding affinity to the same target molecule as an antibody that comprises the same light and heavy chain variable regions. In other embodiments the linker is a linking sequence between the transmembrane domain and one or more intracellular domains.

CARs contemplated herein, may comprise one, two, three, four, or five or more linkers. In some embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long.

Illustrative examples of linkers include glycine polymers (G)n; glycine-serine polymers (G₁₋₅S₁₋₅)ₙ, where n is an integer of at least one, two, three, four, or five; glycine-alanine polymers; alanine-serine polymers; and other flexible linkers known in the art. Glycine and glycine-serine polymers are relatively unstructured, and therefore may be able to serve as a neutral tether between domains of fusion proteins such as the CARs described herein. Glycine accesses significantly more phi-psi space than even alanine, and is much less restricted than residues with longer side chains (see Scheraga, Rev. Computational Chem. 11173-142 (1992)). The ordinarily skilled artisan will recognize that design of a CAR can include linkers that are all or partially flexible, such that the linker can include a flexible linker as well as one or more portions that confer less flexible structure to provide for a desired CAR structure.

Other exemplary linkers include, but are not limited to the following amino acid sequences: GGG; DGGGS (SEQ ID NO: 14); TGEKP (SEQ ID NO: 15) (see, e.g., Liu et al., PNAS 5525-5530 (1997)); GGRR (SEQ ID NO: 16) (Pomerantz et al. 1995, supra); (GGGGS)ₙ wherein=1, 2, 3, 4 or 5 (SEQ ID NO: 17) (Kim et al., PNAS 93, 1156-1160 (1996.); EGKSSGSGSESKVD (SEQ ID NO: 18) (Chaudhary et al., 1990, Proc. Natl. Acad. Sci. U.S.A. 87:1066-1070); KESGSVSSEQLAQFRSLD (SEQ ID NO: 19) (Bird et al., 1988, Science 242:423-426), GGRRGGGS (SEQ ID NO: 20); LRQRDGERP (SEQ ID NO: 21); LRQKDGGGSERP (SEQ ID NO: 22); LRQKd(GGGS)₂ ERP (SEQ ID NO: 23). In some embodiments, the linker is GGGGSGGGGSGGGGS (SEQ ID NO: 24). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods. In some embodiments, the linker comprises the following amino acid sequence: GSTSGSGKPGSGEGSTKG (SEQ ID NO: 25) (Cooper et al., Blood, 101(4): 1637-1644 (2003)). In one embodiment, the linker comprises a sequence of LYC.

### 3. Spacer Domain

In some embodiments, the binding domain of the CAR is followed by one or more “spacer domains,” which refers to the region that moves the antigen binding domain away from the effector cell surface to enable proper cell/cell contact, antigen binding and activation (Patel et al., Gene Therapy, 1999; 6: 412-419). The spacer domain may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region.

In some embodiment, the spacer domain comprises the CH2 and CH3 domains of IgG1 or IgG4.

### 4. Hinge Domain

In some embodiments, the binding domain of the CAR is followed by one or more “hinge domains” or “hinge regions”, which plays a role in positioning the antigen binding domain away from the effector cell surface to enable proper cell/cell contact, antigen binding and activation. In some embodiments, a CAR comprises one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region.

An “altered hinge region” refers to (a) a naturally occurring hinge region with up to 30% amino acid changes (e.g., up to 25%, 20%, 15%, 10%, or 5% amino acid substitutions or deletions), (b) a portion of a naturally occurring hinge region that is at least 10 amino acids (e.g., at least 12, 13, 14 or 15 amino acids) in length with up to 30% amino acid changes (e.g., up to 25%, 20%, 15%, 10%, or 5% amino acid substitutions or deletions), or (c) a portion of a naturally occurring hinge region that comprises the core hinge region (which may be 4, 5, 6, 7, 8, 9, 10, 11, 12, 13, 14, or 15, or at least 4, 5, 6, 7, 8, 9, 10, 11, 12, 13, 14, or 15 amino acids in length). In some embodiments, one or more cysteine residues in a naturally occurring immunoglobulin hinge region may be substituted by one or more other amino acid residues (e.g., one or more serine residues). An altered immunoglobulin hinge region may alternatively or additionally have a proline residue of a wild type immunoglobulin hinge region substituted by another amino acid residue (e.g., a serine residue).

Other illustrative hinge domains suitable for use in the CARs described herein include the hinge region derived from the extracellular regions of type 1 membrane proteins such as CD8α, CD4, CD28 and CD7, which may be wild-type hinge regions from these molecules or may be altered. In one embodiment, the hinge domain comprises a CD28 hinge region. In another embodiment, the hinge domain comprises a CD4 hinge region. In another embodiment, the hinge domain comprises a CD8a hinge region.

In various embodiments, the modified hinge region comprises an amino acid sequence having at least 85%, 86%, 87%, 88%, 89%, 90%, 91%, 92%, 93%, 94%, 95%, 96%, 97%, 98%, or 99% amino acid identity to a suitable hinge domain/region as contemplated herein and/or known in the art. In some embodiments, the modified hinge region comprising a hinge sequence as contemplated herein having 4 or fewer, 3 or fewer, or 2 or fewer amino acid substitutions and/or deletions.

In certain embodiments, one or more cysteine residues in a naturally occurring hinge region/domain may be substituted by one or more other amino acid residues to produce a modified hinge domain. In some embodiments, the modified hinge domain comprises one or more cysteine residues substituted with serine(s) or alanine(s). In another embodiment, the modified hinge domain comprises one or more cysteine residues substituted with serine(s). In another embodiment, the modified hinge domain comprises one or more cysteine residues substituted with alanine(s).

In particular embodiments, an altered hinge region comprises substitution of a proline residue by another amino acid residue (e.g., a serine residue).

In some aspects, the hinge region comprises a sequence having at least 90%, 91%, 92%, 93%, 94%, 95%, 96%, 97%, 98%, or 99% identity to, or comprises the sequence of SEQ ID NO: 153. In one embodiment, the hinge region polypeptide comprises a sequence having at least 95% identity to SEQ ID NO: 153. In one embodiment, the hinge region polypeptide comprises SEQ ID NO: 153.

### 5. Transmembrane (TM) Domain

In some embodiments, a CAR contemplated herein comprises a transmembrane domain. The “transmembrane domain” or “TM domain” is the portion of the CAR that fuses the extracellular binding portion and intracellular signaling domain and anchors the CAR to the plasma membrane of the immune effector cell. The TM domain may be derived either from a natural, synthetic, semi-synthetic, or recombinant source. The TM domain may be derived from (i.e., comprise at least the transmembrane region(s) of) the alpha, beta or zeta chain of the T-cell receptor, CD3δ, CD3ε, CD37, CD3ζ, CD4, CD5, CD8α, CD9, CD16, CD22, CD27, CD28, CD33, CD37, CD45, CD64, CD80, CD86, CD134, CD137, CD152, CD 154, and PD1. In a particular embodiment, the TM domain is synthetic and predominantly comprises hydrophobic residues such as leucine and valine.

In some aspects, the CARs contemplated herein comprise a TM domain derived from CD8α. In some embodiments, the transmembrane domain comprises a sequence having at least 90%, 91%, 92%, 93%, 94%, 95%, 96%, 97%, 98%, or 99% identity to, or comprises the sequence of SEQ ID NO: 154. In one embodiment, the transmembrane domain comprises a sequence having at least 95% identity to SEQ ID NO: 154. In one embodiment, the transmembrane domain comprises SEQ ID NO: 154.

In some embodiments, a CAR contemplated herein comprises a TM domain derived from CD8α and a short oligo- or polypeptide linker, preferably between 1, 2, 3, 4, 5, 6, 7, 8, 9, or 10 amino acids in length that links the TM domain and the intracellular signaling domain of the CAR. In some embodiments, the linker is a glycine-serine based linker. In other embodiments, the linker is an LYC linker.

In another aspect, the CARs contemplated herein comprise a TM domain derived from CD28. In another aspect, the CARs contemplated herein comprise a TM domain derived from CD4.

### 6. Intracellular Signaling Domain

In some embodiments, CARs contemplated herein comprise an intracellular signaling domain. An “intracellular signaling domain,” refers to the part of a CAR that participates in transducing the message of effective MUC16 CAR binding to a human MUC16 polypeptide into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain.

The term “effector function” refers to a specialized function of an immune effector cell. Effector function of the T cell, for example, may be cytolytic activity or help or activity including the secretion of a cytokine. Thus, the term “intracellular signaling domain” refers to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal.

It is known that signals generated through the TCR alone are insufficient for full activation of the T cell and that a secondary or co-stimulatory signal is also required. Thus, T cell activation can be said to be mediated by two distinct classes of intracellular signaling domains: primary signaling domains that initiate antigen-dependent primary activation through the TCR (e.g., a TCR/CD3 complex) and co-stimulatory signaling domains that act in an antigen-independent manner to provide a secondary or co-stimulatory signal. In preferred embodiments, a CAR contemplated herein comprises an intracellular signaling domain that comprises one or more “co-stimulatory signaling domain” and a “primary signaling domain.”

Primary signaling domains regulate primary activation of the TCR complex either in a stimulatory way, or in an inhibitory way. Primary signaling domains that act in a stimulatory manner may contain signaling motifs which are known as immunoreceptor tyrosine-based activation motifs or ITAMs.

Illustrative examples of ITAM containing primary signaling domains that are of particular use in the invention include those derived from TCRζ, FcRγ, FcRβ, CD3γ, CD3δ, CD3ε, CD3ζ, CD22, CD79a, CD79b, and CD66d. In some embodiments, a CAR comprises a CD3ζ primary signaling domain and one or more co-stimulatory signaling domains. The intracellular primary signaling and co-stimulatory signaling domains may be linked in any order in tandem to the carboxyl terminus of the transmembrane domain.

CARs contemplated herein comprise one or more co-stimulatory signaling domains to enhance the efficacy and expansion of T cells expressing CAR receptors. As used herein, the term, “co-stimulatory signaling domain,” or “co-stimulatory domain”, refers to an intracellular signaling domain of a co-stimulatory molecule. Co-stimulatory molecules are cell surface molecules other than antigen receptors or Fc receptors that provide a second signal required for efficient activation and function of T lymphocytes upon binding to antigen. Illustrative examples of such co-stimulatory molecules include CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70. In some embodiments, a CAR comprises one or more co-stimulatory signaling domains selected from the group consisting of CD28, CD137, and CD134, and a CD3ζ primary signaling domain.

In some embodiments, a CAR comprises CD28 and CD137 co-stimulatory signaling domains and a CD3ζ primary signaling domain.

In some embodiments, a CAR comprises CD28 and CD134 co-stimulatory signaling domains and a CD3ζ primary signaling domain.

In some embodiments, a CAR comprises CD137 and CD134 co-stimulatory signaling domains and a CD3ζ primary signaling domain.

In some embodiments, a CAR comprises a CD134 co-stimulatory signaling domain and a CD3ζ primary signaling domain.

In some embodiments, a CAR comprises a CD28 co-stimulatory signaling domain and a CD3ζ primary signaling domain.

In some embodiments, a CAR comprises a CD137 co-stimulatory signaling domain and a CD3ζ primary signaling domain.

In some aspects, the one or more co-stimulatory signaling domains is from CD137 (4-1BB). In some aspects, the co-stimulatory signaling domain comprises a sequence having at least 90%, 91%, 92%, 93%, 94%, 95%, 96%, 97%, 98%, or 99% identity to, or comprises the sequence of SEQ ID NO: 155. In one embodiment, the co-stimulatory signaling domain comprises a sequence having at least 95% identity to SEQ ID NO: 155. In one embodiment, the co-stimulatory signaling domain comprises SEQ ID NO: 155.

In any of the foregoing or related aspects, the primary signaling domain is from CD3ζ. In some aspects, the primary signaling domain comprises a sequence having at least 90%, 91%, 92%, 93%, 94%, 95%, 96%, 97%, 98%, or 99% identity to, or comprises the sequence of SEQ ID NO: 156. In one embodiment, the primary signaling domain comprises a sequence having at least 95% identity to SEQ ID NO: 156. In one embodiment, the primary signaling domain comprises SEQ ID NO: 156.

### D. Exemplary Chimeric Antigen Receptors

In some embodiments, a CAR comprises an anti-MUC16 scFv that binds a MUC16 polypeptide; a transmembrane domain derived from a polypeptide selected from the group consisting of: alpha, beta or zeta chain of the T-cell receptor, CD3ε, CD3ζ, CD4, CD5, CD8α, CD9, CD 16, CD22, CD27, CD28, CD33, CD37, CD45, CD64, CD80, CD86, CD 134, CD137, CD152, CD 154, and PD1; and one or more intracellular co-stimulatory signaling domains from a co-stimulatory molecule selected from the group consisting of: CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70; and a primary signaling domain from TCRζ, FcRγ, FcRβ, CD3γ, CD3δ, CD3ε, CD3ζ, CD22, CD79a, CD79b, and CD66d.

In some embodiments, a CAR comprises an anti-MUC16 scFv that binds a MUC16 polypeptide; a hinge domain selected from the group consisting of: IgG1 hinge/CH2/CH3, IgG4 hinge/CH2/CH3, and a CD8α hinge; a transmembrane domain derived from a polypeptide selected from the group consisting of: alpha, beta or zeta chain of the T-cell receptor, CD3ε, CD3ζ, CD4, CD5, CD8α, CD9, CD 16, CD22, CD27, CD28, CD33, CD3γ, CD45, CD64, CD80, CD86, CD 134, CD137, CD152, CD 154, and PD1; and one or more intracellular co-stimulatory signaling domains from a co-stimulatory molecule selected from the group consisting of: CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70; and a primary signaling domain from TCRζ, FcRγ, FcRβ, CD3γ, CD3δ, CD3ε, CD3ζ, CD22, CD79a, CD79b, and CD66d.

In some embodiments, a CAR comprises an anti-MUC16 scFv that binds a MUC16 polypeptide; a hinge domain selected from the group consisting of: IgG1 hinge/CH2/CH3, IgG4 hinge/CH2/CH3, and a CD8α hinge; a transmembrane domain derived from a polypeptide selected from the group consisting of: alpha, beta or zeta chain of the T-cell receptor, CD3ε, CD3ζ, CD4, CD5, CD8α, CD9, CD 16, CD22, CD27, CD28, CD33, CD3γ, CD45, CD64, CD80, CD86, CD 134, CD137, CD152, CD 154, and PD1; a short oligo- or polypeptide linker, preferably between 1, 2, 3, 4, 5, 6, 7, 8, 9, or 10 amino acids in length that links the TM domain to the intracellular signaling domain of the CAR; and one or more intracellular co-stimulatory signaling domains from a co-stimulatory molecule selected from the group consisting of: CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70; and a primary signaling domain from TCRζ, FcRγ, FcRβ, CD3γ, CD3δ, CD3ε, CD3ζ, CD22, CD79a, CD79b, and CD66d.

In some embodiments, a CAR comprises an anti-MUC16 scFv that binds a MUC16 polypeptide; a hinge domain comprising a CD8α polypeptide; a CD8α transmembrane domain optionally comprising a polypeptide linker of about 3 to about 10 amino acids; a CD137 intracellular co-stimulatory signaling domain; and a CD3ζ primary signaling domain.

In some embodiments, a CAR comprises an anti-MUC16 scFv that binds within SEQ ID NO: 39 or 151; a transmembrane domain derived from a polypeptide selected from the group consisting of: alpha, beta or zeta chain of the T-cell receptor, CD3ε, CD3ζ, CD4, CD5, CD8α, CD9, CD 16, CD22, CD27, CD28, CD33, CD3γ, CD45, CD64, CD80, CD86, CD 134, CD137, CD152, CD 154, and PD1; and one or more intracellular co-stimulatory signaling domains from a co-stimulatory molecule selected from the group consisting of: CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70; and a primary signaling domain from TCRζ, FcRγ, FcRβ, CD3γ, CD3δ, CD3ε, CD3ζ, CD22, CD79a, CD79b, and CD66d.

In some embodiments, a CAR comprises an anti-MUC16 scFv that binds within SEQ ID NO: 39 or 151; a hinge domain selected from the group consisting of: IgG1 hinge/CH2/CH3, IgG4 hinge/CH2/CH3, and a CD8α hinge; a transmembrane domain derived from a polypeptide selected from the group consisting of: alpha, beta or zeta chain of the T-cell receptor, CD3ε, CD3ζ, CD4, CD5, CD8α, CD9, CD 16, CD22, CD27, CD28, CD33, CD3γ, CD45, CD64, CD80, CD86, CD 134, CD137, CD152, CD 154, and PD1; and one or more intracellular co-stimulatory signaling domains from a co-stimulatory molecule selected from the group consisting of: CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70; and a primary signaling domain from TCRζ, FcRγ, FcRβ, CD3γ, CD3δ, CD3ε, CD3ζ, CD22, CD79a, CD79b, and CD66d.

In some embodiments, a CAR comprises an anti-MUC16 scFv that binds within SEQ ID NO: 39 or 151; a hinge domain selected from the group consisting of: IgG1 hinge/CH2/CH3, IgG4 hinge/CH2/CH3, and a CD8α hinge; a transmembrane domain derived from a polypeptide selected from the group consisting of: alpha, beta or zeta chain of the T-cell receptor, CD3ε, CD3ζ, CD4, CD5, CD8α, CD9, CD 16, CD22, CD27, CD28, CD33, CD3γ, CD45, CD64, CD80, CD86, CD 134, CD137, CD152, CD 154, and PD1; optionally a short oligo- or polypeptide linker, preferably between 1, 2, 3, 4, 5, 6, 7, 8, 9, or 10 amino acids in length that links the TM domain to the intracellular signaling domain of the CAR; and one or more intracellular co-stimulatory signaling domains from a co-stimulatory molecule selected from the group consisting of: CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70; and a primary signaling domain from TCRζ, FcRγ, FcRβ, CD3γ, CD3δ, CD3ε, CD3ζ, CD22, CD79a, CD79b, and CD66d.

In some embodiments, a CAR comprises an anti-MUC16 scFv that binds within SEQ ID NO: 39 or SEQ ID NO: 151; a hinge domain comprising a CD8α polypeptide; a CD8α transmembrane domain, optionally comprising a polypeptide linker of about 3 to about 10 amino acids, such as LYC; a CD137 intracellular co-stimulatory signaling domain; and a CD3ζ primary signaling domain.

In some embodiments, a CAR comprises an anti-MUC16 scFv comprising light chain CDRs as set forth in SEQ ID NOs: 1-3 and heavy chain CDRs as set forth in SEQ ID NO: 4-6; a hinge domain comprising a CD8α polypeptide; a CD8α transmembrane domain optionally comprising a polypeptide linker of about 3 to about 10 amino acids; a CD137 intracellular co-stimulatory signaling domain; and a CD3ζ primary signaling domain.

In some embodiments, a CAR comprises an anti-MUC16 scFv comprising (a) light chain CDRs as set forth in SEQ ID NOs: 41-43 and heavy chain CDRs as set forth in SEQ ID NO: 44-46; (b) light chain CDRs as set forth in SEQ ID NOs: 53-55 and heavy chain CDRs as set forth in SEQ ID NO: 56-58; (c) light chain CDRs as set forth in SEQ ID NOs: 65-67 and heavy chain CDRs as set forth in SEQ ID NO: 68-70; (d) light chain CDRs as set forth in SEQ ID NOs: 77-79 and heavy chain CDRs as set forth in SEQ ID NO: 80-82; (e) light chain CDRs as set forth in SEQ ID NOs: 89-91 and heavy chain CDRs as set forth in SEQ ID NO: 92-94; (f) light chain CDRs as set forth in SEQ ID NOs: 101-103 and heavy chain CDRs as set forth in SEQ ID NO: 104-106; (g) light chain CDRs as set forth in SEQ ID NOs: 113-115 and heavy chain CDRs as set forth in SEQ ID NO: 116-118; (h) light chain CDRs as set forth in SEQ ID NOs: 127-129 and heavy chain CDRs as set forth in SEQ ID NO: 130-132; or (i) light chain CDRs as set forth in SEQ ID NOs: 139-141 and heavy chain CDRs as set forth in SEQ ID NO: 142-144; a hinge domain comprising a CD8α polypeptide; a CD8α transmembrane domain optionally comprising a polypeptide linker of about 3 to about 10 amino acids, such as LYC; a CD137 intracellular co-stimulatory signaling domain; and a CD3ζ primary signaling domain.

In some embodiments, a CD8α signal polypeptide, a CAR comprises an anti-MUC16 scFv comprising light chain CDRs as set forth in SEQ ID Nos: 1-3 and heavy chain CDRs as set forth in SEQ ID NO: 4-6; a hinge domain comprising a CD8α polypeptide; a CD8α transmembrane domain optionally comprising a polypeptide linker of about 3 to about 10 amino acids, such as LYC; a CD137 intracellular co-stimulatory signaling domain; and a CD3ζ primary signaling domain.

In some embodiments, a CD8α signal polypeptide, a CAR comprises an anti-MUC16 scFv comprising (a) light chain CDRs as set forth in SEQ ID NOs: 41-43 and heavy chain CDRs as set forth in SEQ ID NO: 44-46; (b) light chain CDRs as set forth in SEQ ID NOs: 53-55 and heavy chain CDRs as set forth in SEQ ID NO: 56-58; (c) light chain CDRs as set forth in SEQ ID NOs: 65-67 and heavy chain CDRs as set forth in SEQ ID NO: 68-70; (d) light chain CDRs as set forth in SEQ ID NOs: 77-79 and heavy chain CDRs as set forth in SEQ ID NO: 80-82; (e) light chain CDRs as set forth in SEQ ID NOs: 89-91 and heavy chain CDRs as set forth in SEQ ID NO: 92-94; (f) light chain CDRs as set forth in SEQ ID NOs: 101-103 and heavy chain CDRs as set forth in SEQ ID NO: 104-106; (g) light chain CDRs as set forth in SEQ ID NOs: 113-115 and heavy chain CDRs as set forth in SEQ ID NO: 116-118; (h) light chain CDRs as set forth in SEQ ID NOs: 127-129 and heavy chain CDRs as set forth in SEQ ID NO: 130-132; or (i) light chain CDRs as set forth in SEQ ID NOs: 139-141 and heavy chain CDRs as set forth in SEQ ID NO: 142-144; and a hinge domain comprising a CD8α polypeptide; a CD8α transmembrane domain optionally comprising a polypeptide linker of about 3 to about 10 amino acids, such as LYC; a CD137 intracellular co-stimulatory signaling domain; and a CD3ζ primary signaling domain.

In some embodiments, a CAR comprises an anti-MUC16 scFv comprising a variable light chain as set forth in SEQ ID NO: 7 and a variable heavy chain as set forth in SEQ ID NO: 8; a hinge domain comprising a CD8α polypeptide; a CD8α transmembrane domain optionally comprising a polypeptide linker of about 3 to about 10 amino acids, such as LYC; a CD137 intracellular co-stimulatory signaling domain; and a CD3ζ primary signaling domain.

In some embodiments, a CAR comprises an anti-MUC16 scFv comprising (a) a variable light chain as set forth in SEQ ID NO: 47 and a variable heavy chain as set forth in SEQ ID NO: 48; (b) a variable light chain as set forth in SEQ ID NO: 59 and a variable heavy chain as set forth in SEQ ID NO: 60; (c) a variable light chain as set forth in SEQ ID NO: 71 and a variable heavy chain as set forth in SEQ ID NO: 72; (d) a variable light chain as set forth in SEQ ID NO: 83 and a variable heavy chain as set forth in SEQ ID NO: 84; (e) a variable light chain as set forth in SEQ ID NO: 95 and a variable heavy chain as set forth in SEQ ID NO: 96; (f) a variable light chain as set forth in SEQ ID NO: 107 and a variable heavy chain as set forth in SEQ ID NO: 108; (g) a variable light chain as set forth in SEQ ID NO: 119 and a variable heavy chain as set forth in SEQ ID NO: 120; (h) a variable light chain as set forth in SEQ ID NO: 133 and a variable heavy chain as set forth in SEQ ID NO: 134; or (i) a variable light chain as set forth in SEQ ID NO: 145 and a variable heavy chain as set forth in SEQ ID NO: 146; and a hinge domain comprising a CD8α polypeptide; a CD8α transmembrane domain optionally comprising a polypeptide linker of about 3 to about 10 amino acids, such as LYC; a CD137 intracellular co-stimulatory signaling domain; and a CD3ζ primary signaling domain.

In some embodiments, a CD8α signal polypeptide, a CAR comprises an anti-MUC16 scFv comprising a variable light chain as set forth in SEQ ID NO: 7 and a variable heavy chain as set forth in SEQ ID NO: 8; a hinge domain comprising a CD8α polypeptide; a CD8α transmembrane domain optionally comprising a polypeptide linker of about 3 to about 10 amino acids, such as LYC; a CD137 intracellular co-stimulatory signaling domain; and a CD3ζ primary signaling domain.

In some embodiments, a CD8α signal polypeptide, a CAR comprises an anti-MUC16 scFv comprising (a) a variable light chain as set forth in SEQ ID NO: 47 and a variable heavy chain as set forth in SEQ ID NO: 48; (b) a variable light chain as set forth in SEQ ID NO: 59 and a variable heavy chain as set forth in SEQ ID NO: 60; (c) a variable light chain as set forth in SEQ ID NO: 71 and a variable heavy chain as set forth in SEQ ID NO: 72; (d) a variable light chain as set forth in SEQ ID NO: 83 and a variable heavy chain as set forth in SEQ ID NO: 84; (e) a variable light chain as set forth in SEQ ID NO: 95 and a variable heavy chain as set forth in SEQ ID NO: 96; (f) a variable light chain as set forth in SEQ ID NO: 107 and a variable heavy chain as set forth in SEQ ID NO: 108; (g) a variable light chain as set forth in SEQ ID NO: 119 and a variable heavy chain as set forth in SEQ ID NO: 120; (h) a variable light chain as set forth in SEQ ID NO: 133 and a variable heavy chain as set forth in SEQ ID NO: 134; or (i) a variable light chain as set forth in SEQ ID NO: 145 and a variable heavy chain as set forth in SEQ ID NO: 146; and a CD8α transmembrane domain optionally comprising a polypeptide linker of about 3 to about 10 amino acids, such as LYC; a CD137 intracellular co-stimulatory signaling domain; and a CD3ζ primary signaling domain.

In some embodiments, a CAR comprises the amino acid sequence set forth in SEQ ID NO: 9. In some embodiments, a CAR comprises an amino acid sequence having at least 90% identity to SEQ ID NO: 9. In some embodiments, a CAR comprises an amino acid sequence having at least 95% identity to SEQ ID NO: 9. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 9.

In some embodiments, a CAR comprises the amino acid sequence set forth in SEQ ID NO: 49. In some embodiments, a CAR comprises an amino acid sequence having at least 90% identity to SEQ ID NO: 49. In some embodiments, a CAR comprises an amino acid sequence having at least 95% identity to SEQ ID NO: 49. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 49.

In some embodiments, a CAR comprises the amino acid sequence set forth in SEQ ID NO: 51. In some embodiments, a CAR comprises an amino acid sequence having at least 90% identity to SEQ ID NO: 51. In some embodiments, a CAR comprises an amino acid sequence having at least 95% identity to SEQ ID NO: 51. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 51.

In some embodiments, a CAR comprises the amino acid sequence set forth in SEQ ID NO: 61. In some embodiments, a CAR comprises an amino acid sequence having at least 90% identity to SEQ ID NO: 61. In some embodiments, a CAR comprises an amino acid sequence having at least 95% identity to SEQ ID NO: 61. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 61.

In some embodiments, a CAR comprises the amino acid sequence set forth in SEQ ID NO: 63. In some embodiments, a CAR comprises an amino acid sequence having at least 90% identity to SEQ ID NO: 63. In some embodiments, a CAR comprises an amino acid sequence having at least 95% identity to SEQ ID NO: 63. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 63.

In some embodiments, a CAR comprises the amino acid sequence set forth in SEQ ID NO: 73. In some embodiments, a CAR comprises an amino acid sequence having at least 90% identity to SEQ ID NO: 73. In some embodiments, a CAR comprises an amino acid sequence having at least 95% identity to SEQ ID NO: 73. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 73.

In some embodiments, a CAR comprises the amino acid sequence set forth in SEQ ID NO: 75. In some embodiments, a CAR comprises an amino acid sequence having at least 90% identity to SEQ ID NO: 75. In some embodiments, a CAR comprises an amino acid sequence having at least 95% identity to SEQ ID NO: 75. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 75.

In some embodiments, a CAR comprises the amino acid sequence set forth in SEQ ID NO: 85. In some embodiments, a CAR comprises an amino acid sequence having at least 90% identity to SEQ ID NO: 85. In some embodiments, a CAR comprises an amino acid sequence having at least 95% identity to SEQ ID NO: 85. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 85.

In some embodiments, a CAR comprises the amino acid sequence set forth in SEQ ID NO: 87. In some embodiments, a CAR comprises an amino acid sequence having at least 90% identity to SEQ ID NO: 87. In some embodiments, a CAR comprises an amino acid sequence having at least 95% identity to SEQ ID NO: 87. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 87.

In some embodiments, a CAR comprises the amino acid sequence set forth in SEQ ID NO: 97. In some embodiments, a CAR comprises an amino acid sequence having at least 90% identity to SEQ ID NO: 97. In some embodiments, a CAR comprises an amino acid sequence having at least 95% identity to SEQ ID NO: 97. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 97.

In some embodiments, a CAR comprises the amino acid sequence set forth in SEQ ID NO: 99. In some embodiments, a CAR comprises an amino acid sequence having at least 90% identity to SEQ ID NO: 99. In some embodiments, a CAR comprises an amino acid sequence having at least 95% identity to SEQ ID NO: 99. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 99.

In some embodiments, a CAR comprises the amino acid sequence set forth in SEQ ID NO: 109. In some embodiments, a CAR comprises an amino acid sequence having at least 90% identity to SEQ ID NO: 109. In some embodiments, a CAR comprises an amino acid sequence having at least 95% identity to SEQ ID NO: 109. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 109.

In some embodiments, a CAR comprises the amino acid sequence set forth in SEQ ID NO: 111. In some embodiments, a CAR comprises an amino acid sequence having at least 90% identity to SEQ ID NO: 111. In some embodiments, a CAR comprises an amino acid sequence having at least 95% identity to SEQ ID NO: 111. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 111.

In some embodiments, a CAR comprises the amino acid sequence set forth in SEQ ID NO: 121. In some embodiments, a CAR comprises an amino acid sequence having at least 90% identity to SEQ ID NO: 121. In some embodiments, a CAR comprises an amino acid sequence having at least 95% identity to SEQ ID NO: 121. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 121.

In some embodiments, a CAR comprises the amino acid sequence set forth in SEQ ID NO: 123. In some embodiments, a CAR comprises an amino acid sequence having at least 90% identity to SEQ ID NO: 123. In some embodiments, a CAR comprises an amino acid sequence having at least 95% identity to SEQ ID NO: 123. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 123.

In some embodiments, a CAR comprises the amino acid sequence set forth in SEQ ID NO: 125. In some embodiments, a CAR comprises an amino acid sequence having at least 90% identity to SEQ ID NO: 125. In some embodiments, a CAR comprises an amino acid sequence having at least 95% identity to SEQ ID NO: 125. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 125.

In some embodiments, a CAR comprises the amino acid sequence set forth in SEQ ID NO: 135. In some embodiments, a CAR comprises an amino acid sequence having at least 90% identity to SEQ ID NO: 135. In some embodiments, a CAR comprises an amino acid sequence having at least 95% identity to SEQ ID NO: 135. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 135.

In some embodiments, a CAR comprises the amino acid sequence set forth in SEQ ID NO: 137. In some embodiments, a CAR comprises an amino acid sequence having at least 90% identity to SEQ ID NO: 137. In some embodiments, a CAR comprises an amino acid sequence having at least 95% identity to SEQ ID NO: 137. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 137.

In some embodiments, a CAR comprises the amino acid sequence set forth in SEQ ID NO: 147. In some embodiments, a CAR comprises an amino acid sequence having at least 90% identity to SEQ ID NO: 147. In some embodiments, a CAR comprises an amino acid sequence having at least 95% identity to SEQ ID NO: 147. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 147.

In some embodiments, a CAR comprises the amino acid sequence set forth in SEQ ID NO: 149. In some embodiments, a CAR comprises an amino acid sequence having at least 90% identity to SEQ ID NO: 149. In some embodiments, a CAR comprises an amino acid sequence having at least 95% identity to SEQ ID NO: 149. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 149.

In some embodiments, a CAR comprises the amino acid sequence set forth in SEQ ID NO: 11. In some embodiments, a CAR comprises an amino acid sequence having at least 90% identity to SEQ ID NO: 11. In some embodiments, a CAR comprises an amino acid sequence having at least 95% identity to SEQ ID NO: 11. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 11.

In some embodiments, a CAR comprises the amino acid sequence set forth in SEQ ID NO: 50. In some embodiments, a CAR comprises an amino acid sequence having at least 90% identity to SEQ ID NO: 50. In some embodiments, a CAR comprises an amino acid sequence having at least 95% identity to SEQ ID NO: 50. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 50.

In some embodiments, a CAR comprises the amino acid sequence set forth in SEQ ID NO: 52. In some embodiments, a CAR comprises an amino acid sequence having at least 90% identity to SEQ ID NO: 52. In some embodiments, a CAR comprises an amino acid sequence having at least 95% identity to SEQ ID NO: 52. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 52.

In some embodiments, a CAR comprises the amino acid sequence set forth in SEQ ID NO: 62. In some embodiments, a CAR comprises an amino acid sequence having at least 90% identity to SEQ ID NO: 62. In some embodiments, a CAR comprises an amino acid sequence having at least 95% identity to SEQ ID NO: 62. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 62.

In some embodiments, a CAR comprises the amino acid sequence set forth in SEQ ID NO: 64. In some embodiments, a CAR comprises an amino acid sequence having at least 90% identity to SEQ ID NO: 64. In some embodiments, a CAR comprises an amino acid sequence having at least 95% identity to SEQ ID NO: 64. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 64.

In some embodiments, a CAR comprises the amino acid sequence set forth in SEQ ID NO: 74. In some embodiments, a CAR comprises an amino acid sequence having at least 90% identity to SEQ ID NO: 74. In some embodiments, a CAR comprises an amino acid sequence having at least 95% identity to SEQ ID NO: 74. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 74.

In some embodiments, a CAR comprises the amino acid sequence set forth in SEQ ID NO: 76. In some embodiments, a CAR comprises an amino acid sequence having at least 90% identity to SEQ ID NO: 76. In some embodiments, a CAR comprises an amino acid sequence having at least 95% identity to SEQ ID NO: 76. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 76.

In some embodiments, a CAR comprises the amino acid sequence set forth in SEQ ID NO: 86. In some embodiments, a CAR comprises an amino acid sequence having at least 90% identity to SEQ ID NO: 86. In some embodiments, a CAR comprises an amino acid sequence having at least 95% identity to SEQ ID NO: 86. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 86.

In some embodiments, a CAR comprises the amino acid sequence set forth in SEQ ID NO: 88. In some embodiments, a CAR comprises an amino acid sequence having at least 90% identity to SEQ ID NO: 88. In some embodiments, a CAR comprises an amino acid sequence having at least 95% identity to SEQ ID NO: 88. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 88.

In some embodiments, a CAR comprises the amino acid sequence set forth in SEQ ID NO: 98. In some embodiments, a CAR comprises an amino acid sequence having at least 90% identity to SEQ ID NO: 98. In some embodiments, a CAR comprises an amino acid sequence having at least 95% identity to SEQ ID NO: 98. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 98.

In some embodiments, a CAR comprises the amino acid sequence set forth in SEQ ID NO: 100. In some embodiments, a CAR comprises an amino acid sequence having at least 90% identity to SEQ ID NO: 100. In some embodiments, a CAR comprises an amino acid sequence having at least 95% identity to SEQ ID NO: 100. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 100.

In some embodiments, a CAR comprises the amino acid sequence set forth in SEQ ID NO: 110. In some embodiments, a CAR comprises an amino acid sequence having at least 90% identity to SEQ ID NO: 110. In some embodiments, a CAR comprises an amino acid sequence having at least 95% identity to SEQ ID NO: 110. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 110.

In some embodiments, a CAR comprises the amino acid sequence set forth in SEQ ID NO: 112. In some embodiments, a CAR comprises an amino acid sequence having at least 90% identity to SEQ ID NO: 112. In some embodiments, a CAR comprises an amino acid sequence having at least 95% identity to SEQ ID NO: 112. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 112.

In some embodiments, a CAR comprises the amino acid sequence set forth in SEQ ID NO: 122. In some embodiments, a CAR comprises an amino acid sequence having at least 90% identity to SEQ ID NO: 122. In some embodiments, a CAR comprises an amino acid sequence having at least 95% identity to SEQ ID NO: 122. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 122.

In some embodiments, a CAR comprises the amino acid sequence set forth in SEQ ID NO: 124. In some embodiments, a CAR comprises an amino acid sequence having at least 90% identity to SEQ ID NO: 124. In some embodiments, a CAR comprises an amino acid sequence having at least 95% identity to SEQ ID NO: 124. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 124.

In some embodiments, a CAR comprises the amino acid sequence set forth in SEQ ID NO: 126. In some embodiments, a CAR comprises an amino acid sequence having at least 90% identity to SEQ ID NO: 126. In some embodiments, a CAR comprises an amino acid sequence having at least 95% identity to SEQ ID NO: 126. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 126.

In some embodiments, a CAR comprises the amino acid sequence set forth in SEQ ID NO: 136. In some embodiments, a CAR comprises an amino acid sequence having at least 90% identity to SEQ ID NO: 136. In some embodiments, a CAR comprises an amino acid sequence having at least 95% identity to SEQ ID NO: 136. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 136.

In some embodiments, a CAR comprises the amino acid sequence set forth in SEQ ID NO: 138. In some embodiments, a CAR comprises an amino acid sequence having at least 90% identity to SEQ ID NO: 138. In some embodiments, a CAR comprises an amino acid sequence having at least 95% identity to SEQ ID NO: 138. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 138.

In some embodiments, a CAR comprises the amino acid sequence set forth in SEQ ID NO: 148. In some embodiments, a CAR comprises an amino acid sequence having at least 90% identity to SEQ ID NO: 148. In some embodiments, a CAR comprises an amino acid sequence having at least 95% identity to SEQ ID NO: 148. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 148.

In some embodiments, a CAR comprises the amino acid sequence set forth in SEQ ID NO: 150. In some embodiments, a CAR comprises an amino acid sequence having at least 90% identity to SEQ ID NO: 150. In some embodiments, a CAR comprises an amino acid sequence having at least 95% identity to SEQ ID NO: 150. In some embodiments, a CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 150.

Moreover, the design of the CARs contemplated herein enable improved expansion, long-term persistence, and tolerable cytotoxic properties in T cells expressing the CARs compared to non-modified T cells or T cells modified to express other CARs.

### E. Polypeptides

The present disclosure contemplates, in part, CAR polypeptides and fragments thereof, cells and compositions comprising the same, and vectors that express polypeptides. In some embodiments, a polypeptide comprising one or more CARs as set forth in SEQ ID NO: 9 is provided. In some embodiments, a polypeptide comprising one or more CARs as set forth in SEQ ID NO: 11 is provided.

“Polypeptide,” “polypeptide fragment,” “peptide” and “protein” are used interchangeably, unless specified to the contrary, and according to conventional meaning, i.e., as a sequence of amino acids. Polypeptides are not limited to a specific length, e.g., they may comprise a full length protein sequence or a fragment of a full length protein, and may include post-translational modifications of the polypeptide, for example, glycosylations, acetylations, phosphorylations and the like, as well as other modifications known in the art, both naturally occurring and non-naturally occurring. In various embodiments, the CAR polypeptides contemplated herein comprise a signal (or leader) polypeptide sequence at the N-terminal end of the protein, which co-translationally or post-translationally directs transfer of the protein. Illustrative examples of suitable signal polypeptide sequences useful in CARs disclosed herein include, but are not limited to a IgG1 heavy chain signal polypeptide sequence, a granulocyte-macrophage colony stimulating factor receptor 2 (GM-CSFR2) signal polypeptide sequence, a Igκ signal polypeptide sequence, or a CD8α signal polypeptide sequence. Polypeptides can be prepared using any of a variety of well-known recombinant and/or synthetic techniques. Polypeptides contemplated herein specifically encompass the CARs of the present disclosure, or sequences that have deletions from, additions to, and/or substitutions of one or more amino acid of a CAR as disclosed herein.

An “isolated peptide” or an “isolated polypeptide” and the like, as used herein, refer to in vitro isolation and/or purification of a peptide or polypeptide molecule from a cellular environment, and from association with other components of the cell, i.e., it is not significantly associated with in vivo substances. Similarly, an “isolated cell” refers to a cell that has been obtained from an in vivo tissue or organ and is substantially free of extracellular matrix.

Polypeptides include “polypeptide variants.” Polypeptide variants may differ from a naturally occurring polypeptide in one or more substitutions, deletions, additions and/or insertions. Such variants may be naturally occurring or may be synthetically generated, for example, by modifying one or more of the above polypeptide sequences. For example, in particular embodiments, it may be desirable to improve the binding affinity and/or other biological properties of the CARs by introducing one or more substitutions, deletions, additions and/or insertions into a binding domain, hinge, TM domain, co-stimulatory signaling domain or primary signaling domain of a CAR polypeptide. Preferably, polypeptides of the invention include polypeptides having at least about 65%, 70%, 75%, 85%, 90%, 95%, 98%, or 99% amino acid identity thereto.

Polypeptides include “polypeptide fragments.” Polypeptide fragments refer to a polypeptide, which can be monomeric or multimeric, that has an amino-terminal deletion, a carboxyl-terminal deletion, and/or an internal deletion or substitution of a naturally-occurring or recombinantly-produced polypeptide. In certain embodiments, a polypeptide fragment can comprise an amino acid chain at least 5 to about 500 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, or 450 amino acids long. Particularly useful polypeptide fragments include functional domains, including antigen-binding domains or fragments of antibodies. In the case of a murine anti-BCMA antibody, useful fragments include, but are not limited to: a CDR region, a CDR3 region of the heavy or light chain; a variable region of a heavy or light chain; a portion of an antibody chain or variable region including two CDRs; and the like.

The polypeptide may also be fused in-frame or conjugated to a linker or other sequence for ease of synthesis, purification or identification of the polypeptide (e.g., poly-His), or to enhance binding of the polypeptide to a solid support.

As noted above, polypeptides of the invention may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

In some embodiments, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides of the present invention and still obtain a functional molecule that encodes a variant or derivative polypeptide with desirable characteristics. When it is desired to alter the amino acid sequence of a polypeptide to create an equivalent, or even an improved, variant polypeptide of the invention, one skilled in the art, for example, can change one or more of the codons of the encoding DNA sequence, e.g., according to Table 1.

TABLE 1
Amino Acid Codons

| | One | Three | | | | | | |
|---|---|---|---|---|---|---|---|---|
| Amino | letter | letter | | | | | | |
| Acids | code | code | Codons | | | | | |
| Alanine | A | Ala | GCA | GCC | GCG | GCU | | |
| Cysteine | C | Cys | UGC | UGU | | | | |
| Aspartic | D | Asp | GAC | GAU | | | | |
| acid | | | | | | | | |
| Glutamic | E | Glu | GAA | GAG | | | | |
| acid | | | | | | | | |
| Phenylalanine | F | Phe | UUC | UUU | | | | |
| Glycine | G | Gly | GGA | GGC | GGG | GGU | | |
| Histidine | H | His | CAC | CAU | | | | |
| Isoleucine | I | Iso | AUA | AUC | AUU | | | |
| Lysine | K | Lys | AAA | AAG | | | | |
| Leucine | L | Leu | UUA | UUG | CUA | CUC | CUG | CUU |
| Methionine | M | Met | AUG | | | | | |
| Asparagine | N | Asn | AAC | AAU | | | | |
| Proline | P | Pro | CCA | CCC | CCG | CCU | | |
| Glutamine | Q | Gln | CAA | CAG | | | | |
| Arginine | R | Arg | AGA | AGG | CGA | CGC | CGG | CGU |
| Serine | S | Ser | AGC | AGU | UCA | UCC | UCG | UCU |
| Threonine | T | Thr | ACA | ACC | ACG | ACU | | |
| Valine | V | Val | GUA | GUC | GUG | GUU | | |
| Tryptophan | W | Trp | UGG | | | | | |
| Tyrosine | Y | Tyr | UAC | UAU | | | | |

Guidance in determining which amino acid residues can be substituted, inserted, or deleted without abolishing biological activity can be found using computer programs well known in the art, such as DNASTAR™ software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Exemplary conservative substitutions are described in U.S. Provisional Patent Application No. 61/241,647, the disclosure of which is herein incorporated by reference.

In making such changes, the hydropathic index of amino acids may be considered. The importance of the hydropathic amino acid index in conferring interactive biologic function on a protein is generally understood in the art (Kyte and Doolittle, 1982, incorporated herein by reference). Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

Polypeptide variants further include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

In some embodiments, where expression of two or more polypeptides is desired, the polynucleotide sequences encoding them can be separated by and IRES sequence as discussed elsewhere herein. In another embodiment, two or more polypeptides can be expressed as a fusion protein that comprises one or more self-cleaving polypeptide sequences.

Polypeptides of the present invention include fusion polypeptides. In some embodiments, fusion polypeptides and polynucleotides encoding fusion polypeptides are provided, e.g., CARs. Fusion polypeptides and fusion proteins refer to a polypeptide having at least two, three, four, five, six, seven, eight, nine, or ten or more polypeptide segments. Fusion polypeptides are typically linked C-terminus to N-terminus, although they can also be linked C-terminus to C-terminus, N-terminus to N-terminus, or N-terminus to C-terminus. The polypeptides of the fusion protein can be in any order or a specified order. Fusion polypeptides or fusion proteins can also include conservatively modified variants, polymorphic variants, alleles, mutants, subsequences, and interspecies homologs, so long as the desired transcriptional activity of the fusion polypeptide is preserved. Fusion polypeptides may be produced by chemical synthetic methods or by chemical linkage between the two moieties or may generally be prepared using other standard techniques. Ligated DNA sequences comprising the fusion polypeptide are operably linked to suitable transcriptional or translational control elements as discussed elsewhere herein.

In one embodiment, a fusion polypeptide comprises a sequence that assists in expressing the protein (an expression enhancer) at higher yields than the native recombinant protein. Other fusion partners may be selected so as to increase the solubility of the protein or to enable the protein to be targeted to desired intracellular compartments or to facilitate transport of the fusion protein through the cell membrane.

Fusion polypeptides may further comprise a polypeptide cleavage signal between each of the polypeptide domains described herein. In addition, polypeptide site can be put into any linker peptide sequence. Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5(8); 616-26).

Suitable protease cleavages sites and self-cleaving peptides are known to the skilled person (see, e.g., in Ryan et al., 1997. J. Gener. Virol. 78, 699-722; Scymczak et al. (2004) Nature Biotech. 5, 589-594). Exemplary protease cleavage sites include, but are not limited to the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are preferred in one embodiment, e.g., EXXYXQ(G/S) (SEQ ID NO: 36), for example, ENLYFQG (SEQ ID NO: 37) and ENLYFQS (SEQ ID NO: 38), wherein X represents any amino acid (cleavage by TEV occurs between Q and G or Q and S).

In particular embodiments, the polypeptide cleavage signal is a viral self-cleaving peptide.

In some embodiments, self-cleaving peptides include those polypeptide sequences obtained from aphthovirus, potyvirus and cardiovirus 2A peptides, FMDV (foot-and-mouth disease virus), equine rhinitis A virus, Thosea asigna virus and porcine teschovirus.

In some embodiments, the self-cleaving polypeptide site comprises a 2A or 2A-like site, sequence or domain (Donnelly et al., 2001. J. Gen. Virol. 82:1027-1041). Illustrative examples of 2A sites are provided in Table 2.

TABLE 2

| | |
|---|---|
| SEQ ID NO: 26 | LLNFDLLKLAGDVESNPGP |
| SEQ ID NO: 27 | TLNFDLLKLAGDVESNPGP |
| SEQ ID NO: 28 | LLKLAGDVESNPGP |
| SEQ ID NO: 29 | NFDLLKLAGDVESNPGP |
| SEQ ID NO: 30 | QLLNFDLLKLAGDVESNPGP |
| SEQ ID NO: 31 | APVKQTLNFDLLKLAGDVESNPGP |
| SEQ ID NO: 32 | VTELLYRMKRAETYCPRPLLAIHP |
| | TEARHKQKIVAPVKQT |
| SEQ ID NO: 33 | LNFDLLKLAGDVESNPGP |
| SEQ ID NO: 34 | LLAIHPTEARHKQKIVAPVKQTLN |
| | FDLLKLAGDVESNPGP |
| SEQ ID NO: 35 | EARHKQKIVAPVKQTLNFDLLKLA |
| | GDVESNPGP |

In preferred embodiments, a polypeptide contemplated herein comprises a CAR polypeptide.

### F. Polynucleotides

In some embodiments, a polynucleotide encoding one or more CAR polypeptides is provided, e.g., SEQ ID NOs: 10 and 12. As used herein, the terms “polynucleotide” or “nucleic acid” refers to pre-messenger RNA (pre-mRNA), messenger RNA (mRNA), RNA, genomic RNA (gRNA), plus strand RNA (RNA(+)), minus strand RNA (RNA(−)), genomic DNA (gDNA), PCR amplified DNA, complementary DNA (cDNA), synthetic DNA, or recombinant DNA. Polynucleotides include single and double stranded polynucleotides.

Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxyribonucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc. Preferably, polynucleotides of the invention include polynucleotides or variants having at least about 50%, 55%, 60%, 65%, 70%, 75%, 80%, 85%, 90%, 95%, 96%, 97%, 98%, 99% or 100% sequence identity to any of the reference sequences described herein (see, e.g., Sequence Listing), typically where the variant maintains at least one biological activity of the reference sequence. In various illustrative embodiments, the present disclosure contemplates, in part, polynucleotides comprising expression vectors, viral vectors, and transfer plasmids, and compositions, and cells comprising the same.

In some embodiments, polynucleotides are provided by this disclosure that encode at least about 5, 10, 25, 50, 100, 150, 200, 250, 300, 350, 400, 500, 1000, 1250, 1500, 1750, or 2000 or more contiguous amino acid residues of a polypeptide of the invention, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted, or replaced with different nucleotides compared to a reference polynucleotide. In this regard, it is well understood in the art that certain alterations or modifications inclusive of mutations, additions, deletions and substitutions can be made to a reference polynucleotide whereby the altered or modified polynucleotide retains the biological function or activity of the reference polynucleotide. The term “polynucleotide fragment” may refer to a polynucleotide at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, 450, 500, 550, 600, 650, 700, 750, 800, 850, 900, 950, 1000, 1100, 1200, 1300, 1400, 1500, 1600, 1700 or more nucleotides in length that encodes a polypeptide variant that retains at least 100%, at least 90%, at least 80%, at least 70%, at least 60%, at least 50%, at least 40%, at least 30%, at least 20%, at least 10%, or at least 5% of the naturally occurring polypeptide activity.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 86%, 97%, 98%, 99%, or 100% sequence identity to any of the reference sequences described herein, typically where the polypeptide variant maintains at least one biological activity of the reference polypeptide.

Terms used to describe sequence relationships between two or more polynucleotides or polypeptides include “reference sequence,” “comparison window,” “sequence identity,” “percentage of sequence identity,” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc, 1994-1998, Chapter 15.

In some embodiments, an anti-MUC16 CAR is encoded by the nucleotide sequence set forth in SEQ ID NO: 10. In some embodiments, an anti-MUC16 CAR is encoded by a nucleotide sequence having at least 90% identity to SEQ ID NO: 10. In some embodiments, an anti-MUC16 CAR is encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 10. In some embodiments, an anti-MUC16 CAR comprising the amino acid sequence of SEQ ID NO: 9 is encoded by a nucleotide sequence having at least 90% identity to SEQ ID NO: 10.

In some embodiments, an anti-MUC16 CAR is encoded by the nucleotide sequence set forth in SEQ ID NO: 12. In some embodiments, an anti-MUC16 CAR is encoded by a nucleotide sequence having at least 90% identity to SEQ ID NO: 12. In some embodiments, an anti-MUC16 CAR is encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 12. In some embodiments, an anti-MUC16 CAR comprising the amino acid sequence of SEQ ID NO: 11 is encoded by a nucleotide sequence having at least 90% identity to SEQ ID NO: 12.

In some embodiments, disclosed herein is a nucleotide sequence encoding an anti-MUC16 CAR comprising the sequence of any one of SEQ ID NOs: 49, 50, 51, 52, 61, 62, 63, 64, 73, 74, 75, 76, 85, 86, 87, 88, 97, 98, 99, 100, 109, 110, 111, 112, 121, 122, 123, 124, 125, 126, 135, 136, 137, 138, 147, 148, 149 or 150. In some embodiments, disclosed herein is a nucleotide sequence encoding an anti-MUC16 CAR having at least 90%, 91%, 92%, 93%, 94%, 95%, 96%, 97%, 98%, or 99% identity to any one of SEQ ID NOs: 49, 50, 51, 52, 61, 62, 63, 64, 73, 74, 75, 76, 85, 86, 87, 88, 97, 98, 99, 100, 109, 110, 111, 112, 121, 122, 123, 124, 125, 126, 135, 136, 137, 138, 147, 148, 149 or 150.

As used herein, “isolated polynucleotide” refers to a polynucleotide that has been purified from the sequences which flank it in a naturally-occurring state, e.g., a DNA fragment that has been removed from the sequences that are normally adjacent to the fragment. An “isolated polynucleotide” also refers to a complementary DNA (cDNA), a recombinant DNA, or other polynucleotide that does not exist in nature and that has been made by the hand of man. In particular embodiments, an isolated polynucleotide is a synthetic polynucleotide, a semi-synthetic polynucleotide, or a polynucleotide obtained or derived from a recombinant source.

In various embodiments, a polynucleotide comprises an mRNA encoding a polypeptide contemplated herein. In certain embodiments, the mRNA comprises a cap, one or more nucleotides, and a poly(A) tail.

In particular embodiments, polynucleotides may be codon-optimized. As used herein, the term “codon-optimized” refers to substituting codons in a polynucleotide encoding a polypeptide in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, (x) systematic variation of codon sets for each amino acid, and/or (xi) isolated removal of spurious translation initiation sites.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the premessenger (premRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

The terms “complementary” and “complementarity” refer to polynucleotides (i.e., a sequence of nucleotides) related by the base-pairing rules. For example, the complementary strand of the DNA sequence5′ A G T C A T G 3′ is 3′ T C A G T A C 5′. The latter sequence is often written as the reverse complement with the 5′ end on the left and the 3′ end on the right, 5′ C A T G A C T 3′. A sequence that is equal to its reverse complement is said to be a palindromic sequence. Complementarity can be “partial,” in which only some of the nucleic acids' bases are matched according to the base pairing rules. Or, there can be “complete” or “total” complementarity between the nucleic acids.

Moreover, it will be appreciated by those of ordinary skill in the art that, as a result of the degeneracy of the genetic code, there are many nucleotide sequences that encode a polypeptide, or fragment of variant thereof, as described herein. Some of these polynucleotides bear minimal homology to the nucleotide sequence of any native gene. Nonetheless, polynucleotides that vary due to differences in codon usage are specifically contemplated by the present invention, for example polynucleotides that are optimized for human and/or primate codon selection. Further, alleles of the genes comprising the polynucleotide sequences provided herein may also be used. Alleles are endogenous genes that are altered as a result of one or more mutations, such as deletions, additions and/or substitutions of nucleotides.

The term “nucleic acid cassette” or “expression cassette” as used herein refers to genetic sequences within a vector which can express a RNA, and subsequently a protein. The nucleic acid cassette contains the gene of interest, e.g., a polynucleotide of interest. The nucleic acid cassette contains one or more expression control sequences, e.g., a promoter, enhancer, poly(A) sequence, and a gene of interest, e.g., a polynucleotide of interest. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. In some embodiments, the cassette has its 3′ and 5′ ends adapted for ready insertion into a vector, e.g., it has restriction endonuclease sites at each end. In some embodiments, the nucleic acid cassette contains the sequence of a chimeric antigen receptor. The cassette can be removed and inserted into a plasmid or viral vector as a single unit.

In some embodiments, polynucleotides include at least one polynucleotide-of-interest. As used herein, the term “polynucleotide-of-interest” refers to a polynucleotide encoding a polypeptide (i.e., a polypeptide-of-interest), inserted into an expression vector that is desired to be expressed. A vector may comprise 1, 2, 3, 4, 5, 6, 7, 8, 9, or 10 polynucleotides-of-interest. In some embodiments, the polynucleotide-of-interest encodes a polypeptide that provides a therapeutic effect in the treatment or prevention of a disease or disorder. Polynucleotides-of-interest, and polypeptides encoded therefrom, include both polynucleotides that encode wild-type polypeptides, as well as functional variants and fragments thereof. In particular embodiments, a functional variant has at least 80%, at least 90%, at least 95%, or at least 99% identity to a corresponding wild-type reference polynucleotide or polypeptide sequence. In some embodiments, a functional variant or fragment has at least 50%, at least 60%, at least 70%, at least 80%, or at least 90% of a biological activity of a corresponding wild-type polypeptide.

The polynucleotides of the present disclosure, regardless of the length of the coding sequence itself, may be combined with other DNA sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed, with the total length preferably being limited by the ease of preparation and use in the intended recombinant DNA protocol.

In some embodiments, a polynucleotide or cell harboring the polynucleotide utilizes a suicide gene, including an inducible suicide gene to reduce the risk of direct toxicity and/or uncontrolled proliferation. In some embodiments, the suicide gene is not immunogenic to the host harboring the polynucleotide or cell. A certain example of a suicide gene that may be used is caspase-9 or caspase-8 or cytosine deaminase. Caspase-9 can be activated using a specific chemical inducer of dimerization (CID).

In some embodiments, vectors comprise gene segments that cause the immune effector cells of the invention, e.g., T cells, to be susceptible to negative selection in vivo. By “negative selection” is meant that the infused cell can be eliminated as a result of a change in the in vivo condition of the individual. The negative selectable phenotype may result from the insertion of a gene that confers sensitivity to an administered agent, for example, a compound. Negative selectable genes are known in the art, and include, inter alia the following: the Herpes simplex virus type I thymidine kinase (HSV-I TK) gene (Wigler et al., Cell 11:223, 1977) which confers ganciclovir sensitivity; the cellular hypoxanthine phosphoribosyltransferase (HPRT) gene, the cellular adenine phosphoribosyltransferase (APRT) gene, and bacterial cytosine deaminase, (Mullen et al., Proc. Natl. Acad. Sci. USA. 89:33 (1992)).

In some embodiments, genetically modified immune effector cells, such as T cells, comprise a polynucleotide further comprising a positive marker that enables the selection of cells of the negative selectable phenotype in vitro. The positive selectable marker may be a gene which, upon being introduced into the host cell expresses a dominant phenotype permitting positive selection of cells carrying the gene. Genes of this type are known in the art, and include, inter alia, hygromycin-B phosphotransferase gene (hph) which confers resistance to hygromycin B, the amino glycoside phosphotransferase gene (neo or aph) from Tn5 which codes for resistance to the antibiotic G418, the dihydrofolate reductase (DHFR) gene, the adenosine deaminase gene (ADA), and the multi-drug resistance (MDR) gene.

In some embodiments, the positive selectable marker and the negative selectable element are linked such that loss of the negative selectable element necessarily also is accompanied by loss of the positive selectable marker. In some embodiments, the positive and negative selectable markers are fused so that loss of one obligatorily leads to loss of the other. An example of a fused polynucleotide that yields as an expression product a polypeptide that confers both the desired positive and negative selection features described above is a hygromycin phosphotransferase thymidine kinase fusion gene (HyTK). Expression of this gene yields a polypeptide that confers hygromycin B resistance for positive selection in vitro, and ganciclovir sensitivity for negative selection in vivo. See Lupton S. D., et al, Mol. and Cell. Biology 1 1:3374-3378, 1991. In some embodiments, the polynucleotides encoding the CARs are in retroviral vectors containing the fused gene, particularly those that confer hygromycin B resistance for positive selection in vitro, and ganciclovir sensitivity for negative selection in vivo, for example the HyTK retroviral vector described in Lupton, S. D. et al. (1991), supra. See also the publications of PCT US91/08442 and PCT/US94/05601, by S. D. Lupton, describing the use of bifunctional selectable fusion genes derived from fusing a dominant positive selectable markers with negative selectable markers.

In some embodiments, positive selectable markers are derived from genes selected from the group consisting of hph, nco, and gpt, and negative selectable markers are derived from genes selected from the group consisting of cytosine deaminase, HSV-I TK, VZV TK, HPRT, APRT and gpt. In some embodiments, markers are bifunctional selectable fusion genes wherein the positive selectable marker is derived from hph or neo, and the negative selectable marker is derived from cytosine deaminase or a TK gene or selectable marker.

### Inducible Suicide Genes

### G. Vectors

Polynucleotides can be prepared, manipulated and/or expressed using any of a variety of well-established techniques known and available in the art. In order to express a desired polypeptide, a nucleotide sequence encoding the polypeptide, can be inserted into appropriate vector.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA.

Exemplary vectors include, without limitation, autonomously replicating sequences, mRNA, plasmids (e.g., DNA plasmids or RNA plasmids), transposons, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. In some embodiments, the coding sequences of the chimeric proteins disclosed herein can be ligated into such expression vectors for the expression of the chimeric protein in mammalian cells.

In some embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally. The vector is engineered to harbor the sequence coding for the origin of DNA replication or “ori” from a lymphotrophic herpes virus or a gamma herpesvirus, an adenovirus, SV40, a bovine papilloma virus, or a yeast, specifically a replication origin of a lymphotrophic herpes virus or a gamma herpesvirus corresponding to oriP of EBV. In a particular aspect, the lymphotrophic herpes virus may be Epstein Barr virus (EBV), Kaposi's sarcoma herpes virus (KSHV), Herpes virus saimiri (HS), or Marek's disease virus (MDV). Epstein Barr virus (EBV) and Kaposi's sarcoma herpes virus (KSHV) are also examples of a gamma herpesvirus. Typically, the host cell comprises the viral replication transactivator protein that activates the replication.

The “control elements” or “regulatory sequences” present in an expression vector are those non-translated regions of the vector-origin of replication, selection cassettes, promoters, enhancers, translation initiation signals (Shine Dalgarno sequence or Kozak sequence) introns, a polyadenylation sequence, 5′ and 3′ untranslated regions-which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In some embodiments, vectors include, but are not limited to expression vectors and viral vectors, will include exogenous, endogenous, or heterologous control sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In some embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments of the invention include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (O-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, dl587rev primer-binding site substituted (MND) promoter (Challita et al., J Virol. 69(2):748-55 (1995)).

In some embodiments, a vector comprises a MND promoter. In some embodiments, a vector comprises an EF1a promoter comprising the first intron of the human EF1a gene. In some embodiments, a vector comprises an EF1a promoter that lacks the first intron of the human EF1a gene.

In a particular embodiment, it may be desirable to express a polynucleotide comprising a CAR from a T cell specific promoter.

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. In some embodiments, conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Conditional expression can also be achieved by using a site specific DNA recombinase. According to certain embodiments of the invention the vector comprises at least one (typically two) site(s) for recombination mediated by a site specific recombinase. As used herein, the terms “recombinase” or “site specific recombinase” include excisive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases suitable include, but are not limited to: Cre, Int, IHF, Xis, Flp, Fis, Hin, Gin, ϕC31, Cin, Tn3 resolvase, TndX, XerC, XerD, TnpX, Hjc, Gin, SpCCE1, and ParA.

The vectors may comprise one or more recombination sites for any of a wide variety of site specific recombinases. It is to be understood that the target site for a site specific recombinase is in addition to any site(s) required for integration of a vector, e.g., a retroviral vector or lentiviral vector. As used herein, the terms “recombination sequence,” “recombination site,” or “site specific recombination site” refer to a particular nucleic acid sequence to which a recombinase recognizes and binds.

For example, one recombination site for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Other exemplary loxP sites include, but are not limited to: lox511 (Hoess et al., 1996; Bethke and Sauer, 1997), lox5171 (Lee and Saito, 1998), lox2272 (Lee and Saito, 1998), m2 (Langer et al., 2002), lox71 (Albert et al., 1995), and lox66 (Albert et al., 1995).

Suitable recognition sites for the FLP recombinase include, but are not limited to: FRT (McLeod, et al., 1996), F₁, F₂, F₃ (Schlake and Bode, 1994), F₄, F₅ (Schlake and Bode, 1994), FRT(LE) (Senecoff et al., 1988), FRT(RE) (Senecoff et al., 1988).

Other examples of recognition sequences are the attB, attP, attL, and attR sequences, which are recognized by the recombinase enzyme λ Integrase, e.g., phi-c31. The φC31 SSR mediates recombination only between the heterotypic sites attB (34 bp in length) and attP (39 bp in length) (Groth et al., 2000). attB and attP, named for the attachment sites for the phage integrase on the bacterial and phage genomes, respectively, both contain imperfect inverted repeats that are likely bound by pC31 homodimers (Groth et al., 2000). The product sites, attL and attR, are effectively inert to further pC31-mediated recombination (Belteki et al., 2003), making the reaction irreversible. For catalyzing insertions, it has been found that attB-bearing DNA inserts into a genomic attP site more readily than an attP site into a genomic attB site (Thyagarajan et al., 2001; Belteki et al., 2003). Thus, typical strategies position by homologous recombination an attP-bearing “docking site” into a defined locus, which is then partnered with an attB-bearing incoming sequence for insertion.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. In some embodiments, vectors include one or more polynucleotides-of-interest that encode one or more polypeptides. In some embodiments, to achieve efficient translation of each of the plurality of polypeptides, the polynucleotide sequences can be separated by one or more IRES sequences or polynucleotide sequences encoding self-cleaving polypeptides.

As used herein, the term “Kozak sequence” refers to a short nucleotide sequence that greatly facilitates the initial binding of mRNA to the small subunit of the ribosome and increases translation. The consensus Kozak sequence is (GCC)RCCATGG, where R is a purine (A or G) (Kozak, 1986. Cell. 44(2):283-92, and Kozak, 1987. Nucleic Acids Res. 15(20):8125-48). In some embodiments, the vectors comprise polynucleotides that have a consensus Kozak sequence and that encode a desired polypeptide, e.g., a CAR.

### 1. Viral Vectors

In some embodiments, a cell (e.g., an immune effector cell) is transduced with a viral vector, e.g., a lentiviral vector, encoding a CAR. For example, an immune effector cell is transduced with a vector encoding a CAR that comprises an anti-MUC16 antibody or antigen binding fragment thereof that binds a MUC16 polypeptide, with an intracellular signaling domain of CD3ζ, CD28, 4-1BB, Ox40, or any combinations thereof. Thus, these transduced cells can elicit a CAR-mediated cytotoxic response.

Illustrative examples of viral vector systems suitable for use in particular embodiments contemplated herein include but are not limited to adeno-associated virus (AAV), retrovirus, herpes simplex virus, adenovirus, and vaccinia virus vectors.

Adenoviral based vectors are capable of very high transduction efficiency in many cell types and do not require cell division. With such vectors, high titer and high levels of expression have been obtained. This vector can be produced in large quantities in a relatively simple system. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity.

Generation and propagation of the current adenovirus vectors, which are replication deficient, may utilize a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones & Shenk, 1978), the current adenovirus vectors, with the help of 293 cells, carry foreign DNA in either the E1, the D3 or both regions (Graham & Prevec, 1991). Adenovirus vectors have been used in eukaryotic gene expression (Levrero et al., 1991; Gomez-Foix et al., 1992) and vaccine development (Grunhaus & Horwitz, 1992; Graham & Prevec, 1992). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993). An example of the use of an Ad vector in a clinical trial involved polynucleotide therapy for antitumor immunization with intramuscular injection (Sterman et al., Hum. Gene Ther. 7:1083-9 (1998)).

In some embodiments, one or more polynucleotides encoding a polycistronic message encoding an anti-MUC16 CAR are introduced into an immune effector cell, e.g., a T cell, by transducing the cell with a recombinant adeno-associated virus (rAAV), comprising the one or more polynucleotides.

AAV is a small (˜26 nm) replication-defective, primarily episomal, non-enveloped virus. AAV can infect both dividing and non-dividing cells and may incorporate its genome into that of the host cell. Recombinant AAV (rAAV) are typically composed of, at a minimum, a transgene and its regulatory sequences, and 5′ and 3′ AAV inverted terminal repeats (ITRs). The ITR sequences are about 145 bp in length. In some embodiments, the rAAV comprises ITRs and capsid sequences isolated from AAV1, AAV2, AAV3, AAV4, AAV5, AAV6, AAV7, AAV8, AAV9, or AAV10.

In some embodiments, a chimeric rAAV is used the ITR sequences are isolated from one AAV serotype and the capsid sequences are isolated from a different AAV serotype. For example, a rAAV with ITR sequences derived from AAV2 and capsid sequences derived from AAV6 is referred to as AAV2/AAV6. In particular embodiments, the rAAV vector may comprise ITRs from AAV2, and capsid proteins from any one of AAV1, AAV2, AAV3, AAV4, AAV5, AAV6, AAV7, AAV8, AAV9, or AAV10. In some embodiments, the rAAV comprises ITR sequences derived from AAV2 and capsid sequences derived from AAV6. In some embodiments, the rAAV comprises ITR sequences derived from AAV2 and capsid sequences derived from AAV2.

In some embodiments, engineering and selection methods can be applied to AAV capsids to make them more likely to transduce cells of interest.

Construction of rAAV vectors, production, and purification thereof have been disclosed, e.g., in U.S. Pat. Nos. 9,169,494; 9,169,492; 9,012,224; 8,889,641; 8,809,058; and 8,784,799, each of which is incorporated by reference herein, in its entirety.

In various embodiments, one or more polynucleotides encoding a CAR are introduced into an immune effector cell by transducing the cell with a herpes simplex virus, e.g., HSV-1, HSV-2, comprising the one or more polynucleotides. In some embodiments, one or more polynucleotides encoding a polycistronic message encoding a CAR are introduced into an immune effector cell by transducing the cell with a herpes simplex virus, e.g., HSV-1, HSV-2, comprising the one or more polynucleotides.

The mature HSV virion consists of an enveloped icosahedral capsid with a viral genome consisting of a linear double-stranded DNA molecule that is 152 kb. In one embodiment, the HSV based viral vector is deficient in one or more essential or non-essential HSV genes. In one embodiment, the HSV based viral vector is replication deficient. Most replication deficient HSV vectors contain a deletion to remove one or more intermediate-early, early, or late HSV genes to prevent replication. For example, the HSV vector may be deficient in an immediate early gene selected from the group consisting of: ICP4, ICP22, ICP27, ICP47, and a combination thereof. Advantages of the HSV vector are its ability to enter a latent stage that can result in long-term DNA expression and its large viral DNA genome that can accommodate exogenous DNA inserts of up to 25 kb. HSV-based vectors are described in, for example, U.S. Pat. Nos. 5,837,532, 5,846,782, and 5,804,413, and International Patent Applications WO 91/02788, WO 96/04394, WO 98/15637, and WO 99/06583, each of which are incorporated by reference herein in its entirety.

Retroviruses are a common tool for gene delivery (Miller, 2000, Nature. 357: 455-460). In particular embodiments, a retrovirus is used to deliver a polynucleotide encoding a chimeric antigen receptor (CAR) to a cell. As used herein, the term “retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred. In some embodiments, a lentivirus is used to deliver a polynucleotide comprising a CAR to a cell.

Retroviral vectors and more particularly lentiviral vectors may be used in practicing particular embodiments of the present disclosure. Accordingly, the term “retrovirus” or “retroviral vector”, as used herein is meant to include “lentivirus” and “lentiviral vectors” respectively.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus. The term “hybrid vector” refers to a vector, LTR or other nucleic acid containing both retroviral, e.g., lentiviral, sequences and non-lentiviral viral sequences. In one embodiment, a hybrid vector refers to a vector or transfer plasmid comprising retroviral e.g., lentiviral, sequences for reverse transcription, replication, integration and/or packaging.

In some embodiments, the terms “lentiviral vector,” “lentiviral expression vector” may be used to refer to lentiviral transfer plasmids and/or infectious lentiviral particles. Where reference is made herein to elements such as cloning sites, promoters, regulatory elements, heterologous nucleic acids, etc., it is to be understood that the sequences of these elements are present in RNA form in the lentiviral particles and are present in DNA form in the DNA plasmids.

At each end of the provirus are structures called “long terminal repeats” or “LTRs.” The term “long terminal repeat (LTR)” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer binding site and the R region and contains the polyadenylation sequence. The R (repeat) region is flanked by the U3 and U5 regions. The LTR composed of U3, R and U5 regions and appears at both the 5′ and 3′ ends of the viral genome. Adjacent to the 5′ LTR are sequences necessary for reverse transcription of the genome (the tRNA primer binding site) and for efficient packaging of viral RNA into particles (the Psi site).

As used herein, the term “packaging signal” or “packaging sequence” refers to sequences located within the retroviral genome which are required for insertion of the viral RNA into the viral capsid or particle, see e.g., Clever et al., 1995. J. of Virology, Vol. 69, No. 4; pp. 2101-2109. Several retroviral vectors use the minimal packaging signal (also referred to as the psi [Ψ] sequence) needed for encapsidation of the viral genome. Thus, as used herein, the terms “packaging sequence,” “packaging signal,” “psi” and the symbol “T,” are used in reference to the non-coding sequence required for encapsidation of retroviral RNA strands during viral particle formation.

In various embodiments, vectors comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective. As used herein, the term “replication-defective” refers to virus that is not capable of complete, effective replication such that infective virions are not produced (e.g., replication-defective lentiviral progeny). The term “replication-competent” refers to wild-type virus or mutant virus that is capable of replication, such that viral replication of the virus is capable of producing infective virions (e.g., replication-competent lentiviral progeny).

“Self-inactivating” (SIN) vectors refers to replication-defective vectors, e.g., retroviral or lentiviral vectors, in which the right (3′) LTR enhancer-promoter region, known as the U3 region, has been modified (e.g., by deletion or substitution) to prevent viral transcription beyond the first round of viral replication. This is because the right (3′) LTR U3 region is used as a template for the left (5′) LTR U3 region during viral replication and, thus, the viral transcript cannot be made without the U3 enhancer-promoter. In a further embodiment of the invention, the 3′ LTR is modified such that the U5 region is replaced, for example, with an ideal poly(A) sequence. It should be noted that modifications to the LTRs such as modifications to the 3′ LTR, the 5′ LTR, or both 3′ and 5′ LTRs, are also included in the invention.

An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR element. The term “TAR” refers to the “trans-activation response” genetic element located in the R region of lentiviral (e.g., HIV) LTRs. This element interacts with the lentiviral trans-activator (tat) genetic element to enhance viral replication. However, this element is not required in embodiments wherein the U3 region of the 5′ LTR is replaced by a heterologous promoter.

The “R region” refers to the region within retroviral LTRs beginning at the start of the capping group (i.e., the start of transcription) and ending immediately prior to the start of the poly A tract. The R region is also defined as being flanked by the U3 and U5 regions. The R region plays a role during reverse transcription in permitting the transfer of nascent DNA from one end of the genome to the other.

As used herein, the term “FLAP element” or “cPPT/FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173. During HIV-1 reverse transcription, central initiation of the plus-strand DNA at the central polypurine tract (cPPT) and central termination at the central termination sequence (CTS) lead to the formation of a three-stranded DNA structure: the HIV-1 central DNA flap. While not wishing to be bound by any theory, the DNA flap may act as a cis-active determinant of lentiviral genome nuclear import and/or may increase the titer of the virus. In particular embodiments, the retroviral or lentiviral vector backbones comprise one or more FLAP elements upstream or downstream of the heterologous genes of interest in the vectors. For example, in particular embodiments a transfer plasmid includes a FLAP element. In one embodiment, a vector of the invention comprises a FLAP element isolated from HIV-1. In another embodiment, a lentiviral vector contains a FLAP element with one or more mutations in the cPPT and/or CTS elements. In yet another embodiment, a lentiviral vector comprises either a cPPT or CTS element. In yet another embodiment, a lentiviral vector does not comprise a cPPT or CTS element.

In some embodiments, retroviral or lentiviral transfer vectors comprise one or more export elements. The term “export element” refers to a cis-acting posttranscriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus posttranscriptional regulatory element (HPRE). Generally, the RNA export element is placed within the 3′ UTR of a gene, and can be inserted as one or multiple copies.

In some embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766). In some embodiments, vectors comprise a posttranscriptional regulatory element such as a WPRE or HPRE

In some embodiments, vectors lack or do not comprise a posttranscriptional regulatory element (PTE) such as a WPRE or HPRE because in some instances these elements increase the risk of cellular transformation and/or do not substantially or significantly increase the amount of mRNA transcript or increase mRNA stability. Therefore, in some embodiments, vectors lack or do not comprise a PTE. In some embodiments, vectors lack or do not comprise a WPRE or HPRE as an added safety measure.

Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed. The term “polyA site” or “polyA sequence” as used herein denotes a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Efficient polyadenylation of the recombinant transcript is desirable as transcripts lacking a poly A tail are unstable and are rapidly degraded. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. Illustrative examples of polyA signals that can be used in a vector of the invention, includes an ideal polyA sequence (e.g., AATAAA, ATTAAA, AGTAAA), a bovine growth hormone polyA sequence (BGHpA), a rabbit (3-globin polyA sequence (rpgpA), or another suitable heterologous or endogenous polyA sequence known in the art.

In some embodiments, a retroviral or lentiviral vector further comprises one or more insulator elements. Insulators elements may contribute to protecting lentivirus-expressed sequences, e.g., therapeutic polypeptides, from integration site effects, which may be mediated by cis-acting elements present in genomic DNA and lead to deregulated expression of transferred sequences (i.e., position effect; see, e.g., Burgess-Beusse et al., 2002, Proc. Natl. Acad. Sci., USA, 99:16433; and Zhan et al., 2001, Hum. Genet., 109:471). In some embodiments, transfer vectors comprise one or more insulator element the 3′ LTR and upon integration of the provirus into the host genome, the provirus comprises the one or more insulators at both the 5′ LTR or 3′ LTR, by virtue of duplicating the 3′ LTR. Suitable insulators for use in the invention include, but are not limited to, the chicken β-globin insulator (see Chung et al., 1993. Cell 74:505; Chung et al., 1997. PNAS 94:575; and Bell et al., 1999. Cell 98:387, incorporated by reference herein). Examples of insulator elements include, but are not limited to, an insulator from an β-globin locus, such as chicken HS4.

In some embodiments, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid.

In some embodiments, vectors comprise a promoter operably linked to a polynucleotide encoding a CAR polypeptide. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Ψ) packaging signal, RRE), and/or other elements that increase therapeutic gene expression (e.g., poly (A) sequences), and may optionally comprise a WPRE or HPRE.

In various embodiments, the vector is an integrating viral vector.

In various other embodiments, the vector is an episomal or non-integrating viral vector.

In various embodiments, vectors contemplated herein, comprise non-integrating or integration defective retrovirus. In one embodiment, an “integration defective” retrovirus or lentivirus refers to retrovirus or lentivirus having an integrase that lacks the capacity to integrate the viral genome into the genome of the host cells. In various embodiments, the integrase protein is mutated to specifically decrease its integrase activity. Integration-incompetent lentiviral vectors are obtained by modifying the pol gene encoding the integrase protein, resulting in a mutated pol gene encoding an integrative deficient integrase. Such integration-incompetent viral vectors have been described in patent application WO 2006/010834, which is herein incorporated by reference in its entirety.

Illustrative mutations in the HIV-1 pol gene suitable to reduce integrase activity include, but are not limited to: H12N, H12C, H16C, H16V, S81R, D41A, K42A, H51A, Q53C, D55V, D64E, D64V, E69A, K71A, E85A, E87A, D116N, D1161, D116A, N120G, N1201, N120E, E152G, E152A, D35E, K156E, K156A, E157A, K159E, K159A, K160A, R166A, D167A, E170A, H171A, K173A, K186Q, K186T, K188T, E198A, R199c, R199T, R199A, D202A, K211A, Q214L, Q216L, Q221 L, W235F, W235E, K236S, K236A, K246A, G247W, D253A, R262A, R263A and K264H.

Illustrative mutations in the HIV-1 pol gene suitable to reduce integrase activity include, but are not limited to: D64E, D64V, E92K, D116N, D1161, D116A, N120G, N1201, N120E, E152G, E152A, D35E, K156E, K156A, E157A, K159E, K159A, W235F, and W235E.

In a particular embodiment, an integrase comprises a mutation in one or more of amino acids, D64, D116 or E152. In one embodiment, an integrase comprises a mutation in the amino acids, D64, D116 and E152. In a particular embodiment, a defective HIV-1 integrase comprises a D64V mutation.

A “host cell” includes cells electroporated, transfected, infected, or transduced in vivo, ex vivo, or in vitro with a recombinant vector or a polynucleotide of the invention. Host cells may include packaging cells, producer cells, and cells infected with viral vectors. In particular embodiments, host cells infected with viral vector of the invention are administered to a subject in need of therapy. In certain embodiments, the term “target cell” is used interchangeably with host cell and refers to transfected, infected, or transduced cells of a desired cell type. In preferred embodiments, the target cell is a T cell.

Viral vectors comprising polynucleotides contemplated in particular embodiments can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., mobilized peripheral blood, lymphocytes, bone marrow aspirates, tissue biopsy, etc.) or universal donor hematopoietic stem cells, followed by reimplantation of the cells into a patient.

In one embodiment, a viral vector comprising a polynucleotide encoding a CAR is administered directly to an organism for transduction of cells in vivo. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Large scale viral particle production is often necessary to achieve a reasonable viral titer. Viral particles are produced by transfecting a transfer vector into a packaging cell line that comprises viral structural and/or accessory genes, e.g., gag, pol, env, tat, rev, vif, vpr, vpu, vpx, or nef genes or other retroviral genes.

As used herein, the term “packaging vector” refers to an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. Methods for transfection, transduction or infection are well known by those of skill in the art. A retroviral/lentiviral transfer vector of the present invention can be introduced into a packaging cell line, via transfection, transduction or infection, to generate a producer cell or cell line. The packaging vectors of the present invention can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides.

Viral envelope proteins (env) determine the range of host cells which can ultimately be infected and transformed by recombinant retroviruses generated from the cell lines. In the case of lentiviruses, such as HIV-1, HIV-2, SIV, FIV and EIV, the env proteins include gp41 and gp120. Preferably, the viral env proteins expressed by packaging cells of the invention are encoded on a separate vector from the viral gag and pol genes, as has been previously described.

Illustrative examples of retroviral-derived env genes which can be employed in the invention include, but are not limited to: MLV envelopes, 10A1 envelope, BAEV, FeLV-B, RD114, SSAV, Ebola, Sendai, FPV (Fowl plague virus), and influenza virus envelopes. Similarly, genes encoding envelopes from RNA viruses (e.g., RNA virus families of Picornaviridae, Calciviridae, Astroviridae, Togaviridae, Flaviviridae, Coronaviridae, Paramyxoviridae, Rhabdoviridae, Filoviridae, Orthomyxoviridae, Bunyaviridae, Arenaviridae, Reoviridae, Birnaviridae, Retroviridae) as well as from the DNA viruses (families of Hepadnaviridae, Circoviridae, Parvoviridae, Papovaviridae, Adenoviridae, Herpesviridae, Poxyiridae, and Iridoviridae) may be utilized. Representative examples include, FeLV, VEE, HFVW, WDSV, SFV, Rabies, ALV, BIV, BLV, EBV, CAEV, SNV, ChTLV, STLV, MPMV, SMRV, RAV, FuSV, MH2, AEV, AMV, CT10, and EIAV.

In other embodiments, envelope proteins for pseudotyping a virus of present invention include, but are not limited to any of the following virus: Influenza A such as H1N1, H1N2, H3N2 and H5N1 (bird flu), Influenza B, Influenza C virus, Hepatitis A virus, Hepatitis B virus, Hepatitis C virus, Hepatitis D virus, Hepatitis E virus, Rotavirus, any virus of the Norwalk virus group, enteric adenoviruses, parvovirus, Dengue fever virus, Monkey pox, Mononegavirales, Lyssavirus such as rabies virus, Lagos bat virus, Mokola virus, Duvenhage virus, European bat virus 1 & 2 and Australian bat virus, Ephemerovirus, Vesiculovirus, Vesicular Stomatitis Virus (VSV), Herpesviruses such as Herpes simplex virus types 1 and 2, varicella zoster, cytomegalovirus, Epstein-Bar virus (EBV), human herpesviruses (HHV), human herpesvirus type 6 and 8, Human immunodeficiency virus (HIV), papilloma virus, murine gammaherpesvirus, Arenaviruses such as Argentine hemorrhagic fever virus, Bolivian hemorrhagic fever virus, Sabia-associated hemorrhagic fever virus, Venezuelan hemorrhagic fever virus, Lassa fever virus, Machupo virus, Lymphocytic choriomeningitis virus (LCMV), Bunyaviridiae such as Crimean-Congo hemorrhagic fever virus, Hantavirus, hemorrhagic fever with renal syndrome causing virus, Rift Valley fever virus, Filoviridae (filovirus) including Ebola hemorrhagic fever and Marburg hemorrhagic fever, Flaviviridae including Kaysanur Forest disease virus, Omsk hemorrhagic fever virus, Tick-borne encephalitis causing virus and Paramyxoviridae such as Hendra virus and Nipah virus, variola major and variola minor (smallpox), alphaviruses such as Venezuelan equine encephalitis virus, eastern equine encephalitis virus, western equine encephalitis virus, SARS-associated coronavirus (SARS-CoV), West Nile virus, any encephaliltis causing virus.

In one embodiment, the invention provides packaging cells which produce recombinant retrovirus, e.g., lentivirus, pseudotyped with the VSV-G glycoprotein.

The terms “pseudotype” or “pseudotyping” as used herein, refer to a virus whose viral envelope proteins have been substituted with those of another virus possessing preferable characteristics. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4+ presenting cells. In a preferred embodiment of the invention, lentiviral envelope proteins are pseudotyped with VSV-G. In one embodiment, the invention provides packaging cells which produce recombinant retrovirus, e.g., lentivirus, pseudotyped with the VSV-G envelope glycoprotein.

As used herein, the term “packaging cell lines” is used in reference to cell lines that do not contain a packaging signal, but do stably or transiently express viral structural proteins and replication enzymes (e.g., gag, pol and env) which are necessary for the correct packaging of viral particles. Any suitable cell line can be employed to prepare packaging cells of the invention. Generally, the cells are mammalian cells. In a particular embodiment, the cells used to produce the packaging cell line are human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In preferred embodiments, the packaging cells are 293 cells, 293T cells, or A549 cells. In another preferred embodiment, the cells are A549 cells.

As used herein, the term “producer cell line” refers to a cell line which is capable of producing recombinant retroviral particles, comprising a packaging cell line and a transfer vector construct comprising a packaging signal. The production of infectious viral particles and viral stock solutions may be carried out using conventional techniques. Methods of preparing viral stock solutions are known in the art and are illustrated by, e.g., Y. Soneoka et al. (1995) Nucl. Acids Res. 23:628-633, and N. R. Landau et al. (1992) J. Virol. 66:5110-5113. Infectious virus particles may be collected from the packaging cells using conventional techniques. For example, the infectious particles can be collected by cell lysis, or collection of the supernatant of the cell culture, as is known in the art. Optionally, the collected virus particles may be purified if desired. Suitable purification techniques are well known to those skilled in the art.

The delivery of a gene(s) or other polynucleotide sequence using a retroviral or lentiviral vector by means of viral infection rather than by transfection is referred to as “transduction.” In one embodiment, retroviral vectors are transduced into a cell through infection and provirus integration. In certain embodiments, a target cell, e.g., a T cell, is “transduced” if it comprises a gene or other polynucleotide sequence delivered to the cell by infection using a viral or retroviral vector. In particular embodiments, a transduced cell comprises one or more genes or other polynucleotide sequences delivered by a retroviral or lentiviral vector in its cellular genome.

In particular embodiments, host cells transduced with viral vector of the invention that expresses one or more polypeptides, are administered to a subject to treat and/or prevent disease. Other methods relating to the use of viral vectors in gene therapy, which may be utilized according to certain embodiments of the present invention, can be found in, e.g., Kay, M. A. (1997) Chest 111(6 Supp.):138S-142S; Ferry, N. and Heard, J. M. (1998) Hum. Gene Ther. 9:1975-81; Shiratory, Y. et al. (1999) Liver 19:265-74; Oka, K. et al. (2000) Curr. Opin. Lipidol. 11:179-86; Thule, P. M. and Liu, J. M. (2000) Gene Ther. 7:1744-52; Yang, N. S. (1992) Crit. Rev. Biotechnol. 12:335-56; Alt, M. (1995) J. Hepatol. 23:746-58; Brody, S. L. and Crystal, R. G. (1994) Ann. N.Y. Acad. Sci. 716:90-101; Strayer, D. S. (1999) Expert Opin. Investig. Drugs 8:2159-2172; Smith-Arica, J. R. and Bartlett, J. S. (2001) Curr. Cardiol. Rep. 3:43-49; and Lee, H. C. et al. (2000) Nature 408:483-8.

### H. Genetically Modified Cells

In some embodiments, the disclosure provides cells genetically modified to express the CARs contemplated herein. In some embodiments, the genetically modified cells are for use in the treatment of cancer (e.g., cancer expressing MUC16). As used herein, the term “genetically engineered” or “genetically modified” refers to the addition of extra genetic material in the form of DNA or RNA into the total genetic material in a cell. The terms, “genetically modified cells,” “modified cells,” and, “redirected cells,” are used interchangeably. As used herein, the term “gene therapy” refers to the introduction of extra genetic material in the form of DNA or RNA into the total genetic material in a cell that restores, corrects, or modifies expression of a gene, or for the purpose of expressing a therapeutic polypeptide, e.g., a CAR.

In some embodiments, the CARs contemplated herein are introduced and expressed in immune effector cells so as to redirect their specificity to a target antigen of interest, e.g., a MUC16 polypeptide. An “immune effector cell,” is any cell of the immune system that has one or more effector functions (e.g., cytotoxic cell killing activity, secretion of cytokines, induction of ADCC and/or CDC).

Immune effector cells of the invention can be autologous/autogeneic (“self”) or non-autologous (“non-self,” e.g., allogeneic, syngeneic or xenogeneic). “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells of the invention are allogeneic.

Illustrative immune effector cells used with the CARs contemplated herein include T lymphocytes. The terms “T cell” or “T lymphocyte” are art-recognized and are intended to include thymocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), CD4⁺CD8⁺ T cell, CD4-CD8⁻ T cell, or any other subset of T cells. Other illustrative populations of T cells suitable for use in particular embodiments include naïve T cells (TN), memory T cells, T memory stem cells (TSCM), central memory T cells (TCM), effector memory T cells (TEM), and effector T cells (TEFF).

As would be understood by the skilled person, other cells may also be used as immune effector cells with the CARs as described herein. In particular, immune effector cells also include NK cells, NKT cells, neutrophils, and macrophages. Immune effector cells also include progenitors of effector cells wherein such progenitor cells can be induced to differentiate into an immune effector cells in vivo or in vitro. Thus, in some embodiments, immune effector cell includes progenitors of immune effectors cells such as hematopoietic stem cells (HSCs) contained within the CD34⁺ population of cells derived from cord blood, bone marrow or mobilized peripheral blood which upon administration in a subject differentiate into mature immune effector cells, or which can be induced in vitro to differentiate into mature immune effector cells.

As used herein, immune effector cells genetically engineered to contain MUC16-specific CAR may be referred to as, “MUC16-specific redirected immune effector cells.”

The term, “CD34+ cell,” as used herein refers to a cell expressing the CD34 protein on its cell surface. “CD34,” as used herein refers to a cell surface glycoprotein (e.g., sialomucin protein) that often acts as a cell-cell adhesion factor and is involved in T cell entrance into lymph nodes. The CD34₊ cell population contains hematopoietic stem cells (HSC), which upon administration to a patient differentiate and contribute to all hematopoietic lineages, including T cells, NK cells, NKT cells, neutrophils and cells of the monocyte/macrophage lineage.

In some embodiments, the present disclosure provides methods for making the immune effector cells which express the CAR contemplated herein. In some embodiments, the method comprises transfecting or transducing immune effector cells isolated from an individual such that the immune effector cells express one or more CAR as described herein. In some embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a CAR. In this regard, the immune effector cells may be cultured before and/or after being genetically modified (i.e., transduced or transfected to express a CAR contemplated herein).

In some embodiments, prior to in vitro manipulation or genetic modification of the immune effector cells described herein, the source of cells is obtained from a subject. In some embodiments, the CAR-modified immune effector cells comprise T cells. T cells can be obtained from a number of sources including, but not limited to, peripheral blood mononuclear cells, bone marrow, lymph nodes tissue, cord blood, thymus issue, tissue from a site of infection, ascites, pleural effusion, spleen tissue, and tumors. In some embodiments, T cells can be obtained from a unit of blood collected from a subject using any number of techniques known to the skilled person, such as sedimentation, e.g., FICOLL™ separation. In some embodiments, cells from the circulating blood of an individual are obtained by apheresis. The apheresis product typically contains lymphocytes, including T cells, monocytes, granulocyte, B cells, other nucleated white blood cells, red blood cells, and platelets. In some embodiments, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. As would be appreciated by those of ordinary skill in the art, a washing step may be accomplished by methods known to those in the art, such as by using a semiautomated flowthrough centrifuge. For example, the Cobe 2991 cell processor, the Baxter CytoMate, or the like. After washing, the cells may be resuspended in a variety of biocompatible buffers or other saline solution with or without buffer. In some embodiments, the undesirable components of the apheresis sample may be removed in the cell directly resuspended culture media.

In some embodiments, T cells are isolated from peripheral blood mononuclear cells (PBMCs) by lysing the red blood cells and depleting the monocytes, for example, by centrifugation through a PERCOLL™ gradient. A specific subpopulation of T cells, expressing one or more of the following markers: CD3, CD28, CD4, CD8, CD45RA, and CD45RO, can be further isolated by positive or negative selection techniques. In one embodiment, a specific subpopulation of T cells, expressing CD3, CD28, CD4, CD8, CD45RA, and CD45RO is further isolated by positive or negative selection techniques. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. Flow cytometry and cell sorting may also be used to isolate cell populations of interest for use in the present invention.

PBMCs may be directly genetically modified to express CARs using methods contemplated herein. In some embodiments, after isolation of PBMC, T lymphocytes are further isolated and in certain embodiments, both cytotoxic and helper T lymphocytes can be sorted into naïve, memory, and effector T cell subpopulations either before or after genetic modification and/or expansion.

CD8⁺ cells can be obtained by using standard methods. In some embodiments, CD8⁺ cells are further sorted into naive, central memory, and effector cells by identifying cell surface antigens that are associated with each of those types of CD8⁺ cells.

In some embodiments, naive CD8⁺ T lymphocytes are characterized by the expression of phenotypic markers of naive T cells including CD62L, CCR7, CD28, CD3, CD 127, and CD45RA.

In some embodiments, memory T cells are present in both CD62L⁺ and CD62L subsets of CD8⁺ peripheral blood lymphocytes. PBMC are sorted into CD62L-CD8⁺ and CD62L*CD8⁺ fractions after staining with anti-CD8 and anti-CD62L antibodies. In some embodiments, the expression of phenotypic markers of central memory T cells include CD45RO, CD62L, CCR7, CD28, CD3, and CD127 and are negative for granzyme B. In some embodiments, central memory T cells are CD45RO⁺, CD62L⁺, CD8⁺ T cells.

In some embodiments, effector T cells are negative for CD62L, CCR7, CD28, and CD127, and positive for granzyme B and perforin.

In some embodiments, CD4⁺ T cells are further sorted into subpopulations. For example, CD4⁺ T helper cells can be sorted into naive, central memory, and effector cells by identifying cell populations that have cell surface antigens. CD4⁺ lymphocytes can be obtained by standard methods. In some embodiments, naïve CD4⁺ T lymphocytes are CD45RO⁻, CD45RA⁺, CD62L⁺CD4⁺ T cell. In some embodiments, central memory CD4⁺ cells are CD62L positive and CD45RO positive. In some embodiments, effector CD4⁺ cells are CD62L and CD45RO negative.

The immune effector cells, such as T cells, can be genetically modified following isolation using known methods, or the immune effector cells can be activated and expanded (or differentiated in the case of progenitors) in vitro prior to being genetically modified. In some embodiments, the immune effector cells, such as T cells, are genetically modified with the chimeric antigen receptors contemplated herein (e.g., transduced with a viral vector comprising a nucleic acid encoding a CAR) and then are activated and expanded in vitro. In various embodiments, T cells can be activated and expanded before or after genetic modification to express a CAR, using methods as described, for example, in U.S. Pat. Nos. 6,352,694; 6,534,055; 6,905,680; 6,692,964; 5,858,358; 6,887,466; 6,905,681; 7, 144,575; 7,067,318; 7, 172,869; 7,232,566; 7, 175,843; 5,883,223; 6,905,874; 6,797,514; 6,867,041; and U.S. Patent Application Publication No. 20060121005.

Generally, the T cells are expanded by contact with a surface having attached thereto an agent that stimulates a CD3 TCR complex associated signal and a ligand that stimulates a co-stimulatory molecule on the surface of the T cells. T cell populations may be stimulated by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. Co-stimulation of accessory molecules on the surface of T cells, is also contemplated.

In some embodiments, PBMCs or isolated T cells are contacted with a stimulatory agent and costimulatory agent, such as anti-CD3 and anti-CD28 antibodies, generally attached to a bead or other surface, in a culture medium with appropriate cytokines, such as IL-2, IL-7, and/or IL-15. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diacione, Besancon, France) can be used as can other methods commonly known in the art (Berg et al., Transplant Proc. 30(8):3975-3977, 1998; Haanen et al., J. Exp. Med. 190(9): 13191328, 1999; Garland et al., J. Immunol Meth. 227(1-2):53-63, 1999). Anti-CD3 and anti-CD28 antibodies attached to the same bead serve as a “surrogate” antigen presenting cell (APC). In other embodiments, the T cells may be activated and stimulated to proliferate with feeder cells and appropriate antibodies and cytokines using methods such as those described in U.S. Pat. Nos. 6,040,177; 5,827,642; and WO2012129514.

In some embodiments, artificial APC (aAPC) made by engineering K562, U937, 721.221, T2, and C1R cells to direct the stable expression and secretion, of a variety of co-stimulatory molecules and cytokines. In a particular embodiment K32 or U32 aAPCs are used to direct the display of one or more antibody-based stimulatory molecules on the AAPC cell surface. Expression of various combinations of genes on the aAPC enables the precise determination of human T-cell activation requirements, such that aAPCs can be tailored for the optimal propagation of T-cell subsets with specific growth requirements and distinct functions. The aAPCs support ex vivo growth and long-term expansion of functional human CD8 T cells without requiring the addition of exogenous cytokines, in contrast to the use of natural APCs. Populations of T cells can be expanded by aAPCs expressing a variety of costimulatory molecules including, but not limited to, CD137L (4-1BBL), CD134L (OX40L), and/or CD80 or CD86. Finally, the aAPCs provide an efficient platform to expand genetically modified T cells and to maintain CD28 expression on CD8 T cells. aAPCs provided in WO 03/057171 and US2003/0147869 are hereby incorporated by reference in their entirety.

In some embodiments, CD34⁺ cells are transduced with a nucleic acid construct contemplated herein. In some embodiments, the transduced CD34⁺ cells differentiate into mature immune effector cells in vivo following administration into a subject, generally the subject from whom the cells were originally isolated. In some embodiments, CD34⁺ cells may be stimulated in vitro prior to exposure to or after being genetically modified with a CAR as described herein, with one or more of the following cytokines: Flt-3 ligand (FLT3), stem cell factor (SCF), megakaryocyte growth and differentiation factor (TPO), IL-3 and IL-6 according to the methods described previously (Asheuer et al., 2004; Imren, et al., 2004).

In some embodiments, the disclosure provides a population of modified immune effector cells for the treatment of cancer, the modified immune effector cells comprising a CAR as disclosed herein. For example, a population of modified immune effector cells are prepared from peripheral blood mononuclear cells (PBMCs) obtained from a patient diagnosed with a cancer described herein (autologous donors). The PBMCs form a heterogeneous population of T lymphocytes that can be CD4₊, CD8₊, or CD4₊ and CD8₊.

The PBMCs also can include other cytotoxic lymphocytes such as NK cells or NKT cells. An expression vector carrying the coding sequence of a CAR contemplated herein can be introduced into a population of human donor T cells, NK cells or NKT cells. Successfully transduced T cells that carry the expression vector can be sorted using flow cytometry to isolate CD3 positive T cells and then further propagated to increase the number of these CAR protein expressing T cells in addition to cell activation using anti-CD3 antibodies and or anti-CD28 antibodies and IL-2 or any other methods known in the art as described elsewhere herein. Standard procedures are used for cryopreservation of T cells expressing the CAR protein T cells for storage and/or preparation for use in a human subject. In one embodiment, the in vitro transduction, culture and/or expansion of T cells are performed in the absence of non-human animal derived products such as fetal calf serum and fetal bovine serum. Since a heterogeneous population of PBMCs is genetically modified, the resultant transduced cells are a heterogeneous population of modified cells comprising a MUC16 targeting CAR as contemplated herein.

In some embodiments, a mixture of, e.g., one, two, three, four, five or more, different expression vectors can be used in genetically modifying a donor population of immune effector cells wherein each vector encodes a different chimeric antigen receptor protein as contemplated herein. The resulting modified immune effector cells forms a mixed population of modified cells, with a proportion of the modified cells expressing more than one different CAR proteins.

### I. Cell Manufacturing Methods

In some embodiments, the disclosure provides methods for manufacturing cells genetically engineered to express an anti-MUC16 CAR described herein. The cells manufactured by the methods contemplated herein provide improved adoptive immunotherapy compositions. Without wishing to be bound to any particular theory, it is believed that the cell compositions manufactured by the methods contemplated herein are imbued with superior properties, including increased survival, expansion in the relative absence of differentiation, and persistence in vivo.

In some embodiments, the disclosure provides methods for manufacturing T cells genetically engineered to express an anti-MUC16 CAR described herein. In some embodiments, a method of manufacturing T cells comprises contacting the cells with one or more agents that modulate a PI3K cell signaling pathway. In some embodiments, a method of manufacturing T cells comprises contacting the cells with one or more agents that modulate a PI3K/Akt/mTOR cell signaling pathway. In some embodiments, the T cells may be obtained from any source and contacted with the agent during the activation and/or expansion phases of the manufacturing process. The resulting T cell compositions are enriched in developmentally potent T cells that have the ability to proliferate and express one or more of the following biomarkers: CD62L, CCR7, CD28, CD27, CD122, CD127, CD197, and CD38. In some embodiments, populations of cell comprising T cells, that have been treated with one or more PI3K inhibitors is enriched for a population of CD8⁺ T cells co-expressing one or more or, or all of, the following biomarkers: CD62L, CD127, CD197, and CD38.

In some embodiments, modified T cells comprising maintained levels of proliferation and decreased differentiation are manufactured. In some embodiments, T cells are manufactured by stimulating T cells to become activated and to proliferate in the presence of one or more stimulatory signals and an agent that is an inhibitor of a PI3K cell signaling pathway.

To achieve sufficient therapeutic doses of T cell compositions, T cells are often subject to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

The T cells can then be modified to express an anti-MUC16 CAR. In some embodiments, the T cells are modified by transducing the T cells with a viral vector comprising an anti-MUC16 CAR contemplated herein. In some embodiments, the T cells are modified prior to stimulation and activation in the presence of an inhibitor of a PI3K cell signaling pathway. In some embodiments, T cells are modified after stimulation and activation in the presence of an inhibitor of a PI3K cell signaling pathway. In some embodiments, T cells are modified within 12 hours, 24 hours, 36 hours, or 48 hours of stimulation and activation in the presence of an inhibitor of a PI3K cell signaling pathway.

After T cells are activated, the cells are cultured to proliferate. T cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

In some embodiments, T cell compositions are manufactured in the presence of one or more inhibitors of the PI3K pathway. The inhibitors may target one or more activities in the pathway or a single activity. Without wishing to be bound to any particular theory, it is contemplated that treatment or contacting T cells with one or more inhibitors of the PI3K pathway during the stimulation, activation, and/or expansion phases of the manufacturing process preferentially increases young T cells, thereby producing superior therapeutic T cell compositions.

In some embodiments, a method for increasing the proliferation of T cells expressing an engineered T cell receptor is provided. Such methods may comprise, for example, harvesting a source of T cells from a subject, stimulating and activating the T cells in the presence of one or more inhibitors of the PI3K pathway, modification of the T cells to express an anti-MUC16 CAR, and expanding the T cells in culture.

In some embodiments, genetically modified T cells are expanded by contact with an agent that stimulates a CD3 TCR complex associated signal and a ligand that stimulates a co-stimulatory molecule on the surface of the T cells.

In some embodiments, PBMCs or isolated T cells are contacted with a stimulatory agent and co-stimulatory agent, such as soluble anti-CD3 and anti-CD28 antibodies, or antibodies attached to a bead or other surface, in a culture medium with appropriate cytokines, such as IL-2, IL-7, and/or IL-15.

In some embodiments, PBMCs or isolated T cells are contacted with a stimulatory agent and co-stimulatory agent, such as soluble anti-CD3 and anti-CD28 antibodies, or antibodies attached to a bead or other surface, in a culture medium with appropriate cytokines, such as IL-2, IL-7, and/or IL-15 and/or a PI3K inhibitor.

In some embodiments, a method for producing populations of T cells enriched for expression of one or more of the following biomarkers: CD62L, CCR7, CD28, CD27, CD122, CD127, CD197, and CD38. In some embodiments, young T cells comprise one or more of, or all of the following biological markers: CD62L, CD127, CD197, and CD38. In one embodiment, the young T cells lack expression of CD57, CD244, CD160, PD-1, CTLA4, TIM3, and LAG3 are provided.

In some embodiments, peripheral blood mononuclear cells (PBMCs) are used as the source of T cells in the T cell manufacturing methods contemplated herein. PBMCs form a heterogeneous population of T lymphocytes that can be CD4⁺, CD8⁺, or CD4⁺ and CD8⁺ and can include other mononuclear cells such as monocytes, B cells, NK cells and NKT cells. An expression vector comprising a polynucleotide encoding an engineered TCR or CAR contemplated herein can be introduced into a population of human donor T cells, NK cells or NKT cells. Successfully transduced T cells that carry the expression vector can be sorted using flow cytometry to isolate CD3 positive T cells and then further propagated to increase the number of the modified T cells in addition to cell activation using anti-CD3 antibodies and or anti-CD28 antibodies and IL-2, IL-7, and/or IL-15 or any other methods known in the art as described elsewhere herein.

As used herein, the term “PI3K inhibitor” refers to a nucleic acid, peptide, compound, or small organic molecule that binds to and inhibits at least one activity of PI3K. The PI3K proteins can be divided into three classes, class 1 PI3Ks, class 2 PI3Ks, and class 3 PI3Ks. Class 1 PI3Ks exist as heterodimers consisting of one of four p110 catalytic subunits (p110α, p110β, p110δ, and p110γ) and one of two families of regulatory subunits. A PI3K inhibitor preferably targets the class 1 PI3K inhibitors. In one embodiment, a PI3K inhibitor will display selectivity for one or more isoforms of the class 1 PI3K inhibitors (i.e., selectivity for p110α, p110β, p110δ, and p110γ or one or more of p110α, p110β, p110δ, and p110γ). In another aspect, a PI3K inhibitor will not display isoform selectivity and be considered a “pan-PI3K inhibitor.” In one embodiment, a PI3K inhibitor will compete for binding with ATP to the PI3K catalytic domain.

In some embodiments, a PI3K inhibitor can, for example, target PI3K as well as additional proteins in the PI3K-AKT-mTOR pathway. In some embodiments, a PI3K inhibitor that targets both mTOR and PI3K can be referred to as either an mTOR inhibitor or a PI3K inhibitor. A PI3K inhibitor that only targets PI3K can be referred to as a selective PI3K inhibitor. In some embodiments, a selective PI3K inhibitor can be understood to refer to an agent that exhibits a 50% inhibitory concentration with respect to PI3K that is at least 10-fold, at least 20-fold, at least 30-fold, at least 50-fold, at least 100-fold, at least 1000-fold, or more, lower than the inhibitor's IC50 with respect to mTOR and/or other proteins in the pathway.

In some embodiments, exemplary PI3K inhibitors inhibit PI3K with an IC50 (concentration that inhibits 50% of the activity) of about 200 nM or less, preferably about 100 nm or less, even more preferably about 60 nM or less, about 25 nM, about 10 nM, about 5 nM, about 1 nM, 100 μM, 50 μM, 25 μM, 10 μM, 1 μM, or less. In one embodiment, a PI3K inhibitor inhibits PI3K with an IC50 from about 2 nM to about 100 nm, more preferably from about 2 nM to about 50 nM, even more preferably from about 2 nM to about 15 nM.

Illustrative examples of PI3K inhibitors suitable for use in the T cell manufacturing methods contemplated in particular embodiments include, but are not limited to, BKM120 (class 1 PI3K inhibitor, Novartis), XL147 (class 1 PI3K inhibitor, Exelixis), (pan-PI3K inhibitor, GlaxoSmithKline), and PX-866 (class 1 PI3K inhibitor; p110α, p110β, and p110γ isoforms, Oncothyreon).

Other illustrative examples of selective PI3K inhibitors include, but are not limited to BYL719, GSK2636771, TGX-221, AS25242, CAL-101, ZSTK474, and IPI-145. Further illustrative examples of pan-PI3K inhibitors include, but are not limited to BEZ235, LY294002, GSK1059615, TG100713, and GDC-0941. some embodiments, the PI3K inhibitor is ZSTK474.

In some embodiments, a method for increasing the proliferation of T cells expressing an engineered T cell receptor is provided. Such methods may comprise, for example, harvesting a source of T cells from a subject, stimulating and activating the T cells, modification of the T cells to express a CAR, and expanding the T cells in culture wherein the T cells are manufactured in the presence of one or more PI3K inhibitors in any one of more steps of the manufacturing process.

Manufacturing methods contemplated herein may further comprise cryopreservation of modified T cells for storage and/or preparation for use in a human subject. T cells are cryopreserved such that the cells remain viable upon thawing. When needed, the cryopreserved transformed immune effector cells can be thawed, grown and expanded for more such cells. As used herein, “cryopreserving,” refers to the preservation of cells by cooling to sub-zero temperatures, such as (typically) 77 K or −196° C. (the boiling point of liquid nitrogen). Cryoprotective agents are often used at sub-zero temperatures to prevent the cells being preserved from damage due to freezing at low temperatures or warming to room temperature. Cryopreservative agents and optimal cooling rates can protect against cell injury. Cryoprotective agents which can be used include but are not limited to dimethyl sulfoxide (DMSO) (Lovelock and Bishop, Nature, 1959; 183: 1394-1395; Ashwood-Smith, Nature, 1961; 190: 1204-1205), glycerol, polyvinylpyrrolidine (Rinfret, Ann. N.Y. Acad. Sci., 1960; 85: 576), and polyethylene glycol (Sloviter and Ravdin, Nature, 1962; 196: 48). The preferred cooling rate is 1° to 3° C./minute. After at least two hours, the T cells have reached a temperature of −80° C. and can be placed directly into liquid nitrogen (−196° C.) for permanent storage such as in a long-term cryogenic storage vessel.

### J. Compositions and Formulations

In some embodiments, the disclosure provides compositions comprising one or more polypeptides, polynucleotides, vectors comprising same, genetically modified immune effector cells, etc., as contemplated herein. Compositions include, but are not limited to pharmaceutical compositions. A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions of the disclosure may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically or physiologically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In some embodiments, a composition comprises an amount of CAR-expressing immune effector cells contemplated herein. As used herein, the term “amount” refers to “an amount effective” or “an effective amount” of a genetically modified therapeutic cell, e.g., T cell, to achieve a beneficial or desired prophylactic or therapeutic result, including clinical results.

A “prophylactically effective amount” refers to an amount of a genetically modified therapeutic cell effective to achieve the desired prophylactic result. Typically but not necessarily, since a prophylactic dose is used in subjects prior to or at an earlier stage of disease, the prophylactically effective amount is less than the therapeutically effective amount.

A “therapeutically effective amount” of a genetically modified therapeutic cell may vary according to factors such as the disease state, age, sex, and weight of the individual, and the ability of the stem and progenitor cells to elicit a desired response in the individual. A therapeutically effective amount is also one in which any toxic or detrimental effects of the virus or transduced therapeutic cells are outweighed by the therapeutically beneficial effects. The term “therapeutically effective amount” includes an amount that is effective to “treat” a subject (e.g., a patient). When a therapeutic amount is indicated, the precise amount of the compositions of the present invention to be administered can be determined by a physician with consideration of individual differences in age, weight, tumor size, extent of infection or metastasis, and condition of the patient (subject).

It can generally be stated that a pharmaceutical composition comprising the T cells described herein may be administered at a dosage of 10² to 10¹⁰ cells/kg body weight, preferably 10⁵ to 10⁶ cells/kg body weight, including all integer values within those ranges. The number of cells will depend upon the ultimate use for which the composition is intended as will the type of cells included therein. For uses provided herein, the cells are generally in a volume of a liter or less, can be 500 mL or less, even 250 mL or 100 mL or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present invention, particularly since all the infused cells will be redirected to a particular target antigen (e.g., x or X light chain), lower numbers of cells, in the range of 10⁶/kilogram (10⁶-10¹¹ per patient) may be administered. CAR expressing cell compositions may be administered multiple times at dosages within these ranges. The cells may be allogeneic, syngeneic, xenogeneic, or autologous to the patient undergoing therapy. If desired, the treatment may also include administration of mitogens (e.g., PHA) or lymphokines, cytokines, and/or chemokines (e.g., IFN-γ, IL-2, IL-12, TNF-alpha, IL-18, and TNF-beta, GM-CSF, IL-4, IL-13, Flt3-L, RANTES, MIP1α, etc.) as described herein to enhance induction of the immune response.

Generally, compositions comprising the cells activated and expanded as described herein may be utilized in the treatment and prevention of diseases that arise in individuals who are immunocompromised. In particular, compositions comprising the CAR-modified T cells contemplated herein are used in the treatment of cancers expressing MUC16. The CAR-modified T cells of the present invention may be administered either alone, or as a pharmaceutical composition in combination with carriers, diluents, excipients, and/or with other components such as IL-2 or other cytokines or cell populations. In some embodiments, pharmaceutical compositions contemplated herein comprise an amount of genetically modified T cells, in combination with one or more pharmaceutically or physiologically acceptable carriers, diluents or excipients.

Pharmaceutical compositions of the present invention comprising a CAR-expressing immune effector cell population, such as T cells, may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In some embodiments, compositions are formulated for parenteral administration, e.g., intravascular (intravenous or intraarterial), intraperitoneal or intramuscular administration.

The liquid pharmaceutical compositions, whether they be solutions, suspensions or other like form, may include one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In some embodiments, the T cell compositions contemplated herein are formulated in a pharmaceutically acceptable cell culture medium. Such compositions are suitable for administration to human subjects. In some embodiments, the pharmaceutically acceptable cell culture medium is a serum free medium.

Serum-free medium has several advantages over serum containing medium, including a simplified and better-defined composition, a reduced degree of contaminants, elimination of a potential source of infectious agents, and lower cost. In some embodiments, the serum-free medium is animal-free, and may optionally be protein-free. Optionally, the medium may contain biopharmaceutically acceptable recombinant proteins. “Animal-free” medium refers to medium wherein the components are derived from non-animal sources. Recombinant proteins replace native animal proteins in animal-free medium and the nutrients are obtained from synthetic, plant or microbial sources. “Protein-free” medium, in contrast, is defined as substantially free of protein.

Illustrative examples of serum-free media used in particular compositions includes, but is not limited to QBSF-60 (Quality Biological, Inc.), StemPro-34 (Life Technologies), and X-VIVO 10.

In some embodiments, compositions comprising immune effector cells contemplated herein are formulated in a solution comprising PlasmaLyte A.

In some embodiments, compositions comprising immune effector cells contemplated herein are formulated in a solution comprising a cryopreservation medium. For example, cryopreservation media with cryopreservation agents may be used to maintain a high cell viability outcome post-thaw. Illustrative examples of cryopreservation media used in particular compositions includes, but is not limited to, CryoStor CS10, CryoStor CS5, and CryoStor CS2.

In some embodiments, compositions comprising immune effector cells contemplated herein are formulated in a solution comprising 50:50 PlasmaLyte A to CryoStor CS10.

### K. Kits

In some embodiments, the disclosure provides a kit comprising a population of immune effector cells expressing an anti-MUC16 CAR, or a pharmaceutically composition thereof. In some embodiments, the kit comprises instructions for administering the population of immune effector cells or pharmaceutical composition thereof to a subject in need thereof. In some embodiments, the subject in need has cancer cells expressing MUC16.

In some embodiments, the disclosure provides a kit comprising a population of immune effector cells expressing an anti-MUC16 CAR, or a pharmaceutically composition thereof, and instructions for administering the cells or composition to a subject in need thereof that has received or receiving an immune checkpoint inhibitor. In some embodiments, the disclosure provides a kit comprising a population of immune effector cells expressing an anti-MUC16 CAR, or a pharmaceutically composition thereof, and instructions for administering the cells or composition to a subject in need thereof that has received or receiving nivolumab, pembrolizumab, atezolizumab, or cemiplimab.

In some embodiments, the disclosure provides a kit comprising a population of immune effector cells expressing an anti-MUC16 CAR, or a pharmaceutically composition thereof, and instructions for administering the cells or composition to a subject in need thereof that has received or receiving a bispecific antibody comprising a first binding domain specific for a tumor associated antigen and a second binding domain specific for CD3. In some embodiments, the disclosure provides a kit comprising a population of immune effector cells expressing an anti-MUC16 CAR, or a pharmaceutically composition thereof, and instructions for administering the cells or composition to a subject in need thereof that has received or receiving a bispecific antibody comprising a first binding domain specific for a tumor associated antigen and a second binding domain specific for CD28.

### L. Therapeutic Methods

The genetically modified immune effector cells expressing a CAR contemplated herein provide improved methods of adoptive immunotherapy for use in the prevention, treatment, and amelioration of a cancer, or for preventing, treating, or ameliorating at least one symptom associated with a cancer.

In some embodiments, the genetically modified immune effector cells contemplated herein provide improved methods of adoptive immunotherapy for use in increasing the cytotoxicity in cancer cells in a subject or for use in decreasing the number of cancer cells in a subject.

In some embodiments, the specificity of a primary immune effector cell is redirected to cells expressing a particular antigen, e.g., MUC16, by genetically modifying the primary immune effector cell with an anti-MUC16 CAR as contemplated herein. In some embodiments, a viral vector is used to genetically modify an immune effector cell with a particular polynucleotide encoding an anti-MUC16 CAR.

In some embodiments, a type of cellular therapy where T cells are genetically modified to express an anti-MUC16 CAR target MUC16 expressing cancer cells, and the T cells are infused to a recipient in need thereof is provided. The infused cell is able to kill disease causing cells in the recipient. Unlike antibody therapies, T cell therapies are able to replicate in vivo resulting in long-term persistence that can lead to sustained cancer therapy.

In some embodiments, T cells that express an anti-MUC16 CAR undergo robust in vivo T cell expansion and can persist for an extended amount of time. In some embodiments, T cells that express an anti-MUC16 CAR evolve into specific memory T cells or stem cell memory T cells that can be reactivated to inhibit any additional tumor formation or growth.

Illustrative examples of conditions that can be treated, prevented or ameliorated using the immune effector cells that express an anti-MUC16 CAR are contemplated in particular embodiments. In some embodiments, the disclosure provides methods for treating a cancer comprising administering to a subject in need thereof anti-MUC16 CAR expressing immune effector cells or a composition comprising the same. In some embodiments, the cancer is selected from: ovarian cancer, endometrial cancer, cervical cancer, fallopian tube cancer, breast cancer, pancreatic cancer, non-small cell lung cancer, small cell lung cancer, mesothelioma, intrahepatic cholangiocarcinoma-mass forming type, adenocarcinoma of the uterine cervix and adenocarcinoma of the gastric tract.

In some embodiments, methods comprising administering a therapeutically effective amount of immune effector cells that express an anti-MUC16 CAR or a composition comprising the same, to a patient in need thereof, alone or in combination with one or more therapeutic agents, are provided. In some embodiments, the cells are used in the treatment of patients at risk for developing a condition associated with cancer cells. Thus, in particular embodiments, methods for the treatment or prevention or amelioration of at least one symptom of cancer comprising administering to a subject in need thereof, a therapeutically effective amount of the modified T cells that express an anti-MUC16 CAR.

As used herein, the terms “individual” and “subject” are often used interchangeably and refer to any animal that exhibits a symptom of a disease, disorder, or condition that can be treated with the gene therapy vectors, cell-based therapeutics, and methods contemplated elsewhere herein. In some embodiments, a subject includes any animal that exhibits symptoms of a disease, disorder, or condition related to cancer that can be treated with the gene therapy vectors, cell-based therapeutics, and methods contemplated elsewhere herein. Suitable subjects (e.g., patients) include laboratory animals (such as mouse, rat, rabbit, or guinea pig), farm animals, and domestic animals or pets (such as a cat or dog). Non-human primates and, preferably, human patients, are included. Typical subjects include human patients that have a cancer, have been diagnosed with a cancer, or are at risk or having a cancer.

As used herein, the term “patient” refers to a subject that has been diagnosed with a particular disease, disorder, or condition that can be treated with the gene therapy vectors, cell-based therapeutics, and methods disclosed elsewhere herein.

As used herein “treatment” or “treating,” includes any beneficial or desirable effect on the symptoms or pathology of a disease or pathological condition, and may include even minimal reductions in one or more measurable markers of the disease or condition being treated. Treatment can involve optionally either the reduction the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

As used herein, the phrase “ameliorating at least one symptom of” refers to decreasing one or more symptoms of the disease or condition for which the subject is being treated. some embodiments, the disease or condition being treated is a cancer, wherein the one or more symptoms ameliorated include, but are not limited to, weakness, fatigue, shortness of breath, easy bruising and bleeding, frequent infections, enlarged lymph nodes, distended or painful abdomen (due to enlarged abdominal organs), bone or joint pain, fractures, unplanned weight loss, poor appetite, night sweats, persistent mild fever, and decreased urination (due to impaired kidney function).

By “enhance” or “promote,” or “increase” or “expand” refers generally to the ability of a composition contemplated herein, e.g., a genetically modified T cells that express an anti-MUC16 CAR, to produce, elicit, or cause a greater physiological response (i.e., downstream effects) compared to the response caused by either vehicle or a control molecule/composition. A measurable physiological response may include an increase in T cell expansion, activation, persistence, and/or an increase in cancer cell killing ability, among others apparent from the understanding in the art and the description herein. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. A “decrease” or “reduced” amount is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

By “maintain,” or “preserve,” or “maintenance,” or “no change,” or “no substantial change,” or “no substantial decrease” refers generally to the ability of a composition contemplated herein to produce, elicit, or cause a similar physiological response (i.e., downstream effects) in a cell, as compared to the response caused by either vehicle, a control molecule/composition, or the response in a particular cell lineage. A comparable response is one that is not significantly different or measurable different from the reference response.

In some embodiments, a method of treating cancer in a subject in need thereof comprises administering an effective amount, e.g., therapeutically effective amount of a composition comprising genetically modified immune effector cells contemplated herein. The quantity and frequency of administration will be determined by such factors as the condition of the patient, and the type and severity of the patient's disease, although appropriate dosages may be determined by clinical trials.

In some embodiments, the amount of immune effector cells, e.g., T cells that express an anti-MUC16 CAR, in the composition administered to a subject is at least 0.1×10⁵ cells, at least 0.5×10⁵ cells, at least 1×10⁵ cells, at least 5×10⁵ cells, at least 1×10⁶ cells, at least 0.5×10⁷ cells, at least 1×10⁷ cells, at least 0.5×10⁸ cells, at least 1×10⁸ cells, at least 0.5×10⁹ cells, at least 1×10⁹ cells, at least 2×10⁹ cells, at least 3×10⁹ cells, at least 4×10⁹ cells, at least 5×10⁹ cells, or at least 1×10¹⁰ cells.

In some embodiments, about 1×10⁷ T cells to about 1×10⁹ T cells, about 2×10⁷ T cells to about 0.9×10⁹ T cells, about 3×10⁷ T cells to about 0.8×10⁹ T cells, about 4×10⁷ T cells to about 0.7×10⁹ T cells, about 5×10⁷ T cells to about 0.6×10⁹ T cells, or about 5×10⁷ T cells to about 0.5×10⁹ T cells are administered to a subject.

In some embodiments the amount of immune effector cells, e.g., T cells that express an anti-MUC16 CAR, in the composition administered to a subject is at least 0.1×10⁴ cells/kg of bodyweight, at least 0.5×10⁴ cells/kg of bodyweight, at least 1×10⁴ cells/kg of bodyweight, at least 5×10⁴ cells/kg of bodyweight, at least 1×10⁵ cells/kg of bodyweight, at least 0.5×10⁶ cells/kg of bodyweight, at least 1×10⁶ cells/kg of bodyweight, at least 0.5×10⁷ cells/kg of bodyweight, at least 1×10⁷ cells/kg of bodyweight, at least 0.5×10⁸ cells/kg of bodyweight, at least 1×10⁸ cells/kg of bodyweight, at least 2×10⁸ cells/kg of bodyweight, at least 3×10⁸ cells/kg of bodyweight, at least 4×10⁸ cells/kg of bodyweight, at least 5×10⁸ cells/kg of bodyweight, or at least 1×10⁹ cells/kg of bodyweight.

In some embodiments, about 1×10⁶ T cells/kg of bodyweight to about 1×10⁸ T cells/kg of bodyweight, about 2×10⁶ T cells/kg of bodyweight to about 0.9×10⁸ T cells/kg of bodyweight, about 3×10⁶ T cells/kg of bodyweight to about 0.8×10⁸ T cells/kg of bodyweight, about 4×10⁶ T cells/kg of bodyweight to about 0.7×10⁸ T cells/kg of bodyweight, about 5×10⁶ T cells/kg of bodyweight to about 0.6×10⁸ T cells/kg of bodyweight, or about 5×10⁶ T cells/kg of bodyweight to about 0.5×10⁸ T cells/kg of bodyweight are administered to a subject.

One of ordinary skill in the art would recognize that multiple administrations of the compositions contemplated herein may be required to affect the desired therapy. For example a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

In some embodiments, it may be desirable to administer activated immune effector cells to a subject and then subsequently redraw blood (or have an apheresis performed), activate immune effector cells therefrom, and reinfuse the patient with these activated and expanded immune effector cells. This process can be carried out multiple times every few weeks. In some embodiments, immune effector cells can be activated from blood draws of from 10 cc to 400 cc. some embodiments, immune effector cells are activated from blood draws of 20 cc, 30 cc, 40 cc, 50 cc, 60 cc, 70 cc, 80 cc, 90 cc, 100 cc, 150 cc, 200 cc, 250 cc, 300 cc, 350 cc, or 400 cc or more. Not to be bound by theory, using this multiple blood draw/multiple reinfusion protocol may serve to select out certain populations of immune effector cells.

The administration of the compositions contemplated herein may be carried out in any convenient manner, including by aerosol inhalation, injection, ingestion, transfusion, implantation or transplantation. In some embodiments, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In some embodiments, the compositions contemplated herein are administered to a subject by direct injection into a tumor, lymph node, or site of infection.

In some embodiments, a subject in need thereof is administered an effective amount of a composition to increase a cellular immune response to a MUC16-expressing cell or tumor in the subject. The immune response may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, NY, N.Y.

In some embodiments, a method of treating a subject diagnosed with a cancer is provided comprising removing immune effector cells from the subject, genetically modifying said immune effector cells with a vector comprising a nucleic acid encoding an anti-MUC16 CAR, thereby producing a population of modified immune effector cells, and administering the population of modified immune effector cells to the same subject. In some embodiments, the immune effector cells comprise T cells.

In some embodiments, methods for stimulating an immune effector cell mediated immune modulator response to a target cell population in a subject are provided comprising the steps of administering to the subject an immune effector cell population expressing a nucleic acid construct encoding an anti-MUC16 CAR.

The methods for administering the cell compositions contemplated in particular embodiments includes any method which is effective to result in reintroduction of ex vivo genetically modified immune effector cells that either directly express an anti-MUC16 CAR in the subject or on reintroduction of the genetically modified progenitors of immune effector cells that on introduction into a subject differentiate into mature immune effector cells that express the CAR. One method comprises transducing peripheral blood T cells ex vivo with a nucleic acid construct contemplated herein and returning the transduced cells into the subject.

### 1. Combination Treatment

In some embodiments, compositions contemplated herein comprise an effective amount of CAR-expressing immune effector cells, in combination therapy, e.g., with one or more therapeutic agents. As used herein, “combination therapy” embraces administration of each agent or therapy in a sequential manner in a regimen that will provide beneficial effects of the combination, and co-administration of these agents or therapies in a substantially simultaneous manner, such as in a single capsule having a fixed ratio of these active agents or in multiple, separate capsules for each agent. Combination therapy also includes combinations where individual elements may be administered at different times and/or by different routes but which act in combination to provide a beneficial effect by co-action or pharmacokinetic and pharmacodynamics effect of each agent or tumor treatment approaches of the combination therapy.

Thus, the CAR-expressing immune effector cell compositions may be administered in combination with other known cancer treatments, such as radiation therapy, chemotherapy, transplantation, immunotherapy, hormone therapy, photodynamic therapy, etc. The compositions may also be administered in combination with antibiotics. Such therapeutic agents may be accepted in the art as a standard treatment for a particular disease state as described herein, such as a particular cancer. Exemplary therapeutic agents contemplated include cytokines, growth factors, steroids, NSAIDs, DMARDs, anti-inflammatories, chemotherapeutics, radiotherapeutics, therapeutic antibodies, oncolytic viruses, or other active and ancillary agents.

In some embodiments, compositions comprising CAR-expressing immune effector cells disclosed herein may be administered in conjunction with any number of chemotherapeutic agents. Illustrative examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®., Rhne-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on cancers such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; and pharmaceutically acceptable salts, acids or derivatives of any of the above.

A variety of other therapeutic agents may be used in conjunction with the compositions described herein. In some embodiments, the composition comprising CAR-expressing immune effector cells is administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate.

Other exemplary NSAIDs are chosen from the group consisting of ibuprofen, naproxen, naproxen sodium, Cox-2 inhibitors such as VIOXX® (rofecoxib) and CELEBREX® (celecoxib), and sialylates. Exemplary analgesics are chosen from the group consisting of acetaminophen, oxycodone, tramadol of proporxyphene hydrochloride. Exemplary glucocorticoids are chosen from the group consisting of cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

Illustrative examples of therapeutic antibodies suitable for combination with the CAR modified T cells contemplated herein, include but are not limited to, bavituximab, bevacizumab (avastin), bivatuzumab, blinatumomab, conatumumab, daratumumab, duligotumab, dacetuzumab, dalotuzumab, elotuzumab (HuLuc63), gemtuzumab, ibritumomab, indatuximab, inotuzumab, lorvotuzumab, lucatumumab, milatuzumab, moxetumomab, ocaratuzumab, ofatumumab, rituximab, siltuximab, teprotumumab, and ublituximab.

In some embodiments, the compositions described herein are administered in conjunction with a cytokine. By “cytokine” as used herein is meant a generic term for proteins released by one cell population that act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and —II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, IL-21, a tumor necrosis factor such as TNF-alpha or TNF-beta; and other polypeptide factors including LIF and kit ligand (KL). In one embodiment, the term cytokine includes “masked” cytokines, including but not limited to, masked IL-2. As used herein, the term cytokine includes proteins from natural sources or from recombinant cell culture, and biologically active equivalents of the native sequence cytokines.

In some embodiments, the compositions described herein are administered in combination with an immune checkpoint inhibitor.

As used herein, “immune checkpoint” refers to co-stimulatory and inhibitory signals that regulate the amplitude and quality of T cell receptor recognition of an antigen. In some embodiments, the immune checkpoint is an inhibitory signal. In some embodiments, the inhibitory signal is the interaction between PD-1 and PD-L1. In some embodiments, the inhibitory signal is the interaction between CTLA-4 and CD80 or CD86 to displace CD28 binding. In some embodiments the inhibitory signal is the interaction between LAG3 and MI-IC class II molecules. In some embodiments, the inhibitory signal is the interaction between TIM3 and galectin 9. In some embodiments, the inhibitory signal is the interaction between OX40 and OX40L.

T cell activation and effector functions are balanced by co-stimulatory and inhibitory signals, referred to as “immune checkpoints.” Inhibitory ligands and receptors that regulate T cell effector functions are overexpressed on tumor cells. Subsequently, agonists of co-stimulatory receptors or antagonists of inhibitory signals, result in the amplification of antigen-specific T cell responses. In contrast to therapeutic antibodies which target tumor cells directly, immune checkpoint inhibitor enhances endogenous anti-tumor activity. In certain embodiments, an immune checkpoint inhibitors suitable for use in the methods disclosed herein, is an antagonist of inhibitory signals, e.g., an antibody which targets, for example, PD-1, PD-L1, CTLA-4, LAG3, B7-H3, B7-H4, or TIM3. These ligands and receptors are reviewed in Pardoll, D., Nature. 12: 252-264, 2012. In some embodiments, an immune checkpoint inhibitor targets a molecule selected from the group consisting of: PD-1, PD-L1, PD-L2, CLT A4, GFTR, ICOS, ICOSL, B7H3, B7H4, TIM3, LAG3, OX40, CD27, CD70, CD4⁷ and CD137.

As used herein, “immune checkpoint inhibitor” refers to a molecule that totally or partially reduces, inhibits, interferes with or modulates one or more checkpoint proteins. In some embodiments, the immune checkpoint inhibitor prevents inhibitory signals associated with the immune checkpoint. In some embodiments, the immune checkpoint inhibitor is an antibody. or fragment thereof that disrupts inhibitory signaling associated with the immune checkpoint. In some embodiments, the immune checkpoint inhibitor is a small molecule that disrupts inhibitory signaling. In some embodiments, the immune checkpoint inhibitor is an antibody, fragment thereof, or antibody mimic, that prevents the interaction between checkpoint blocker proteins, e.g., an antibody, or fragment thereof, that prevents the interaction between PD-i and PD-L1. In some embodiments, the immune checkpoint inhibitor is an antibody, or fragment thereof, that prevents the interaction between CTLA-4 and CD80 or CD86. In some embodiments, the immune checkpoint inhibitor is an antibody, or fragment thereof, that prevents the interaction between LAG3 and its ligands, or TIM-3 and its ligands. In some embodiments, the immune checkpoint inhibitor is an antibody, or fragment thereof, that prevents the interaction between OX40 and its ligands. The checkpoint blocker may also be in the form of the soluble form of the molecules (or variants thereof) themselves, e.g., a soluble PD-L1 or PD-L1 fusion.

Disclosed herein are methods for treating a subject afflicted with diseases such as cancer, which methods comprise administering to the subject a population of immune effector cells genetically modified to express an anti-MUC16 CAR and an immune checkpoint inhibitor. In some embodiments, the immune checkpoint inhibitor is an antibody or an antigen-binding portion thereof, that disrupts or inhibits signaling from an inhibitory immunoregulator. In some embodiments, the immune checkpoint inhibitor is a small molecule that disrupts or inhibits signaling from an inhibitory immunoregulator.

In some embodiments, the inhibitory immunoregulator (immune checkpoint inhibitor) is a component of the PD-1/PD-L1 signaling pathway. Accordingly, certain embodiments of the disclosure provide methods for immunotherapy of a subject afflicted with cancer, which methods comprise administering to the subject a therapeutically effective amount of an antibody or an antigen-binding portion thereof that disrupts the interaction between the PD-1 receptor and its ligand, PD-L1. Antibodies known in the art which bind to PD-1 and disrupt the interaction between the PD-1 and its ligand, PD-L1, and stimulates an anti-tumor immune response, are suitable for use in the methods disclosed herein. In certain embodiments, the antibody or antigen-binding portion thereof binds specifically to PD-1. For example, antibodies that target PD-1 include, e.g., nivolumab (BMS-936558, Bristol-Myers Squibb), pembrolizumab (lambrolizumab, MK03475, Merck) and ceniplimab (REGN-2810, Regeneron). Other suitable antibodies for use in the methods disclosed herein are anti-PD-1 antibodies disclosed in U.S. Pat. No. 8,008,449, herein incorporated by reference. In some embodiments, the antibody or antigen-binding portion thereof binds specifically to PD-L1 and inhibits its interaction with PD-1, thereby increasing immune activity. Antibodies known in the art which bind to PD-L1 and disrupt the interaction between the PD-1 and PD-L1, and stimulates an anti-tumor immune response, are suitable for use in the methods disclosed herein. For example, antibodies that target PD-L1 include BMS-936559 (Bristol-Myers Squibb) and MPDL3280A (Genetech). Other suitable antibodies that target PD-L1 are disclosed in U.S. Pat. No. 7,943,743. It will be understood by one of ordinary skill that any antibody which binds to PD-1 or PD-L1, disrupts the PD-1/PD-L1 interaction, and stimulates an anti-tumor immune response, is suitable for use in the methods disclosed herein.

It should be understood that antibodies targeting immune checkpoints suitable for use in the methods disclosed herein are not limited to those described supra. Moreover, it will be understood by one of ordinary skill in the art that other immune checkpoint targets can also be targeted by antagonists or antibodies in the methods described herein, provided that the targeting results in the stimulation of an anti-tumor immune response as reflected in, e.g., an increase in T cell proliferation, enhanced T cell activation, and/or increased cytokine production (e.g., IFN-g, IL-2).

In some embodiments, the compositions described herein are administered in combination with a bispecific antibody specific for a tumor associated antigen (TAA) and a stimulatory molecule. In some embodiments, the bispecific antibody is specific for a TAA and CD3. In some embodiments, the bispecific antibody is specific for a TAA and CD28.

As used herein, “tumor associated antigen” and “TAA” refer to tumor-specific antigens (i.e., antigens found only on cancer cells) and antigens expressed at elevated on cancer or tumor cells relative to healthy cells. Non-limiting examples of a tumor associated antigen include AFP, ALK, BAGE proteins, β-catenin, brc-abl, BRCA1, BORIS, CA9, carbonic anhydrase IX, caspase-8, CCR5, CD40, CDK4, CEA, CTLA4, cyclin-B1, CYP1B1, ErbB3, ErbB4, ETV6-AML, Fra-1, FOLR1, GAGE proteins (e.g., GAGE-1, -2), GD2, GloboH, glypican-3, GM3, gp100, Her2, HLA/B-raf, HLA/k-ras, HLA/MAGE-A3, hTERT, LMP2, MART-1, ML-IAP, Muc1, Muc2, Muc3, Muc4, Muc5, Muc16, MUM1, NA17, NY-BR1, NY-BR62, NY-BR85, OX40, p15, p53, PAP, PAX3, PAX5, PCTA-1, PRLR, RAGE proteins, Ras, RGS5, Rho, SART-1, SART-3, Steap-1, Steap-2, survivin, TGF-β, TMPRSS2, Tn, TRP-1, TRP-2, tyrosinase, uroplakin-3, alpha folate receptor (FRα), αvβ6 integrin, B cell maturation antigen (BCMA), B7-H3 (CD276), B7-H6, carbonic anhydrase IX (CAIX), CCR1, CD16, CD19, CD20, CD22, CD30, CD33, CD3γ, CD38, CD44, CD44v6, CD44v7/8, CD70, CD79a, CD79b, CD123, CD133, CD135 (also known as fmc like tyrosine kinase 3; FLT3), CD138, CD171, carcinoembryonic antigen (CEA), Claudin-6 (CLDN6), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), IL-11Rα, IL-13Rα2, Kappa, cancer/testis antigen 2 (LAGE-1A), Lambda, Lewis-Y (LeY), L1 cell adhesion molecule (L1-CAM), Leukocyte immunoglobulin-like receptor subfamily B member 2 (LILRB2); melanoma antigen gene (MAGE)-A1, MAGE-A3, MAGE-A4, MAGE-A6, MAGEA10, melanoma antigen recognized by T cells 1 (MelanA or MART1), Mesothelin (MSLN), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), tumor associated glycoprotein 72 (TAG72), T-cell immunoglobulin and mucin-domain containing-3 (TIM-3), tumor endothelial marker 1 (TEM1/CD248), tumor endothelial marker 7-related (TEM7R), trophoblast glycoprotein (TPBG), NKG2D ligands, vascular endothelial growth factor receptor 2 (VEGFR2), and Wilms tumor 1 (WT-1).

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Initial Design of Anti-Muc16 Chimeric Antigen Receptors (Cars)

Chimeric antigen receptors (CARs) targeting MUC16 were generated having scFv binders targeting a fragment of MUC16 remaining on the surface of a cell after proteolytic cleavage (see MUC16 fragment SEQ ID NO: 39). FIG. 1 is a schematic of the MUC16 polypeptide and shows the fragment (“nub”) remaining on the cell surface after cleavage. Specifically, the CARs were designed to have anti-MUC16 scFv binders in both VH-VL (H-L) and VL-VH (L-H) orientations, a CD8α hinge, a CD8α transmembrane domain, a 4-1BB co-stimulatory signaling domain, and a CD3 signaling domain (SEQ ID NOs: 1-12 and 41-150). CARs BB4020 and BB4021, comprising a known MUC16 binder, a 41BB or CD28 costimulatory domain, respectively, and a CD3 signaling domain, were also designed.

TABLE 3

| Anti- | | | | |
|---|---|---|---|---|
| MUC16 | | | Binder | Epitope |
| CARs | Description | Titers | Kd | Bin |
| pBB4000 | 7135 H-L; BBz | 8.57 × 10⁷ | 1.05e−8 | 1 |
| pBB4001 | 7135 L-H; BBz | 1.03 × 10⁷ | 1.05e−8 | 1 |
| pBB4002 | 7138 H-L; BBz | 6.55 × 10⁷ | 3.21e−10 | 2 |
| pBB4003 | 7138 L-H; BBz | 4.54 × 10⁷ | 3.21e−10 | 2 |
| pBB4004 | 8755 H-L; BBz | 1.10 × 10⁸ | 1.24e−10 | 3 |
| pBB4005 | 8755 L-H; BBz | 2.38 × 10⁸ | 1.24e−10 | 3 |
| pBB4006 | 8767 H-L; BBz | 2.74 × 10⁸ | 1.01e−9 | 1 |
| pBB4007 | 8767 L-H; BBz | 7.17 × 10⁷ | 1.01e−9 | 1 |
| pBB4008 | 8794 H-L; BBz | 2.98 × 10⁸ | 5.54e−10 | 3 |
| pBB4009 | 8794 L-H; BBz | 4.62 × 10⁵ | 5.54e−10 | 3 |
| pBB4010 | 8799 H-L; BBz | 2.24 × 10⁸ | 8.26e−10 | 2 |
| pBB4011 | 8799 L-H; BBz | 1.06 × 10⁸ | 8.26e−10 | 2 |
| pBB4012 | 8804 H-L; BBz | 7.86 × 10⁷ | 1.86e−9 | 1 |
| pBB4013 | 8804 L-H; BBz | 1.32 × 10⁷ | 1.86e−9 | 1 |
| pBB4014 | 8808 H-L; BBz | 9.67 × 10⁵ | 2.77e−10 | 4 |
| pBB4015 | 8808 L-H; BBz | 4.23 × 10⁷ | 2.77e−10 | 4 |
| pBB4016 | 8810 H-L; BBz | 1.68 × 10⁷ | 1.12e−9 | 2 |
| pBB4017 | 8810 L-H; BBz | 2.55 × 10⁷ | 1.12e−9 | 2 |
| pBB4018 | 8813 H-L; BBz | 5.35 × 10⁷ | 3.2e−10 | 5 |
| pBB4019 | 8813 L-H; BBz | 3.46 × 10⁷ | 3.2e−10 | 5 |
| pBB4020 | aMUC16 CAR; BBz | 7.42 × 10⁷ | | |
| pBB4021 | aMUC16 CAR; CD28z | 6.14 × 10⁷ | | |

### Example 2

### Assessment of Anti-MUC16 Car In Vitro Characteristics

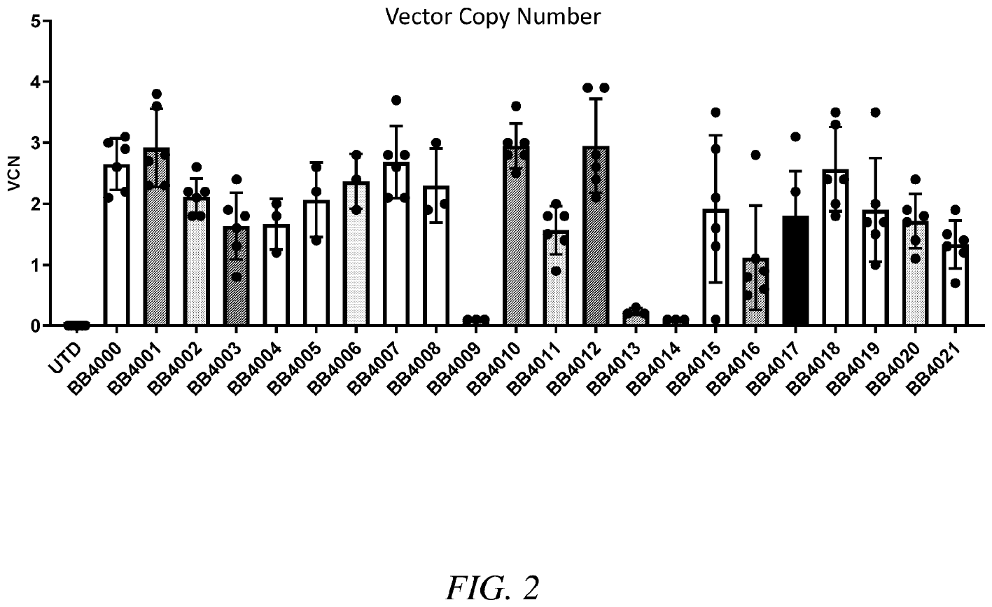

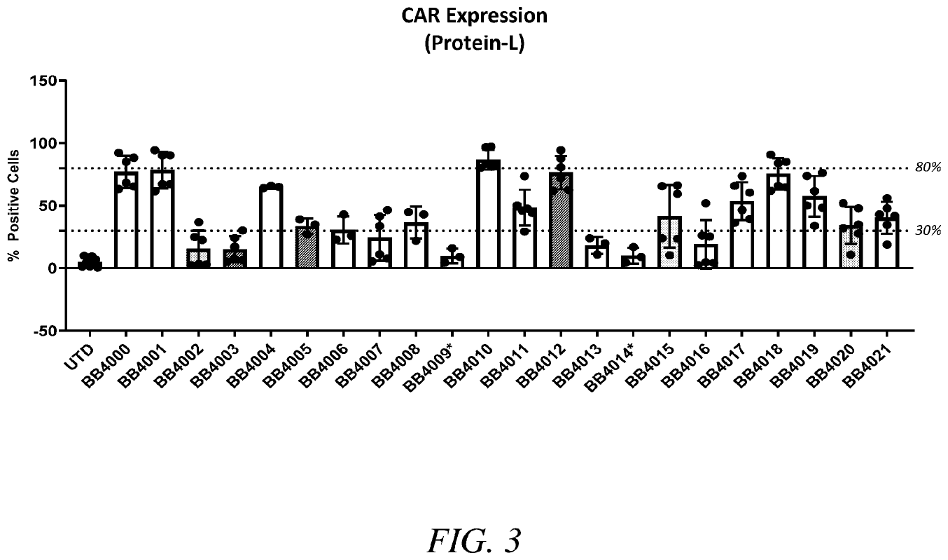

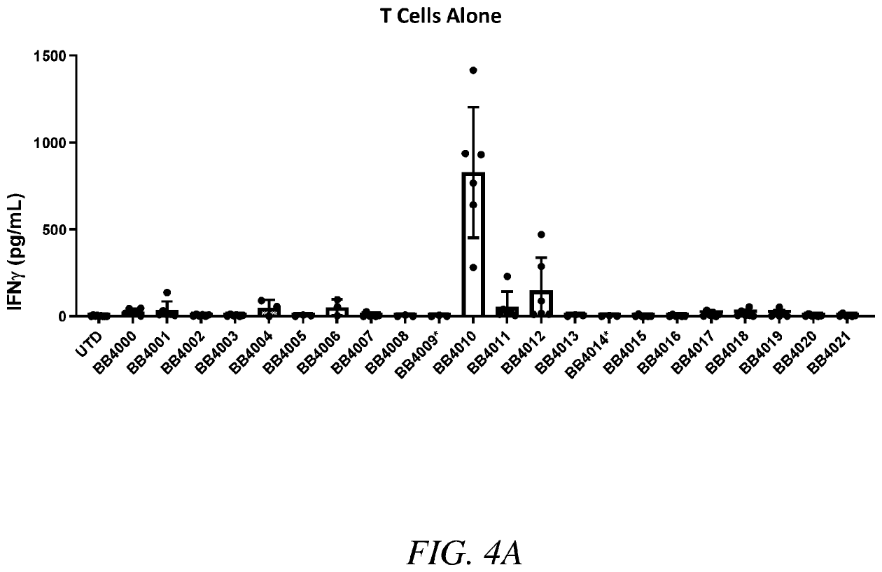

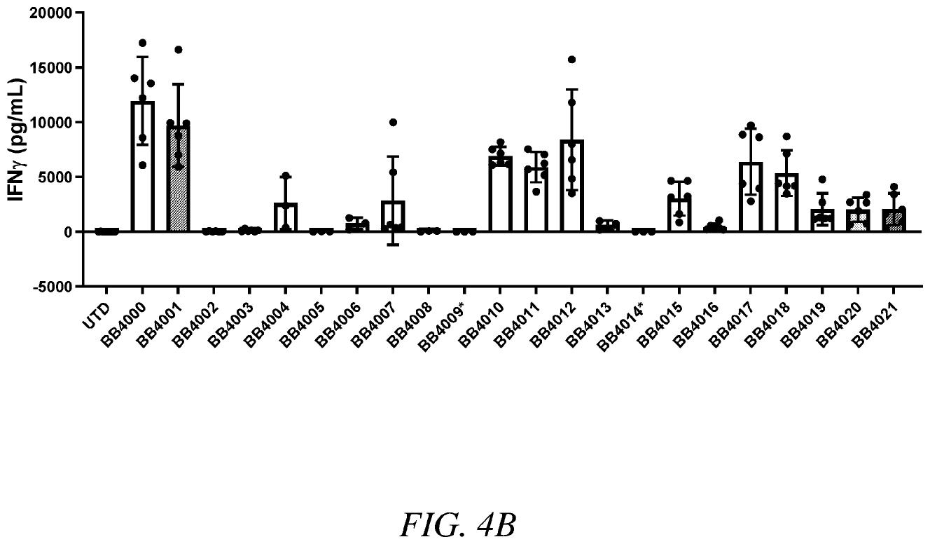

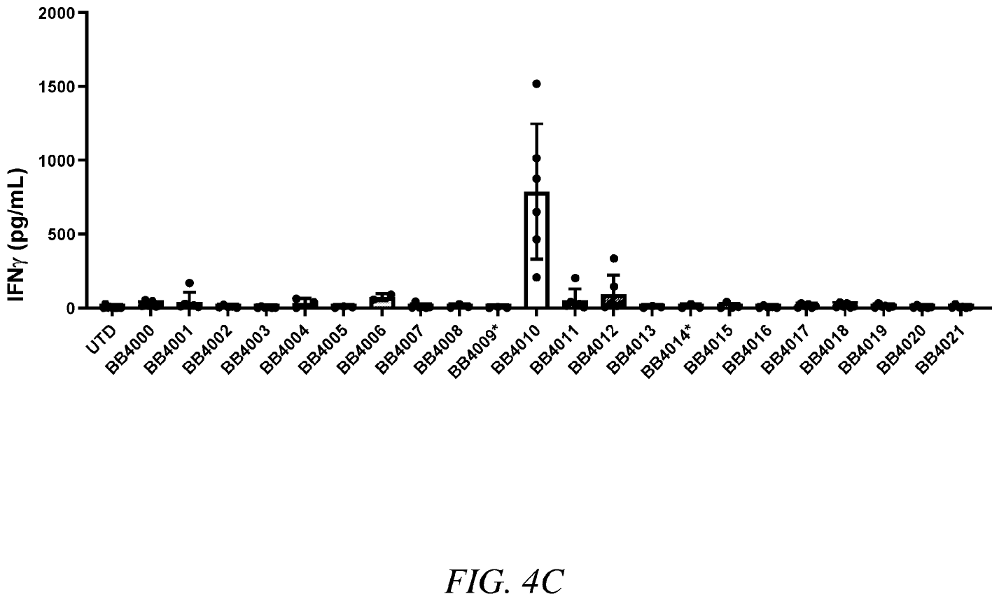

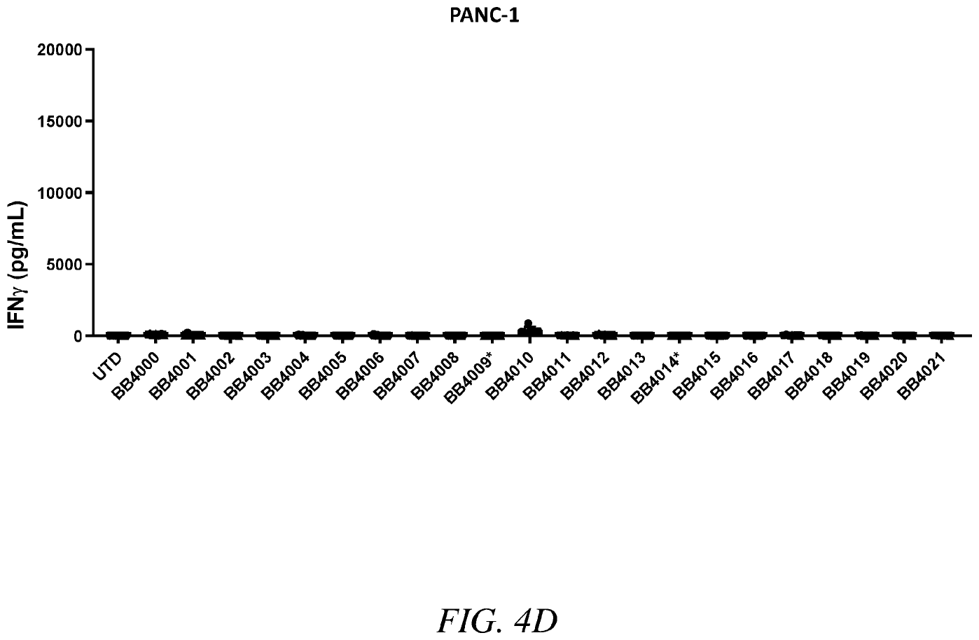

Ten antibody scFvs were converted into CARs in the light-heavy, heavy-light orientation to yield 20 anti-MUC16 CAR constructs (Table 4). Each construct was transduced via lentiviral vectors (LVVs) into T cells derived from healthy donors and were screened for vector copy number (VCN) (FIG. 2), CAR expression (FIG. 3) and antigen-dependent and independent activity (FIGS. 4A-4D). Antigen-dependent activity was evaluated by IFNγ released in co-cultures of CAR-T cells with antigen positive (OVCAR3) or negative (Jurkat, PANC-1) tumor cells, whereas antigen-independent activity was evaluated by IFNγ released in T cell only cultures. Vector copy number (VCN) was evaluated to assess transduction efficiency of the LVV. Of the 20 CARs screened, 6 constructs were deprioritized: 3 constructs minimally transduced T cells (FIG. 2: BB4009, BB4013, BB4014); and of the constructs that transduced well (i.e., VCN>1), 3 CAR-T products exhibited low (<30%) CAR expression (FIG. 3: BB4002, BB4003, BB4016). Of the remaining 14 CARs, BB4010 was deprioritized due to relatively higher antigen-independent IFNγ release (FIG. 4A). Of the remaining 13 CARs, 8 exhibited antigen-dependent activity, as these CARs released IFNγ when co-cultured with OVCAR3 (FIG. 4B) cells but not Jurkat (FIG. 4C) or PANC-1 cells (FIG. 4D). From these 8 CARs, BB4000, BB4011, and BB4015 were selected for additional in vitro and in vivo characterization based upon their mAb affinity, binding epitope, and range of antigen dependent reactivity, >30% CAR expression, little to no antigen independent reactivity and >1 VCN.

### Example 3

### Assessment of Anti-MUC16 Car Sensitivity to Muc16 Expression

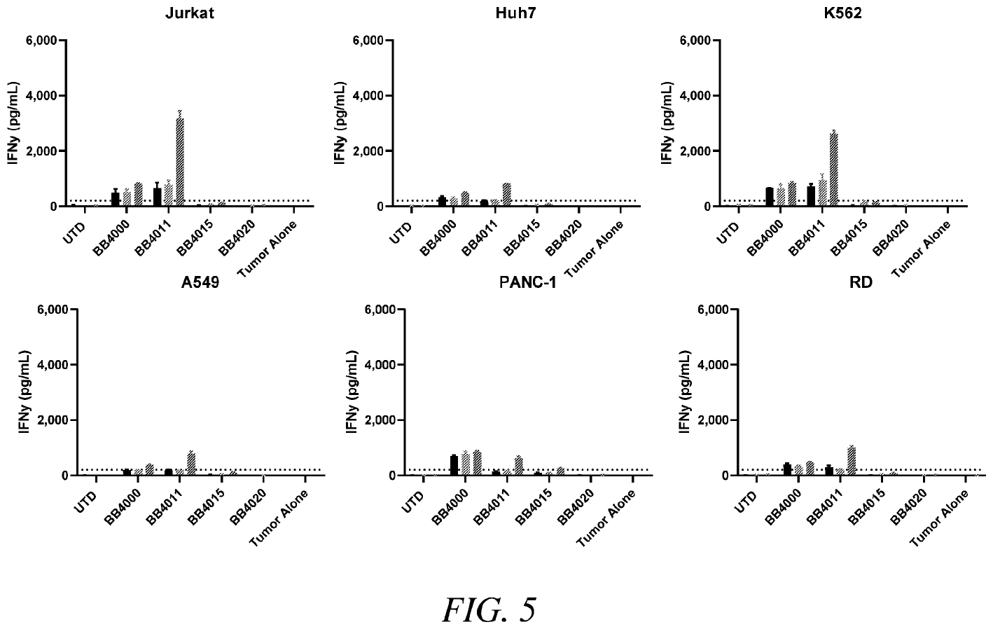

The pharmacological sensitivity of BB4000, BB4011, and BB4015 was evaluated against six antigen negative tumor cells (FIG. 5: Jurkat, PANC-1, HUH7, K562, A549, and RD), to demonstrate antigen specificity. IFNγ release was measured from supernatants collected from co-cultures of CAR T or control untransduced T cells (UTD) with each antigen negative tumor line at a 1:1 ratio for 24 hours. BB4015 demonstrated the lowest tonic activity across all donors of the assessed CARs.

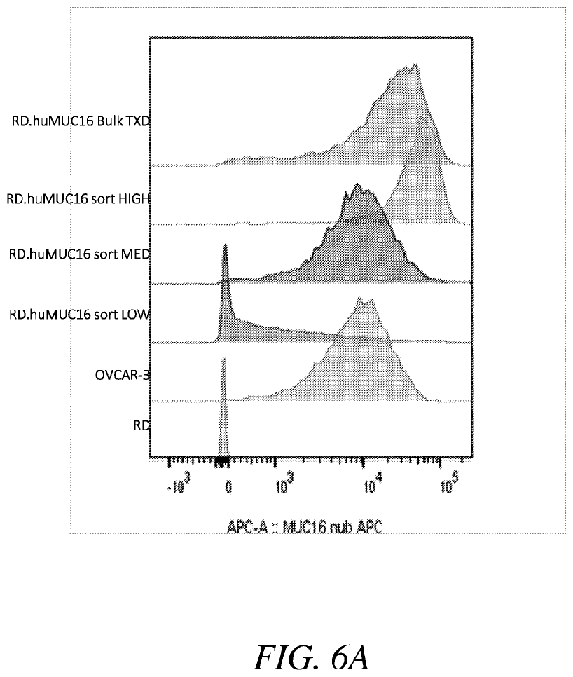

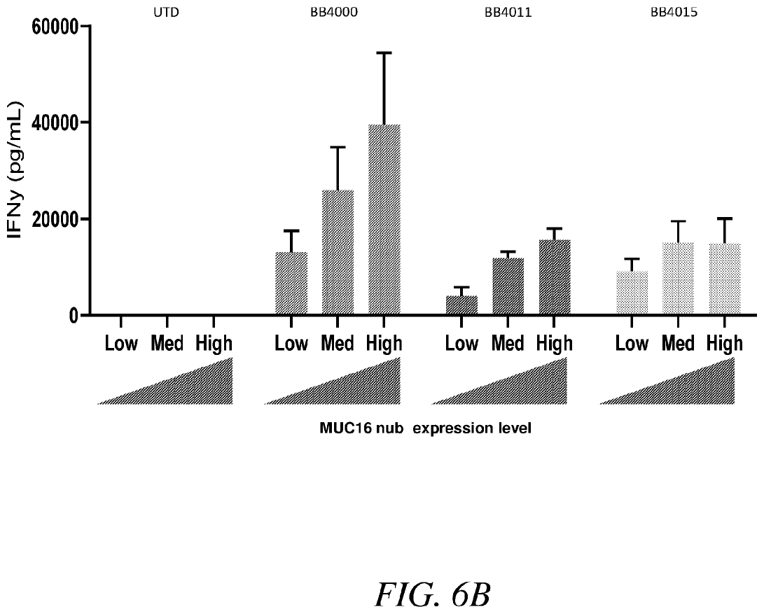

Next, BB4000, BB4011 and BB4015 were evaluated against engineered RD tumor cells, stably expressing high, medium, or low levels of the MUC16 ectodomain (FIG. 6A) for antigen-dependent activity by IFNγ released into co-culture. CAR T cells or UTD T cells (from three donors) were co-cultured with MUC16-expressing RD tumor cells for 24 hours at a 1:1 ratio. BB4000 resulted in the highest IFNγ release followed by BB4015 and BB4011. Titratable antigen-dependent responses were observed across all antigen-positive conditions (FIG. 6B).

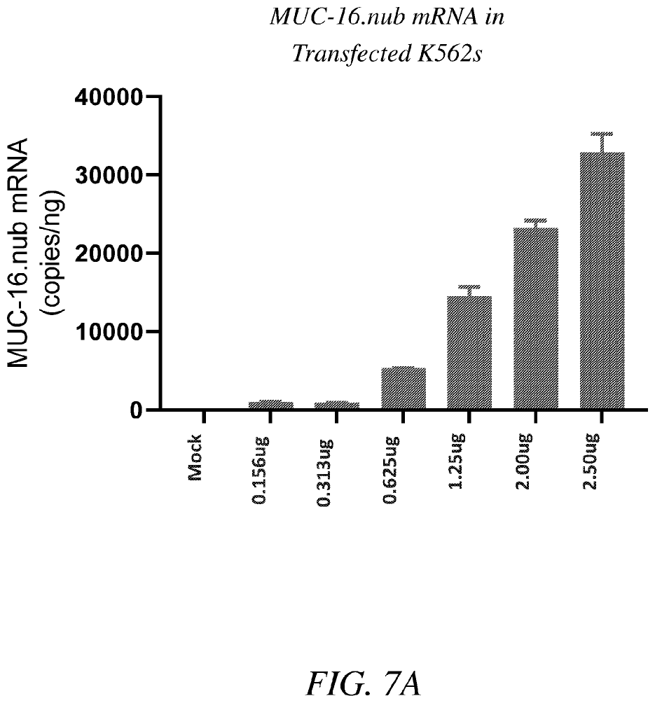

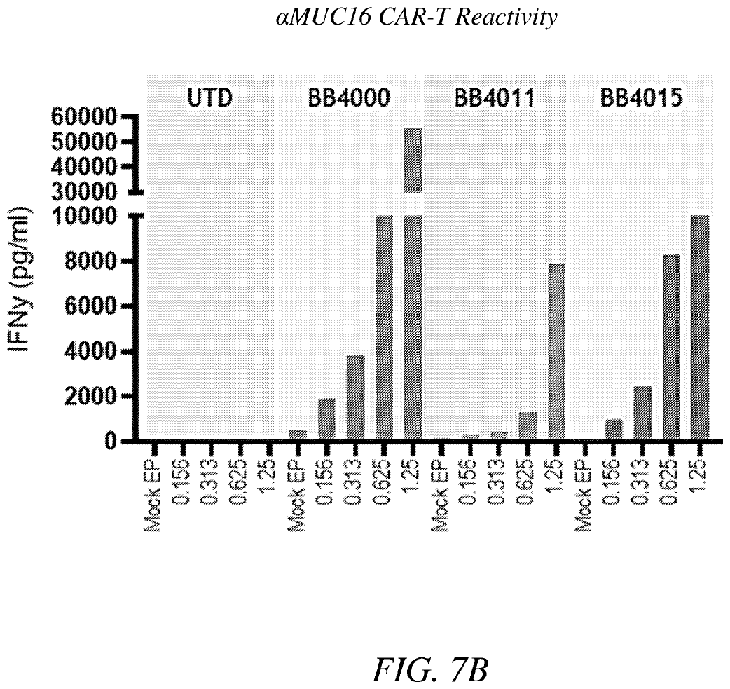

Antigen-dependent activity was further evaluated by IFNγ released into co-culture of CAR T cells or control UTD T cells with K562 tumor cells expressing titrated amount of MUC16 antigen. mRNA coding for the ectodomain of MUC16 (SEQ ID NO: 39) was electroporated into K562 tumor cells ranging from 0.156 μg to 2.5 μg of mRNA (FIG. 7A). CAR T cells and UTD T cells from three healthy donors were co-cultured at a 1:1 ratio with transfected K562s for 24 hours. K562s demonstrated titratable IFNγ release against electroporated tumor cell conditions (FIG. 7B). BB4000, BB4011, and BB4015 all exhibited antigen-dependent IFNγ release in a dose-dependent manner.

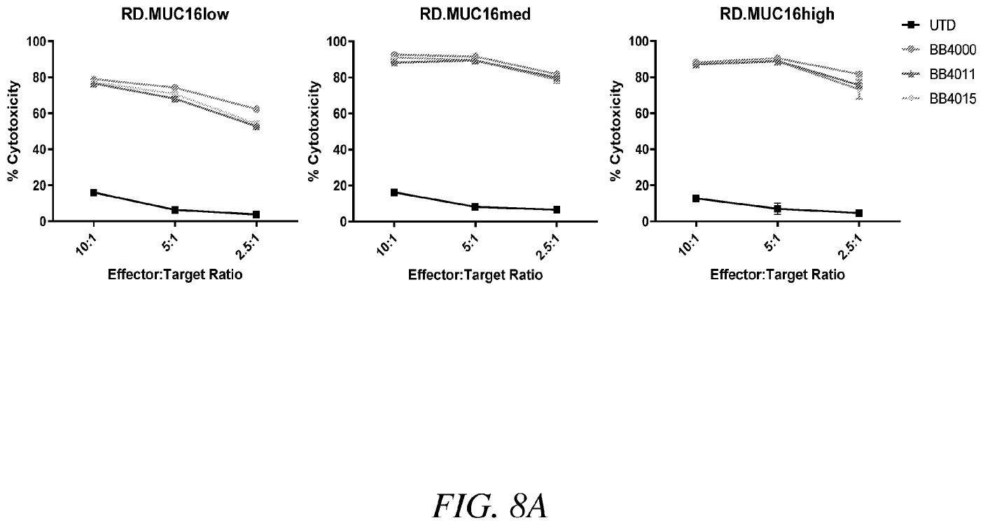

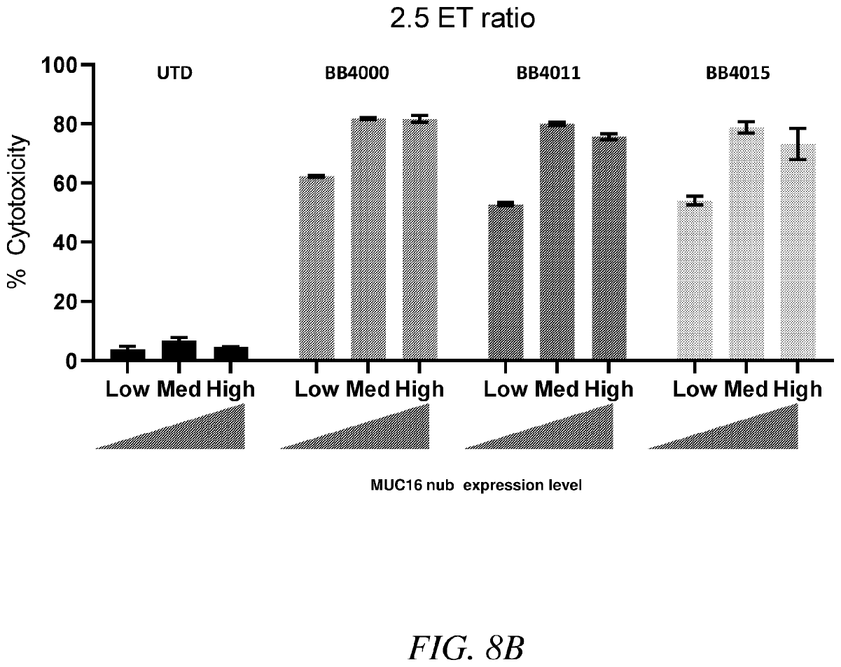

Lastly, antigen dependent cytotoxicity of BB4000, BB4011, and BB4015 was evaluated by percent (%) of tumor cell killing. CAR T cells or UTD T cells, from three healthy donors, and above-mentioned engineered RD tumor cells were co-cultured at 10:1, 5:1, and 2.5:1 effector to T cells (E:T) ratios for 72 hours on an impedance analyzer. BB4015, BB4000, and BB4011 CARs exhibited >70% cytotoxicity when cultured with RD medium or high antigen positive tumor cells across all E:T ratios (FIG. 8A). BB4011 and BB4015 exhibited <60% cytotoxicity when cultured with RD low antigen positive tumor cells at a 2.5:1 E:T ratio, whereas BB4000 exhibited >60% (FIG. 8B). B4000, BB4011, and BB4015 all exhibited antigen-dependent cytotoxicity in a dose-dependent manner.

### Example 4

### Assessment of Anti-MUC16 Car T Cell Differentiation

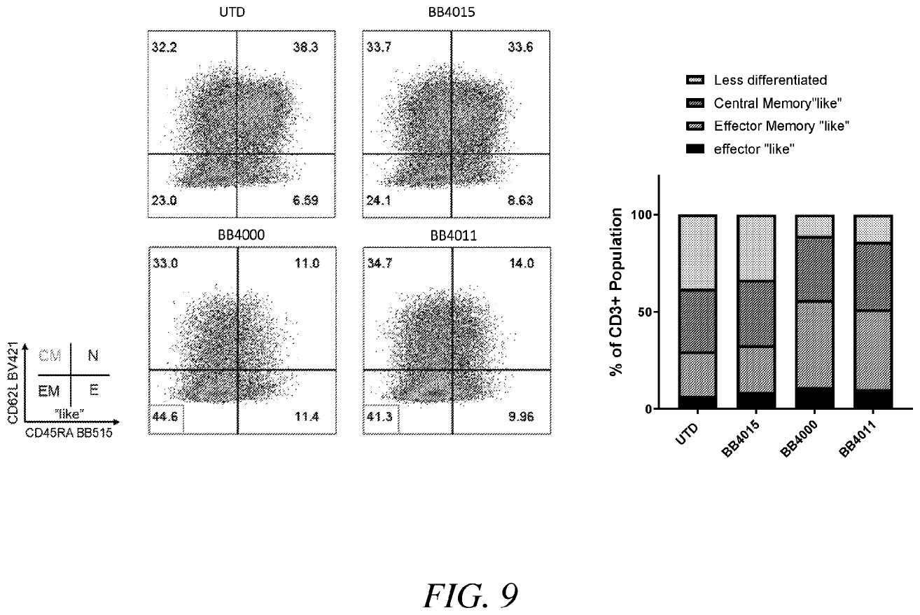

BB4000, BB4011, and BB4015 CAR T cells were characterized for phenotypic markers via flow cytometry. The markers included CD3, CD4, CD8, CD62L and CD45RA. BB4000 resulted in an average of 45% CD4+ T cells and an average of 36.4% CD8+ T cells; BB4011, 47.1% CD4+ T cells and 38% CD8+ T cells; BB4015, 60.9% CD4+ T cells and 29% CD8+ T cells. When compared to UTD controls, BB4015 exhibited increased expression of CD62L and CD45RA, pointing to a greater naïve like phenotype with potential for greater in vivo persistence, whereas both BB4000 and BB4011 had decreased CD62L expression (FIG. 9).

### Example 5

### Anti-MUC16 Car Binding to MUC16 Ectodomain

Peripheral blood mononuclear cells (PBMCs) extracted from human blood, were activated using anti-CD3 and anti-CD28 antibodies. Activated cells from the PBMCs, were then transduced with a lentiviral vector comprising a polynucleotide encoding the anti-MUC16 CAR construct BB4015 (see SEQ ID NOs: 11 and 12). UTD cells were used as a control condition. The transduced and UTD cells were expanded in T cell growth media for 10 days in the presence of IL2.

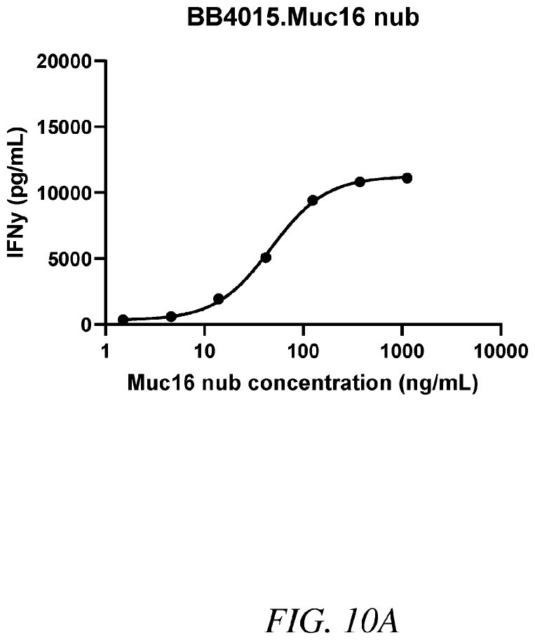

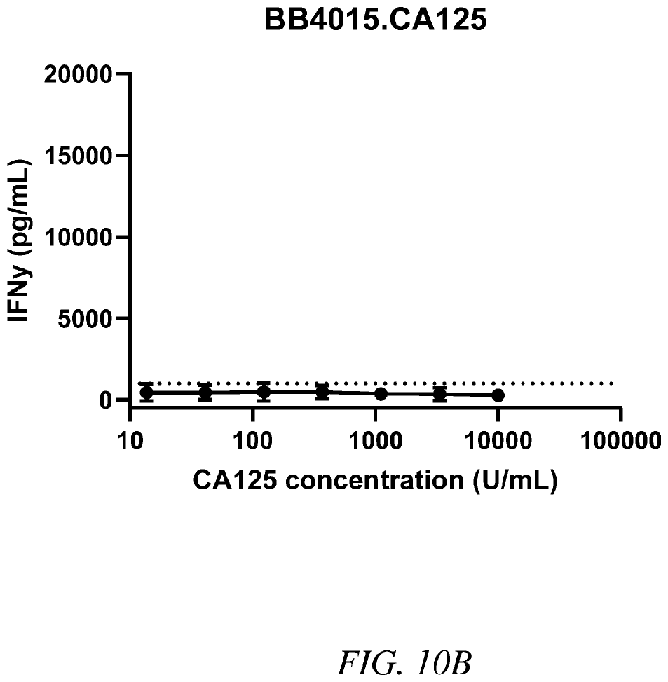

Post expansion, the anti-MUC16 CAR transduced cells were cultured in 96 well plates coated with MUC16-nub ectodomain antigen (SEQ ID NO: 39) at a concentration ranging between 1.54 ng/ml to 1125 ng/ml or CA125 (human MUC16 polypeptide lacking the nub ectodomain antigen; SEQ ID NO: 40) protein at concentrations ranging from 1.37 U/ml to 10,000U/ml. The anti-MUC16 CAR T cells released increasing levels of IFNγ proportional to the MUC16 nub ectodomain concentration (FIG. 10A), whereas the CA125 protein did not elicit any functional response from the anti-MUC16 CAR T cells (FIG. 10B). The UTD controls did not exhibit any functional response to MUC16 nub or CA125. This illustrates the specificity of the anti-MUC16 CAR to the MUC16 ectodomain over the shed CA125 portion.

### Example 6

### Anti-Tumor Activity of Anti-MUC16 Car T Cells

Healthy human PBMCs were harvested and activated using anti-CD3 and anti-CD28 antibodies. The cells were then transduced with lentiviral vector comprising a polynucleotide encoding the anti-MUC16 CAR BB4015 construct (see SEQ ID NOs: 11 and 12). UTD cells were used as control condition. The transduced and UTD cells were expanded in T cell growth media for 10 days in the presence of IL2.

UTD or anti-MUC16 CAR T cells were co-cultured in a 1:1 ratio with OVCAR3 tumor cells which express MUC16 antigen. 24 hours post co-culture, the supernatants were harvested and analyzed for IFNγ concentration using Luminex. The anti-MUC16 CAR T demonstrated MUC16 antigen specific IFNγ release compared to UTD control condition (data not shown).

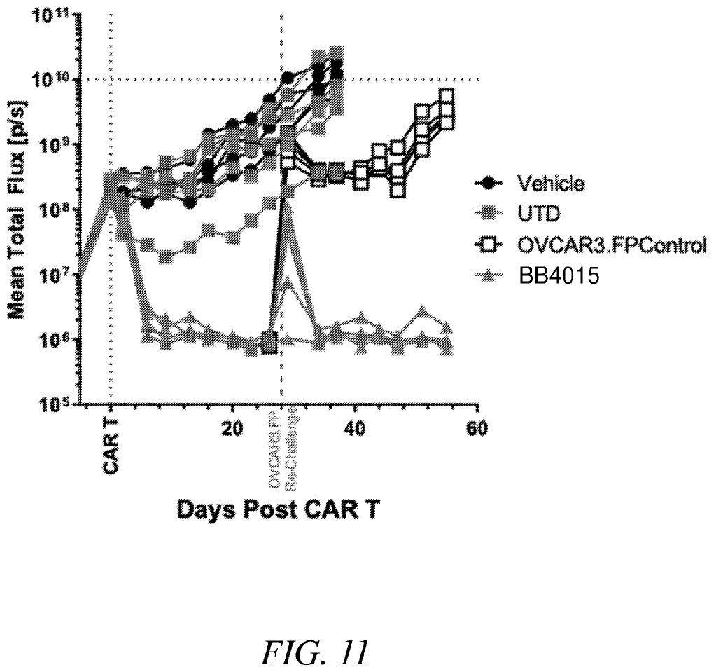

In vivo female NSG mice were inoculated intraperitoneally with 20×10⁶ luciferase expressing OVCAR3.FP cells. After approximately 14 days, when tumors averaged a tumor burden of 1.5×10⁸ p/s, the mice were either injected with vehicle, 20×10⁶ UTD or 20×10⁶ anti-MUC16 CAR T cells. The OVCAR3.FP tumor control was observed in animals injected with anti-MUC16 CAR T cells by ˜day 5 post CAR T cell injections (FIG. 11). The anti-MUC16 CAR T group of n=5 are represented as individual data points per each animal of the group. At 28 days post anti-MUC16 CAR T injection, the n=5 group of mice were re-challenged by OVCAR3.FP tumor injection along with a control naïve group of NSG mice that were re-injected with OVCAR3.FP tumor cells. Tumor rejection was observed post re-challenge. FIG. 11 displays vehicle, UTD and OVCAR3.FP groups for n=5 mice represented as mean luminescence flux per group.

### Example 7

### Anti-Tumor Activity of Anti-MUC16 Car T Cells in OVCAR3 Model

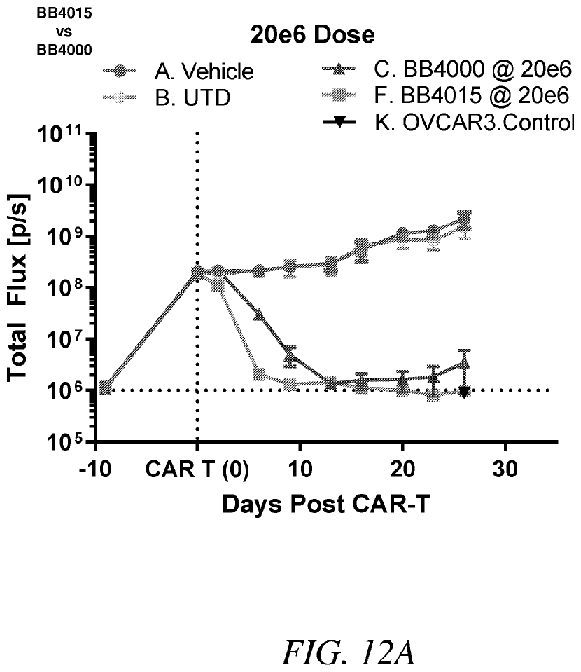

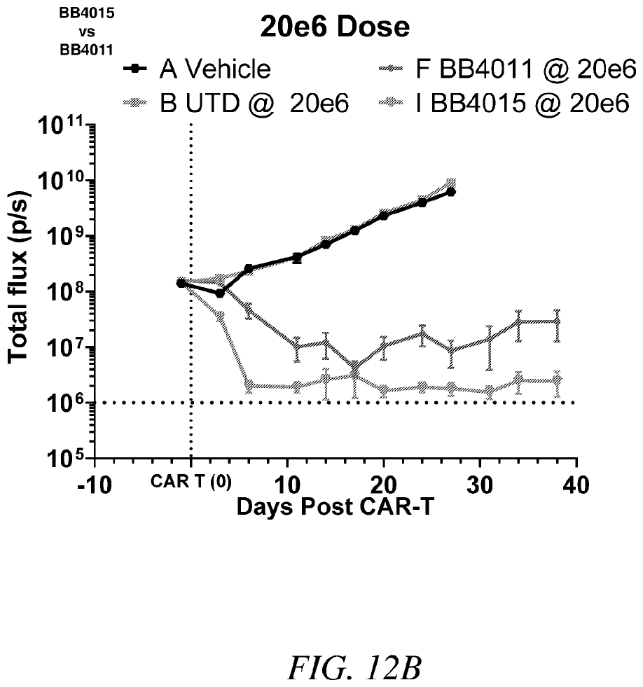

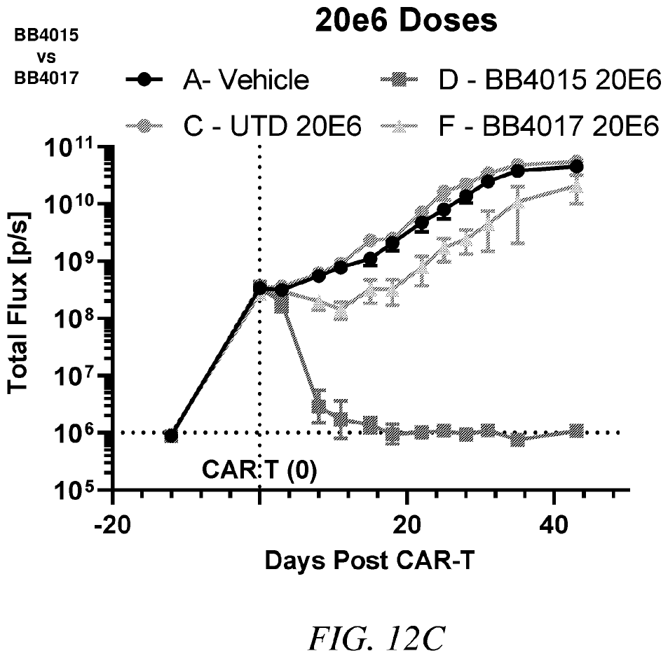

In vivo studies were performed in female NSG mice inoculated with OVCAR3 tumor cells tagged with Firefly luciferase allowing for tumor burden monitoring via an in vivo imaging system (IVIS®; PerkinElmer*). 20×10⁶ tumor cells were injected into the intraperitoneal space of female mice and monitored until appropriate tumor volume was reached for CAR T injection. BB4000, BB4011, BB4017 and BB4015 T cells were injected IV in a total volume of 0.4 mL via tail vein. Mice were monitored for T cell efficacy over the course of the study. When tested head-to-head in vivo, BB4015 demonstrated a faster and more durable response than BB4011 (FIG. 12A), BB4000 (FIG. 12B), and BB4017 (FIG. 12C).

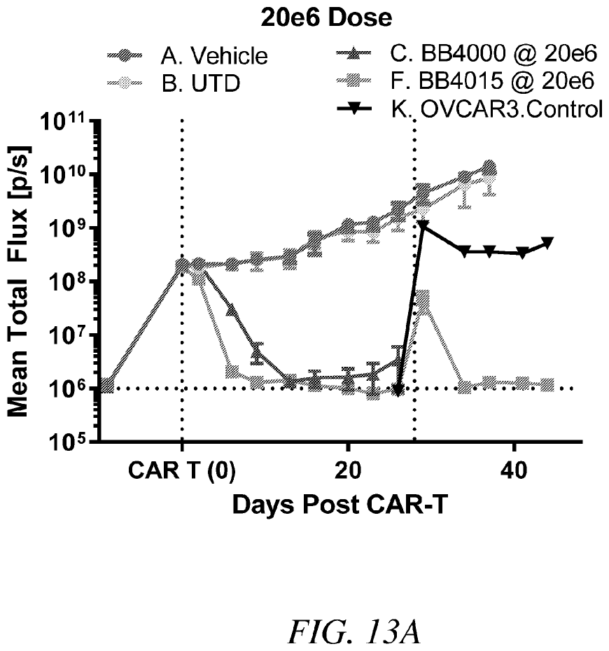

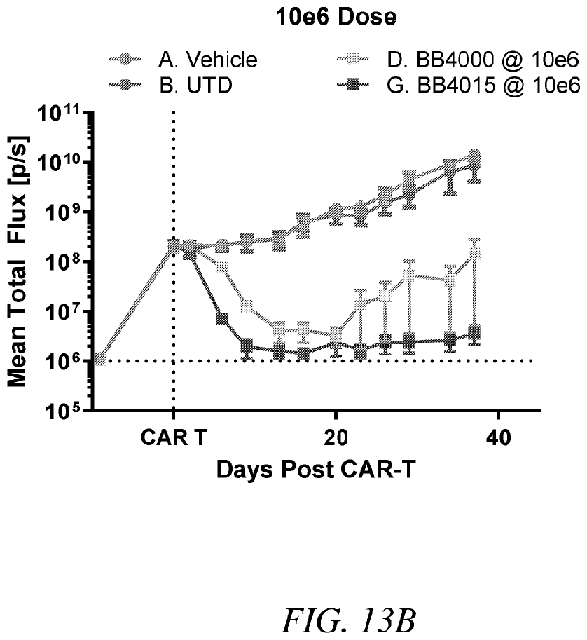

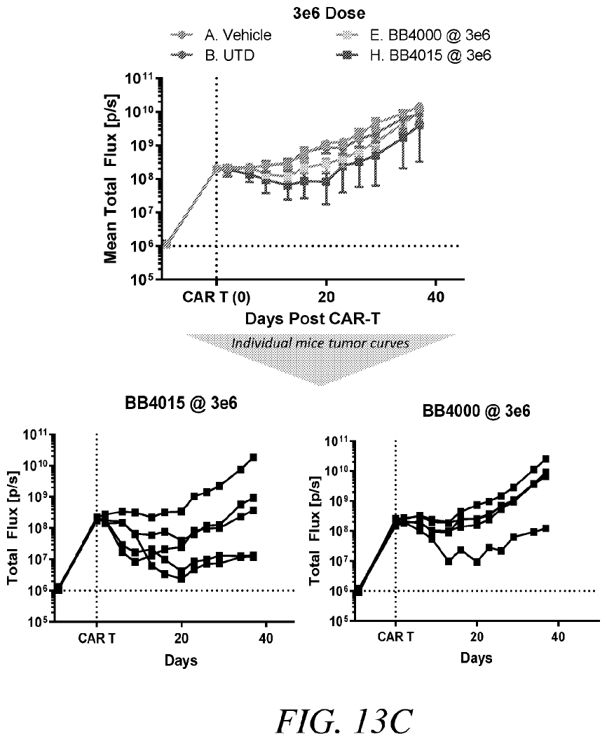

Next, the OVCAR3.FP IP tumor model was employed as described above. On day 0, animals were treated with a single IV injection of BB4015 and BB4000 at 20×10⁶, 10×10⁶, or 3×10⁶ CAR+ cells/mouse, UTD cells, or RPMI vehicle (control). Vehicle alone and UTD control cells had no effect on the tumor burden. BB4015 showed superior dose-dependent anti-tumor potency when compared to BB4000 at all doses with the highest dose of 20×10⁶ CAR+ cells/mouse eliminated tumors by approximately day 6 (FIGS. 13A-13C). Overall, these results demonstrate that BB4015 controls OVCAR3.FP tumor growth with greater dose dependent anti-tumor potency than other constructs.

TABLE 4
SEQUENCE LISTING

| SEQ | | |
|---|---|---|
| NO: | Description | Sequence |
| 1 | Anti-MUC16 8808 | QSLSSNY |
| | CDRL1 | |
| 2 | Anti-MUC16 8808 | GIS |
| | CDRL2 | |
| 3 | Anti-MUC16 8808 | QQYGSSPWT |
| | CDRL3 | |
| 4 | Anti-MUC16 8808 | GFTFSNYG |
| | CDRH1 | |
| 5 | Anti-MUC16 8808 | ISDDGSFK |
| | CDRH2 | |
| 6 | Anti-MUC16 8808 | AKWQHNWNDGGFDY |
| | CDRH3 | |
| 7 | Anti-MUC16 8808 | EIVLTQSPDTLSLSPGERATLSCRASQSLSSNYLAWYRQKPGQAPRLLIYGI |
| | variable light | SSRATGIPDRFSGSGSGTDFTLTISRLEPEDFAVYYCQQYGSSPWTFGQGTK |
| | chain | VEIK |
| 8 | Anti-MUC16 8808 | QVQLVESGGGVVQPGRSLRLSCVASGFTFSNYGIHWVRQAPGKGLEWVAVIS |
| | variable heavy | DDGSFKFYADSVKGRFTISRDNSKNTLYLQMNSLRVEDSAVYHCAKWQHNWN |
| | chain | DGGFDYWGQGTLVTVSS |
| 9 | pBB4015 CAR | EIVLTQSPDTLSLSPGERATLSCRASQSLSSNYLAWYRQKPGQAPRLLIYGI |
| | a.a. sequence | SSRATGIPDRFSGSGSGTDFTLTISRLEPEDFAVYYCQQYGSSPWTFGQGTK |
| | without signal | VEIKGGGGSGGGGSGGGGSQVQLVESGGGVVQPGRSLRLSCVASGFTFSNYG |
| | peptide | IHWVRQAPGKGLEWVAVISDDGSFKFYADSVKGRFTISRDNSKNTLYLQMNS |
| | | LRVEDSAVYHCAKWQHNWNDGGFDYWGQGTLVTVSSTTTPAPRPPTPAPTIA |
| | | SQPLSLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKFSRSAD |
| | | APAYQQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQALPPR |
| 10 | pBB4015 CAR | GAAATTGTGCTGACCCAGAGCCCCGATACCCTTTCCCTTTCCCCAGGCGAGC |
| | n.a. sequence | GCGCCACACTGTCTTGCAGGGCGTCACAGAGCCTGAGCAGCAACTATCTCGC |
| | without signal | GTGGTATAGACAGAAACCAGGGCAAGCCCCACGGCTGCTGATCTATGGAATT |
| | peptide | AGCTCACGGGCAACAGGAATCCCCGACAGATTCAGTGGGTCTGGGAGCGGAA |
| | | CGGATTTTACCCTGACAATTAGTAGATTGGAACCGGAAGACTTCGCTGTGTA |
| | | CTATTGCCAGCAGTACGGCTCATCCCCGTGGACCTTCGGACAAGGCACTAAG |
| | | GTAGAGATCAAAGGTGGAGGGGGCTCAGGGGGAGGCGGAAGCGGAGGCGGAG |
| | | GATCTCAGGTGCAGCTGGTTGAGTCTGGAGGCGGAGTCGTTCAGCCAGGTAG |
| | | GAGCCTCCGACTCTCCTGCGTCGCAAGTGGGTTTACGTTTTCCAATTACGGG |
| | | ATTCACTGGGTCCGACAGGCCCCCGGGAAGGGGCTCGAGTGGGTGGCCGTTA |
| | | TTAGCGATGACGGGTCTTTCAAGTTCTATGCCGATTCAGTCAAAGGTAGATT |
| | | CACAATTTCAAGGGATAATAGTAAAAATACACTGTACTTGCAAATGAACTCT |
| | | CTGCGCGTCGAGGATTCAGCCGTGTACCATTGCGCGAAATGGCAGCACAATT |
| | | GGAATGACGGCGGATTCGATTATTGGGGCCAGGGCACACTTGTAACTGTGTC |
| | | AAGCACCACAACACCTGCTCCAAGGCCCCCCACACCCGCTCCAACTATAGCC |
| | | AGCCAACCATTGAGCCTCAGACCTGAAGCTTGCAGGCCCGCAGCAGGAGGCG |
| | | CCGTCCATACGCGAGGCCTGGACTTCGCGTGTGATATTTATATTTGGGCCCC |
| | | TTTGGCCGGAACATGTGGGGTGTTGCTTCTCTCCCTTGTGATCACTCTGTAT |
| | | TGTAAGCGCGGGAGAAAGAAGCTCCTGTACATCTTCAAGCAGCCTTTTATGC |
| | | GACCTGTGCAAACCACTCAGGAAGAAGATGGGTGTTCATGCCGCTTCCCCGA |
| | | GGAGGAAGAAGGAGGGTGTGAACTGAGGGTGAAATTTTCTAGAAGCGCCGAT |
| | | GCTCCCGCATATCAGCAGGGTCAGAATCAGCTCTACAATGAATTGAATCTCG |
| | | GCAGGCGAGAAGAGTACGATGTTCTGGACAAGAGACGGGGCAGGGATCCCGA |
| | | GATGGGGGGAAAGCCCCGGAGAAAAAATCCTCAGGAGGGGTTGTACAATGAG |
| | | CTGCAGAAGGACAAGATGGCTGAAGCCTATAGCGAGATCGGAATGAAAGGCG |
| | | AAAGACGCAGAGGCAAGGGGCATGACGGTCTGTACCAGGGTCTCTCTACAGC |
| | | CACCAAGGACACTTATGATGCGTTGCATATGCAAGCCTTGCCACCCCGC |
| 11 | pBB4015 CAR | MALPVTALLLPLALLLHAARPEIVLTQSPDTLSLSPGERATLSCRASQSLSS |
| | a.a. sequence | NYLAWYRQKPGQAPRLLIYGISSRATGIPDRFSGSGSGTDFTLTISRLEPED |
| | with signal | FAVYYCQQYGSSPWTFGQGTKVEIKGGGGSGGGGSGGGGSQVQLVESGGGVV |
| | peptide | QPGRSLRLSCVASGFTFSNYGIHWVRQAPGKGLEWVAVISDDGSFKFYADSV |
| | | KGRFTISRDNSKNTLYLQMNSLRVEDSAVYHCAKWQHNWNDGGFDYWGQGTL |
| | | VTVSSTTTPAPRPPTPAPTIASQPLSLRPEACRPAAGGAVHTRGLDFACDIY |
| | | IWAPLAGTCGVLLLSLVITLYCKRGRKKLLYIFKQPFMRPVQTTQEEDGCSC |
| | | RFPEEEEGGCELRVKFSRSADAPAYQQGQNQLYNELNLGRREEYDVLDKRRG |
| | | RDPEMGGKPRRKNPQEGLYNELQKDKMAEAYSEIGMKGERRRGKGHDGLYQG |
| | | LSTATKDTYDALHMQALPPR |
| 12 | pBB4015 CAR | ATGGCCCTGCCGGTTACCGCTCTCTTGCTGCCCCTGGCGCTGCTTCTGCACG |
| | n.a. sequence | CGGCAAGACCCGAAATTGTGCTGACCCAGAGCCCCGATACCCTTTCCCTTTC |
| | with signal | CCCAGGCGAGCGCGCCACACTGTCTTGCAGGGCGTCACAGAGCCTGAGCAGC |
| | peptide | AACTATCTCGCGTGGTATAGACAGAAACCAGGGCAAGCCCCACGGCTGCTGA |
| | | TCTATGGAATTAGCTCACGGGCAACAGGAATCCCCGACAGATTCAGTGGGTC |
| | | TGGGAGCGGAACGGATTTTACCCTGACAATTAGTAGATTGGAACCGGAAGAC |
| | | TTCGCTGTGTACTATTGCCAGCAGTACGGCTCATCCCCGTGGACCTTCGGAC |
| | | AAGGCACTAAGGTAGAGATCAAAGGTGGAGGGGGCTCAGGGGGAGGCGGAAG |
| | | CGGAGGCGGAGGATCTCAGGTGCAGCTGGTTGAGTCTGGAGGCGGAGTCGTT |
| | | CAGCCAGGTAGGAGCCTCCGACTCTCCTGCGTCGCAAGTGGGTTTACGTTTT |
| | | CCAATTACGGGATTCACTGGGTCCGACAGGCCCCCGGGAAGGGGCTCGAGTG |
| | | GGTGGCCGTTATTAGCGATGACGGGTCTTTCAAGTTCTATGCCGATTCAGTC |
| | | AAAGGTAGATTCACAATTTCAAGGGATAATAGTAAAAATACACTGTACTTGC |
| | | AAATGAACTCTCTGCGCGTCGAGGATTCAGCCGTGTACCATTGCGCGAAATG |
| | | GCAGCACAATTGGAATGACGGCGGATTCGATTATTGGGGCCAGGGCACACTT |
| | | GTAACTGTGTCAAGCACCACAACACCTGCTCCAAGGCCCCCCACACCCGCTC |
| | | CAACTATAGCCAGCCAACCATTGAGCCTCAGACCTGAAGCTTGCAGGCCCGC |
| | | AGCAGGAGGCGCCGTCCATACGCGAGGCCTGGACTTCGCGTGTGATATTTAT |
| | | ATTTGGGCCCCTTTGGCCGGAACATGTGGGGTGTTGCTTCTCTCCCTTGTGA |
| | | TCACTCTGTATTGTAAGCGCGGGAGAAAGAAGCTCCTGTACATCTTCAAGCA |
| | | GCCTTTTATGCGACCTGTGCAAACCACTCAGGAAGAAGATGGGTGTTCATGC |
| | | CGCTTCCCCGAGGAGGAAGAAGGAGGGTGTGAACTGAGGGTGAAATTTTCTA |
| | | GAAGCGCCGATGCTCCCGCATATCAGCAGGGTCAGAATCAGCTCTACAATGA |
| | | ATTGAATCTCGGCAGGCGAGAAGAGTACGATGTTCTGGACAAGAGACGGGGC |
| | | AGGGATCCCGAGATGGGGGGAAAGCCCCGGAGAAAAAATCCTCAGGAGGGGT |
| | | TGTACAATGAGCTGCAGAAGGACAAGATGGCTGAAGCCTATAGCGAGATCGG |
| | | AATGAAAGGCGAAAGACGCAGAGGCAAGGGGCATGACGGTCTGTACCAGGGT |
| | | CTCTCTACAGCCACCAAGGACACTTATGATGCGTTGCATATGCAAGCCTTGC |
| | | CACCCCGC |
| 13 | Human MUC16 a.a | MLKPSGLPGSSSPTRSLMTGSRSTKATPEMDSGLTGATLSPKTSTGAIVVTE |
| | sequence | HTLPFTSPDKTLASPTSSVVGRTTQSLGVMSSALPESTSRGMTHSEQRTSPS |
| | | LSPQVNGTPSRNYPATSMVSGLSSPRTRTSSTEGNFTKEASTYTLIVETTSG |
| | | PVTEKYTVPTETSTTEGDSTETPWDTRYIPVKITSPMKTFADSTASKENAPV |
| | | SMTPAETTVTDSHTPGRTNPSFGTLYSSFLDLSPKGTPNSRGETSLELILST |
| | | TGYPFSSPEPGSAGHSRISTSAPLSSSASVLDNKISETSIFSGQSLTSPLSP |
| | | GVPEARASTMPNSAIPFSMTLSNAETSAERVRSTISSLGTPSISTKQTAETI |
| | | LTFHAFAETMDIPSTHIAKTLASEWLGSPGTLGGTSTSALTTTSPSTTLVSE |
| | | ETNTHHSTSGKETEGTLNTSMTPLETSAPGEESEMTATLVPTLGFTTLDSKI |
| | | RSPSQVSSSHPTRELRTTGSTSGRQSSSTAAHGSSDILRATTSSTSKASSWT |
| | | SESTAQQFSEPQHTQWVETSPSMKTERPPASTSVAAPITTSVPSVVSGFTTL |
| | | KTSSTKGIWLEETSADTLIGESTAGPTTHQFAVPTGISMTGGSSTRGSQGTT |
| | | HLLTRATASSETSADLTLATNGVPVSVSPAVSKTAAGSSPPGGTKPSYTMVS |
| | | SVIPETSSLQSSAFREGTSLGLTPLNTRHPFSSPEPDSAGHTKISTSIPLLS |
| | | SASVLEDKVSATSTFSHHKATSSITTGTPEISTKTKPSSAVLSSMTLSNAAT |
| | | SPERVRNATSPLTHPSPSGEETAGSVLTLSTSAETTDSPNTHPTGTLTSESS |
| | | ESPSTLSLPSVSGVKTTFSSSTPSTHLFTSGEETEETSNPSVSQPETSVSRV |
| | | RTTLASTSVPTPVFPTMDTWPTRSAQFSSSHLVSELRATSSTSVINSTGSAL |
| | | PKISHLTGTATMSQTNRDTENDSAAPQSTTWPETSPRFKTGLPSATTTVSTS |
| | | ATSLSATVMVSKFTSPATSSMEATSIREPSTTILTTETTNGPGSMAVASTNI |
| | | PIGKGYITEGRLDTSHLPIGTTASSETSMDFTMAKESVSMSVSPSQSMDAAG |
| | | SSTPGRTSQFVDTFSDDVYHLTSREITIPRDGTSSALTPQMTATHPPSPDPG |
| | | SARSTWLGILSSSPSSPTPKVTMSSTFSTQRVTTSMIMDTVETSRWNMPNLP |
| | | STTSLIPSNIPTSGAIGKSTLVPLDTPSPATSLEASEGGLPTLSTYPESTNT |
| | | PSIHLGAHASSESPSTIKLTMASVVKPGSYTPLTFPSIETHIHVSTARMAYS |
| | | SGSSPEMTAPGETNTGSTWDPTTYITTTDPKDTSSAQVSTPHSVRTLRTTEN |
| | | HPKTESATPAAYSGSPKISSSPNLTSPATKAWTITDTTEHSTQLHYTKLAEK |
| | | SSGFETQSAPGPVSVVIPTSPTIGSSTLELTSDVPGEPLVLAPSEQTTITLP |
| | | MATWLSTSLTEEMASTDLDISSPSSPMSTFAIFPPMSTPSHELSKSEADTSA |
| | | IRNTDSTTLDQHLGIRSLGRTGDLTTVPITPLITTWTSVIEHSTQAQDTLSA |
| | | TMSPTHVTQSLKDQTSIPASASPSHLTEVYPELGTQGRSSSEATTFWKPSTD |
| | | TLSREIETGPTNIQSTPPMDNTTTGSSSSGVTLGIAHLPIGTSSPAETSTNM |
| | | ALERRSSTATVSMAGTMGLLVTSAPGRSISQSLGRVSSVLSESTTEGVTDSS |
| | | KGSSPRLNTQGNTALSSSLEPSYAEGSQMSTSIPLTSSPTTPDVEFIGGSTF |
| | | WTKEVTTVMTSDISKSSARTESSSATLMSTALGSTENTGKEKLRTASMDLPS |
| | | PTPSMEVTPWISLTLSNAPNTTDSLDLSHGVHTSSAGTLATDRSLNTGVTRA |
| | | SRLENGSDTSSKSLSMGNSTHTSMTYTEKSEVSSSIHPRPETSAPGAETTLT |
| | | STPGNRAISLTLPFSSIPVEEVISTGITSGPDINSAPMTHSPITPPTIVWTS |
| | | TGTIEQSTQPLHAVSSEKVSVQTQSTPYVNSVAVSASPTHENSVSSGSSTSS |
| | | PYSSASLESLDSTISRRNAITSWLWDLTTSLPTTTWPSTSLSEALSSGHSGV |
| | | SNPSSTTTEFPLFSAASTSAAKQRNPETETHGPQNTAASTLNTDASSVTGLS |
| | | ETPVGASISSEVPLPMAITSRSDVSGLTSESTANPSLGTASSAGTKLTRTIS |
| | | LPTSESLVSFRMNKDPWTVSIPLGSHPTTNTETSIPVNSAGPPGLSTVASDV |
| | | IDTPSDGAESIPTVSFSPSPDTEVTTISHFPEKTTHSFRTISSLTHELTSRV |
| | | TPIPGDWMSSAMSTKPTGASPSITLGERRTITSAAPTTSPIVLTASFTETST |
| | | VSLDNETTVKTSDILDARKTNELPSDSSSSSDLINTSIASSTMDVTKTASIS |
| | | PTSISGMTASSSPSLFSSDRPQVPTSTTETNTATSPSVSSNTYSLDGGSNVG |
| | | GTPSTLPPFTITHPVETSSALLAWSRPVRTFSTMVSTDTASGENPTSSNSVV |
| | | TSVPAPGTWTSVGSTTDLPAMGFLKTSPAGEAHSLLASTIEPATAFTPHLSA |
| | | AVVTGSSATSEASLLTTSESKAIHSSPQTPTTPTSGANWETSATPESLLVVT |
| | | ETSDTTLTSKILVTDTILFSTVSTPPSKFPSTGTLSGASFPTLLPDTPAIPL |
| | | TATEPTSSLATSFDSTPLVTIASDSLGTVPETTLTMSETSNGDALVLKTVSN |
| | | PDRSIPGITIQGVTESPLHPSSTSPSKIVAPRNTTYEGSITVALSTLPAGTT |
| | | GSLVFSQSSENSETTALVDSSAGLERASVMPLTTGSQGMASSGGIRSGSTHS |
| | | TGTKTFSSLPLTMNPGEVTAMSEITTNRLTATQSTAPKGIPVKPTSAESGLL |
| | | TPVSASSSPSKAFASLTTAPPTWGIPQSTLTFEFSEVPSLDTKSASLPTPGQ |
| | | SLNTIPDSDASTASSSLSKSPEKNPRARMMTSTKAISASSFQSTGFTETPEG |
| | | SASPSMAGHEPRVPTSGTGDPRYASESMSYPDPSKASSAMTSTSLASKLTTL |
| | | FSTGQAARSGSSSSPISLSTEKETSFLSPTASTSRKTSLFLGPSMARQPNIL |
| | | VHLQTSALTLSPTSTLNMSQEEPPELTSSQTIAEEEGTTAETQTLTFTPSET |
| | | PTSLLPVSSPTEPTARRKSSPETWASSISVPAKTSLVETTDGTLVTTIKMSS |
| | | QAAQGNSTWPAPAEETGSSPAGTSPGSPEMSTTLKIMSSKEPSISPEIRSTV |
| | | RNSPWKTPETTVPMETTVEPVTLQSTALGSGSTSISHLPTGTTSPTKSPTEN |
| | | MLATERVSLSPSPPEAWTNLYSGTPGGTRQSLATMSSVSLESPTARSITGTG |
| | | QQSSPELVSKTTGMEFSMWHGSTGGTTGDTHVSLSTSSNILEDPVTSPNSVS |
| | | SLTDKSKHKTETWVSTTAIPSTVLNNKIMAAEQQTSRSVDEAYSSTSSWSDQ |
| | | TSGSDITLGASPDVTNTLYITSTAQTTSLVSLPSGDQGITSLTNPSGGKTSS |
| | | ASSVTSPSIGLETLRANVSAVKSDIAPTAGHLSQTSSPAEVSILDVTTAPTP |
| | | GISTTITTMGINSISTTTPNPEVGMSTMDSTPATERRTTSTEHPSTWSSTAA |
| | | SDSWTVTDMTSNLKVARSPGTISTMHTTSFLASSTELDSMSTPHGRITVIGT |
| | | SLVTPSSDASAVKTETSTSERTLSPSDTTASTPISTFSRVQRMSISVPDILS |
| | | TSWTPSSTEAEDVPVSMVSTDHASTKTDPNTPLSTFLFDSLSTLDWDTGRSL |
| | | SSATATTSAPQGATTPQELTLETMISPATSQLPFSIGHITSAVTPAAMARSS |
| | | GVTFSRPDPTSKKAEQTSTQLPTTTSAHPGQVPRSAATTLDVIPHTAKTPDA |
| | | TFQRQGQTALTTEARATSDSWNEKEKSTPSAPWITEMMNSVSEDTIKEVTSS |
| | | SSVLRTLNTLDINLESGTTSSPSWKSSPYERIAPSESTTDKEAIHPSTNTVE |
| | | TTGWVTSSEHASHSTIPAHSASSKLTSPVVTTSTREQAIVSMSTTTWPESTR |
| | | ARTEPNSFLTIELRDVSPYMDTSSTTQTSIISSPGSTAITKGPRTEITSSKR |
| | | ISSSFLAQSMRSSDSPSEAITRLSNFPAMTESGGMILAMQTSPPGATSLSAP |
| | | TLDTSATASWTGTPLATTQRFTYSEKTTLFSKGPEDTSQPSPPSVEETSSSS |
| | | SLVPIHATTSPSNILLTSQGHSPSSTPPVTSVFLSETSGLGKTTDMSRISLE |
| | | PGTSLPPNLSSTAGEALSTYEASRDTKAIHHSADTAVINMEATSSEYSPIPG |
| | | HTKPSKATSPLVTSHIMGDITSSTSVFGSSETTEIETVSSVNQGLQERSTSQ |
| | | VASSATETSTVITHVSSGDATTHVTKTQATFSSGTSISSPHQFITSTNTFTD |
| | | VSTNPSTSLIMTESSGVTITTQTGPTGAATQGPYLLDTSTMPYLTETPLAVT |
| | | PDFMQSEKTTLISKGPKDVSWTSPPSVAETSYPSSLTPFLVTTIPPATSTLQ |
| | | GQHTSSPVSATSVLTSGLVKTTDMLNTSMEPVINSPQNLNNPSNEILATLAA |
| | | TTDIETIHPSINKAVINMGTASSAHVLHSTLPVSSEPSTATSPMVPASSMGD |
| | | ALASISIPGSETTDIEGEPTSSLTAGRKENSTLQEMNSTTESNIILSNVSVG |
| | | AITEATKMEVPSFDATFIPTPAQSTKFPDIFSVASSRLSNSPPMTISTHMTT |
| | | TQTGSSGATSKIPLALDTSTLETSAGTPSVVTEGFAHSKITTAMNNDVKDVS |
| | | QTNPPFQDEASSPSSQAPVLVTTLPSSVAFTPQWHSTSSPVSMSSVLTSSLV |
| | | KTAGKVDTSLETVTSSPQSMSNTLDDISVTSAATTDIETTHPSINTVVTNVG |
| | | TTGSAFESHSTVSAYPEPSKVTSPNVTTSTMEDTTISRSIPKSSKTTRTETE |
| | | TTSSLTPKLRETSISQEITSSTETSTVPYKELTGATTEVSRTDVTSSSSTSF |
| | | PGPDQSTVSLDISTETNTRLSTSPIMTESAEITITTQTGPHGATSQDTFTMD |
| | | PSNTTPQAGIHSAMTHGFSQLDVTTLMSRIPQDVSWTSPPSVDKTSSPSSFL |
| | | SSPAMTTPSLISSTLPEDKLSSPMTSLLTSGLVKITDILRTRLEPVTSSLPN |
| | | FSSTSDKILATSKDSKDTKEIFPSINTEETNVKANNSGHESHSPALADSETP |
| | | KATTQMVITTTVGDPAPSTSMPVHGSSETTNIKREPTYFLTPRLRETSTSQE |
| | | SSFPTDTSFLLSKVPTGTITEVSSTGVNSSSKISTPDHDKSTVPPDTFTGEI |
| | | PRVFTSSIKTKSAEMTITTQASPPESASHSTLPLDTSTTLSQGGTHSTVTQG |
| | | FPYSEVTTLMGMGPGNVSWMTTPPVEETSSVSSLMSSPAMTSPSPVSSTSPQ |
| | | SIPSSPLPVTALPTSVLVTTTDVLGTTSPESVTSSPPNLSSITHERPATYKD |
| | | TAHTEAAMHHSTNTAVTNVGTSGSGHKSQSSVLADSETSKATPLMSTTSTLG |
| | | DTSVSTSTPNISQTNQIQTEPTASLSPRLRESSTSEKTSSTTETNTAFSYVP |
| | | TGAITQASRTEISSSRTSISDLDRPTIAPDISTGMITRLFTSPIMTKSAEMT |
| | | VTTQTTTPGATSQGILPWDTSTTLFQGGTHSTVSQGFPHSEITTLRSRTPGD |
| | | VSWMTTPPVEETSSGFSLMSPSMTSPSPVSSTSPESIPSSPLPVTALLTSVL |
| | | VTTTNVLGTTSPEPVTSSPPNLSSPTQERLTTYKDTAHTEAMHASMHTNTAV |
| | | ANVGTSISGHESQSSVPADSHTSKATSPMGITFAMGDTSVSTSTPAFFETRI |
| | | QTESTSSLIPGLRDTRISEEINTVTETSTVLSEVPTTTTTEVSRTEVITSSR |
| | | TTISGPDHSKMSPYISTETITRLSTFPFVTGSTEMAITNQTGPIGTISQATL |
| | | TLDTSSTASWEGTHSPVTQRFPHSEETTTMSRSTKGVSWQSPPSVEETSSPS |
| | | SPVPLPAITSHSSLYSAVSGSSPTSALPVTSLLTSGRRKTIDMLDTHSELVT |
| | | SSLPSASSFSGEILTSEASTNTETIHFSENTAETNMGTTNSMHKLHSSVSIH |
| | | SQPSGHTPPKVTGSMMEDAIVSTSTPGSPETKNVDRDSTSPLTPELKEDSTA |
| | | LVMNSTTESNTVFSSVSLDAATEVSRAEVTYYDPTFMPASAQSTKSPDISPE |
| | | ASSSHSNSPPLTISTHKTIATQTGPSGVTSLGQLTLDTSTIATSAGTPSART |
| | | QDFVDSETTSVMNNDLNDVLKTSPFSAEEANSLSSQAPLLVTTSPSPVTSTL |
| | | QEHSTSSLVSVTSVPTPTLAKITDMDTNLEPVTRSPQNLRNTLATSEATTDT |
| | | HTMHPSINTAVANVGTTSSPNEFYFTVSPDSDPYKATSAVVITSTSGDSIVS |
| | | TSMPRSSAMKKIESETTFSLIFRLRETSTSQKIGSSSDTSTVEDKAFTAATT |
| | | EVSRTELTSSSRTSIQGTEKPTMSPDTSTRSVTMLSTFAGLTKSEERTIATQ |
| | | TGPHRATSQGTLTWDTSITTSQAGTHSAMTHGFSQLDLSTLTSRVPEYISGT |
| | | SPPSVEKTSSSSSLLSLPAITSPSPVPTTLPESRPSSPVHLTSLPTSGLVKT |
| | | TDMLASVASLPPNLGSTSHKIPTTSEDIKDTEKMYPSTNIAVTNVGTTTSEK |
| | | ESYSSVPAYSEPPKVTSPMVTSFNIRDTIVSTSMPGSSEITRIEMESTESLA |
| | | HGLKGTSTSQDPIVSTEKSAVLHKLTTGATETSRTEVASSRRTSIPGPDHST |
| | | ESPDISTEVIPSLPISLGITESSNMTIITRTGPPLGSTSQGTFTLDTPTTSS |
| | | RAGTHSMATQEFPHSEMTTVMNKDPEILSWTIPPSIEKTSFSSSLMPSPAMT |
| | | SPPVSSTLPKTIHTTPSPMTSLLTPSLVMTTDTLGTSPEPTTSSPPNLSSTS |
| | | HEILTTDEDTTAIEAMHPSTSTAATNVETTSSGHGSQSSVLADSEKTKATAP |
| | | MDTTSTMGHTTVSTSMSVSSETTKIKRESTYSLTPGLRETSISQNASFSTDT |
| | | SIVLSEVPTGTTAEVSRTEVTSSGRTSIPGPSQSTVLPEISTRTMTRLFASP |
| | | TMTESAEMTIPTQTGPSGSTSQDTLTLDTSTTKSQAKTHSTLTQRFPHSEMT |
| | | TLMSRGPGDMSWQSSPSLENPSSLPSLLSLPATTSPPPISSTLPVTISSSPL |
| | | PVTSLLTSSPVTTTDMLHTSPELVTSSPPKLSHTSDERLTTGKDTTNTEAVH |
| | | PSTNTAASNVEIPSSGHESPSSALADSETSKATSPMFITSTQEDTTVAISTP |
| | | HFLETSRIQKESISSLSPKLRETGSSVETSSAIETSAVLSEVSIGATTEISR |
| | | TEVTSSSRTSISGSAESTMLPEISTTRKIIKFPTSPILAESSEMTIKTQTSP |
| | | PGSTSESTFTLDTSTTPSLVITHSTMTQRLPHSEITTLVSRGAGDVPRPSSL |
| | | PVEETSPPSSQLSLSAMISPSPVSSTLPASSHSSSASVTSLLTPGQVKTTEV |
| | | LDASAEPETSSPPSLSSTSVEILATSEVTTDTEKIHPFSNTAVTKVGTSSSG |
| | | HESPSSVLPDSETTKATSAMGTISIMGDTSVSTLTPALSNTRKIQSEPASSL |
| | | TTRLRETSTSEETSLATEANTVLSKVSTGATTEVSRTEAISFSRTSMSGPEQ |
| | | STMSQDISIGTIPRISASSVLTESAKMTITTQTGPSESTLESTLNLNTATTP |
| | | SWVETHSIVIQGFPHPEMTTSMGRGPGGVSWPSPPFVKETSPPSSPLSLPAV |
| | | TSPHPVSTTFLAHIPPSPLPVTSLLTSGPATTTDILGTSTEPGTSSSSSLST |
| | | TSHERLTTYKDTAHTEAVHPSTNTGGTNVATTSSGYKSQSSVLADSSPMCTT |
| | | STMGDTSVLTSTPAFLETRRIQTELASSLTPGLRESSGSEGTSSGTKMSTVL |
| | | SKVPTGATTEISKEDVTSIPGPAQSTISPDISTRTVSWFSTSPVMTESAEIT |
| | | MNTHTSPLGATTQGTSTLDTSSTTSLTMTHSTISQGFSHSQMSTLMRRGPED |
| | | VSWMSPPLLEKTRPSFSLMSSPATTSPSPVSSTLPESISSSPLPVTSLLISG |
| | | LAKTTDMLHKSSEPVTNSPANLSSTSVEILATSEVTTDTEKTHPSSNRTVTD |
| | | VGTSSSGHESTSFVLADSQTSKVTSPMVITSTMEDTSVSTSTPGFFETSRIQ |
| | | TEPTSSLTLGLRKTSSSEGTSLATEMSTVLSGVPTGATAEVSRTEVTSSSRT |
| | | SISGFAQLTVSPETSTETITRLPTSSIMTESAEMMIKTQTDPPGSTPESTHT |
| | | VDISTTPNWVETHSTVTQRFSHSEMTTLVSRSPGDMLWPSQSSVEETSSASS |
| | | LLSLPATTSPSPVSSTLVEDFPSASLPVTSLLNPGLVITTDRMGISREPGTS |
| | | STSNLSSTSHERLTTLEDTVDTEDMQPSTHTAVINVRTSISGHESQSSVLSD |
| | | SETPKATSPMGTTYTMGETSVSISTSDFFETSRIQIEPTSSLTSGLRETSSS |
| | | ERISSATEGSTVLSEVPSGATTEVSRTEVISSRGTSMSGPDQFTISPDISTE |
| | | AITRLSTSPIMTESAESAITIETGSPGATSEGTLTLDTSTTTFWSGTHSTAS |
| | | PGFSHSEMTTLMSRTPGDVPWPSLPSVEEASSVSSSLSSPAMTSTSFFSTLP |
| | | ESISSSPHPVTALLTLGPVKTTDMLRTSSEPETSSPPNLSSTSAEILATSEV |
| | | TKDREKIHPSSNTPVVNVGTVIYKHLSPSSVLADLVTTKPTSPMATTSTLGN |
| | | TSVSTSTPAFPETMMTQPTSSLTSGLREISTSQETSSATERSASLSGMPTGA |
| | | TTKVSRTEALSLGRTSTPGPAQSTISPEISTETITRISTPLTTTGSAEMTIT |
| | | PKTGHSGASSQGTFTLDTSSRASWPGTHSAATHRSPHSGMTTPMSRGPEDVS |
| | | WPSRPSVEKTSPPSSLVSLSAVTSPSPLYSTPSESSHSSPLRVTSLFTPVMM |
| | | KTTDMLDTSLEPVTTSPPSMNITSDESLATSKATMETEAIQLSENTAVTQMG |
| | | TISARQEFYSSYPGLPEPSKVTSPVVTSSTIKDIVSTTIPASSEITRIEMES |
| | | TSTLTPTPRETSTSQEIHSATKPSTVPYKALTSATIEDSMTQVMSSSRGPSP |
| | | DQSTMSQDISTEVITRLSTSPIKTESTEMTITTQTGSPGATSRGTLTLDTST |
| | | TFMSGTHSTASQGFSHSQMTALMSRTPGDVPWLSHPSVEEASSASFSLSSPV |
| | | MTSSSPVSSTLPDSIHSSSLPVTSLLTSGLVKTTELLGTSSEPETSSPPNLS |
| | | STSAEILAITEVTTDTEKLEMTNVVTSGYTHESPSSVLADSVTTKATSSMGI |
| | | TYPTGDTNVLTSTPAFSDTSRIQTKSKLSLTPGLMETSISEETSSATEKSTV |
| | | LSSVPTGATTEVSRTEAISSSRTSIPGPAQSTMSSDTSMETITRISTPLTRK |
| | | ESTDMAITPKTGPSGATSQGTFTLDSSSTASWPGTHSATTQRFPQSVVTTPM |
| | | SRGPEDVSWPSPLSVEKNSPPSSLVSSSSVTSPSPLYSTPSGSSHSSPVPVT |
| | | SLFTSIMMKATDMLDASLEPETTSAPNMNITSDESLAASKATTETEAIHVFE |
| | | NTAASHVETTSATEELYSSSPGFSEPTKVISPVVTSSSIRDNMVSTTMPGSS |
| | | GITRIEIESMSSLIPGLRETRISQDITSSTETSTVLYKMPSGATPEVSRTEV |
| | | MPSSRTSIPGPAQSTMSLDISDEVVTRLSTSPIMTESAEITITTQTGYSLAT |
| | | SQVTLPLGTSMTFLSGTHSTMSQGLSHSEMTNLMSRGPESLSWTSPRFVETT |
| | | RSSSSLTSLPLTTSLSPVSSTLLDSSPSSPLPVTSLILPGLVKTTEVLDTSS |
| | | EPKTSSSPNLSSTSVEIPATSEIMTDTEKIHPSSNTAVAKVRTSSSVHESHS |
| | | SVLADSETTITIPSMGITSAVDDTTVFTSNPAFSETRRIPTEPTFSLTPGFR |
| | | ETSTSEETTSITETSAVLYGVPTSATTEVSMTEIMSSNRIHIPDSDQSTMSP |
| | | DIITEVITRLSSSSMMSESTQMTITTQKSSPGATAQSTLTLATTTAPLARTH |
| | | STVPPRFLHSEMTTLMSRSPENPSWKSSLFVEKTSSSSSLLSLPVTTSPSVS |
| | | STLPQSIPSSSFSVTSLLTPGMVKTTDTSTEPGTSLSPNLSGTSVEILAASE |
| | | VTTDTEKIHPSSSMAVINVGTTSSGHELYSSVSIHSEPSKATYPVGTPSSMA |
| | | ETSISTSMPANFETTGFEAEPFSHLTSGFRKINMSLDTSSVTPTNTPSSPGS |
| | | THLLQSSKTDFTSSAKTSSPDWPPASQYTEIPVDIITPFNASPSITESTGIT |
| | | SFPESRFTMSVTESTHHLSTDLLPSAETISTGTVMPSLSEAMTSFATTGVPR |
| | | AISGSGSPFSRTESGPGDATLSTIAESLPSSTPVPFSSSTFTTTDSSTIPAL |
| | | HEITSSSATPYRVDTSLGTESSTTEGRLVMVSTLDTSSQPGRTSSSPILDTR |
| | | MTESVELGTVTSAYQVPSLSTRLTRTDGIMEHITKIPNEAAHRGTIRPVKGP |
| | | QTSTSPASPKGLHTGGTKRMETTTTALKTTTTALKTTSRATLTTSVYTPTLG |
| | | TLTPLNASMQMASTIPTEMMITTPYVFPDVPETTSSLATSLGAETSTALPRT |
| | | TPSVENRESETTASLVSRSGAERSPVIQTLDVSSSEPDTTASWVIHPAETIP |
| | | TVSKTTPNFFHSELDTVSSTATSHGADVSSAIPTNISPSELDALTPLVTISG |
| | | TDTSTTFPTLTKSPHETETRTTWLTHPAETSSTIPRTIPNFSHHESDATPSI |
| | | ATSPGAETSSAIPIMTVSPGAEDLVTSQVTSSGTDRNMTIPTLTLSPGEPKT |
| | | IASLVTHPEAQTSSAIPTSTISPAVSRLVTSMVTSLAAKTSTTNRALTNSPG |
| | | EPATTVSLVTHPAQTSPTVPWTTSIFFHSKSDTTPSMTTSHGAESSSAVPTP |
| | | TVSTEVPGVVTPLVISSRAVISTTIPILTLSPGEPETTPSMATSHGEEASSA |
| | | IPTPTVSPGVPGVVTSLVTSSRAVTSTTIPILTFSLGEPETTPSMATSHGTE |
| | | AGSAVPTVLPEVPGMVTSLVASSRAVTSTTLPTLTLSPGEPETTPSMATSHG |
| | | AEASSTVPTVSPEVPGVVTSLVTSSSGVNSTSIPTLILSPGELETTPSMATS |
| | | HGAEASSAVPTPTVSPGVSGVVTPLVTSSRAVTSTTIPILTLSSSEPETTPS |
| | | MATSHGVEASSAVLTVSPEVPGMVTSLVTSSRAVISTTIPTLTISSDEPETT |
| | | TSLVTHSEAKMISAIPTLAVSPTVQGLVTSLVTSSGSETSAFSNLTVASSQP |
| | | ETIDSWVAHPGTEASSVVPTLTVSTGEPFTNISLVTHPAESSSTLPRTTSRF |
| | | SHSELDTMPSTVTSPEAESSSAISTTISPGIPGVLTSLVTSSGRDISATFPT |
| | | VPESPHESEATASWVTHPAVTSTTVPRTTPNYSHSEPDTTPSIATSPGAEAT |
| | | SDFPTITVSPDVPDMVTSQVTSSGTDTSITIPTLTLSSGEPETITSFITYSE |
| | | THTSSAIPTLPVSPGASKMLTSLVISSGTDSTTTFPTLTETPYEPETTAIQL |
| | | IHPAETNTMVPRTTPKFSHSKSDTTLPVAITSPGPEASSAVSTTTISPDMSD |
| | | LVTSLVPSSGTDTSTTFPTLSETPYEPETTATWLTHPAETSTTVSGTIPNFS |
| | | HRGSDTAPSMVTSPGVDTRSGVPTTTIPPSIPGVVTSQVTSSATDTSTAIPT |
| | | LTPSPGEPETTASSATHPGTQTGFTVPIRTVPSSEPDTMASWVTHPPQTSTP |
| | | VSRTTSSFSHSSPDATPVMATSPRTEASSAVLTTISPGAPEMVTSQITSSGA |
| | | ATSTTVPTLTHSPGMPETTALLSTHPRTETSKTFPASTVFPQVSETTASLTI |
| | | RPGAETSTALPTQTTSSLFTLLVTGTSRVDLSPTASPGVSAKTAPLSTHPGT |
| | | ETSTMIPTSTLSLGLLETTGLLATSSSAETSTSTLTLTVSPAVSGLSSASIT |
| | | TDKPQTVTSWNTETSPSVTSVGPPEFSRTVTGTTMTLIPSEMPTPPKTSHGE |
| | | GVSPTTILRTTMVEATNLATTGSSPTVAKTTTTFNTLAGSLFTPLTTPGMST |
| | | LASESVTSRTSYNHRSWISTTSSYNRRYWTPATSTPVTSTFSPGISTSSIPS |
| | | STAATVPFMVPFTLNFTITNLQYEEDMRHPGSRKFNATERELQGLLKPLERN |
| | | SSLEYLYSGCRLASLRPEKDSSATAVDAICTHRPDPEDLGLDRERLYWELSN |
| | | LINGIQELGPYTLDRNSLYVNGFTHRSSMPTTSTPGTSTVDVGTSGTPSSSP |
| | | SPTTAGPLLMPFTLNFTITNLQYEEDMRRTGSRKFNTMESVLQGLLKPLFKN |
| | | TSVGPLYSGCRLTLLRPEKDGAATGVDAICTHRLDPKSPGLNREQLYWELSK |
| | | LTNDIEELGPYTLDRNSLYVNGFTHQSSVSTTSTPGTSTVDLRTSGTPSSLS |
| | | SPTIMAAGPLLVPFTLNFTITNLQYGEDMGHPGSRKENTTERVLQGLLGPIF |
| | | KNTSVGPLYSGCRLTSLRSEKDGAATGVDAICIHHLDPKSPGLNRERLYWEL |
| | | SQLTNGIKELGPYTLDRNSLYVNGFTHRTSVPTSSTPGTSTVDLGTSGTPFS |
| | | LPSPATAGPLLVLFTLNFTITNLKYEEDMHRPGSRKFNTTERVLQTLLGPMF |
| | | KNTSVGLLYSGCRLTLLRSEKDGAATGVDAICTHRLDPKSPGVDREQLYWEL |
| | | SQLTNGIKELGPYTLDRNSLYVNGFTHWIPVPTSSTPGTSTVDLGSGTPSSL |
| | | PSPTTAGPLLVPFTLNFTITNLKYEEDMHCPGSRKENTTERVLQSLLGPMFK |
| | | NTSVGPLYSGCRLTLLRSEKDGAATGVDAICTHRLDPKSPGVDREQLYWELS |
| | | QLTNGIKELGPYTLDRNSLYVNGFTHQTSAPNTSTPGTSTVDLGTSGTPSSL |
| | | PSPTSAGPLLVPFTLNFTITNLQYEEDMHHPGSRKFNTTERVLQGLLGPMEK |
| | | NTSVGLLYSGCRLTLLRPEKNGAATGMDAICSHRLDPKSPGLNREQLYWELS |
| | | QLTHGIKELGPYTLDRNSLYVNGFTHRSSVAPTSTPGTSTVDLGTSGTPSSL |
| | | PSPTTAVPLLVPFTLNFTITNLQYGEDMRHPGSRKFNTTERVLQGLLGPLFK |
| | | NSSVGPLYSGCRLISLRSEKDGAATGVDAICTHHLNPQSPGLDREQLYWQLS |
| | | QMTNGIKELGPYTLDRNSLYVNGFTHRSSGLTTSTPWTSTVDLGTSGTPSPV |
| | | PSPTTTGPLLVPFTLNFTITNLQYEENMGHPGSRKFNITESVLQGLLKPLEK |
| | | STSVGPLYSGCRLTLLRPEKDGVATRVDAICTHRPDPKIPGLDRQQLYWELS |
| | | QLTHSITELGPYTLDRDSLYVNGFTQRSSVPTTSTPGTFTVQPETSETPSSL |
| | | PGPTATGPVLLPFTLNFTITNLQYEEDMRRPGSRKENTTERVLQGLLMPLFK |
| | | NTSVSSLYSGCRLTLLRPEKDGAATRVDAVCTHRPDPKSPGLDRERLYWKLS |
| | | QLTHGITELGPYTLDRHSLYVNGFTHQSSMTTTRTPDTSTMHLATSRTPASL |
| | | SGPMTASPLLVLFTINFTITNLRYEENMHHPGSRKENTTERVLQGLLRPVFK |
| | | NTSVGPLYSGCRLTLLRPKKDGAATKVDAICTYRPDPKSPGLDREQLYWELS |
| | | QLTHSITELGPYTLDRDSLYVNGFTQRSSVPTTSIPGTPTVDLGTSGTPVSK |
| | | PGPSAASPLLVLFTLNFTITNLRYEENMQHPGSRKENTTERVLQGLLRSLFK |
| | | STSVGPLYSGCRLTLLRPEKDGTATGVDAICTHHPDPKSPRLDREQLYWELS |
| | | QLTHNITELGPYALDNDSLFVNGFTHRSSVSTTSTPGTPTVYLGASKTPASI |
| | | FGPSAASHLLILFTLNFTITNLRYEENMWPGSRKFNTTERVLQGLLRPLFKN |
| | | TSVGPLYSGCRLTLLRPEKDGEATGVDAICTHRPDPTGPGLDREQLYLELSQ |
| | | LTHSITELGPYTLDRDSLYVNGFTHRSSVPTTSTGVVSEEPFTLNFTINNLR |
| | | YMADMGQPGSLKFNITDNVMQHLLSPLFQRSSLGARYTGCRVIALRSVKNGA |
| | | ETRVDLLCTYLQPLSGPGLPIKQVFHELSQQTHGITRLGPYSLDKDSLYLNG |
| | | YNEPGPDEPPTTPKPATTFLPPLSEATTAMGYHLKTLTLNFTISNLQYSPDM |
| | | GKGSATFNSTEGVLQHLLRPLFQKSSMGPFYLGCQLISLRPEKDGAATGVDT |
| | | TCTYHPDPVGPGLDIQQLYWELSQLTHGVTQLGFYVLDRDSLFINGYAPQNL |
| | | SIRGEYQINFHIVNWNLSNPDPTSSEYITLLRDIQDKVTTLYKGSQLHDTER |
| | | FCLVTNLTMDSVLVTVKALFSSNLDPSLVEQVFLDKTLNASFHWLGSTYQLV |
| | | DIHVTEMESSVYQPTSSSSTQHFYLNFTITNLPYSQDKAQPGTTNYQRNKRN |
| | | IEDALNQLFRNSSIKSYFSDCQVSTFRSVPNRHHTGVDSLCNFSPLARRVDR |
| | | VAIYEEFLRMTRNGTQLQNFTLDRSSVLVDGYSPNRNEPLIGNSDLPFWAVI |
| | | LIGLAGLLGVITCLICGVLVTTRRRKKEGEYNVQQQCPGYYQSHLDLEDLQ |
| 14 | Linker | DGGGS |
| 15 | Linker | TGEKP |
| 16 | Linker | GGRR |
| 17 | Linker | (GGGGS)ₙ; wherein n = 1, 2, 3, 4 or 5 |
| 18 | Linker | EGKSSGSGSESKVD |
| 19 | Linker | KESGSVSSEQLAQFRSLD |
| 20 | Linker | GGRRGGGS |
| 21 | Linker | LRQRDGERP |
| 22 | Linker | LRQKDGGGSERP |
| 23 | Linker | LRQKD(GGGS)₂ ERP |
| 24 | Linker | GGGGSGGGGSGGGGS |
| 25 | Linker | GSTSGSGKPGSGEGSTKG |
| 26 | 2A cleaving | LLNFDLLKLAGDVESNPGP |
| | site | |
| 27 | 2A cleaving | TLNFDLLKLAGDVESNPGP |
| | site | |
| 28 | 2A cleaving | LLKLAGDVESNPGP |
| | site | |
| 29 | 2A cleaving | NFDLLKLAGDVESNPGP |
| | site | |
| 30 | 2A cleaving | QLLNFDLLKLAGDVESNPGP |
| | site | |
| 31 | 2A cleaving | APVKQTLNFDLLKLAGDVESNPGP |
| | site | |
| 32 | 2A cleaving | VTELLYRMKRAETYCPRPLLAIHPTEARHKQKIVAPVKQT |
| | site | |
| 33 | 2A cleaving | LNFDLLKLAGDVESNPGP |
| | site | |
| 34 | 2A cleaving | LLAIHPTEARHKQKIVAPVKQTLNFDLLKLAGDVESNPGP |
| | site | |
| 35 | 2A cleaving | EARHKQKIVAPVKQTLNFDLLKLAGDVESNPGP |
| | site | |
| 36 | Cleavage site | EXXYXQ(G/S) |
| 37 | Cleavage site | ENLYFQG |
| 38 | Cleavage site | ENLYFQS |
| 39 | Human MUC16 | PGSRKFNTTERVLQGLLRPLFKNTSVGPLYSGCRLTLLRPEKDGEATGVDAI |
| | | CTHRPDPTGPGLDREQLYLELSQLTHSITELGPYTLDRDSLYVNGFTHRSSV |
| | | PTTSTGVVSEEPFTLNFTINNLRYMADMGQPGSLKFNITDNVMQHLLSPLFQ |
| | | RSSLGARYTGCRVIALRSVKNGAETRVDLLCTYLQPLSGPGLPIKQVFHELS |
| | | QQTHGITRLGPYSLDKDSLYLNGYNEPGPDEPPTTPKPATTFLPPLSEATTA |
| | | MGYHLKTLTLNFTISNLQYSPDMGKGSATFNSTEGVLQHLLRPLFQKSSMGP |
| | | FYLGCQLISLRPEKDGAATGVDTTCTYHPDPVGPGLDIQQLYWELSQLTHGV |
| | | TQLGFYVLDRDSLFINGYAPQNLSIRGEYQINFHIVNWNLSNPDPTSSEYIT |
| | | LLRDIQDKVTTLYKGSQLHDTFRFCLVTNLTMDSVLVTVKALFSSNLDPSLV |
| | | EQVFLDKTLNASFHWLGSTYQLVDIHVTEMESSVYQPTSSSSTQHFYLNFTI |
| | | TNLPYSQDKAQPGTTNYQRNKRNIEDALNQLERNSSIKSYFSDCQVSTFRSV |
| | | PNRHHTGVDSLCNFSPLARRVDRVAIYEEFLRMTRNGTQLQNFTLDRSSVLV |
| | | DGYSPNRNEPLIGNSDLPEQKLISEEDLGGEQKLISEEDLHHHHHH |
| 40 | ectodomain with | MLKPSGLPGSSSPTRSLMTGSRSTKATPEMDSGLTGATLSPKTSTGAIVVTE |
| | myc-myc-his tag | HTLPFTSPDKTLASPTSSVVGRTTQSLGVMSSALPESTSRGMTHSEQRTSPS |
| | MUC16 | LSPQVNGTPSRNYPATSMVSGLSSPRTRTSSTEGNFTKEASTYTLTVETTSG |
| | polypeptide | PVTEKYTVPTETSTTEGDSTETPWDTRYIPVKITSPMKTFADSTASKENAPV |
| | lacking the nub | SMTPAETTVTDSHTPGRTNPSFGTLYSSFLDLSPKGTPNSRGETSLELILST |
| | (CA125) | TGYPFSSPEPGSAGHSRISTSAPLSSSASVLDNKISETSIFSGQSLTSPLSP |
| | | GVPEARASTMPNSAIPFSMTLSNAETSAERVRSTISSLGTPSISTKQTAETI |
| | | LTFHAFAETMDIPSTHIAKTLASEWLGSPGTLGGTSTSALTTTSPSTTLVSE |
| | | ETNTHHSTSGKETEGTLNTSMTPLETSAPGEESEMTATLVPTLGFTTLDSKI |
| | | RSPSQVSSSHPTRELRTTGSTSGRQSSSTAAHGSSDILRATTSSTSKASSWT |
| | | SESTAQQFSEPQHTQWVETSPSMKTERPPASTSVAAPITTSVPSVVSGFTTL |
| | | KTSSTKGIWLEETSADTLIGESTAGPTTHQFAVPTGISMTGGSSTRGSQGTT |
| | | HLLTRATASSETSADLTLATNGVPVSVSPAVSKTAAGSSPPGGTKPSYTMVS |
| | | SVIPETSSLQSSAFREGTSLGLTPLNTRHPFSSPEPDSAGHTKISTSIPLLS |
| | | SASVLEDKVSATSTFSHHKATSSITTGTPEISTKTKPSSAVLSSMTLSNAAT |
| | | SPERVRNATSPLTHPSPSGEETAGSVLTLSTSAETTDSPNIHPTGTLTSESS |
| | | ESPSTLSLPSVSGVKTTFSSSTPSTHLFTSGEETEETSNPSVSQPETSVSRV |
| | | RTTLASTSVPTPVFPTMDTWPTRSAQFSSSHLVSELRATSSTSVINSTGSAL |
| | | PKISHLTGTATMSQTNRDTENDSAAPQSTTWPETSPRFKTGLPSATTTVSTS |
| | | ATSLSATVMVSKFTSPATSSMEATSIREPSTTILTTETTNGPGSMAVASTNI |
| | | PIGKGYITEGRLDTSHLPIGTTASSETSMDFTMAKESVSMSVSPSQSMDAAG |
| | | SSTPGRTSQFVDTFSDDVYHLTSREITIPRDGTSSALTPQMTATHPPSPDPG |
| | | SARSTWLGILSSSPSSPTPKVTMSSTFSTQRVTTSMIMDTVETSRWNMPNLP |
| | | STTSLTPSNIPTSGAIGKSTLVPLDTPSPATSLEASEGGLPTLSTYPESTNT |
| | | PSIHLGAHASSESPSTIKLTMASVVKPGSYTPLTFPSIETHIHVSTARMAYS |
| | | SGSSPEMTAPGETNTGSTWDPTTYITTTDPKDTSSAQVSTPHSVRTLRTTEN |
| | | HPKTESATPAAYSGSPKISSSPNLTSPATKAWTITDTTEHSTQLHYTKLAEK |
| | | SSGFETQSAPGPVSVVIPTSPTIGSSTLELTSDVPGEPLVLAPSEQTTITLP |
| | | MATWLSTSLTEEMASTDLDISSPSSPMSTFAIFPPMSTPSHELSKSEADTSA |
| | | IRNTDSTTLDQHLGIRSLGRTGDLTTVPITPLTTTWTSVIEHSTQAQDTLSA |
| | | TMSPTHVTQSLKDQTSIPASASPSHLTEVYPELGTQGRSSSEATTFWKPSTD |
| | | TLSREIETGPTNIQSTPPMDNTTTGSSSSGVTLGIAHLPIGTSSPAETSTNM |
| | | ALERRSSTATVSMAGTMGLLVTSAPGRSISQSLGRVSSVLSESTTEGVTDSS |
| | | KGSSPRLNTQGNTALSSSLEPSYAEGSQMSTSIPLTSSPTTPDVEFIGGSTF |
| | | WTKEVTTVMTSDISKSSARTESSSATLMSTALGSTENTGKEKLRTASMDLPS |
| | | PTPSMEVTPWISLTLSNAPNTTDSLDLSHGVHTSSAGTLATDRSLNTGVTRA |
| | | SRLENGSDTSSKSLSMGNSTHTSMTYTEKSEVSSSIHPRPETSAPGAETTLT |
| | | STPGNRAISLTLPFSSIPVEEVISTGITSGPDINSAPMTHSPITPPTIVWTS |
| | | TGTIEQSTQPLHAVSSEKVSVQTQSTPYVNSVAVSASPTHENSVSSGSSTSS |
| | | PYSSASLESLDSTISRRNAITSWLWDLTTSLPTTTWPSTSLSEALSSGHSGV |
| | | SNPSSTTTEFPLFSAASTSAAKQRNPETETHGPQNTAASTLNTDASSVTGLS |
| | | ETPVGASISSEVPLPMAITSRSDVSGLTSESTANPSLGTASSAGTKLTRTIS |
| | | LPTSESLVSFRMNKDPWTVSIPLGSHPTTNTETSIPVNSAGPPGLSTVASDV |
| | | IDTPSDGAESIPTVSFSPSPDTEVTTISHFPEKTTHSFRTISSLTHELTSRV |
| | | TPIPGDWMSSAMSTKPTGASPSITLGERRTITSAAPTTSPIVLTASFTETST |
| | | VSLDNETTVKTSDILDARKTNELPSDSSSSSDLINTSIASSTMDVTKTASIS |
| | | PTSISGMTASSSPSLFSSDRPQVPTSTTETNTATSPSVSSNTYSLDGGSNVG |
| | | GTPSTLPPFTITHPVETSSALLAWSRPVRTFSTMVSTDTASGENPTSSNSVV |
| | | TSVPAPGTWTSVGSTTDLPAMGFLKTSPAGEAHSLLASTIEPATAFTPHLSA |
| | | AVVTGSSATSEASLLTTSESKAIHSSPQTPTTPTSGANWETSATPESLLVVT |
| | | ETSDTTLTSKILVTDTILFSTVSTPPSKFPSTGTLSGASFPTLLPDTPAIPL |
| | | TATEPTSSLATSFDSTPLVTIASDSLGTVPETTLTMSETSNGDALVLKTVSN |
| | | PDRSIPGITIQGVTESPLHPSSTSPSKIVAPRNTTYEGSITVALSTLPAGTT |
| | | GSLVFSQSSENSETTALVDSSAGLERASVMPLTTGSQGMASSGGIRSGSTHS |
| | | TGTKTFSSLPLTMNPGEVTAMSEITTNRLTATQSTAPKGIPVKPTSAESGLL |
| | | TPVSASSSPSKAFASLTTAPPTWGIPQSTLTFEFSEVPSLDTKSASLPTPGQ |
| | | SLNTIPDSDASTASSSLSKSPEKNPRARMMTSTKAISASSFQSTGFTETPEG |
| | | SASPSMAGHEPRVPTSGTGDPRYASESMSYPDPSKASSAMTSTSLASKLTTL |
| | | FSTGQAARSGSSSSPISLSTEKETSFLSPTASTSRKTSLFLGPSMARQPNIL |
| | | VHLQTSALTLSPTSTLNMSQEEPPELTSSQTIAEEEGTTAETQTLTFTPSET |
| | | PTSLLPVSSPTEPTARRKSSPETWASSISVPAKTSLVETTDGTLVTTIKMSS |
| | | QAAQGNSTWPAPAEETGSSPAGTSPGSPEMSTTLKIMSSKEPSISPEIRSTV |
| | | RNSPWKTPETTVPMETTVEPVTLQSTALGSGSTSISHLPTGTTSPTKSPTEN |
| | | MLATERVSLSPSPPEAWTNLYSGTPGGTRQSLATMSSVSLESPTARSITGTG |
| | | QQSSPELVSKTTGMEFSMWHGSTGGTTGDTHVSLSTSSNILEDPVTSPNSVS |
| | | SLTDKSKHKTETWVSTTAIPSTVLNNKIMAAEQQTSRSVDEAYSSTSSWSDQ |
| | | TSGSDITLGASPDVTNTLYITSTAQTTSLVSLPSGDQGITSLTNPSGGKTSS |
| | | ASSVTSPSIGLETLRANVSAVKSDIAPTAGHLSQTSSPAEVSILDVTTAPTP |
| | | GISTTITTMGINSISTTTPNPEVGMSTMDSTPATERRTTSTEHPSTWSSTAA |
| | | SDSWTVTDMTSNLKVARSPGTISTMHTTSFLASSTELDSMSTPHGRITVIGT |
| | | SLVTPSSDASAVKTETSTSERTLSPSDTTASTPISTFSRVQRMSISVPDILS |
| | | TSWTPSSTEAEDVPVSMVSTDHASTKTDPNTPLSTFLFDSLSTLDWDTGRSL |
| | | SSATATTSAPQGATTPQELTLETMISPATSQLPFSIGHITSAVTPAAMARSS |
| | | GVTFSRPDPTSKKAEQTSTQLPTTTSAHPGQVPRSAATTLDVIPHTAKTPDA |
| | | TFQRQGQTALTTEARATSDSWNEKEKSTPSAPWITEMMNSVSEDTIKEVTSS |
| | | SSVLRTLNTLDINLESGTTSSPSWKSSPYERIAPSESTTDKEAIHPSTNTVE |
| | | TTGWVTSSEHASHSTIPAHSASSKLTSPVVTTSTREQAIVSMSTTTWPESTR |
| | | ARTEPNSFLTIELRDVSPYMDTSSTTQTSIISSPGSTAITKGPRTEITSSKR |
| | | ISSSFLAQSMRSSDSPSEAITRLSNFPAMTESGGMILAMQTSPPGATSLSAP |
| | | TLDTSATASWTGTPLATTQRFTYSEKTTLFSKGPEDTSQPSPPSVEETSSSS |
| | | SLVPIHATTSPSNILLTSQGHSPSSTPPVTSVFLSETSGLGKTTDMSRISLE |
| | | PGTSLPPNLSSTAGEALSTYEASRDTKAIHHSADTAVINMEATSSEYSPIPG |
| | | HTKPSKATSPLVTSHIMGDITSSTSVFGSSETTEIETVSSVNQGLQERSTSQ |
| | | VASSATETSTVITHVSSGDATTHVTKTQATFSSGTSISSPHQFITSTNTFTD |
| | | VSTNPSTSLIMTESSGVTITTQTGPTGAATQGPYLLDTSTMPYLTETPLAVT |
| | | PDFMQSEKTTLISKGPKDVSWTSPPSVAETSYPSSLTPFLVTTIPPATSTLQ |
| | | GQHTSSPVSATSVLTSGLVKTTDMLNTSMEPVTNSPQNLNNPSNEILATLAA |
| | | TTDIETIHPSINKAVTNMGTASSAHVLHSTLPVSSEPSTATSPMVPASSMGD |
| | | ALASISIPGSETTDIEGEPTSSLTAGRKENSTLQEMNSTTESNIILSNVSVG |
| | | AITEATKMEVPSFDATFIPTPAQSTKFPDIFSVASSRLSNSPPMTISTHMTT |
| | | TQTGSSGATSKIPLALDTSTLETSAGTPSVVTEGFAHSKITTAMNNDVKDVS |
| | | QTNPPFQDEASSPSSQAPVLVTTLPSSVAFTPQWHSTSSPVSMSSVLTSSLV |
| | | KTAGKVDTSLETVTSSPQSMSNTLDDISVTSAATTDIETTHPSINTVVTNVG |
| | | TTGSAFESHSTVSAYPEPSKVTSPNVTTSTMEDTTISRSIPKSSKTTRTETE |
| | | TTSSLTPKLRETSISQEITSSTETSTVPYKELTGATTEVSRTDVTSSSSTSF |
| | | PGPDQSTVSLDISTETNTRLSTSPIMTESAEITITTQTGPHGATSQDTFTMD |
| | | PSNTTPQAGIHSAMTHGFSQLDVTTLMSRIPQDVSWTSPPSVDKTSSPSSFL |
| | | SSPAMTTPSLISSTLPEDKLSSPMTSLLTSGLVKITDILRTRLEPVTSSLPN |
| | | FSSTSDKILATSKDSKDTKEIFPSINTEETNVKANNSGHESHSPALADSETP |
| | | KATTQMVITTTVGDPAPSTSMPVHGSSETTNIKREPTYFLTPRLRETSTSQE |
| | | SSFPTDTSFLLSKVPTGTITEVSSTGVNSSSKISTPDHDKSTVPPDTFTGEI |
| | | PRVFTSSIKTKSAEMTITTQASPPESASHSTLPLDTSTTLSQGGTHSTVTQG |
| | | FPYSEVTTLMGMGPGNVSWMTTPPVEETSSVSSLMSSPAMTSPSPVSSTSPQ |
| | | SIPSSPLPVTALPTSVLVTTTDVLGTTSPESVTSSPPNLSSITHERPATYKD |
| | | TAHTEAAMHHSTNTAVTNVGTSGSGHKSQSSVLADSETSKATPLMSTTSTLG |
| | | DTSVSTSTPNISQTNQIQTEPTASLSPRLRESSTSEKTSSTTETNTAFSYVP |
| | | TGAITQASRTEISSSRTSISDLDRPTIAPDISTGMITRLFTSPIMTKSAEMT |
| | | VTTQTTTPGATSQGILPWDTSTTLFQGGTHSTVSQGFPHSEITTLRSRTPGD |
| | | VSWMTTPPVEETSSGFSLMSPSMTSPSPVSSTSPESIPSSPLPVTALLTSVL |
| | | VTTTNVLGTTSPEPVTSSPPNLSSPTQERLTTYKDTAHTEAMHASMHTNTAV |
| | | ANVGTSISGHESQSSVPADSHTSKATSPMGITFAMGDTSVSTSTPAFFETRI |
| | | QTESTSSLIPGLRDTRTSEEINTVTETSTVLSEVPTTTTTEVSRTEVITSSR |
| | | TTISGPDHSKMSPYISTETITRLSTFPFVTGSTEMAITNQTGPIGTISQATL |
| | | TLDTSSTASWEGTHSPVTQRFPHSEETTTMSRSTKGVSWQSPPSVEETSSPS |
| | | SPVPLPAITSHSSLYSAVSGSSPTSALPVTSLLTSGRRKTIDMLDTHSELVT |
| | | SSLPSASSFSGEILTSEASINTETIHFSENTAETNMGTTNSMHKLHSSVSIH |
| | | SQPSGHTPPKVTGSMMEDAIVSTSTPGSPETKNVDRDSTSPLTPELKEDSTA |
| | | LVMNSTTESNTVFSSVSLDAATEVSRAEVTYYDPTFMPASAQSTKSPDISPE |
| | | ASSSHSNSPPLTISTHKTIATQTGPSGVTSLGQLTLDTSTIATSAGTPSART |
| | | QDFVDSETTSVMNNDLNDVLKTSPFSAEEANSLSSQAPLLVTTSPSPVTSTL |
| | | QEHSTSSLVSVTSVPTPTLAKITDMDTNLEPVTRSPQNLRNTLATSEATTDT |
| | | HTMHPSINTAVANVGTTSSPNEFYFTVSPDSDPYKATSAVVITSTSGDSIVS |
| | | TSMPRSSAMKKIESETTFSLIFRLRETSTSQKIGSSSDTSTVEDKAFTAATT |
| | | EVSRTELTSSSRTSIQGTEKPTMSPDTSTRSVTMLSTFAGLTKSEERTIATQ |
| | | TGPHRATSQGTLTWDTSITTSQAGTHSAMTHGFSQLDLSTLTSRVPEYISGT |
| | | SPPSVEKTSSSSSLLSLPAITSPSPVPTTLPESRPSSPVHLTSLPTSGLVKT |
| | | TDMLASVASLPPNLGSTSHKIPTTSEDIKDTEKMYPSTNIAVTNVGTTTSEK |
| | | ESYSSVPAYSEPPKVTSPMVTSFNIRDTIVSTSMPGSSEITRIEMESTFSLA |
| | | HGLKGTSTSQDPIVSTEKSAVLHKLTTGATETSRTEVASSRRTSIPGPDHST |
| | | ESPDISTEVIPSLPISLGITESSNMTIITRTGPPLGSTSQGTFTLDTPTTSS |
| | | RAGTHSMATQEFPHSEMTTVMNKDPEILSWTIPPSIEKTSFSSSLMPSPAMT |
| | | SPPVSSTLPKTIHTTPSPMTSLLTPSLVMTTDTLGTSPEPTTSSPPNLSSTS |
| | | HEILTTDEDTTAIEAMHPSTSTAATNVETTSSGHGSQSSVLADSEKTKATAP |
| | | MDTTSTMGHTTVSTSMSVSSETTKIKRESTYSLTPGLRETSISQNASFSTDT |
| | | SIVLSEVPTGTTAEVSRTEVISSGRTSIPGPSQSTVLPEISTRTMTRLFASP |
| | | TMTESAEMTIPTQTGPSGSTSQDTLTLDTSTTKSQAKTHSTLTQRFPHSEMT |
| | | TLMSRGPGDMSWQSSPSLENPSSLPSLLSLPATTSPPPISSTLPVTISSSPL |
| | | PVTSLLTSSPVTTTDMLHTSPELVTSSPPKLSHTSDERLTTGKDTTNTEAVH |
| | | PSTNTAASNVEIPSSGHESPSSALADSETSKATSPMFITSTQEDTTVAISTP |
| | | HFLETSRIQKESISSLSPKLRETGSSVETSSAIETSAVLSEVSIGATTEISR |
| | | TEVTSSSRTSISGSAESTMLPEISTTRKIIKFPTSPILAESSEMTIKTQTSP |
| | | PGSTSESTFTLDTSTTPSLVITHSTMTQRLPHSEITTLVSRGAGDVPRPSSL |
| | | PVEETSPPSSQLSLSAMISPSPVSSTLPASSHSSSASVTSLLTPGQVKTTEV |
| | | LDASAEPETSSPPSLSSTSVEILATSEVTTDTEKIHPFSNTAVTKVGTSSSG |
| | | HESPSSVLPDSETTKATSAMGTISIMGDTSVSTLTPALSNTRKIQSEPASSL |
| | | TTRLRETSTSEETSLATEANTVLSKVSTGATTEVSRTEAISFSRTSMSGPEQ |
| | | STMSQDISIGTIPRISASSVLTESAKMTITTQTGPSESTLESTLNLNTATTP |
| | | SWVETHSIVIQGFPHPEMTTSMGRGPGGVSWPSPPFVKETSPPSSPLSLPAV |
| | | TSPHPVSTTFLAHIPPSPLPVTSLLTSGPATTTDILGTSTEPGTSSSSSLST |
| | | TSHERLTTYKDTAHTEAVHPSTNTGGTNVATTSSGYKSQSSVLADSSPMCTT |
| | | STMGDTSVLTSTPAFLETRRIQTELASSLTPGLRESSGSEGTSSGTKMSTVL |
| | | SKVPTGATTEISKEDVTSIPGPAQSTISPDISTRTVSWFSTSPVMTESAEIT |
| | | MNTHTSPLGATTQGTSTLDTSSTTSLTMTHSTISQGFSHSQMSTLMRRGPED |
| | | VSWMSPPLLEKTRPSFSLMSSPATTSPSPVSSTLPESISSSPLPVTSLLTSG |
| | | LAKTTDMLHKSSEPVINSPANLSSTSVEILATSEVTTDTEKTHPSSNRTVTD |
| | | VGTSSSGHESTSFVLADSQTSKVTSPMVITSTMEDTSVSTSTPGFFETSRIQ |
| | | TEPTSSLTLGLRKTSSSEGTSLATEMSTVLSGVPTGATAEVSRTEVTSSSRT |
| | | SISGFAQLTVSPETSTETITRLPTSSIMTESAEMMIKTQTDPPGSTPESTHT |
| | | VDISTTPNWVETHSTVTQRFSHSEMTTLVSRSPGDMLWPSQSSVEETSSASS |
| | | LLSLPATTSPSPVSSTLVEDFPSASLPVTSLLNPGLVITTDRMGISREPGTS |
| | | STSNLSSTSHERLTTLEDTVDTEDMQPSTHTAVINVRTSISGHESQSSVLSD |
| | | SETPKATSPMGTTYTMGETSVSISTSDFFETSRIQIEPTSSLTSGLRETSSS |
| | | ERISSATEGSTVLSEVPSGATTEVSRTEVISSRGTSMSGPDQFTISPDISTE |
| | | AITRLSTSPIMTESAESAITIETGSPGATSEGTLTLDTSTTTFWSGTHSTAS |
| | | PGFSHSEMTTLMSRTPGDVPWPSLPSVEEASSVSSSLSSPAMTSTSFFSTLP |
| | | ESISSSPHPVTALLTLGPVKTTDMLRTSSEPETSSPPNLSSTSAEILATSEV |
| | | TKDREKIHPSSNTPVVNVGTVIYKHLSPSSVLADLVTTKPTSPMATTSTLGN |
| | | TSVSTSTPAFPETMMTQPTSSLTSGLREISTSQETSSATERSASLSGMPTGA |
| | | TTKVSRTEALSLGRTSTPGPAQSTISPEISTETITRISTPLTTTGSAEMTIT |
| | | PKTGHSGASSQGTFTLDTSSRASWPGTHSAATHRSPHSGMTTPMSRGPEDVS |
| | | WPSRPSVEKTSPPSSLVSLSAVTSPSPLYSTPSESSHSSPLRVTSLFTPVMM |
| | | KTTDMLDTSLEPVTTSPPSMNITSDESLATSKATMETEAIQLSENTAVTQMG |
| | | TISARQEFYSSYPGLPEPSKVTSPVVTSSTIKDIVSTTIPASSEITRIEMES |
| | | TSTLTPTPRETSTSQEIHSATKPSTVPYKALTSATIEDSMTQVMSSSRGPSP |
| | | DQSTMSQDISTEVITRLSTSPIKTESTEMTITTQTGSPGATSRGTLTLDTST |
| | | TFMSGTHSTASQGFSHSQMTALMSRTPGDVPWLSHPSVEEASSASFSLSSPV |
| | | MTSSSPVSSTLPDSIHSSSLPVTSLLTSGLVKTTELLGTSSEPETSSPPNLS |
| | | STSAEILAITEVTTDTEKLEMTNVVTSGYTHESPSSVLADSVTTKATSSMGI |
| | | TYPTGDTNVLISTPAFSDTSRIQTKSKLSLTPGLMETSISEETSSATEKSTV |
| | | LSSVPTGATTEVSRTEAISSSRTSIPGPAQSTMSSDTSMETITRISTPLTRK |
| | | ESTDMAITPKTGPSGATSQGTFTLDSSSTASWPGTHSATTQRFPQSVVTTPM |
| | | SRGPEDVSWPSPLSVEKNSPPSSLVSSSSVTSPSPLYSTPSGSSHSSPVPVT |
| | | SLFTSIMMKATDMLDASLEPETTSAPNMNITSDESLAASKATTETEAIHVFE |
| | | NTAASHVETTSATEELYSSSPGFSEPTKVISPVVTSSSIRDNMVSTTMPGSS |
| | | GITRIEIESMSSLTPGLRETRTSQDITSSTETSTVLYKMPSGATPEVSRTEV |
| | | MPSSRTSIPGPAQSTMSLDISDEVVTRLSTSPIMTESAEITITTQTGYSLAT |
| | | SQVTLPLGTSMTFLSGTHSTMSQGLSHSEMTNLMSRGPESLSWTSPRFVETT |
| | | RSSSSLTSLPLTTSLSPVSSTLLDSSPSSPLPVTSLILPGLVKTTEVLDTSS |
| | | EPKTSSSPNLSSTSVEIPATSEIMTDTEKIHPSSNTAVAKVRTSSSVHESHS |
| | | SVLADSETTITIPSMGITSAVDDTTVFTSNPAFSETRRIPTEPTFSLTPGFR |
| | | ETSTSEETTSITETSAVLYGVPTSATTEVSMTEIMSSNRIHIPDSDQSTMSP |
| | | DIITEVITRLSSSSMMSESTQMTITTQKSSPGATAQSTLTLATTTAPLARTH |
| | | STVPPRFLHSEMTTLMSRSPENPSWKSSLFVEKTSSSSSLLSLPVTTSPSVS |
| | | STLPQSIPSSSFSVTSLLTPGMVKTTDTSTEPGTSLSPNLSGTSVEILAASE |
| | | VTTDTEKIHPSSSMAVTNVGTTSSGHELYSSVSIHSEPSKATYPVGTPSSMA |
| | | ETSISTSMPANFETTGFEAEPFSHLTSGFRKTNMSLDTSSVTPTNTPSSPGS |
| | | THLLQSSKTDFTSSAKTSSPDWPPASQYTEIPVDIITPFNASPSITESTGIT |
| | | SFPESRFTMSVTESTHHLSTDLLPSAETISTGTVMPSLSEAMTSFATTGVPR |
| | | AISGSGSPFSRTESGPGDATLSTIAESLPSSTPVPFSSSTFTTTDSSTIPAL |
| | | HEITSSSATPYRVDTSLGTESSTTEGRLVMVSTLDTSSQPGRTSSSPILDTR |
| | | MTESVELGTVTSAYQVPSLSTRLTRTDGIMEHITKIPNEAAHRGTIRPVKGP |
| | | QTSTSPASPKGLHTGGTKRMETTTTALKTTTTALKTTSRATLTTSVYTPTLG |
| | | TLTPLNASMQMASTIPTEMMITTPYVFPDVPETTSSLATSLGAETSTALPRT |
| | | TPSVFNRESETTASLVSRSGAERSPVIQTLDVSSSEPDTTASWVIHPAETIP |
| | | TVSKTTPNFFHSELDTVSSTATSHGADVSSAIPTNISPSELDALTPLVTISG |
| | | TDTSTTFPTLTKSPHETETRTTWLTHPAETSSTIPRTIPNFSHHESDATPSI |
| | | ATSPGAETSSAIPIMTVSPGAEDLVTSQVTSSGTDRNMTIPTLTLSPGEPKT |
| | | IASLVTHPEAQTSSAIPTSTISPAVSRLVTSMVTSLAAKTSTTNRALTNSPG |
| | | EPATTVSLVTHPAQTSPTVPWTTSIFFHSKSDTTPSMTTSHGAESSSAVPTP |
| | | TVSTEVPGVVTPLVTSSRAVISTTIPILTLSPGEPETTPSMATSHGEEASSA |
| | | IPTPTVSPGVPGVVTSLVTSSRAVTSTTIPILTFSLGEPETTPSMATSHGTE |
| | | AGSAVPTVLPEVPGMVTSLVASSRAVTSTTLPTLTLSPGEPETTPSMATSHG |
| | | AEASSTVPTVSPEVPGVVTSLVTSSSGVNSTSIPTLILSPGELETTPSMATS |
| | | HGAEASSAVPTPTVSPGVSGVVTPLVTSSRAVTSTTIPILTLSSSEPETTPS |
| | | MATSHGVEASSAVLTVSPEVPGMVTSLVTSSRAVTSTTIPTLTISSDEPETT |
| | | TSLVTHSEAKMISAIPTLAVSPTVQGLVTSLVTSSGSETSAFSNITVASSQP |
| | | ETIDSWVAHPGTEASSVVPTLTVSTGEPFTNISLVTHPAESSSTLPRTTSRF |
| | | SHSELDTMPSTVTSPEAESSSAISTTISPGIPGVLTSLVTSSGRDISATFPT |
| | | VPESPHESEATASWVTHPAVTSTTVPRTTPNYSHSEPDTTPSIATSPGAEAT |
| | | SDFPTITVSPDVPDMVTSQVTSSGTDTSITIPTLTLSSGEPETTTSFITYSE |
| | | THTSSAIPTLPVSPGASKMLTSLVISSGTDSTTTFPTLTETPYEPETTAIQL |
| | | IHPAETNTMVPRTTPKFSHSKSDTTLPVAITSPGPEASSAVSTTTISPDMSD |
| | | LVTSLVPSSGTDTSTTFPTLSETPYEPETTATWLTHPAETSTTVSGTIPNFS |
| | | HRGSDTAPSMVTSPGVDTRSGVPTTTIPPSIPGVVTSQVTSSATDTSTAIPT |
| | | LTPSPGEPETTASSATHPGTQTGFTVPIRTVPSSEPDTMASWVTHPPQTSTP |
| | | VSRTTSSFSHSSPDATPVMATSPRTEASSAVLTTISPGAPEMVTSQITSSGA |
| | | ATSTTVPTLTHSPGMPETTALLSTHPRTETSKTFPASTVFPQVSETTASLTI |
| | | RPGAETSTALPTQTTSSLFTLLVTGTSRVDLSPTASPGVSAKTAPLSTHPGT |
| | | ETSTMIPTSTLSLGLLETTGLLATSSSAETSTSTLTLTVSPAVSGLSSASIT |
| | | TDKPQTVTSWNTETSPSVTSVGPPEFSRTVTGTTMTLIPSEMPTPPKTSHGE |
| | | GVSPTTILRTTMVEATNLATTGSSPTVAKTTTTENTLAGSLFTPLTTPGMST |
| | | LASESVTSRTSYNHRSWISTTSSYNRRYWTPATSTPVTSTFSPGISTSSIPS |
| | | STAATVPFMVPFTLNFTITNLQYEEDMRHPGSRKFNATERELQGLLKPLFRN |
| | | SSLEYLYSGCRLASLRPEKDSSATAVDAICTHRPDPEDLGLDRERLYWELSN |
| | | LINGIQELGPYTLDRNSLYVNGFTHRSSMPTTSTPGTSTVDVGTSGTPSSSP |
| | | SPTTAGPLLMPFTLNFTITNLQYEEDMRRTGSRKENTMESVLQGLLKPLFKN |
| | | TSVGPLYSGCRLTLLRPEKDGAATGVDAICTHRLDPKSPGLNREQLYWELSK |
| | | LTNDIEELGPYTLDRNSLYVNGFTHQSSVSTTSTPGTSTVDLRTSGTPSSLS |
| | | SPTIMAAGPLLVPFTLNFTITNLQYGEDMGHPGSRKENTTERVLQGLLGPIF |
| | | SQLTNGIKELGPYTLDRNSLYVNGFTHRTSVPTSSTPGTSTVDLGTSGTPFS |
| | | KNTSVGPLYSGCRLTSLRSEKDGAATGVDAICIHHLDPKSPGLNRERLYWEL |
| | | SQLTNGIKELGPYTLDRNSLYVNGFTHRTSVPTSSTPGTSTVDLGTSGTPFS |
| | | LPSPATAGPLLVLFTLNFTITNLKYEEDMHRPGSRKENTTERVLQTLLGPMF |
| | | KNTSVGLLYSGCRLTLLRSEKDGAATGVDAICTHRLDPKSPGVDREQLYWEL |
| | | SQLTNGIKELGPYTLDRNSLYVNGFTHWIPVPTSSTPGTSTVDLGSGTPSSL |
| | | PSPTTAGPLLVPFTLNFTITNLKYEEDMHCPGSRKENTTERVLQSLLGPMFK |
| | | NTSVGPLYSGCRLTLLRSEKDGAATGVDAICTHRLDPKSPGVDREQLYWELS |
| | | QLTNGIKELGPYTLDRNSLYVNGFTHQTSAPNTSTPGTSTVDLGTSGTPSSL |
| | | PSPTSAGPLLVPFTLNFTITNLQYEEDMHHPGSRKFNTTERVLQGLLGPMFK |
| | | NTSVGLLYSGCRLTLLRPEKNGAATGMDAICSHRLDPKSPGLNREQLYWELS |
| | | QLTHGIKELGPYTLDRNSLYVNGFTHRSSVAPTSTPGTSTVDLGTSGTPSSL |
| | | PSPTTAVPLLVPFTLNFTITNLQYGEDMRHPGSRKENTTERVLQGLLGPLFK |
| | | NSSVGPLYSGCRLISLRSEKDGAATGVDAICTHHLNPQSPGLDREQLYWQLS |
| | | QMTNGIKELGPYTLDRNSLYVNGFTHRSSGLTTSTPWTSTVDLGTSGTPSPV |
| | | PSPTTTGPLLVPFTLNFTITNLQYEENMGHPGSRKFNITESVLQGLLKPLFK |
| | | STSVGPLYSGCRLTLLRPEKDGVATRVDAICTHRPDPKIPGLDRQQLYWELS |
| | | QLTHSITELGPYTLDRDSLYVNGFTQRSSVPTTSTPGTFTVQPETSETPSSL |
| | | PGPTATGPVLLPFTLNFTITNLQYEEDMRRPGSRKFNTTERVLQGLLMPLFK |
| | | NTSVSSLYSGCRLTLLRPEKDGAATRVDAVCTHRPDPKSPGLDRERLYWKLS |
| | | QLTHGITELGPYTLDRHSLYVNGFTHQSSMTTTRTPDTSTMHLATSRTPASL |
| | | SGPMTASPLLVLFTINFTITNLRYEENMHHPGSRKENTTERVLQGLLRPVFK |
| | | NTSVGPLYSGCRLTLLRPKKDGAATKVDAICTYRPDPKSPGLDREQLYWELS |
| | | QLTHSITELGPYTLDRDSLYVNGFTQRSSVPTTSIPGTPTVDLGTSGTPVSK |
| | | PGPSAASPLLVLFTLNFTITNLRYEENMQHPGSRKFNTTERVLQGLLRSLFK |
| | | STSVGPLYSGCRLTLLRPEKDGTATGVDAICTHHPDPKSPRLDREQLYWELS |
| | | QLTHNITELGPYALDNDSLFVNGFTHRSSVSTTSTPGTPTVYLGASKTPASI |
| | | FGPSAASHLLILFTLNFTITNLRYEENMW |
| 41 | Anti-MUC16 7135 | QSISSY |
| | CDRL1 | |
| 42 | Anti-MUC16 7135 | AAS |
| | CDRL2 | |
| 43 | Anti-MUC16 7135 | QQSYSTPPIT |
| | CDRL3 | |
| 44 | Anti-MUC16 7135 | GFTFSYHE |
| | CDRH1 | |
| 45 | Anti-MUC16 7135 | IGSRGTTK |
| | CDRH2 | |
| 46 | Anti-MUC16 7135 | ASEVGSILGYSMDV |
| | CDRH3 | |
| 47 | Anti-MUC16 7135 | DIQMTQSPSSLSASVGDRVTITCRASQSISSYLNWYQQKPGKAPKLLIYAAS |
| | variable light | SLQSGVPSRFSGSGSGTDFTLTISSLQPEDFATYYCQQSYSTPPITFGQGTR |
| | chain | LEIK |
| 48 | Anti-MUC16 7135 | EVQLVESGGGLVQAGGSLRLSCAASGFTFSYHEMNWVRQAPGKGLEWVSYIG |
| | variable heavy | SRGTTKHYADPVKGRFTISRDNAKNSLYLQMNSLRAEDTAVYYCASEVGSIL |
| | chain | GYSMDVWGQGTTVTVSS |
| 49 | pBB4000 CAR | EVQLVESGGGLVQAGGSLRLSCAASGFTFSYHEMNWVRQAPGKGLEWVSYIG |
| | a.a. sequence | SRGTTKHYADPVKGRFTISRDNAKNSLYLQMNSLRAEDTAVYYCASEVGSIL |
| | without signal | GYSMDVWGQGTTVTVSSGGGGSGGGGSGGGGSDIQMTQSPSSLSASVGDRVT |
| | peptide | ITCRASQSISSYLNWYQQKPGKAPKLLIYAASSLQSGVPSRFSGSGSGTDET |
| | | LTISSLQPEDFATYYCQQSYSTPPITFGQGTRLEIKTTTPAPRPPTPAPTIA |
| | | SQPLSLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKFSRSAD |
| | | APAYQQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQALPPR |
| 50 | pBB4000 CAR | MALPVTALLLPLALLLHAARPEVQLVESGGGLVQAGGSLRLSCAASGFTFSY |
| | a.a. sequence | HEMNWVRQAPGKGLEWVSYIGSRGTTKHYADPVKGRFTISRDNAKNSLYLQM |
| | with signal | NSLRAEDTAVYYCASEVGSILGYSMDVWGQGTTVTVSSGGGGSGGGGSGGGG |
| | peptide | SDIQMTQSPSSLSASVGDRVTITCRASQSISSYLNWYQQKPGKAPKLLIYAA |
| | | SSLQSGVPSRFSGSGSGTDFTLTISSLQPEDFATYYCQQSYSTPPITFGQGT |
| | | RLEIKTTTPAPRPPTPAPTIASQPLSLRPEACRPAAGGAVHTRGLDFACDIY |
| | | IWAPLAGTCGVLLLSLVITLYCKRGRKKLLYIFKQPFMRPVQTTQEEDGCSC |
| | | RFPEEEEGGCELRVKFSRSADAPAYQQGQNQLYNELNLGRREEYDVLDKRRG |
| | | RDPEMGGKPRRKNPQEGLYNELQKDKMAEAYSEIGMKGERRRGKGHDGLYQG |
| | | LSTATKDTYDALHMQALPPR |
| 51 | pBB4001 CAR | DIQMTQSPSSLSASVGDRVTITCRASQSISSYLNWYQQKPGKAPKLLIYAAS |
| | a.a. sequence | SLQSGVPSRFSGSGSGTDFTLTISSLQPEDFATYYCQQSYSTPPITFGQGTR |
| | without signal | LEIKGGGGSGGGGSGGGGSEVQLVESGGGLVQAGGSLRLSCAASGFTFSYHE |
| | peptide | MNWVRQAPGKGLEWVSYIGSRGTTKHYADPVKGRFTISRDNAKNSLYLQMNS |
| | | LRAEDTAVYYCASEVGSILGYSMDVWGQGTTVTVSSTTTPAPRPPTPAPTIA |
| | | SQPLSLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKFSRSAD |
| | | APAYQQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQALPPR |
| 52 | pBB4001 CAR | MALPVTALLLPLALLLHAARPDIQMTQSPSSLSASVGDRVTITCRASQSISS |
| | a.a. sequence | YLNWYQQKPGKAPKLLIYAASSLQSGVPSRFSGSGSGTDFTLTISSLQPEDF |
| | with signal | ATYYCQQSYSTPPITFGQGTRLEIKGGGGSGGGGSGGGGSEVQLVESGGGLV |
| | peptide | QAGGSLRLSCAASGFTFSYHEMNWVRQAPGKGLEWVSYIGSRGTTKHYADPV |
| | | KGRFTISRDNAKNSLYLQMNSLRAEDTAVYYCASEVGSILGYSMDVWGQGTT |
| | | VTVSSTTTPAPRPPTPAPTIASQPLSLRPEACRPAAGGAVHTRGLDFACDIY |
| | | IWAPLAGTCGVLLLSLVITLYCKRGRKKLLYIFKQPFMRPVQTTQEEDGCSC |
| | | RFPEEEEGGCELRVKFSRSADAPAYQQGQNQLYNELNLGRREEYDVLDKRRG |
| | | RDPEMGGKPRRKNPQEGLYNELQKDKMAEAYSEIGMKGERRRGKGHDGLYQG |
| | | LSTATKDTYDALHMQALPPR |
| 53 | Anti-MUC16 7138 | QSISSY |
| | CDRL1 | |
| 54 | Anti-MUC16 7138 | AAS |
| | CDRL2 | |
| 55 | Anti-MUC16 7138 | QQSYSTPPIT |
| | CDRL3 | |
| 56 | Anti-MUC16 7138 | GYTFTSSD |
| | CDRH1 | |
| 57 | Anti-MUC16 7138 | MNPNYGYT |
| | CDRH2 | |
| 58 | Anti-MUC16 7138 | ARVTYCSSVTSCFRPFTY |
| | CDRH3 | |
| 59 | Anti-MUC16 7138 | DIQMTQSPSSLSASVGDRVTITCRASQSISSYLNWYQQKPGKAPKLLIYAAS |
| | variable light | SLQSGVPSRFSGSGSGTDFTLTISSLQPEDFATYYCQQSYSTPPITFGQGTR |
| | chain | LEIK |
| 60 | Anti-MUC16 7138 | QVQLVQSGAEVKKPGASVKVSCKASGYTFTSSDINWVRQATGQGLEWVGWMN |
| | variable heavy | PNYGYTGSARKFQDRVTMTRNTSLSTAYLELHSLRSEDTAVYYCARVTYCSS |
| | chain | VTSCFRPFTYWGQGTLVTVSA |
| 61 | pBB4002 CAR | QVQLVQSGAEVKKPGASVKVSCKASGYTFTSSDINWVRQATGQGLEWVGWMN |
| | a.a. sequence | PNYGYTGSARKFQDRVTMTRNTSLSTAYLELHSLRSEDTAVYYCARVTYCSS |
| | without signal | VTSCFRPFTYWGQGTLVTVSAGGGGSGGGGSGGGGSDIQMTQSPSSLSASVG |
| | peptide | DRVTITCRASQSISSYLNWYQQKPGKAPKLLIYAASSLQSGVPSRFSGSGSG |
| | | TDFTLTISSLQPEDFATYYCQQSYSTPPITFGQGTRLEIKTTTPAPRPPTPA |
| | | PTIASQPLSLRPEACRPAAGGAVHTRGLDFACDIYIWAPLAGTCGVLLLSLV |
| | | ITLYCKRGRKKLLYIFKQPFMRPVQTTQEEDGCSCRFPEEEEGGCELRVKFS |
| | | RSADAPAYQQGQNQLYNELNLGRREEYDVLDKRRGRDPEMGGKPRRKNPQEG |
| | | LYNELQKDKMAEAYSEIGMKGERRRGKGHDGLYQGLSTATKDTYDALHMQAL |
| | | PPR |
| 62 | pBB4002 CAR | MALPVTALLLPLALLLHAARPQVQLVQSGAEVKKPGASVKVSCKASGYTFTS |
| | a.a. sequence | SDINWVRQATGQGLEWVGWMNPNYGYTGSARKFQDRVTMTRNTSLSTAYLEL |
| | with signal | HSLRSEDTAVYYCARVTYCSSVTSCFRPFTYWGQGTLVTVSAGGGGSGGGGS |
| | peptide | GGGGSDIQMTQSPSSLSASVGDRVTITCRASQSISSYLNWYQQKPGKAPKLL |
| | | IYAASSLQSGVPSRFSGSGSGTDFTLTISSLQPEDFATYYCQQSYSTPPITF |
| | | GQGTRLEIKTTTPAPRPPTPAPTIASQPLSLRPEACRPAAGGAVHTRGLDFA |
| | | CDIYIWAPLAGTCGVLLLSLVITLYCKRGRKKLLYIFKQPFMRPVQTTQEED |
| | | GCSCRFPEEEEGGCELRVKFSRSADAPAYQQGQNQLYNELNLGRREEYDVLD |
| | | KRRGRDPEMGGKPRRKNPQEGLYNELQKDKMAEAYSEIGMKGERRRGKGHDG |
| | | LYQGLSTATKDTYDALHMQALPPR |
| 63 | pBB4003 CAR | DIQMTQSPSSLSASVGDRVTITCRASQSISSYLNWYQQKPGKAPKLLIYAAS |
| | a.a. sequence | SLQSGVPSRFSGSGSGTDFTLTISSLQPEDFATYYCQQSYSTPPITFGQGTR |
| | without signal | LEIKGGGGSGGGGSGGGGSQVQLVQSGAEVKKPGASVKVSCKASGYTFTSSD |
| | peptide | INWVRQATGQGLEWVGWMNPNYGYTGSARKFQDRVTMTRNTSLSTAYLELHS |
| | | LRSEDTAVYYCARVTYCSSVTSCFRPFTYWGQGTLVTVSATTTPAPRPPTPA |
| | | PTIASQPLSLRPEACRPAAGGAVHTRGLDFACDIYIWAPLAGTCGVLLLSLV |
| | | ITLYCKRGRKKLLYIFKQPFMRPVQTTQEEDGCSCRFPEEEEGGCELRVKFS |
| | | RSADAPAYQQGQNQLYNELNLGRREEYDVLDKRRGRDPEMGGKPRRKNPQEG |
| | | LYNELQKDKMAEAYSEIGMKGERRRGKGHDGLYQGLSTATKDTYDALHMQAL |
| | | PPR |
| 64 | pBB4003 CAR | MALPVTALLLPLALLLHAARPDIQMTQSPSSLSASVGDRVTITCRASQSISS |
| | a.a. sequence | YLNWYQQKPGKAPKLLIYAASSLQSGVPSRFSGSGSGTDFTLTISSLQPEDF |
| | with signal | ATYYCQQSYSTPPITFGQGTRLEIKGGGGSGGGGSGGGGSQVQLVQSGAEVK |
| | peptide | KPGASVKVSCKASGYTFTSSDINWVRQATGQGLEWVGWMNPNYGYTGSARKF |
| | | QDRVTMTRNTSLSTAYLELHSLRSEDTAVYYCARVTYCSSVTSCFRPFTYWG |
| | | QGTLVTVSATTTPAPRPPTPAPTIASQPLSLRPEACRPAAGGAVHTRGLDFA |
| | | CDIYIWAPLAGTCGVLLLSLVITLYCKRGRKKLLYIFKQPFMRPVQTTQEED |
| | | GCSCRFPEEEEGGCELRVKFSRSADAPAYQQGQNQLYNELNLGRREEYDVLD |
| | | KRRGRDPEMGGKPRRKNPQEGLYNELQKDKMAEAYSEIGMKGERRRGKGHDG |
| | | LYQGLSTATKDTYDALHMQALPPR |
| 65 | Anti-MUC16 8755 | QSINIY |
| | CDRL1 | |
| 66 | Anti-MUC16 8755 | AAS |
| | CDRL2 | |
| 67 | Anti-MUC16 8755 | QHSYSTPPIT |
| | CDRL3 | |
| 68 | Anti-MUC16 8755 | GFTENDYA |
| | CDRH1 | |
| 69 | Anti-MUC16 8755 | IDWNGGSI |
| | CDRH2 | |
| 70 | Anti-MUC16 8755 | AKDIGHWNYIGGMDV |
| | CDRH3 | |
| 71 | Anti-MUC16 8755 | DIQMTQSPSSLSASVGDRVTITCRASQSINIYLNWYQQKPGKAPKFLIYAAS |
| | variable light | SLQTGVPSRFSGSGSGTDFTLTISSLEPEDFATYYCQHSYSTPPITFGQGTR |
| | chain | LEIK |
| 72 | Anti-MUC16 8755 | EVQLVESGGGLVQPGRSLRLSCAASGFTENDYAMHWVRQSPGKGLEWVSGID |
| | variable heavy | WNGGSIDYADSVKGRFTISRDTAKNSLYLQMNSLKVEDTALYYCAKDIGHWN |
| | chain | YIGGMDVWGQGTTVTVSS |
| 73 | pBB4004 CAR | EVQLVESGGGLVQPGRSLRLSCAASGFTENDYAMHWVRQSPGKGLEWVSGID |
| | a.a. sequence | WNGGSIDYADSVKGRFTISRDTAKNSLYLQMNSLKVEDTALYYCAKDIGHWN |
| | without signal | YIGGMDVWGQGTTVTVSSGGGGSGGGGSGGGGSDIQMTQSPSSLSASVGDRV |
| | peptide | TITCRASQSINIYLNWYQQKPGKAPKFLIYAASSLQTGVPSRFSGSGSGTDF |
| | | TLTISSLEPEDFATYYCQHSYSTPPITFGQGTRLEIKTTTPAPRPPTPAPTI |
| | | ASQPLSLRPEACRPAAGGAVHTRGLDFACDIYIWAPLAGTCGVLLLSLVITL |
| | | YCKRGRKKLLYIFKQPFMRPVQTTQEEDGCSCRFPEEEEGGCELRVKFSRSA |
| | | DAPAYQQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQALPPR |
| 74 | pBB4004 CAR | MALPVTALLLPLALLLHAARPEVQLVESGGGLVQPGRSLRLSCAASGFTEND |
| | a.a. sequence | YAMHWVRQSPGKGLEWVSGIDWNGGSIDYADSVKGRFTISRDTAKNSLYLQM |
| | with signal | NSLKVEDTALYYCAKDIGHWNYIGGMDVWGQGTTVTVSSGGGGSGGGGSGGG |
| | peptide | GSDIQMTQSPSSLSASVGDRVTITCRASQSINIYLNWYQQKPGKAPKFLIYA |
| | | ASSLQTGVPSRFSGSGSGTDFTLTISSLEPEDFATYYCQHSYSTPPITFGQG |
| | | TRLEIKTTTPAPRPPTPAPTIASQPLSLRPEACRPAAGGAVHTRGLDFACDI |
| | | YIWAPLAGTCGVLLLSLVITLYCKRGRKKLLYIFKQPFMRPVQTTQEEDGCS |
| | | CRFPEEEEGGCELRVKFSRSADAPAYQQGQNQLYNELNLGRREEYDVLDKRR |
| | | GRDPEMGGKPRRKNPQEGLYNELQKDKMAEAYSEIGMKGERRRGKGHDGLYQ |
| | | GLSTATKDTYDALHMQALPPR |
| 75 | pBB4005 CAR | DIQMTQSPSSLSASVGDRVTITCRASQSINIYLNWYQQKPGKAPKFLIYAAS |
| | a.a. sequence | SLQTGVPSRFSGSGSGTDFTLTISSLEPEDFATYYCQHSYSTPPITFGQGTR |
| | without signal | LEIKGGGGSGGGGSGGGGSEVQLVESGGGLVQPGRSLRLSCAASGFTENDYA |
| | peptide | MHWVRQSPGKGLEWVSGIDWNGGSIDYADSVKGRFTISRDTAKNSLYLQMNS |
| | | LKVEDTALYYCAKDIGHWNYIGGMDVWGQGTTVTVSSTTTPAPRPPTPAPTI |
| | | ASQPLSLRPEACRPAAGGAVHTRGLDFACDIYIWAPLAGTCGVLLLSLVITL |
| | | YCKRGRKKLLYIFKQPFMRPVQTTQEEDGCSCRFPEEEEGGCELRVKFSRSA |
| | | DAPAYQQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQALPPR |
| 76 | pBB4005 CAR | MALPVTALLLPLALLLHAARPDIQMTQSPSSLSASVGDRVTITCRASQSINI |
| | a.a. sequence | YLNWYQQKPGKAPKFLIYAASSLQTGVPSRFSGSGSGTDFTLTISSLEPEDF |
| | with signal | ATYYCQHSYSTPPITFGQGTRLEIKGGGGSGGGGSGGGGSEVQLVESGGGLV |
| | peptide | QPGRSLRLSCAASGFTFNDYAMHWVRQSPGKGLEWVSGIDWNGGSIDYADSV |
| | | KGRFTISRDTAKNSLYLQMNSLKVEDTALYYCAKDIGHWNYIGGMDVWGQGT |
| | | TVTVSSTTTPAPRPPTPAPTIASQPLSLRPEACRPAAGGAVHTRGLDFACDI |
| | | YIWAPLAGTCGVLLLSLVITLYCKRGRKKLLYIFKQPFMRPVQTTQEEDGCS |
| | | CRFPEEEEGGCELRVKFSRSADAPAYQQGQNQLYNELNLGRREEYDVLDKRR |
| | | GRDPEMGGKPRRKNPQEGLYNELQKDKMAEAYSEIGMKGERRRGKGHDGLYQ |
| | | GLSTATKDTYDALHMQALPPR |
| 77 | Anti-MUC16 8767 | QSISTY |
| | CDRL1 | |
| 78 | Anti-MUC16 8767 | TAS |
| | CDRL2 | |
| 79 | Anti-MUC16 8767 | QQSYSTPPIT |
| | CDRL3 | |
| 80 | Anti-MUC16 8767 | GFTFSNYY |
| | CDRH1 | |
| 81 | Anti-MUC16 8767 | ISGRGSTI |
| | CDRH2 | |
| 82 | Anti-MUC16 8767 | VKDRGGYSPY |
| | CDRH3 | |
| 83 | Anti-MUC16 8767 | DIQMTQSPSSLSASVGDRVTITCRASQSISTYLNWYQQKPGKAPKLLIYTAS |
| | variable light | SLQSGVPSRFSGSGSGTDFTLTISSLQPEDFATYYCQQSYSTPPITFGQGTR |
| | chain | LEIK |
| 84 | Anti-MUC16 8767 | QVQLVESGGGLVKPGGSLRLSCAASGFTFSNYYMSWVRQAPGKGLEWISYIS |
| | variable heavy | GRGSTIFYADSVKGRITISRDNAKNSLFLQMNSLRAEDTAVYFCVKDRGGYS |
| | chain | PYWGQGTLVTVSS |
| 85 | pBB4006 CAR | QVQLVESGGGLVKPGGSLRLSCAASGFTFSNYYMSWVRQAPGKGLEWISYIS |
| | a.a. sequence | GRGSTIFYADSVKGRITISRDNAKNSLFLQMNSLRAEDTAVYFCVKDRGGYS |
| | without signal | PYWGQGTLVTVSSGGGGSGGGGSGGGGSDIQMTQSPSSLSASVGDRVTITCR |
| | peptide | ASQSISTYLNWYQQKPGKAPKLLIYTASSLQSGVPSRFSGSGSGTDFTLTIS |
| | | SLQPEDFATYYCQQSYSTPPITFGQGTRLEIKTTTPAPRPPTPAPTIASQPL |
| | | SLRPEACRPAAGGAVHTRGLDFACDIYIWAPLAGTCGVLLLSLVITLYCKRG |
| | | RKKLLYIFKQPFMRPVQTTQEEDGCSCRFPEEEEGGCELRVKFSRSADAPAY |
| | | QQGQNQLYNELNLGRREEYDVLDKRRGRDPEMGGKPRRKNPQEGLYNELQKD |
| | | KMAEAYSEIGMKGERRRGKGHDGLYQGLSTATKDTYDALHMQALPPR |
| 86 | pBB4006 CAR | MALPVTALLLPLALLLHAARPQVQLVESGGGLVKPGGSLRLSCAASGFTFSN |
| | a.a. sequence | YYMSWVRQAPGKGLEWISYISGRGSTIFYADSVKGRITISRDNAKNSLFLQM |
| | with signal | NSLRAEDTAVYFCVKDRGGYSPYWGQGTLVTVSSGGGGSGGGGSGGGGSDIQ |
| | peptide | MTQSPSSLSASVGDRVTITCRASQSISTYLNWYQQKPGKAPKLLIYTASSLQ |
| | | SGVPSRFSGSGSGTDFTLTISSLQPEDFATYYCQQSYSTPPITFGQGTRLEI |
| | | KTTTPAPRPPTPAPTIASQPLSLRPEACRPAAGGAVHTRGLDFACDIYIWAP |
| | | LAGTCGVLLLSLVITLYCKRGRKKLLYIFKQPFMRPVQTTQEEDGCSCREPE |
| | | EEEGGCELRVKFSRSADAPAYQQGQNQLYNELNLGRREEYDVLDKRRGRDPE |
| | | MGGKPRRKNPQEGLYNELQKDKMAEAYSEIGMKGERRRGKGHDGLYQGLSTA |
| | | TKDTYDALHMQALPPR |
| 87 | pBB4007 CAR | DIQMTQSPSSLSASVGDRVTITCRASQSISTYLNWYQQKPGKAPKLLIYTAS |
| | a.a. sequence | SLQSGVPSRFSGSGSGTDFTLTISSLQPEDFATYYCQQSYSTPPITFGQGTR |
| | without signal | LEIKGGGGSGGGGSGGGGSQVQLVESGGGLVKPGGSLRLSCAASGFTFSNYY |
| | peptide | MSWVRQAPGKGLEWISYISGRGSTIFYADSVKGRITISRDNAKNSLFLQMNS |
| | | LRAEDTAVYFCVKDRGGYSPYWGQGTLVTVSSTTTPAPRPPTPAPTIASQPL |
| | | SLRPEACRPAAGGAVHTRGLDFACDIYIWAPLAGTCGVLLLSLVITLYCKRG |
| | | RKKLLYIFKQPFMRPVQTTQEEDGCSCRFPEEEEGGCELRVKFSRSADAPAY |
| | | QQGQNQLYNELNLGRREEYDVLDKRRGRDPEMGGKPRRKNPQEGLYNELQKD |
| | | KMAEAYSEIGMKGERRRGKGHDGLYQGLSTATKDTYDALHMQALPPR |
| 88 | pBB4007 CAR | MALPVTALLLPLALLLHAARPDIQMTQSPSSLSASVGDRVTITCRASQSIST |
| | a.a. sequence | YLNWYQQKPGKAPKLLIYTASSLQSGVPSRFSGSGSGTDFTLTISSLQPEDF |
| | with signal | ATYYCQQSYSTPPITFGQGTRLEIKGGGGSGGGGSGGGGSQVQLVESGGGLV |
| | peptide | KPGGSLRLSCAASGFTFSNYYMSWVRQAPGKGLEWISYISGRGSTIFYADSV |
| | | KGRITISRDNAKNSLFLQMNSLRAEDTAVYFCVKDRGGYSPYWGQGTLVTVS |
| | | STTTPAPRPPTPAPTIASQPLSLRPEACRPAAGGAVHTRGLDFACDIYIWAP |
| | | LAGTCGVLLLSLVITLYCKRGRKKLLYIFKQPFMRPVQTTQEEDGCSCREPE |
| | | EEEGGCELRVKFSRSADAPAYQQGQNQLYNELNLGRREEYDVLDKRRGRDPE |
| | | MGGKPRRKNPQEGLYNELQKDKMAEAYSEIGMKGERRRGKGHDGLYQGLSTA |
| | | TKDTYDALHMQALPPR |
| 89 | Anti-MUC16 8794 | QSINSY |
| | CDRL1 | |
| 90 | Anti-MUC16 8794 | AAS |
| | CDRL2 | |
| 91 | Anti-MUC16 8794 | QQSYSSPPIT |
| | CDRL3 | |
| 92 | Anti-MUC16 8794 | GFTFRDYS |
| | CDRH1 | |
| 93 | Anti-MUC16 8794 | VTFFNSAI |
| | CDRH2 | |
| 94 | Anti-MUC16 8794 | AREREPIVGGFDY |
| | CDRH3 | |
| 95 | Anti-MUC16 8794 | DIQMTQSPSSLSASVGDRVTITCRASQSINSYLNWYQQKPGKAPKLLIYAAS |
| | variable light | SLQGGVPSRFSGGGSGTDFTLTITSLQPEDFATFYCQQSYSSPPITFGQGTR |
| | chain | LEIK |
| 96 | Anti-MUC16 8794 | QVQLVESGGGLVKPGGSLRLSCAASGFTFRDYSMSWIRQAPGKGLEWVSYVT |
| | variable heavy | FFNSAIYYADSVKGRFTISRDNAKNSLYLQMNSLRAEDTAVYYCAREREPIV |
| | chain | GGFDYWGQGTLVTVSS |
| 97 | pBB4008 CAR | QVQLVESGGGLVKPGGSLRLSCAASGFTFRDYSMSWIRQAPGKGLEWVSYVT |
| | a.a. sequence | FFNSAIYYADSVKGRFTISRDNAKNSLYLQMNSLRAEDTAVYYCAREREPIV |
| | without signal | GGFDYWGQGTLVTVSSGGGGSGGGGSGGGGSDIQMTQSPSSLSASVGDRVTI |
| | peptide | TCRASQSINSYLNWYQQKPGKAPKLLIYAASSLQGGVPSRFSGGGSGTDFTL |
| | | TITSLQPEDFATFYCQQSYSSPPITFGQGTRLEIKTTTPAPRPPTPAPTIAS |
| | | QPLSLRPEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKFSRSADA |
| | | PAYQQGQNQLYNELNLGRREEYDVLDKRRGRDPEMGGKPRRKNPQEGLYNEL |
| | | QKDKMAEAYSEIGMKGERRRGKGHDGLYQGLSTATKDTYDALHMQALPPR |
| 98 | pBB4008 CAR | MALPVTALLLPLALLLHAARPQVQLVESGGGLVKPGGSLRLSCAASGFTERD |
| | a.a. sequence | YSMSWIRQAPGKGLEWVSYVTFFNSAIYYADSVKGRFTISRDNAKNSLYLQM |
| | with signal | NSLRAEDTAVYYCAREREPIVGGFDYWGQGTLVTVSSGGGGSGGGGSGGGGS |
| | peptide | DIQMTQSPSSLSASVGDRVTITCRASQSINSYLNWYQQKPGKAPKLLIYAAS |
| | | SLQGGVPSRFSGGGSGTDFTLTITSLQPEDFATFYCQQSYSSPPITFGQGTR |
| | | LEIKTTTPAPRPPTPAPTIASQPLSLRPEACRPAAGGAVHTRGLDFACDIYI |
| | | WAPLAGTCGVLLLSLVITLYCKRGRKKLLYIFKQPFMRPVQTTQEEDGCSCR |
| | | FPEEEEGGCELRVKFSRSADAPAYQQGQNQLYNELNLGRREEYDVLDKRRGR |
| | | DPEMGGKPRRKNPQEGLYNELQKDKMAEAYSEIGMKGERRRGKGHDGLYQGL |
| | | STATKDTYDALHMQALPPR |
| 99 | pBB4009 CAR | DIQMTQSPSSLSASVGDRVTITCRASQSINSYLNWYQQKPGKAPKLLIYAAS |
| | a.a. sequence | SLQGGVPSRFSGGGSGTDFTLTITSLQPEDFATFYCQQSYSSPPITFGQGTR |
| | without signal | LEIKGGGGSGGGGSGGGGSQVQLVESGGGLVKPGGSLRLSCAASGFTERDYS |
| | peptide | MSWIRQAPGKGLEWVSYVTFFNSAIYYADSVKGRFTISRDNAKNSLYLQMNS |
| | | LRAEDTAVYYCAREREPIVGGFDYWGQGTLVTVSSTTTPAPRPPTPAPTIAS |
| | | QPLSLRPEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKFSRSADA |
| | | PAYQQGQNQLYNELNLGRREEYDVLDKRRGRDPEMGGKPRRKNPQEGLYNEL |
| | | QKDKMAEAYSEIGMKGERRRGKGHDGLYQGLSTATKDTYDALHMQALPPR |
| 100 | pBB4009 CAR | MALPVTALLLPLALLLHAARPDIQMTQSPSSLSASVGDRVTITCRASQSINS |
| | a.a. sequence | YLNWYQQKPGKAPKLLIYAASSLQGGVPSRFSGGGSGTDFTLTITSLQPEDF |
| | with signal | ATFYCQQSYSSPPITFGQGTRLEIKGGGGSGGGGSGGGGSQVQLVESGGGLV |
| | peptide | KPGGSLRLSCAASGFTFRDYSMSWIRQAPGKGLEWVSYVTFFNSAIYYADSV |
| | | KGRFTISRDNAKNSLYLQMNSLRAEDTAVYYCAREREPIVGGFDYWGQGTLV |
| | | TVSSTTTPAPRPPTPAPTIASQPLSLRPEACRPAAGGAVHTRGLDFACDIYI |
| | | WAPLAGTCGVLLLSLVITLYCKRGRKKLLYIFKQPFMRPVQTTQEEDGCSCR |
| | | FPEEEEGGCELRVKFSRSADAPAYQQGQNQLYNELNLGRREEYDVLDKRRGR |
| | | DPEMGGKPRRKNPQEGLYNELQKDKMAEAYSEIGMKGERRRGKGHDGLYQGL |
| | | STATKDTYDALHMQALPPR |
| 101 | Anti-MUC16 8799 | QSITSSY |
| | CDRL1 | |
| 102 | Anti-MUC16 8799 | GAS |
| | CDRL2 | |
| 103 | Anti-MUC16 8799 | QQYGSSPWT |
| | CDRL3 | |
| 104 | Anti-MUC16 8799 | GFAFGDHT |
| | CDRH1 | |
| 105 | Anti-MUC16 8799 | IRSRAYGGTT |
| | CDRH2 | |
| 106 | Anti-MUC16 8799 | TSGGYDSSLHYYYYYH |
| | CDRH3 | |
| 107 | Anti-MUC16 8799 | EIVLTQSPGTLSLSPGERATLSCRASQSITSSYLAWYQQRPGQAPRLLIYGA |
| | variable light | SSRATGIPDRFSGSGSGTDFTLTISRLEPEDFAVYYCQQYGSSPWTFGQGTK |
| | chain | VEIK |
| 108 | Anti-MUC16 8799 | EVQLVESGGGLEQPGRSLRLSCTASGFAFGDHTMSWVRQAPGKGLEWVGFIR |
| | variable heavy | SRAYGGTTEYAASVKGRFTISRDDSKSIAYLQMDSLKTEDTAVYYCTSGGYD |
| | chain | SSLHYYYYYHGMDVWGRGTTVTVSS |
| 109 | pBB4010 CAR | EVQLVESGGGLEQPGRSLRLSCTASGFAFGDHTMSWVRQAPGKGLEWVGFIR |
| | a.a. sequence | SRAYGGTTEYAASVKGRFTISRDDSKSIAYLQMDSLKTEDTAVYYCTSGGYD |
| | without signal | SSLHYYYYYHGMDVWGRGTTVTVSSGGGGSGGGGSGGGGSEIVLTQSPGTLS |
| | peptide | LSPGERATLSCRASQSITSSYLAWYQQRPGQAPRLLIYGASSRATGIPDRES |
| | | GSGSGTDFTLTISRLEPEDFAVYYCQQYGSSPWTFGQGTKVEIKTTTPAPRP |
| | | PTPAPTIASQPLSLRPEACRPAAGGAVHTRGLDFACDIYIWAPLAGTCGVLL |
| | | LSLVITLYCKRGRKKLLYIFKQPFMRPVQTTQEEDGCSCRFPEEEEGGCELR |
| | | VKFSRSADAPAYQQGQNQLYNELNLGRREEYDVLDKRRGRDPEMGGKPRRKN |
| | | PQEGLYNELQKDKMAEAYSEIGMKGERRRGKGHDGLYQGLSTATKDTYDALH |
| | | MQALPPR |
| 110 | pBB4010 CAR | MALPVTALLLPLALLLHAARPEVQLVESGGGLEQPGRSLRLSCTASGFAFGD |
| | a.a. sequence | HTMSWVRQAPGKGLEWVGFIRSRAYGGTTEYAASVKGRFTISRDDSKSIAYL |
| | with signal | QMDSLKTEDTAVYYCTSGGYDSSLHYYYYYHGMDVWGRGTTVTVSSGGGGSG |
| | peptide | GGGSGGGGSEIVLTQSPGTLSLSPGERATLSCRASQSITSSYLAWYQQRPGQ |
| | | APRLLIYGASSRATGIPDRFSGSGSGTDFTLTISRLEPEDFAVYYCQQYGSS |
| | | PWTFGQGTKVEIKTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQTT |
| | | QEEDGCSCRFPEEEEGGCELRVKFSRSADAPAYQQGQNQLYNELNLGRREEY |
| | | DVLDKRRGRDPEMGGKPRRKNPQEGLYNELQKDKMAEAYSEIGMKGERRRGK |
| | | GHDGLYQGLSTATKDTYDALHMQALPPR |
| 111 | pBB4011 CAR | EIVLTQSPGTLSLSPGERATLSCRASQSITSSYLAWYQQRPGQAPRLLIYGA |
| | a.a. sequence | SSRATGIPDRFSGSGSGTDFTLTISRLEPEDFAVYYCQQYGSSPWTFGQGTK |
| | without signal | VEIKGGGGSGGGGSGGGGSEVQLVESGGGLEQPGRSLRLSCTASGFAFGDHT |
| | peptide | MSWVRQAPGKGLEWVGFIRSRAYGGTTEYAASVKGRFTISRDDSKSIAYLQM |
| | | DSLKTEDTAVYYCTSGGYDSSLHYYYYYHGMDVWGRGTTVTVSSTTTPAPRP |
| | | PTPAPTIASQPLSLRPEACRPAAGGAVHTRGLDFACDIYIWAPLAGTCGVLL |
| | | LSLVITLYCKRGRKKLLYIFKQPFMRPVQTTQEEDGCSCRFPEEEEGGCELR |
| | | VKFSRSADAPAYQQGQNQLYNELNLGRREEYDVLDKRRGRDPEMGGKPRRKN |
| | | PQEGLYNELQKDKMAEAYSEIGMKGERRRGKGHDGLYQGLSTATKDTYDALH |
| | | MQALPPR |
| 112 | pBB4011 CAR | MALPVTALLLPLALLLHAARPEIVLTQSPGTLSLSPGERATLSCRASQSITS |
| | a.a. sequence | SYLAWYQQRPGQAPRLLIYGASSRATGIPDRESGSGSGTDFTLTISRLEPED |
| | with signal | FAVYYCQQYGSSPWTFGQGTKVEIKGGGGSGGGGSGGGGSEVQLVESGGGLE |
| | peptide | QPGRSLRLSCTASGFAFGDHTMSWVRQAPGKGLEWVGFIRSRAYGGTTEYAA |
| | | SVKGRFTISRDDSKSIAYLQMDSLKTEDTAVYYCTSGGYDSSLHYYYYYHGM |
| | | DVWGRGTTVTVSSTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQTT |
| | | QEEDGCSCRFPEEEEGGCELRVKFSRSADAPAYQQGQNQLYNELNLGRREEY |
| | | DVLDKRRGRDPEMGGKPRRKNPQEGLYNELQKDKMAEAYSEIGMKGERRRGK |
| | | GHDGLYQGLSTATKDTYDALHMQALPPR |
| 113 | Anti-MUC16 8804 | QTVSSSY |
| | CDRL1 | |
| 114 | Anti-MUC16 8804 | GAS |
| | CDRL2 | |
| 115 | Anti-MUC16 8804 | QQYGSSPWT |
| | CDRL3 | |
| 116 | Anti-MUC16 8804 | GLTFGDYG |
| | CDRH1 | |
| 117 | Anti-MUC16 8804 | IRSKGYGGTT |
| | CDRH2 | |
| 118 | Anti-MUC16 8804 | TSGGYDSSVHYYYYYY |
| | CDRH3 | |
| 119 | Anti-MUC16 8804 | EIVLTQSPGTLSLSPGERATLSCRASQTVSSSYLAWYQQKPGQAPRLLIYGA |
| | variable light | SSRATGIPDRFSGSGSGTDFTLTISRLEPEDFAVYYCQQYGSSPWTFGQGTK |
| | chain | VEIK |
| 120 | Anti-MUC16 8804 | EVQLVESGGGLVQPGRSLRLSCTGSGLTFGDYGMSWVRQAPGKGLEWVGFIR |
| | variable heavy | SKGYGGTTEYAASVKGRFTISRDDSKSTAYLQMNSLKTEDTAVYYCTSGGYD |
| | chain | SSVHYYYYYYAMDVWGQGTTVTVSS |
| 121 | pBB4012 CAR | EVQLVESGGGLVQPGRSLRLSCTGSGLTFGDYGMSWVRQAPGKGLEWVGFIR |
| | a.a. sequence | SKGYGGTTEYAASVKGRFTISRDDSKSTAYLQMNSLKTEDTAVYYCTSGGYD |
| | without signal | SSVHYYYYYYAMDVWGQGTTVTVSSGGGGSGGGGSGGGGSEIVLTQSPGTLS |
| | peptide | LSPGERATLSCRASQTVSSSYLAWYQQKPGQAPRLLIYGASSRATGIPDRES |
| | | GSGSGTDFTLTISRLEPEDFAVYYCQQYGSSPWTFGQGTKVEIKTTTPAPRP |
| | | PTPAPTIASQPLSLRPEACRPAAGGAVHTRGLDFACDIYIWAPLAGTCGVLL |
| | | LSLVITLYCKRGRKKLLYIFKQPFMRPVQTTQEEDGCSCRFPEEEEGGCELR |
| | | VKFSRSADAPAYQQGQNQLYNELNLGRREEYDVLDKRRGRDPEMGGKPRRKN |
| | | PQEGLYNELQKDKMAEAYSEIGMKGERRRGKGHDGLYQGLSTATKDTYDALH |
| | | MQALPPR |
| 122 | pBB4012 CAR | MALPVTALLLPLALLLHAARPEVQLVESGGGLVQPGRSLRLSCTGSGLTFGD |
| | a.a. sequence | YGMSWVRQAPGKGLEWVGFIRSKGYGGTTEYAASVKGRFTISRDDSKSTAYL |
| | with signal | QMNSLKTEDTAVYYCTSGGYDSSVHYYYYYYAMDVWGQGTTVTVSSGGGGSG |
| | peptide | GGGSGGGGSEIVLTQSPGTLSLSPGERATLSCRASQTVSSSYLAWYQQKPGQ |
| | | APRLLIYGASSRATGIPDRFSGSGSGTDFTLTISRLEPEDFAVYYCQQYGSS |
| | | PWTFGQGTKVEIKTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQTT |
| | | QEEDGCSCRFPEEEEGGCELRVKFSRSADAPAYQQGQNQLYNELNLGRREEY |
| | | DVLDKRRGRDPEMGGKPRRKNPQEGLYNELQKDKMAEAYSEIGMKGERRRGK |
| | | GHDGLYQGLSTATKDTYDALHMQALPPR |
| 123 | pBB4013 CAR | EIVLTQSPGTLSLSPGERATLSCRASQTVSSSYLAWYQQKPGQAPRLLIYGA |
| | a.a. sequence | SSRATGIPDRFSGSGSGTDFTLTISRLEPEDFAVYYCQQYGSSPWTFGQGTK |
| | without signal | VEIKGGGGSGGGGSGGGGSEVQLVESGGGLVQPGRSLRLSCTGSGLTFGDYG |
| | peptide | MSWVRQAPGKGLEWVGFIRSKGYGGTTEYAASVKGRFTISRDDSKSTAYLQM |
| | | NSLKTEDTAVYYCTSGGYDSSVHYYYYYYAMDVWGQGTTVTVSSTTTPAPRP |
| | | PTPAPTIASQPLSLRPEACRPAAGGAVHTRGLDFACDIYIWAPLAGTCGVLL |
| | | LSLVITLYCKRGRKKLLYIFKQPFMRPVQTTQEEDGCSCRFPEEEEGGCELR |
| | | VKFSRSADAPAYQQGQNQLYNELNLGRREEYDVLDKRRGRDPEMGGKPRRKN |
| | | PQEGLYNELQKDKMAEAYSEIGMKGERRRGKGHDGLYQGLSTATKDTYDALH |
| | | MQALPPR |
| 124 | pBB4013 CAR | MALPVTALLLPLALLLHAARPEIVLTQSPGTLSLSPGERATLSCRASQTVSS |
| | a.a. sequence | SYLAWYQQKPGQAPRLLIYGASSRATGIPDRFSGSGSGTDFTLTISRLEPED |
| | with signal | FAVYYCQQYGSSPWTFGQGTKVEIKGGGGSGGGGSGGGGSEVQLVESGGGLV |
| | peptide | QPGRSLRLSCTGSGLTFGDYGMSWVRQAPGKGLEWVGFIRSKGYGGTTEYAA |
| | | SVKGRFTISRDDSKSTAYLQMNSLKTEDTAVYYCTSGGYDSSVHYYYYYYAM |
| | | DVWGQGTTVTVSSTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQTT |
| | | QEEDGCSCRFPEEEEGGCELRVKFSRSADAPAYQQGQNQLYNELNLGRREEY |
| | | DVLDKRRGRDPEMGGKPRRKNPQEGLYNELQKDKMAEAYSEIGMKGERRRGK |
| | | GHDGLYQGLSTATKDTYDALHMQALPPR |
| 125 | pBB4014 CAR | QVQLVESGGGVVQPGRSLRLSCVASGFTFSNYGIHWVRQAPGKGLEWVAVIS |
| | a.a. sequence | DDGSFKFYADSVKGRFTISRDNSKNTLYLQMNSLRVEDSAVYHCAKWQHNWN |
| | without signal | DGGFDYWGQGTLVTVSSGGGGSGGGGSGGGGSEIVLTQSPDTLSLSPGERAT |
| | peptide | LSCRASQSLSSNYLAWYRQKPGQAPRLLIYGISSRATGIPDRFSGSGSGTDF |
| | | TLTISRLEPEDFAVYYCQQYGSSPWTFGQGTKVEIKTTTPAPRPPTPAPTIA |
| | | SQPLSLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKFSRSAD |
| | | APAYQQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQALPPR |
| 126 | pBB4014 CAR | MALPVTALLLPLALLLHAARPQVQLVESGGGVVQPGRSLRLSCVASGFTFSN |
| | a.a. sequence | YGIHWVRQAPGKGLEWVAVISDDGSFKFYADSVKGRFTISRDNSKNTLYLQM |
| | with signal | NSLRVEDSAVYHCAKWQHNWNDGGFDYWGQGTLVTVSSGGGGSGGGGSGGGG |
| | peptide | SEIVLTQSPDTLSLSPGERATLSCRASQSLSSNYLAWYRQKPGQAPRLLIYG |
| | | ISSRATGIPDRFSGSGSGTDFTLTISRLEPEDFAVYYCQQYGSSPWTFGQGT |
| | | KVEIKTTTPAPRPPTPAPTIASQPLSLRPEACRPAAGGAVHTRGLDFACDIY |
| | | IWAPLAGTCGVLLLSLVITLYCKRGRKKLLYIFKQPFMRPVQTTQEEDGCSC |
| | | RFPEEEEGGCELRVKFSRSADAPAYQQGQNQLYNELNLGRREEYDVLDKRRG |
| | | RDPEMGGKPRRKNPQEGLYNELQKDKMAEAYSEIGMKGERRRGKGHDGLYQG |
| | | LSTATKDTYDALHMQALPPR |
| 127 | Anti-MUC16 8810 | QSVSNRY |
| | CDRL1 | |
| 128 | Anti-MUC16 8810 | GAS |
| | CDRL2 | |
| 129 | Anti-MUC16 8810 | HQYGSSPWT |
| | CDRL3 | |
| 130 | Anti-MUC16 8810 | GFTFSSYA |
| | CDRH1 | |
| 131 | Anti-MUC16 8810 | ISYNGGTT |
| | CDRH2 | |
| 132 | Anti-MUC16 8810 | ARAAAP |
| | CDRH3 | |
| 133 | Anti-MUC16 8810 | EIVLTQSPDTLSLSPGERATLSCRASQSVSNRYLAWYQQKPGQAPRLLIYGA |
| | variable light | SSRATGIPDRFSGSGSGTDFTLTISRLEPEDFAVYYCHQYGSSPWTFGQGTK |
| | chain | VEIK |
| 134 | Anti-MUC16 8810 | EVQLVESGGALVQPGGSLRLSCAASGFTFSSYAMHWVRQAPGKGLEFVSTIS |
| | variable heavy | YNGGTTYYADSVKGRFTVSRDNSKNTVFLRMGSLRTEDMAVYYCARAAAPWG |
| | chain | QGTLVTVSS |
| 135 | pBB4016 CAR | EVQLVESGGALVQPGGSLRLSCAASGFTFSSYAMHWVRQAPGKGLEFVSTIS |
| | a.a. sequence | YNGGTTYYADSVKGRFTVSRDNSKNTVFLRMGSLRTEDMAVYYCARAAAPWG |
| | without signal | QGTLVTVSSGGGGSGGGGSGGGGSEIVLTQSPDTLSLSPGERATLSCRASQS |
| | peptide | VSNRYLAWYQQKPGQAPRLLIYGASSRATGIPDRFSGSGSGTDFTLTISRLE |
| | | PEDFAVYYCHQYGSSPWTFGQGTKVEIKTTTPAPRPPTPAPTIASQPLSLRP |
| | | EACRPAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYQQGQ |
| | | NQLYNELNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| 136 | pBB4016 CAR | MALPVTALLLPLALLLHAARPEVQLVESGGALVQPGGSLRLSCAASGFTFSS |
| | a.a. sequence | YAMHWVRQAPGKGLEFVSTISYNGGTTYYADSVKGRFTVSRDNSKNTVFLRM |
| | with signal | GSLRTEDMAVYYCARAAAPWGQGTLVTVSSGGGGSGGGGSGGGGSEIVLTQS |
| | peptide | PDTLSLSPGERATLSCRASQSVSNRYLAWYQQKPGQAPRLLIYGASSRATGI |
| | | PDRFSGSGSGTDFTLTISRLEPEDFAVYYCHQYGSSPWTFGQGTKVEIKTTT |
| | | PAPRPPTPAPTIASQPLSLRPEACRPAAGGAVHTRGLDFACDIYIWAPLAGT |
| | | CGVLLLSLVITLYCKRGRKKLLYIFKQPFMRPVQTTQEEDGCSCRFPEEEEG |
| | | GCELRVKFSRSADAPAYQQGQNQLYNELNLGRREEYDVLDKRRGRDPEMGGK |
| | | PRRKNPQEGLYNELQKDKMAEAYSEIGMKGERRRGKGHDGLYQGLSTATKDT |
| | | YDALHMQALPPR |
| 137 | pBB4017 CAR | EIVLTQSPDTLSLSPGERATLSCRASQSVSNRYLAWYQQKPGQAPRLLIYGA |
| | a.a. sequence | SSRATGIPDRFSGSGSGTDFTLTISRLEPEDFAVYYCHQYGSSPWTFGQGTK |
| | without signal | VEIKGGGGSGGGGSGGGGSEVQLVESGGALVQPGGSLRLSCAASGFTESSYA |
| | peptide | MHWVRQAPGKGLEFVSTISYNGGTTYYADSVKGRFTVSRDNSKNTVFLRMGS |
| | | LRTEDMAVYYCARAAAPWGQGTLVTVSSTTTPAPRPPTPAPTIASQPLSLRP |
| | | EACRPAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYQQGQ |
| | | NQLYNELNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| 138 | pBB4017 CAR | MALPVTALLLPLALLLHAARPEIVLTQSPDTLSLSPGERATLSCRASQSVSN |
| | a.a. sequence | RYLAWYQQKPGQAPRLLIYGASSRATGIPDRESGSGSGTDFTLTISRLEPED |
| | with signal | FAVYYCHQYGSSPWTFGQGTKVEIKGGGGSGGGGSGGGGSEVQLVESGGALV |
| | peptide | QPGGSLRLSCAASGFTFSSYAMHWVRQAPGKGLEFVSTISYNGGTTYYADSV |
| | | KGRFTVSRDNSKNTVFLRMGSLRTEDMAVYYCARAAAPWGQGTLVTVSSTTT |
| | | PAPRPPTPAPTIASQPLSLRPEACRPAAGGAVHTRGLDFACDIYIWAPLAGT |
| | | CGVLLLSLVITLYCKRGRKKLLYIFKQPFMRPVQTTQEEDGCSCRFPEEEEG |
| | | GCELRVKFSRSADAPAYQQGQNQLYNELNLGRREEYDVLDKRRGRDPEMGGK |
| | | PRRKNPQEGLYNELQKDKMAEAYSEIGMKGERRRGKGHDGLYQGLSTATKDT |
| | | YDALHMQALPPR |
| 139 | Anti-MUC16 8813 | QSVSSSY |
| | CDRL1 | |
| 140 | Anti-MUC16 8813 | GAS |
| | CDRL2 | |
| 141 | Anti-MUC16 8813 | QQYGSSPWT |
| | CDRL3 | |
| 142 | Anti-MUC16 8813 | GYTLTGYY |
| | CDRH1 | |
| 143 | Anti-MUC16 8813 | INPNNGGT |
| | CDRH2 | |
| 144 | Anti-MUC16 8813 | ARSLELLPDGMDV |
| | CDRH3 | |
| 145 | Anti-MUC16 8813 | EIVLTQSPGTLSLSPGERATLSCRASQSVSSSYLAWYQQKPGQAPRLLIYGA |
| | variable light | SSRATGIPDRFSGSGSGTDFTLTISRLEPEDFAVYYCQQYGSSPWTFGQGTK |
| | chain | VEIK |
| 146 | Anti-MUC16 8813 | QVQLVQSGAEVKKPGASVKVSCKAFGYTLTGYYIHWVRQAPGQGLEWMGWIN |
| | variable heavy | PNNGGTNYAQKFQGRVTMTRDKSISTAYMELSRLRSDDTAVYYCARSLELLP |
| | chain | DGMDVWGQGTTVTVSS |
| 147 | pBB4018 CAR | QVQLVQSGAEVKKPGASVKVSCKAFGYTLTGYYIHWVRQAPGQGLEWMGWIN |
| | a.a. sequence | PNNGGTNYAQKFQGRVTMTRDKSISTAYMELSRLRSDDTAVYYCARSLELLP |
| | without signal | DGMDVWGQGTTVTVSSGGGGSGGGGSGGGGSEIVLTQSPGTLSLSPGERATL |
| | peptide | SCRASQSVSSSYLAWYQQKPGQAPRLLIYGASSRATGIPDRFSGSGSGTDFT |
| | | LTISRLEPEDFAVYYCQQYGSSPWTFGQGTKVEIKTTTPAPRPPTPAPTIAS |
| | | QPLSLRPEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKFSRSADA |
| | | PAYQQGQNQLYNELNLGRREEYDVLDKRRGRDPEMGGKPRRKNPQEGLYNEL |
| | | QKDKMAEAYSEIGMKGERRRGKGHDGLYQGLSTATKDTYDALHMQALPPR |
| 148 | pBB4018 CAR | MALPVTALLLPLALLLHAARPQVQLVQSGAEVKKPGASVKVSCKAFGYTLTG |
| | a.a. sequence | YYIHWVRQAPGQGLEWMGWINPNNGGTNYAQKFQGRVTMTRDKSISTAYMEL |
| | with signal | SRLRSDDTAVYYCARSLELLPDGMDVWGQGTTVTVSSGGGGSGGGGSGGGGS |
| | peptide | EIVLTQSPGTLSLSPGERATLSCRASQSVSSSYLAWYQQKPGQAPRLLIYGA |
| | | SSRATGIPDRFSGSGSGTDFTLTISRLEPEDFAVYYCQQYGSSPWTFGQGTK |
| | | VEIKTTTPAPRPPTPAPTIASQPLSLRPEACRPAAGGAVHTRGLDFACDIYI |
| | | WAPLAGTCGVLLLSLVITLYCKRGRKKLLYIFKQPFMRPVQTTQEEDGCSCR |
| | | FPEEEEGGCELRVKFSRSADAPAYQQGQNQLYNELNLGRREEYDVLDKRRGR |
| | | DPEMGGKPRRKNPQEGLYNELQKDKMAEAYSEIGMKGERRRGKGHDGLYQGL |
| | | STATKDTYDALHMQALPPR |
| 149 | pBB4019 CAR | EIVLTQSPGTLSLSPGERATLSCRASQSVSSSYLAWYQQKPGQAPRLLIYGA |
| | a.a. sequence | SSRATGIPDRFSGSGSGTDFTLTISRLEPEDFAVYYCQQYGSSPWTFGQGTK |
| | without signal | VEIKGGGGSGGGGSGGGGSQVQLVQSGAEVKKPGASVKVSCKAFGYTLTGYY |
| | peptide | IHWVRQAPGQGLEWMGWINPNNGGTNYAQKFQGRVTMTRDKSISTAYMELSR |
| | | LRSDDTAVYYCARSLELLPDGMDVWGQGTTVTVSSTTTPAPRPPTPAPTIAS |
| | | QPLSLRPEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKFSRSADA |
| | | PAYQQGQNQLYNELNLGRREEYDVLDKRRGRDPEMGGKPRRKNPQEGLYNEL |
| | | QKDKMAEAYSEIGMKGERRRGKGHDGLYQGLSTATKDTYDALHMQALPPR |
| 150 | pBB4019 CAR | MALPVTALLLPLALLLHAARPEIVLTQSPGTLSLSPGERATLSCRASQSVSS |
| | a.a. sequence | SYLAWYQQKPGQAPRLLIYGASSRATGIPDRFSGSGSGTDFTLTISRLEPED |
| | with signal | FAVYYCQQYGSSPWTFGQGTKVEIKGGGGSGGGGSGGGGSQVQLVQSGAEVK |
| | peptide | KPGASVKVSCKAFGYTLTGYYIHWVRQAPGQGLEWMGWINPNNGGTNYAQKF |
| | | QGRVTMTRDKSISTAYMELSRLRSDDTAVYYCARSLELLPDGMDVWGQGTTV |
| | | TVSSTTTPAPRPPTPAPTIASQPLSLRPEACRPAAGGAVHTRGLDFACDIYI |
| | | WAPLAGTCGVLLLSLVITLYCKRGRKKLLYIFKQPFMRPVQTTQEEDGCSCR |
| | | FPEEEEGGCELRVKFSRSADAPAYQQGQNQLYNELNLGRREEYDVLDKRRGR |
| | | DPEMGGKPRRKNPQEGLYNELQKDKMAEAYSEIGMKGERRRGKGHDGLYQGL |
| | | STATKDTYDALHMQALPPR |
| 151 | Human MUC16 | PGSRKFNTTERVLQGLLRPLFKNTSVGPLYSGCRLTLLRPEKDGEATGVDAI |
| | ectodomain | CTHRPDPTGPGLDREQLYLELSQLTHSITELGPYTLDRDSLYVNGFTHRSSV |
| | (“nub”) | PTTSTGVVSEEPFTLNFTINNLRYMADMGQPGSLKFNITDNVMQHLLSPLFQ |
| | | RSSLGARYTGCRVIALRSVKNGAETRVDLLCTYLQPLSGPGLPIKQVFHELS |
| | | QQTHGITRLGPYSLDKDSLYLNGYNEPGPDEPPTTPKPATTFLPPLSEATTA |
| | | MGYHLKTLTLNFTISNLQYSPDMGKGSATFNSTEGVLQHLLRPLFQKSSMGP |
| | | FYLGCQLISLRPEKDGAATGVDTTCTYHPDPVGPGLDIQQLYWELSQLTHGV |
| | | TQLGFYVLDRDSLFINGYAPQNLSIRGEYQINFHIVNWNLSNPDPTSSEYIT |
| | | LLRDIQDKVTTLYKGSQLHDTFRFCLVTNLTMDSVLVTVKALFSSNLDPSLV |
| | | EQVFLDKTLNASFHWLGSTYQLVDIHVTEMESSVYQPTSSSSTQHFYLNFTI |
| | | TNLPYSQDKAQPGTTNYQRNKRNIEDALNQLFRNSSIKSYFSDCQVSTFRSV |
| | | PNRHHTGVDSLCNFSPLARRVDRVAIYEEFLRMTRNGTQLQNFTLDRSSVLV |
| | | DGYSPNRNEPLIGNSDLP |
| 152 | CD8 signal | MALPVTALLLPLALLLHAARP |
| | peptide | |
| 153 | CD8 hinge | TTTPAPRPPTPAPTIASQPLSLRPEACRPAAGGAVHTRGLDFACD |
| | region | |
| 154 | CD8 | IYIWAPLAGTCGVLLLSLVIT |
| | transmembrane | |
| | region | |
| | Tail/linker | LYC |
| 155 | 4-1BB Co- | KRGRKKLLYIFKQPFMRPVQTTQEEDGCSCRFPEEEEGGCEL |
| | stimulatory | |
| | region | |
| 156 | CD3 signaling | RVKFSRSADAPAYQQGQNQLYNELNLGRREEYDVLDKRRGRDPEMGGKPRRK |
| | region | NPQEGLYNELQKDKMAEAYSEIGMKGERRRGKGHDGLYQGLSTATKDTYDAL |
| | | HMQALPPR |

1. A chimeric antigen receptor (CAR) comprising: an extracellular domain comprising an anti-MUC16 antibody or antigen binding fragment thereof that binds one or more epitopes of a human MUC16 polypeptide; a transmembrane domain, one or more intracellular co-stimulatory signaling domains, and a primary signaling domain.

2. The CAR of claim 1, wherein the MUC16 antibody or antigen binding fragment thereof binds a membrane proximal fragment of MUC16 remaining on the cell surface after proteolytic cleavage of a full-length MUC16 polypeptide.

3. The CAR of claim 2, wherein the fragment of MUC16 remaining on the cell surface after proteolytic cleavage comprises SEQ ID NO: 151.

4. The CAR of any one of claims 1-3, wherein the full-length MUC16 polypeptide comprises 16 sea urchin sperm, enterokinase and agrin (SEA) domains, numbered 1-16 from N-terminus to C-terminus, and wherein the fragment of MUC16 comprises SEA domains 12-16.

5. The CAR of any one of claims 1-4, wherein the anti-MUC16 antibody or antigen binding fragment that binds the human MUC16 polypeptide is selected from the group consisting of: Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a Camel Ig, a VHH, Ig NAR, a minibody, a diabody, a triabody, a tetrabody, a disulfide stabilized Fv protein (“dsFv”), and a single-domain antibody (sdAb, Nanobody) or fragment thereof.

6. The CAR of any one of claims 1 to 5, wherein the anti-MUC16 antibody or antigen binding fragment thereof that binds the human MUC16 polypeptide is an scFv.

7. The CAR of any one of claims 1 to 6, wherein the anti-MUC16 antibody or antigen binding fragment thereof comprises CDRL1, CDRL2, and CDRL3 regions within a variable light chain amino acid sequence as set forth in SEQ ID NO: 7.

8. The CAR of any one of claims 1 to 6, wherein the anti-MUC16 antibody or antigen binding fragment thereof comprises CDRL1, CDRL2, and CDRL3 regions within a variable light chain amino acid sequence as set forth in any one of SEQ ID NOs: 47, 59, 71, 83, 95, 107, 119, 133, or 145.

9. The CAR of any one of claims 1 to 8, wherein the anti-MUC16 antibody or antigen binding fragment thereof comprises CDRH1, CDRH2, and CDRH3 regions within a variable heavy chain amino acid sequence as set forth in SEQ ID NO: 8.

10. The CAR of any one of claims 1 to 8, wherein the anti-MUC16 antibody or antigen binding fragment thereof comprises CDRH1, CDRH2, and CDRH3 regions within a variable heavy chain amino acid sequence as set forth in any one of SEQ ID NOs: 48, 60, 72, 84, 96, 108, 120, 134, or 146.

11. The CAR of any one of claims 1 to 10, wherein the anti-MUC16 antibody or antigen binding fragment thereof comprises one or more CDRs as set forth in any one of SEQ ID NOs: 1-3.

12. The CAR of any one of claims 1 to 10, wherein the anti-MUC16 antibody or antigen binding fragment thereof comprises one or more CDRs as set forth in any one of (a) SEQ ID NOs: 41-43, (b) SEQ ID NOs: 53-55, (c) SEQ ID NOs: 65-67, (d) SEQ ID NOs: 77-79, (e) SEQ ID NOs: 89-91, (f) SEQ ID NOs: 101-103, (g) SEQ ID NOs: 113-115, (h) SEQ ID NOs: 127-129, or (i) SEQ ID NOs: 139-141.

13. The CAR of any one of claims 1 to 12, wherein the anti-MUC16 antibody or antigen binding fragment thereof comprises one or more CDRs as set forth in any one of SEQ ID NOs: 4-6.

14. The CAR of any one of claims 1 to 12, wherein the anti-MUC16 antibody or antigen binding fragment thereof comprises one or more CDRs as set forth in any one of (a) SEQ ID NOs: 44-46, (b) SEQ ID NOs: 56-58, (c) SEQ ID NOs: 68-70, (d) SEQ ID NOs: 80-82, (e) SEQ ID NOs: 92-94, (f) SEQ ID NOs: 104-106, (g) SEQ ID NOs: 116-118, (h) SEQ ID NOs: 130-132, or (i) SEQ ID NOs: 142-144.

15. The CAR of any one of claims 1 to 14, wherein the anti-MUC16 antibody or antigen binding fragment thereof comprises a variable light chain amino acid sequence having at least 90%, 91%, 92%, 93%, 94%, 95%, 96%, 97%, 98%, 99% identity to, or comprises SEQ ID NO: 7.

16. The CAR of any one of claims 1 to 14, wherein the anti-MUC16 antibody or antigen binding fragment thereof comprises a variable light chain amino acid sequence having at least 90%, 91%, 92%, 93%, 94%, 95%, 96%, 97%, 98%, 99% identity to, or comprises any one of SEQ ID NOs: 47, 59, 71, 83, 95, 107, 119, 133, or 145.

17. The CAR of any one of claims 1 to 16, wherein the anti-MUC16 antibody or antigen binding fragment thereof comprises a variable heavy chain amino acid sequence having at least 90%, 91%, 92%, 93%, 94%, 95%, 96%, 97%, 98%, 99% identity to, or comprises SEQ ID NO: 8.

18. The CAR of any one of claims 1 to 16, wherein the anti-MUC16 antibody or antigen binding fragment thereof comprises a variable heavy chain amino acid sequence having at least 90%, 91%, 92%, 93%, 94%, 95%, 96%, 97%, 98%, 99% identity to, or comprises any one of SEQ ID NOs: 48, 60, 72, 84, 96, 108, 120, 134, or 146.

19. The CAR of any one of claims 1 to 18, wherein the transmembrane domain is from a polypeptide selected from the group consisting of: alpha, beta or zeta chain of the T-cell receptor, CD3ε, CD3ζ, CD4, CD5, CD8α, CD9, CD 16, CD22, CD27, CD28, CD33, CD3γ, CD45, CD64, CD80, CD86, CD 134, CD137, CD152, CD 154, and PD1.

20. The CAR of any one of claims 1 to 18, wherein the transmembrane domain is from a polypeptide selected from the group consisting of: CD8α, CD4, CD45, PD1, and CD154.

21. The CAR of any one of claims 1 to 18, wherein the transmembrane domain is from CD8α.

22. The CAR of claim 21, wherein the transmembrane domain comprises the sequence of SEQ ID NO: 154.

23. The CAR of any one of claims 1 to 22, wherein the one or more co-stimulatory signaling domains are from a co-stimulatory molecule selected from the group consisting of: CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70.

24. The CAR of any one of claims 1 to 22, wherein the one or more co-stimulatory signaling domains are from a co-stimulatory molecule selected from the group consisting of: CD28, CD134, and CD137 (4-1BB).

25. The CAR of any one of claims 1 to 22, wherein the one or more co-stimulatory signaling domains is from CD137 (4-1BB).

26. The CAR of claim 25, wherein the co-stimulatory signaling domain comprises the sequence of SEQ ID NO: 155.

27. The CAR of any one of claims 1 to 26, wherein the primary signaling domain is from CD3ζ.

28. The CAR of claim 27, wherein the primary signaling domain comprises the sequence of SEQ ID NO: 156.

29. The CAR of any one of claims 1 to 28, further comprising a hinge region polypeptide.

30. The CAR of claim 29, wherein the hinge region polypeptide comprises a hinge region of CD8α.

31. The CAR of claim 30, wherein the hinge region polypeptide comprises the sequence of SEQ ID NO: 153.

32. The CAR of any one of claims 1 to 31, further comprising a signal polypeptide.

33. The CAR of claim 32, wherein the signal polypeptide comprises an IgG1 heavy chain signal polypeptide, granulocyte-macrophage colony stimulating factor receptor 2 (GM-CSFR2) signal polypeptide, Igκ signal polypeptide, or a CD8α signal polypeptide.

34. The CAR of claim 33, wherein the signal polypeptide comprises a CD8α signal polypeptide.

35. The CAR of claim 34, wherein the signal polypeptide comprises the sequence of SEQ ID NO: 152.

36. The CAR of any one of claims 1 to 35, further comprising a polypeptide linker between the variable heavy chain and variable light chain domains.

37. The CAR of claim 36, wherein the polypeptide linker between the variable heavy chain and variable light chain domains comprises an amino acid sequence of any one of SEQ ID NOs: 14-25.

38. The CAR of claim 37, wherein the polypeptide linker between the variable heavy chain and variable light chain domains comprises a 3xG4S amino acid linker as set forth in SEQ ID NO: 24.

39. The CAR of any one of claims 1 to 38, further comprising a polypeptide linker between the transmembrane domain and one or more intracellular co-stimulatory signaling domains.

40. The CAR of claim 39, wherein the polypeptide linker between the transmembrane domain and one or more intracellular co-stimulatory signaling domains comprises the sequence of LYC.

41. The CAR of any one of claims 1-40, wherein the anti-MUC16 antibody or antigen binding fragment thereof comprises CDRL1, CDRL2, and CDRL3 regions within a variable light chain amino acid sequence as set forth in SEQ ID NO: 7 and CDRH1, CDRH2 and CDRH3 regions within a variable heavy chain amino acid sequence as set forth in SEQ ID NO: 8; the transmembrane domain is from a polypeptide selected from the group consisting of: CD8α, CD4, CD45, PD1, and CD154; the one or more co-stimulatory signaling domains are from a co-stimulatory molecule selected from the group consisting of: CD28, CD134, and CD137 (4-1BB); and the primary signaling domain is from CD3ζ.

42. The CAR of any one of claims 1-40, wherein the anti-MUC16 antibody or antigen binding fragment thereof comprises CDRL1, CDRL2, and CDRL3 regions within a variable light chain amino acid sequence as set forth in SEQ ID NO: 7 and CDRH1, CDRH2 and CDRH3 regions within a variable heavy chain amino acid sequence as set forth in SEQ ID NO: 8; the transmembrane domain is from CD8α; the one or more co-stimulatory signaling domains is from CD137 (4-1BB); and the primary signaling domain is from CD3ζ.

43. The CAR of any one of claims 1 to 42, comprising an amino acid sequence having at least 90%, 91%, 92%, 93%, 94%, 95%, 96%, 97%, 98%, or 99% identity to, or comprising SEQ ID NO: 9 or SEQ ID NO: 11.

44. The CAR of any one of claims 1 to 42, comprising an amino acid sequence having at least 90%, 91%, 92%, 93%, 94%, 95%, 96%, 97%, 98%, or 99% identity to, or comprising any one of SEQ ID NOs: 49, 50, 51, 52, 61, 62, 63, 64, 73, 74, 75, 76, 85, 86, 87, 88, 97, 98, 99, 100, 109, 110, 111, 112, 121, 122, 123, 124, 125, 126, 135, 136, 137, 138, 147, 148, 149 or 150.

45. A CAR that competes for binding to one or more epitopes of a human MUC16 polypeptide with the CAR of any one of claims 1 to 44.

46. A polynucleotide encoding the CAR of any one of claims 1 to 45.

47. A polynucleotide encoding a CAR, wherein the polynucleotide sequence encodes a polypeptide having at least 90%, 91%, 92%, 93%, 94%, 95%, 96%, 97%, 98%, or 99% identity to, or comprises SEQ ID NO: 10 or SEQ ID NO: 12.

48. A polynucleotide encoding a CAR, wherein the polynucleotide sequence encodes a polypeptide having at least 90%, 91%, 92%, 93%, 94%, 95%, 96%, 97%, 98%, or 99% identity to, or comprising any one of SEQ ID NOs: 9, 11, 49, 50, 51, 52, 61, 62, 63, 64, 73, 74, 75, 76, 85, 86, 87, 88, 97, 98, 99, 100, 109, 110, 111, 112, 121, 122, 123, 124, 125, 126, 135, 136, 137, 138, 147, 148, 149 or 150.

49. A vector comprising the polynucleotide of any one of claims 46-48.

50. The vector of claim 49, wherein the vector is an expression vector.

51. The vector of claim 49, wherein the vector is an episomal vector.

52. The vector of claim 49, wherein the vector is a viral vector.

53. The vector of claim 49, wherein the vector is a retroviral vector.

54. The vector of claim 49, wherein the vector is a lentiviral vector.

55. The vector of claim 49, wherein the vector is an adeno-associated viral vector.

56. The vector of claim 54, wherein the lentiviral vector is selected from the group consisting of: human immunodeficiency virus 1 (HIV-1); human immunodeficiency virus 2 (HIV-2), visna-maedi virus (VMV) virus; caprine arthritis-encephalitis virus (CAEV); equine infectious anemia virus (EIAV); feline immunodeficiency virus (FIV); bovine immune deficiency virus (BIV); and simian immunodeficiency virus (SIV).

57. The vector according to any one of claims 49 to 56, comprising a left (5′) retroviral LTR, a Psi (Ψ) packaging signal, a central polypurine tract/DNA flap (cPPT/FLAP), a retroviral export element; a promoter operably linked to the polynucleotide of claim 26 or claim 27; and a right (3′) retroviral LTR.

58. The vector of claim 57, further comprising a heterologous polyadenylation sequence.

59. The vector of any of claim 58, wherein the promoter of the 5′ LTR is replaced with a heterologous promoter.

60. The vector of claim 59, wherein the heterologous promoter is a cytomegalovirus (CMV) promoter, a Rous Sarcoma Virus (RSV) promoter, or an Simian Virus 40 (SV40) promoter.

61. The vector of any one of claims 57 to 60, wherein the 5′ LTR or 3′ LTR is a lentivirus LTR.

62. The vector of any one of claims 57 to 61, wherein the 3′ LTR comprises one or more modifications.

63. The vector of any one of claims 57-62 wherein the 3′ LTR comprises one or more deletions.

64. The vector of any one of claims 57 to 63, wherein the 3′ LTR is a self-inactivating (SIN) LTR.

65. The vector of any one of claims 57 to 64, wherein the promoter operably linked to the polynucleotide of any one of claims 47-48 is selected from the group consisting of: a cytomegalovirus immediate early gene promoter (CMV), an elongation factor 1 alpha promoter (EF1-α), a phosphoglycerate kinase-1 promoter (PGK), a ubiquitin-C promoter (UBQ-C), a cytomegalovirus enhancer/chicken beta-actin promoter (CAG), polyoma enhancer/herpes simplex thymidine kinase promoter (MC1), a beta actin promoter (β-ACT), a simian virus 40 promoter (SV40), and a myeloproliferative sarcoma virus enhancer, negative control region deleted, dl587rev primer-binding site substituted (MND) promoter.

66. The vector of claim 65, wherein the promoter operably linked to the polynucleotide of any one of claims 46-48 is an MND promoter.

67. A cell comprising the vector of any one of claims 49 to 66.

68. A cell comprising the CAR of any one of claims 1-45.

69. The cell of claim 67 or 68, wherein the cell is an immune effector cell.

70. The cell of claim 69, wherein the immune effector cell is a cytotoxic T lymphocytes (CTLs), a tumor infiltrating lymphocytes (TILs), or a helper T cell.

71. The cell of claim 67 or 68, wherein the cell is a T cell.

72. The cell of claim 67 or 68, wherein the cell is an αβ T cell, a γδ T cell, a natural killer (NK) cell, or a natural killer T (NKT) cell.

73. A composition comprising the cell of any one of claims 67 to 72.

74. A pharmaceutical composition comprising the cell of any one of claims 67 to 72 and a physiologically acceptable excipient.

75. A method of generating an immune effector cell comprising a CAR according to any one of claims 1 to 45 comprising introducing into an immune effector cell the polynucleotide of any one of claims 46-48, or the vector of any one of claims 49-66.

76. A method of treating a cancer in a subject in need thereof, comprising administering to the subject a therapeutically effective amount of the composition of claim 73 or the pharmaceutical composition of claim 74.

77. The method of claim 76, wherein the cancer is a solid cancer, optionally wherein the solid cancer expresses MUC16.

78. The method of claim 77, wherein the solid cancer is selected from the group consisting of sarcoma, prostate cancer, uterine cancer, thyroid cancer, testicular cancer, renal cancer, pancreatic cancer, ovarian cancer, cervical, mesothelioma, esophageal cancer, lung cancer, non-small-cell lung cancer, small cell lung cancer, melanoma, hepatocellular carcinoma, head and neck cancer, gastric cancer, endometrial cancer, fallopian tube cancer, colorectal cancer, cholangiocarcinoma, breast cancer, and bladder cancer.

79. The method of claim 77, wherein the solid cancer is selected from the group consisting of ovarian, endometrial cancer, cervical cancer, mesothelioma, NSCLC, and SCLC.

80. The method of claim 77, wherein the solid cancer is ovarian cancer.

81. The method of any one of claims 76 to 80, wherein the composition is administered in combination with an additional therapeutic agent.

82. A method of treating a cancer in a subject who has received or is receiving a therapeutic agent, comprising administering to the subject a therapeutically effective amount of the composition of claim 73 or the pharmaceutical composition of claim 74.

83. The method of claim 81 or 82, wherein the composition and therapeutic agent are administered sequentially or simultaneously.

84. The method of any one of claims 81 to 83, wherein the therapeutic agent is an immune checkpoint inhibitor, oncolytic virus, or co-stimulatory antibody.

85. The method of claim 84, wherein the immune checkpoint inhibitor binds a checkpoint protein or corresponding ligand selected from the group consisting of: programmed cell death protein 1 (PD-1; PDCD1), lymphocyte activation gene 3 protein (LAG-3), T cell immunoglobulin domain and mucin domain protein 3 (TIM-3), cytotoxic T lymphocyte antigen-4 (CTLA-4), band T lymphocyte attenuator (BTLA), glucocorticoid-induced tumor necrosis factor receptor (GITR), T cell immunoglobulin and immunoreceptor tyrosine-based inhibitory motif domain (TIGIT), V-domain Ig suppressor of T cell activation (VISTA), killer cell immunoglobulin-like receptor (KIR), ICOS, ICOSL, OX40, B7-H3, B7-H4, CD47, 4-1BB, CD27, and CD70.

86. The method of claim 84 or 85, wherein the immune checkpoint inhibitor is a PD-1 inhibitor or PD-L1 inhibitor.

87. The method of claim 86, wherein the PD-1 inhibitor or PD-L1 inhibitor is an anti-PD-1 or anti-PD-L1 antibody or antigen-binding fragment thereof.

88. The method of claim 87, wherein the PD-1 inhibitor is selected from the group consisting of: nivolumab, pembrolizumab, atezolizumab, and cemiplimab.

89. The method of claim 84, wherein the co-stimulatory antibody is a bi-specific antibody.

90. The method of claim 89, wherein the bi-specific antibody binds a tumor associated antigen (TAA) and CD3.

91. The method of claim 89, wherein the bi-specific antibody binds a TAA and CD28.

92. The method of claim 90 or claim 91, wherein the TAA is selected from the group consisting of AFP, ALK, BAGE proteins, β-catenin, brc-abl, BRCA1, BORIS, CA9, carbonic anhydrase IX, caspase-8, CCR5, CD40, CDK4, CEA, CTLA4, cyclin-B1, CYP1B1, ErbB3, ErbB4, ETV6-AML, Fra-1, FOLR1, GAGE proteins (e.g., GAGE-1, -2), GD2, GloboH, glypican-3, GM3, gp100, Her2, HLA/B-raf, HLA/k-ras, HLA/MAGE-A3, hTERT, LMP2, MART-1, ML-IAP, Muci, Muc2, Muc3, Muc4, Muc5, Muci6, MUM1, NA17, NY-BR1, NY-BR62, NY-BR85, OX40, p15, p53, PAP, PAX3, PAX5, PCTA-1, PRLR, RAGE proteins, Ras, RGS5, Rho, SART-1, SART-3, Steap-1, Steap-2, survivin, TGF-β, TMPRSS2, Tn, TRP-1, TRP-2, tyrosinase, uroplakin-3, alpha folate receptor (FRα), αvβ6 integrin, B cell maturation antigen (BCMA), B7-H3 (CD276), B7-H6, carbonic anhydrase IX (CAIX), CCR1, CD16, CD19, CD20, CD22, CD30, CD33, CD3γ, CD38, CD44, CD44v6, CD44v7/8, CD70, CD79a, CD79b, CD123, CD133, CD135 (also known as fmc like tyrosine kinase 3; FLT3), CD138, CD171, carcinoembryonic antigen (CEA), Claudin-6 (CLDN6), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), IL-11Rα, IL-13Rα2, Kappa, cancer/testis antigen 2 (LAGE-1A), Lambda, Lewis-Y (LeY), L1 cell adhesion molecule (L1-CAM), Leukocyte immunoglobulin-like receptor subfamily B member 2 (LILRB2); melanoma antigen gene (MAGE)-A1, MAGE-A3, MAGE-A4, MAGE-A6, MAGEA10, melanoma antigen recognized by T cells 1 (MelanA or MART1), Mesothelin (MSLN), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), tumor associated glycoprotein 72 (TAG72), T-cell immunoglobulin and mucin-domain containing-3 (TIM-3), tumor endothelial marker 1 (TEM1/CD248), tumor endothelial marker 7-related (TEM7R), trophoblast glycoprotein (TPBG), NKG2D ligands, vascular endothelial growth factor receptor 2 (VEGFR2), and Wilms tumor 1 (WT-1).

93. The method of claim 90 or claim 91, wherein the TAA is MSLN.

94. The method of claim 90 or claim 91, wherein the TAA is alpha folate receptor (FRα).

95. The method of claim 90 or claim 91, wherein the TAA is MUC16.

96. The method of any one of claims 81-83, wherein the therapeutic agent is a vascular endothelial growth factor (VEGF) inhibitor.

97. The method of claim 96, wherein the VEGF inhibitor is bevacizumab.

98. The method of any one of claims 81-83, wherein the therapeutic agent is a cytokine.

99. The method of claim 98, wherein the cytokine is IL-2 or masked IL-2.

100. The method of any one of claims 81-83, wherein the therapeutic agent is an oncolytic virus.

101. The method of claim 100, wherein the oncolytic virus selected from the group consisting of VSV (Voyager V1), HSV, adenovirus, maraba virus, measles virus, NDV, picornavirus, reovirus, or vaccinia virus.

102. The method of claim 100, wherein the oncolytic virus is Voyager V1.


SEQUENCE LISTING (156 sequences)

SEQ ID NO: 1 - protein, 7 aa, synthetic construct
  REGION 1..7: note = Synthetic: Anti-MUC16 8808 CDRL1
QSLSSNY

SEQ ID NO: 2 - skipped

SEQ ID NO: 3 - protein, 9 aa, synthetic construct
  REGION 1..9: note = Synthetic: Anti-MUC16 8808 CDRL3
QQYGSSPWT

SEQ ID NO: 4 - protein, 8 aa, synthetic construct
  REGION 1..8: note = Synthetic: Anti-MUC16 8808 CDRH1
GFTFSNYG

SEQ ID NO: 5 - protein, 8 aa, synthetic construct
  REGION 1..8: note = Synthetic: Anti-MUC16 8808 CDRH2
ISDDGSFK

SEQ ID NO: 6 - protein, 14 aa, synthetic construct
  REGION 1..14: note = Synthetic: Anti-MUC16 8808 CDRH3
AKWQHNWNDGGFDY

SEQ ID NO: 7 - protein, 108 aa, synthetic construct
  REGION 1..108: note = Synthetic: Anti-MUC16 8808 variable light chain
EIVLTQSPDTLSLSPGERATLSCRASQSLSSNYLAWYRQKPGQAPRLLIYGISSRATGIPDRFSGSGSGTDFTLTISRLEPEDFAVYYCQQYGSSPWTFGQGTKVEIK

SEQ ID NO: 8 - protein, 121 aa, synthetic construct
  REGION 1..121: note = Synthetic: Anti-MUC16 8808 variable heavy chain
QVQLVESGGGVVQPGRSLRLSCVASGFTFSNYGIHWVRQAPGKGLEWVAVISDDGSFKFYADSVKGRFTISRDNSKNTLYLQMNSLRVEDSAVYHCAKWQHNWNDGGFDYWGQGTLVTVSS

SEQ ID NO: 9 - protein, 467 aa, synthetic construct
  REGION 1..467: note = Synthetic: pBB4015 CAR a.a. sequence without signal peptide
EIVLTQSPDTLSLSPGERATLSCRASQSLSSNYLAWYRQKPGQAPRLLIYGISSRATGIPDRFSGSGSGTDFTLTISRLEPEDFAVYYCQQYGSSPWTFGQGTKVEIKGGGGSGGGGSGGGGSQVQLVESGGGVVQPGRSLRLSCVASGFTFSNYGIHWVRQAPGKGLEWVAVISDDGSFKFYADSVKGRFTISRDNSKNTLYLQMNSLRVEDSAVYHCAKWQHNWNDGGFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - DNA, 1401 bp, synthetic construct
  misc_feature 1..1401: note = Synthetic: pBB4015 CAR n.a. sequence without signal peptide
  source 1..1401: mol_type = other DNA
gaaattgtgctgacccagagccccgataccctttccctttccccaggcgagcgcgccacactgtcttgcagggcgtcacagagcctgagcagcaactatctcgcgtggtatagacagaaaccagggcaagccccacggctgctgatctatggaattagctcacgggcaacaggaatccccgacagattcagtgggtctgggagcggaacggattttaccctgacaattagtagattggaaccggaagacttcgctgtgtactattgccagcagtacggctcatccccgtggaccttcggacaaggcactaaggtagagatcaaaggtggagggggctcagggggaggcggaagcggaggcggaggatctcaggtgcagctggttgagtctggaggcggagtcgttcagccaggtaggagcctccgactctcctgcgtcgcaagtgggtttacgttttccaattacgggattcactgggtccgacaggcccccgggaaggggctcgagtgggtggccgttattagcgatgacgggtctttcaagttctatgccgattcagtcaaaggtagattcacaatttcaagggataatagtaaaaatacactgtacttgcaaatgaactctctgcgcgtcgaggattcagccgtgtaccattgcgcgaaatggcagcacaattggaatgacggcggattcgattattggggccagggcacacttgtaactgtgtcaagcaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 11 - protein, 488 aa, synthetic construct
  REGION 1..488: note = Synthetic: pBB4015 CAR a.a. sequence with signal peptide
MALPVTALLLPLALLLHAARPEIVLTQSPDTLSLSPGERATLSCRASQSLSSNYLAWYRQKPGQAPRLLIYGISSRATGIPDRFSGSGSGTDFTLTISRLEPEDFAVYYCQQYGSSPWTFGQGTKVEIKGGGGSGGGGSGGGGSQVQLVESGGGVVQPGRSLRLSCVASGFTFSNYGIHWVRQAPGKGLEWVAVISDDGSFKFYADSVKGRFTISRDNSKNTLYLQMNSLRVEDSAVYHCAKWQHNWNDGGFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - DNA, 1464 bp, synthetic construct
  misc_feature 1..1464: note = Synthetic: pBB4015 CAR n.a. sequence with signal peptide
  source 1..1464: mol_type = other DNA
atggccctgccggttaccgctctcttgctgcccctggcgctgcttctgcacgcggcaagacccgaaattgtgctgacccagagccccgataccctttccctttccccaggcgagcgcgccacactgtcttgcagggcgtcacagagcctgagcagcaactatctcgcgtggtatagacagaaaccagggcaagccccacggctgctgatctatggaattagctcacgggcaacaggaatccccgacagattcagtgggtctgggagcggaacggattttaccctgacaattagtagattggaaccggaagacttcgctgtgtactattgccagcagtacggctcatccccgtggaccttcggacaaggcactaaggtagagatcaaaggtggagggggctcagggggaggcggaagcggaggcggaggatctcaggtgcagctggttgagtctggaggcggagtcgttcagccaggtaggagcctccgactctcctgcgtcgcaagtgggtttacgttttccaattacgggattcactgggtccgacaggcccccgggaaggggctcgagtgggtggccgttattagcgatgacgggtctttcaagttctatgccgattcagtcaaaggtagattcacaatttcaagggataatagtaaaaatacactgtacttgcaaatgaactctctgcgcgtcgaggattcagccgtgtaccattgcgcgaaatggcagcacaattggaatgacggcggattcgattattggggccagggcacacttgtaactgtgtcaagcaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 13 - protein, 14507 aa, Homo sapiens
  REGION 1..14507: note = misc_feature - Human MUC16 a.a sequence
MLKPSGLPGSSSPTRSLMTGSRSTKATPEMDSGLTGATLSPKTSTGAIVVTEHTLPFTSPDKTLASPTSSVVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASVLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTYTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWTSVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGSSPAGTSPGSPEMSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLRTLNTLDINLESGTTSSPSWKSSPYERIAPSESTTDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFTYSEKTTLFSKGPEDTSQPSPPSVEETSSSSSLVPIHATTSPSNILLTSQGHSPSSTPPVTSVFLSETSGLGKTTDMSRISLEPGTSLPPNLSSTAGEALSTYEASRDTKAIHHSADTAVTNMEATSSEYSPIPGHTKPSKATSPLVTSHIMGDITSSTSVFGSSETTEIETVSSVNQGLQERSTSQVASSATETSTVITHVSSGDATTHVTKTQATFSSGTSISSPHQFITSTNTFTDVSTNPSTSLIMTESSGVTITTQTGPTGAATQGPYLLDTSTMPYLTETPLAVTPDFMQSEKTTLISKGPKDVSWTSPPSVAETSYPSSLTPFLVTTIPPATSTLQGQHTSSPVSATSVLTSGLVKTTDMLNTSMEPVTNSPQNLNNPSNEILATLAATTDIETIHPSINKAVTNMGTASSAHVLHSTLPVSSEPSTATSPMVPASSMGDALASISIPGSETTDIEGEPTSSLTAGRKENSTLQEMNSTTESNIILSNVSVGAITEATKMEVPSFDATFIPTPAQSTKFPDIFSVASSRLSNSPPMTISTHMTTTQTGSSGATSKIPLALDTSTLETSAGTPSVVTEGFAHSKITTAMNNDVKDVSQTNPPFQDEASSPSSQAPVLVTTLPSSVAFTPQWHSTSSPVSMSSVLTSSLVKTAGKVDTSLETVTSSPQSMSNTLDDISVTSAATTDIETTHPSINTVVTNVGTTGSAFESHSTVSAYPEPSKVTSPNVTTSTMEDTTISRSIPKSSKTTRTETETTSSLTPKLRETSISQEITSSTETSTVPYKELTGATTEVSRTDVTSSSSTSFPGPDQSTVSLDISTETNTRLSTSPIMTESAEITITTQTGPHGATSQDTFTMDPSNTTPQAGIHSAMTHGFSQLDVTTLMSRIPQDVSWTSPPSVDKTSSPSSFLSSPAMTTPSLISSTLPEDKLSSPMTSLLTSGLVKITDILRTRLEPVTSSLPNFSSTSDKILATSKDSKDTKEIFPSINTEETNVKANNSGHESHSPALADSETPKATTQMVITTTVGDPAPSTSMPVHGSSETTNIKREPTYFLTPRLRETSTSQESSFPTDTSFLLSKVPTGTITEVSSTGVNSSSKISTPDHDKSTVPPDTFTGEIPRVFTSSIKTKSAEMTITTQASPPESASHSTLPLDTSTTLSQGGTHSTVTQGFPYSEVTTLMGMGPGNVSWMTTPPVEETSSVSSLMSSPAMTSPSPVSSTSPQSIPSSPLPVTALPTSVLVTTTDVLGTTSPESVTSSPPNLSSITHERPATYKDTAHTEAAMHHSTNTAVTNVGTSGSGHKSQSSVLADSETSKATPLMSTTSTLGDTSVSTSTPNISQTNQIQTEPTASLSPRLRESSTSEKTSSTTETNTAFSYVPTGAITQASRTEISSSRTSISDLDRPTIAPDISTGMITRLFTSPIMTKSAEMTVTTQTTTPGATSQGILPWDTSTTLFQGGTHSTVSQGFPHSEITTLRSRTPGDVSWMTTPPVEETSSGFSLMSPSMTSPSPVSSTSPESIPSSPLPVTALLTSVLVTTTNVLGTTSPEPVTSSPPNLSSPTQERLTTYKDTAHTEAMHASMHTNTAVANVGTSISGHESQSSVPADSHTSKATSPMGITFAMGDTSVSTSTPAFFETRIQTESTSSLIPGLRDTRTSEEINTVTETSTVLSEVPTTTTTEVSRTEVITSSRTTISGPDHSKMSPYISTETITRLSTFPFVTGSTEMAITNQTGPIGTISQATLTLDTSSTASWEGTHSPVTQRFPHSEETTTMSRSTKGVSWQSPPSVEETSSPSSPVPLPAITSHSSLYSAVSGSSPTSALPVTSLLTSGRRKTIDMLDTHSELVTSSLPSASSFSGEILTSEASTNTETIHFSENTAETNMGTTNSMHKLHSSVSIHSQPSGHTPPKVTGSMMEDAIVSTSTPGSPETKNVDRDSTSPLTPELKEDSTALVMNSTTESNTVFSSVSLDAATEVSRAEVTYYDPTFMPASAQSTKSPDISPEASSSHSNSPPLTISTHKTIATQTGPSGVTSLGQLTLDTSTIATSAGTPSARTQDFVDSETTSVMNNDLNDVLKTSPFSAEEANSLSSQAPLLVTTSPSPVTSTLQEHSTSSLVSVTSVPTPTLAKITDMDTNLEPVTRSPQNLRNTLATSEATTDTHTMHPSINTAVANVGTTSSPNEFYFTVSPDSDPYKATSAVVITSTSGDSIVSTSMPRSSAMKKIESETTFSLIFRLRETSTSQKIGSSSDTSTVFDKAFTAATTEVSRTELTSSSRTSIQGTEKPTMSPDTSTRSVTMLSTFAGLTKSEERTIATQTGPHRATSQGTLTWDTSITTSQAGTHSAMTHGFSQLDLSTLTSRVPEYISGTSPPSVEKTSSSSSLLSLPAITSPSPVPTTLPESRPSSPVHLTSLPTSGLVKTTDMLASVASLPPNLGSTSHKIPTTSEDIKDTEKMYPSTNIAVTNVGTTTSEKESYSSVPAYSEPPKVTSPMVTSFNIRDTIVSTSMPGSSEITRIEMESTFSLAHGLKGTSTSQDPIVSTEKSAVLHKLTTGATETSRTEVASSRRTSIPGPDHSTESPDISTEVIPSLPISLGITESSNMTIITRTGPPLGSTSQGTFTLDTPTTSSRAGTHSMATQEFPHSEMTTVMNKDPEILSWTIPPSIEKTSFSSSLMPSPAMTSPPVSSTLPKTIHTTPSPMTSLLTPSLVMTTDTLGTSPEPTTSSPPNLSSTSHEILTTDEDTTAIEAMHPSTSTAATNVETTSSGHGSQSSVLADSEKTKATAPMDTTSTMGHTTVSTSMSVSSETTKIKRESTYSLTPGLRETSISQNASFSTDTSIVLSEVPTGTTAEVSRTEVTSSGRTSIPGPSQSTVLPEISTRTMTRLFASPTMTESAEMTIPTQTGPSGSTSQDTLTLDTSTTKSQAKTHSTLTQRFPHSEMTTLMSRGPGDMSWQSSPSLENPSSLPSLLSLPATTSPPPISSTLPVTISSSPLPVTSLLTSSPVTTTDMLHTSPELVTSSPPKLSHTSDERLTTGKDTTNTEAVHPSTNTAASNVEIPSSGHESPSSALADSETSKATSPMFITSTQEDTTVAISTPHFLETSRIQKESISSLSPKLRETGSSVETSSAIETSAVLSEVSIGATTEISRTEVTSSSRTSISGSAESTMLPEISTTRKIIKFPTSPILAESSEMTIKTQTSPPGSTSESTFTLDTSTTPSLVITHSTMTQRLPHSEITTLVSRGAGDVPRPSSLPVEETSPPSSQLSLSAMISPSPVSSTLPASSHSSSASVTSLLTPGQVKTTEVLDASAEPETSSPPSLSSTSVEILATSEVTTDTEKIHPFSNTAVTKVGTSSSGHESPSSVLPDSETTKATSAMGTISIMGDTSVSTLTPALSNTRKIQSEPASSLTTRLRETSTSEETSLATEANTVLSKVSTGATTEVSRTEAISFSRTSMSGPEQSTMSQDISIGTIPRISASSVLTESAKMTITTQTGPSESTLESTLNLNTATTPSWVETHSIVIQGFPHPEMTTSMGRGPGGVSWPSPPFVKETSPPSSPLSLPAVTSPHPVSTTFLAHIPPSPLPVTSLLTSGPATTTDILGTSTEPGTSSSSSLSTTSHERLTTYKDTAHTEAVHPSTNTGGTNVATTSSGYKSQSSVLADSSPMCTTSTMGDTSVLTSTPAFLETRRIQTELASSLTPGLRESSGSEGTSSGTKMSTVLSKVPTGATTEISKEDVTSIPGPAQSTISPDISTRTVSWFSTSPVMTESAEITMNTHTSPLGATTQGTSTLDTSSTTSLTMTHSTISQGFSHSQMSTLMRRGPEDVSWMSPPLLEKTRPSFSLMSSPATTSPSPVSSTLPESISSSPLPVTSLLTSGLAKTTDMLHKSSEPVTNSPANLSSTSVEILATSEVTTDTEKTHPSSNRTVTDVGTSSSGHESTSFVLADSQTSKVTSPMVITSTMEDTSVSTSTPGFFETSRIQTEPTSSLTLGLRKTSSSEGTSLATEMSTVLSGVPTGATAEVSRTEVTSSSRTSISGFAQLTVSPETSTETITRLPTSSIMTESAEMMIKTQTDPPGSTPESTHTVDISTTPNWVETHSTVTQRFSHSEMTTLVSRSPGDMLWPSQSSVEETSSASSLLSLPATTSPSPVSSTLVEDFPSASLPVTSLLNPGLVITTDRMGISREPGTSSTSNLSSTSHERLTTLEDTVDTEDMQPSTHTAVTNVRTSISGHESQSSVLSDSETPKATSPMGTTYTMGETSVSISTSDFFETSRIQIEPTSSLTSGLRETSSSERISSATEGSTVLSEVPSGATTEVSRTEVISSRGTSMSGPDQFTISPDISTEAITRLSTSPIMTESAESAITIETGSPGATSEGTLTLDTSTTTFWSGTHSTASPGFSHSEMTTLMSRTPGDVPWPSLPSVEEASSVSSSLSSPAMTSTSFFSTLPESISSSPHPVTALLTLGPVKTTDMLRTSSEPETSSPPNLSSTSAEILATSEVTKDREKIHPSSNTPVVNVGTVIYKHLSPSSVLADLVTTKPTSPMATTSTLGNTSVSTSTPAFPETMMTQPTSSLTSGLREISTSQETSSATERSASLSGMPTGATTKVSRTEALSLGRTSTPGPAQSTISPEISTETITRISTPLTTTGSAEMTITPKTGHSGASSQGTFTLDTSSRASWPGTHSAATHRSPHSGMTTPMSRGPEDVSWPSRPSVEKTSPPSSLVSLSAVTSPSPLYSTPSESSHSSPLRVTSLFTPVMMKTTDMLDTSLEPVTTSPPSMNITSDESLATSKATMETEAIQLSENTAVTQMGTISARQEFYSSYPGLPEPSKVTSPVVTSSTIKDIVSTTIPASSEITRIEMESTSTLTPTPRETSTSQEIHSATKPSTVPYKALTSATIEDSMTQVMSSSRGPSPDQSTMSQDISTEVITRLSTSPIKTESTEMTITTQTGSPGATSRGTLTLDTSTTFMSGTHSTASQGFSHSQMTALMSRTPGDVPWLSHPSVEEASSASFSLSSPVMTSSSPVSSTLPDSIHSSSLPVTSLLTSGLVKTTELLGTSSEPETSSPPNLSSTSAEILAITEVTTDTEKLEMTNVVTSGYTHESPSSVLADSVTTKATSSMGITYPTGDTNVLTSTPAFSDTSRIQTKSKLSLTPGLMETSISEETSSATEKSTVLSSVPTGATTEVSRTEAISSSRTSIPGPAQSTMSSDTSMETITRISTPLTRKESTDMAITPKTGPSGATSQGTFTLDSSSTASWPGTHSATTQRFPQSVVTTPMSRGPEDVSWPSPLSVEKNSPPSSLVSSSSVTSPSPLYSTPSGSSHSSPVPVTSLFTSIMMKATDMLDASLEPETTSAPNMNITSDESLAASKATTETEAIHVFENTAASHVETTSATEELYSSSPGFSEPTKVISPVVTSSSIRDNMVSTTMPGSSGITRIEIESMSSLTPGLRETRTSQDITSSTETSTVLYKMPSGATPEVSRTEVMPSSRTSIPGPAQSTMSLDISDEVVTRLSTSPIMTESAEITITTQTGYSLATSQVTLPLGTSMTFLSGTHSTMSQGLSHSEMTNLMSRGPESLSWTSPRFVETTRSSSSLTSLPLTTSLSPVSSTLLDSSPSSPLPVTSLILPGLVKTTEVLDTSSEPKTSSSPNLSSTSVEIPATSEIMTDTEKIHPSSNTAVAKVRTSSSVHESHSSVLADSETTITIPSMGITSAVDDTTVFTSNPAFSETRRIPTEPTFSLTPGFRETSTSEETTSITETSAVLYGVPTSATTEVSMTEIMSSNRIHIPDSDQSTMSPDIITEVITRLSSSSMMSESTQMTITTQKSSPGATAQSTLTLATTTAPLARTHSTVPPRFLHSEMTTLMSRSPENPSWKSSLFVEKTSSSSSLLSLPVTTSPSVSSTLPQSIPSSSFSVTSLLTPGMVKTTDTSTEPGTSLSPNLSGTSVEILAASEVTTDTEKIHPSSSMAVTNVGTTSSGHELYSSVSIHSEPSKATYPVGTPSSMAETSISTSMPANFETTGFEAEPFSHLTSGFRKTNMSLDTSSVTPTNTPSSPGSTHLLQSSKTDFTSSAKTSSPDWPPASQYTEIPVDIITPFNASPSITESTGITSFPESRFTMSVTESTHHLSTDLLPSAETISTGTVMPSLSEAMTSFATTGVPRAISGSGSPFSRTESGPGDATLSTIAESLPSSTPVPFSSSTFTTTDSSTIPALHEITSSSATPYRVDTSLGTESSTTEGRLVMVSTLDTSSQPGRTSSSPILDTRMTESVELGTVTSAYQVPSLSTRLTRTDGIMEHITKIPNEAAHRGTIRPVKGPQTSTSPASPKGLHTGGTKRMETTTTALKTTTTALKTTSRATLTTSVYTPTLGTLTPLNASMQMASTIPTEMMITTPYVFPDVPETTSSLATSLGAETSTALPRTTPSVFNRESETTASLVSRSGAERSPVIQTLDVSSSEPDTTASWVIHPAETIPTVSKTTPNFFHSELDTVSSTATSHGADVSSAIPTNISPSELDALTPLVTISGTDTSTTFPTLTKSPHETETRTTWLTHPAETSSTIPRTIPNFSHHESDATPSIATSPGAETSSAIPIMTVSPGAEDLVTSQVTSSGTDRNMTIPTLTLSPGEPKTIASLVTHPEAQTSSAIPTSTISPAVSRLVTSMVTSLAAKTSTTNRALTNSPGEPATTVSLVTHPAQTSPTVPWTTSIFFHSKSDTTPSMTTSHGAESSSAVPTPTVSTEVPGVVTPLVTSSRAVISTTIPILTLSPGEPETTPSMATSHGEEASSAIPTPTVSPGVPGVVTSLVTSSRAVTSTTIPILTFSLGEPETTPSMATSHGTEAGSAVPTVLPEVPGMVTSLVASSRAVTSTTLPTLTLSPGEPETTPSMATSHGAEASSTVPTVSPEVPGVVTSLVTSSSGVNSTSIPTLILSPGELETTPSMATSHGAEASSAVPTPTVSPGVSGVVTPLVTSSRAVTSTTIPILTLSSSEPETTPSMATSHGVEASSAVLTVSPEVPGMVTSLVTSSRAVTSTTIPTLTISSDEPETTTSLVTHSEAKMISAIPTLAVSPTVQGLVTSLVTSSGSETSAFSNLTVASSQPETIDSWVAHPGTEASSVVPTLTVSTGEPFTNISLVTHPAESSSTLPRTTSRFSHSELDTMPSTVTSPEAESSSAISTTISPGIPGVLTSLVTSSGRDISATFPTVPESPHESEATASWVTHPAVTSTTVPRTTPNYSHSEPDTTPSIATSPGAEATSDFPTITVSPDVPDMVTSQVTSSGTDTSITIPTLTLSSGEPETTTSFITYSETHTSSAIPTLPVSPGASKMLTSLVISSGTDSTTTFPTLTETPYEPETTAIQLIHPAETNTMVPRTTPKFSHSKSDTTLPVAITSPGPEASSAVSTTTISPDMSDLVTSLVPSSGTDTSTTFPTLSETPYEPETTATWLTHPAETSTTVSGTIPNFSHRGSDTAPSMVTSPGVDTRSGVPTTTIPPSIPGVVTSQVTSSATDTSTAIPTLTPSPGEPETTASSATHPGTQTGFTVPIRTVPSSEPDTMASWVTHPPQTSTPVSRTTSSFSHSSPDATPVMATSPRTEASSAVLTTISPGAPEMVTSQITSSGAATSTTVPTLTHSPGMPETTALLSTHPRTETSKTFPASTVFPQVSETTASLTIRPGAETSTALPTQTTSSLFTLLVTGTSRVDLSPTASPGVSAKTAPLSTHPGTETSTMIPTSTLSLGLLETTGLLATSSSAETSTSTLTLTVSPAVSGLSSASITTDKPQTVTSWNTETSPSVTSVGPPEFSRTVTGTTMTLIPSEMPTPPKTSHGEGVSPTTILRTTMVEATNLATTGSSPTVAKTTTTFNTLAGSLFTPLTTPGMSTLASESVTSRTSYNHRSWISTTSSYNRRYWTPATSTPVTSTFSPGISTSSIPSSTAATVPFMVPFTLNFTITNLQYEEDMRHPGSRKFNATERELQGLLKPLFRNSSLEYLYSGCRLASLRPEKDSSATAVDAICTHRPDPEDLGLDRERLYWELSNLTNGIQELGPYTLDRNSLYVNGFTHRSSMPTTSTPGTSTVDVGTSGTPSSSPSPTTAGPLLMPFTLNFTITNLQYEEDMRRTGSRKFNTMESVLQGLLKPLFKNTSVGPLYSGCRLTLLRPEKDGAATGVDAICTHRLDPKSPGLNREQLYWELSKLTNDIEELGPYTLDRNSLYVNGFTHQSSVSTTSTPGTSTVDLRTSGTPSSLSSPTIMAAGPLLVPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTSSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTTGPLLVPFTLNFTITNLQYEENMGHPGSRKFNITESVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTITNLQYEEDMRRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPMTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGPYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGCRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMQHLLSPLFQRSSLGARYTGCRVIALRSVKNGAETRVDLLCTYLQPLSGPGLPIKQVFHELSQQTHGITRLGPYSLDKDSLYLNGYNEPGPDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVILIGLAGLLGVITCLICGVLVTTRRRKKEGEYNVQQQCPGYYQSHLDLEDLQ

SEQ ID NO: 14 - protein, 5 aa, synthetic construct
  REGION 1..5: note = Synthetic: Linker
DGGGS

SEQ ID NO: 15 - protein, 5 aa, synthetic construct
  REGION 1..5: note = Synthetic: Linker
TGEKP

SEQ ID NO: 16 - protein, 4 aa, synthetic construct
  REGION 1..4: note = Synthetic: Linker
GGRR

SEQ ID NO: 17 - protein, 25 aa, synthetic construct
  REGION 1..25: note = Synthetic: Linker
  VARIANT 1..25: note = "GGGGS" is present at least one time but can repeat up to 5 times
GGGGSGGGGSGGGGSGGGGSGGGGS

SEQ ID NO: 18 - protein, 14 aa, synthetic construct
  REGION 1..14: note = Synthetic: Linker
EGKSSGSGSESKVD

SEQ ID NO: 19 - protein, 18 aa, synthetic construct
  REGION 1..18: note = Synthetic: Linker
KESGSVSSEQLAQFRSLD

SEQ ID NO: 20 - protein, 8 aa, synthetic construct
  REGION 1..8: note = Synthetic: Linker
GGRRGGGS

SEQ ID NO: 21 - protein, 9 aa, synthetic construct
  REGION 1..9: note = Synthetic: Linker
LRQRDGERP

SEQ ID NO: 22 - protein, 12 aa, synthetic construct
  REGION 1..12: note = Synthetic: Linker
LRQKDGGGSERP

SEQ ID NO: 23 - protein, 16 aa, synthetic construct
  REGION 1..16: note = Synthetic: Linker
LRQKDGGGSGGGSERP

SEQ ID NO: 24 - protein, 15 aa, synthetic construct
  REGION 1..15: note = Synthetic: Linker
GGGGSGGGGSGGGGS

SEQ ID NO: 25 - protein, 18 aa, synthetic construct
  REGION 1..18: note = Synthetic: Linker
GSTSGSGKPGSGEGSTKG

SEQ ID NO: 26 - protein, 19 aa, synthetic construct
  REGION 1..19: note = Synthetic: 2A cleaving site
LLNFDLLKLAGDVESNPGP

SEQ ID NO: 27 - protein, 19 aa, synthetic construct
  REGION 1..19: note = Synthetic: 2A cleaving site
TLNFDLLKLAGDVESNPGP

SEQ ID NO: 28 - protein, 14 aa, synthetic construct
  REGION 1..14: note = Synthetic: 2A cleaving site
LLKLAGDVESNPGP

SEQ ID NO: 29 - protein, 17 aa, synthetic construct
  REGION 1..17: note = Synthetic: 2A cleaving site
NFDLLKLAGDVESNPGP

SEQ ID NO: 30 - protein, 20 aa, synthetic construct
  REGION 1..20: note = Synthetic: 2A cleaving site
QLLNFDLLKLAGDVESNPGP

SEQ ID NO: 31 - protein, 24 aa, synthetic construct
  REGION 1..24: note = Synthetic: 2A cleaving site
APVKQTLNFDLLKLAGDVESNPGP

SEQ ID NO: 32 - protein, 40 aa, synthetic construct
  REGION 1..40: note = Synthetic: 2A cleaving site
VTELLYRMKRAETYCPRPLLAIHPTEARHKQKIVAPVKQT

SEQ ID NO: 33 - protein, 18 aa, synthetic construct
  REGION 1..18: note = Synthetic: 2A cleaving site
LNFDLLKLAGDVESNPGP

SEQ ID NO: 34 - protein, 40 aa, synthetic construct
  REGION 1..40: note = Synthetic: 2A cleaving site
LLAIHPTEARHKQKIVAPVKQTLNFDLLKLAGDVESNPGP

SEQ ID NO: 35 - protein, 33 aa, synthetic construct
  REGION 1..33: note = Synthetic: 2A cleaving site
EARHKQKIVAPVKQTLNFDLLKLAGDVESNPGP

SEQ ID NO: 36 - skipped

SEQ ID NO: 37 - protein, 7 aa, synthetic construct
  REGION 1..7: note = Synthetic: Cleavage site
ENLYFQG

SEQ ID NO: 38 - protein, 7 aa, synthetic construct
  REGION 1..7: note = Synthetic: Cleavage site
ENLYFQS

SEQ ID NO: 39 - protein, 670 aa, synthetic construct
  REGION 1..670: note = Synthetic: Human MUC16 ectodomain with myc-myc-his tag
PGSRKFNTTERVLQGLLRPLFKNTSVGPLYSGCRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMQHLLSPLFQRSSLGARYTGCRVIALRSVKNGAETRVDLLCTYLQPLSGPGLPIKQVFHELSQQTHGITRLGPYSLDKDSLYLNGYNEPGPDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPEQKLISEEDLGGEQKLISEEDLHHHHHH

SEQ ID NO: 40 - protein, 13809 aa, synthetic construct
  REGION 1..13809: note = Synthetic: MUC16 polypeptide lacking the nub (CA125)
MLKPSGLPGSSSPTRSLMTGSRSTKATPEMDSGLTGATLSPKTSTGAIVVTEHTLPFTSPDKTLASPTSSVVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASVLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTYTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWTSVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGSSPAGTSPGSPEMSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLRTLNTLDINLESGTTSSPSWKSSPYERIAPSESTTDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFTYSEKTTLFSKGPEDTSQPSPPSVEETSSSSSLVPIHATTSPSNILLTSQGHSPSSTPPVTSVFLSETSGLGKTTDMSRISLEPGTSLPPNLSSTAGEALSTYEASRDTKAIHHSADTAVTNMEATSSEYSPIPGHTKPSKATSPLVTSHIMGDITSSTSVFGSSETTEIETVSSVNQGLQERSTSQVASSATETSTVITHVSSGDATTHVTKTQATFSSGTSISSPHQFITSTNTFTDVSTNPSTSLIMTESSGVTITTQTGPTGAATQGPYLLDTSTMPYLTETPLAVTPDFMQSEKTTLISKGPKDVSWTSPPSVAETSYPSSLTPFLVTTIPPATSTLQGQHTSSPVSATSVLTSGLVKTTDMLNTSMEPVTNSPQNLNNPSNEILATLAATTDIETIHPSINKAVTNMGTASSAHVLHSTLPVSSEPSTATSPMVPASSMGDALASISIPGSETTDIEGEPTSSLTAGRKENSTLQEMNSTTESNIILSNVSVGAITEATKMEVPSFDATFIPTPAQSTKFPDIFSVASSRLSNSPPMTISTHMTTTQTGSSGATSKIPLALDTSTLETSAGTPSVVTEGFAHSKITTAMNNDVKDVSQTNPPFQDEASSPSSQAPVLVTTLPSSVAFTPQWHSTSSPVSMSSVLTSSLVKTAGKVDTSLETVTSSPQSMSNTLDDISVTSAATTDIETTHPSINTVVTNVGTTGSAFESHSTVSAYPEPSKVTSPNVTTSTMEDTTISRSIPKSSKTTRTETETTSSLTPKLRETSISQEITSSTETSTVPYKELTGATTEVSRTDVTSSSSTSFPGPDQSTVSLDISTETNTRLSTSPIMTESAEITITTQTGPHGATSQDTFTMDPSNTTPQAGIHSAMTHGFSQLDVTTLMSRIPQDVSWTSPPSVDKTSSPSSFLSSPAMTTPSLISSTLPEDKLSSPMTSLLTSGLVKITDILRTRLEPVTSSLPNFSSTSDKILATSKDSKDTKEIFPSINTEETNVKANNSGHESHSPALADSETPKATTQMVITTTVGDPAPSTSMPVHGSSETTNIKREPTYFLTPRLRETSTSQESSFPTDTSFLLSKVPTGTITEVSSTGVNSSSKISTPDHDKSTVPPDTFTGEIPRVFTSSIKTKSAEMTITTQASPPESASHSTLPLDTSTTLSQGGTHSTVTQGFPYSEVTTLMGMGPGNVSWMTTPPVEETSSVSSLMSSPAMTSPSPVSSTSPQSIPSSPLPVTALPTSVLVTTTDVLGTTSPESVTSSPPNLSSITHERPATYKDTAHTEAAMHHSTNTAVTNVGTSGSGHKSQSSVLADSETSKATPLMSTTSTLGDTSVSTSTPNISQTNQIQTEPTASLSPRLRESSTSEKTSSTTETNTAFSYVPTGAITQASRTEISSSRTSISDLDRPTIAPDISTGMITRLFTSPIMTKSAEMTVTTQTTTPGATSQGILPWDTSTTLFQGGTHSTVSQGFPHSEITTLRSRTPGDVSWMTTPPVEETSSGFSLMSPSMTSPSPVSSTSPESIPSSPLPVTALLTSVLVTTTNVLGTTSPEPVTSSPPNLSSPTQERLTTYKDTAHTEAMHASMHTNTAVANVGTSISGHESQSSVPADSHTSKATSPMGITFAMGDTSVSTSTPAFFETRIQTESTSSLIPGLRDTRTSEEINTVTETSTVLSEVPTTTTTEVSRTEVITSSRTTISGPDHSKMSPYISTETITRLSTFPFVTGSTEMAITNQTGPIGTISQATLTLDTSSTASWEGTHSPVTQRFPHSEETTTMSRSTKGVSWQSPPSVEETSSPSSPVPLPAITSHSSLYSAVSGSSPTSALPVTSLLTSGRRKTIDMLDTHSELVTSSLPSASSFSGEILTSEASTNTETIHFSENTAETNMGTTNSMHKLHSSVSIHSQPSGHTPPKVTGSMMEDAIVSTSTPGSPETKNVDRDSTSPLTPELKEDSTALVMNSTTESNTVFSSVSLDAATEVSRAEVTYYDPTFMPASAQSTKSPDISPEASSSHSNSPPLTISTHKTIATQTGPSGVTSLGQLTLDTSTIATSAGTPSARTQDFVDSETTSVMNNDLNDVLKTSPFSAEEANSLSSQAPLLVTTSPSPVTSTLQEHSTSSLVSVTSVPTPTLAKITDMDTNLEPVTRSPQNLRNTLATSEATTDTHTMHPSINTAVANVGTTSSPNEFYFTVSPDSDPYKATSAVVITSTSGDSIVSTSMPRSSAMKKIESETTFSLIFRLRETSTSQKIGSSSDTSTVFDKAFTAATTEVSRTELTSSSRTSIQGTEKPTMSPDTSTRSVTMLSTFAGLTKSEERTIATQTGPHRATSQGTLTWDTSITTSQAGTHSAMTHGFSQLDLSTLTSRVPEYISGTSPPSVEKTSSSSSLLSLPAITSPSPVPTTLPESRPSSPVHLTSLPTSGLVKTTDMLASVASLPPNLGSTSHKIPTTSEDIKDTEKMYPSTNIAVTNVGTTTSEKESYSSVPAYSEPPKVTSPMVTSFNIRDTIVSTSMPGSSEITRIEMESTFSLAHGLKGTSTSQDPIVSTEKSAVLHKLTTGATETSRTEVASSRRTSIPGPDHSTESPDISTEVIPSLPISLGITESSNMTIITRTGPPLGSTSQGTFTLDTPTTSSRAGTHSMATQEFPHSEMTTVMNKDPEILSWTIPPSIEKTSFSSSLMPSPAMTSPPVSSTLPKTIHTTPSPMTSLLTPSLVMTTDTLGTSPEPTTSSPPNLSSTSHEILTTDEDTTAIEAMHPSTSTAATNVETTSSGHGSQSSVLADSEKTKATAPMDTTSTMGHTTVSTSMSVSSETTKIKRESTYSLTPGLRETSISQNASFSTDTSIVLSEVPTGTTAEVSRTEVTSSGRTSIPGPSQSTVLPEISTRTMTRLFASPTMTESAEMTIPTQTGPSGSTSQDTLTLDTSTTKSQAKTHSTLTQRFPHSEMTTLMSRGPGDMSWQSSPSLENPSSLPSLLSLPATTSPPPISSTLPVTISSSPLPVTSLLTSSPVTTTDMLHTSPELVTSSPPKLSHTSDERLTTGKDTTNTEAVHPSTNTAASNVEIPSSGHESPSSALADSETSKATSPMFITSTQEDTTVAISTPHFLETSRIQKESISSLSPKLRETGSSVETSSAIETSAVLSEVSIGATTEISRTEVTSSSRTSISGSAESTMLPEISTTRKIIKFPTSPILAESSEMTIKTQTSPPGSTSESTFTLDTSTTPSLVITHSTMTQRLPHSEITTLVSRGAGDVPRPSSLPVEETSPPSSQLSLSAMISPSPVSSTLPASSHSSSASVTSLLTPGQVKTTEVLDASAEPETSSPPSLSSTSVEILATSEVTTDTEKIHPFSNTAVTKVGTSSSGHESPSSVLPDSETTKATSAMGTISIMGDTSVSTLTPALSNTRKIQSEPASSLTTRLRETSTSEETSLATEANTVLSKVSTGATTEVSRTEAISFSRTSMSGPEQSTMSQDISIGTIPRISASSVLTESAKMTITTQTGPSESTLESTLNLNTATTPSWVETHSIVIQGFPHPEMTTSMGRGPGGVSWPSPPFVKETSPPSSPLSLPAVTSPHPVSTTFLAHIPPSPLPVTSLLTSGPATTTDILGTSTEPGTSSSSSLSTTSHERLTTYKDTAHTEAVHPSTNTGGTNVATTSSGYKSQSSVLADSSPMCTTSTMGDTSVLTSTPAFLETRRIQTELASSLTPGLRESSGSEGTSSGTKMSTVLSKVPTGATTEISKEDVTSIPGPAQSTISPDISTRTVSWFSTSPVMTESAEITMNTHTSPLGATTQGTSTLDTSSTTSLTMTHSTISQGFSHSQMSTLMRRGPEDVSWMSPPLLEKTRPSFSLMSSPATTSPSPVSSTLPESISSSPLPVTSLLTSGLAKTTDMLHKSSEPVTNSPANLSSTSVEILATSEVTTDTEKTHPSSNRTVTDVGTSSSGHESTSFVLADSQTSKVTSPMVITSTMEDTSVSTSTPGFFETSRIQTEPTSSLTLGLRKTSSSEGTSLATEMSTVLSGVPTGATAEVSRTEVTSSSRTSISGFAQLTVSPETSTETITRLPTSSIMTESAEMMIKTQTDPPGSTPESTHTVDISTTPNWVETHSTVTQRFSHSEMTTLVSRSPGDMLWPSQSSVEETSSASSLLSLPATTSPSPVSSTLVEDFPSASLPVTSLLNPGLVITTDRMGISREPGTSSTSNLSSTSHERLTTLEDTVDTEDMQPSTHTAVTNVRTSISGHESQSSVLSDSETPKATSPMGTTYTMGETSVSISTSDFFETSRIQIEPTSSLTSGLRETSSSERISSATEGSTVLSEVPSGATTEVSRTEVISSRGTSMSGPDQFTISPDISTEAITRLSTSPIMTESAESAITIETGSPGATSEGTLTLDTSTTTFWSGTHSTASPGFSHSEMTTLMSRTPGDVPWPSLPSVEEASSVSSSLSSPAMTSTSFFSTLPESISSSPHPVTALLTLGPVKTTDMLRTSSEPETSSPPNLSSTSAEILATSEVTKDREKIHPSSNTPVVNVGTVIYKHLSPSSVLADLVTTKPTSPMATTSTLGNTSVSTSTPAFPETMMTQPTSSLTSGLREISTSQETSSATERSASLSGMPTGATTKVSRTEALSLGRTSTPGPAQSTISPEISTETITRISTPLTTTGSAEMTITPKTGHSGASSQGTFTLDTSSRASWPGTHSAATHRSPHSGMTTPMSRGPEDVSWPSRPSVEKTSPPSSLVSLSAVTSPSPLYSTPSESSHSSPLRVTSLFTPVMMKTTDMLDTSLEPVTTSPPSMNITSDESLATSKATMETEAIQLSENTAVTQMGTISARQEFYSSYPGLPEPSKVTSPVVTSSTIKDIVSTTIPASSEITRIEMESTSTLTPTPRETSTSQEIHSATKPSTVPYKALTSATIEDSMTQVMSSSRGPSPDQSTMSQDISTEVITRLSTSPIKTESTEMTITTQTGSPGATSRGTLTLDTSTTFMSGTHSTASQGFSHSQMTALMSRTPGDVPWLSHPSVEEASSASFSLSSPVMTSSSPVSSTLPDSIHSSSLPVTSLLTSGLVKTTELLGTSSEPETSSPPNLSSTSAEILAITEVTTDTEKLEMTNVVTSGYTHESPSSVLADSVTTKATSSMGITYPTGDTNVLTSTPAFSDTSRIQTKSKLSLTPGLMETSISEETSSATEKSTVLSSVPTGATTEVSRTEAISSSRTSIPGPAQSTMSSDTSMETITRISTPLTRKESTDMAITPKTGPSGATSQGTFTLDSSSTASWPGTHSATTQRFPQSVVTTPMSRGPEDVSWPSPLSVEKNSPPSSLVSSSSVTSPSPLYSTPSGSSHSSPVPVTSLFTSIMMKATDMLDASLEPETTSAPNMNITSDESLAASKATTETEAIHVFENTAASHVETTSATEELYSSSPGFSEPTKVISPVVTSSSIRDNMVSTTMPGSSGITRIEIESMSSLTPGLRETRTSQDITSSTETSTVLYKMPSGATPEVSRTEVMPSSRTSIPGPAQSTMSLDISDEVVTRLSTSPIMTESAEITITTQTGYSLATSQVTLPLGTSMTFLSGTHSTMSQGLSHSEMTNLMSRGPESLSWTSPRFVETTRSSSSLTSLPLTTSLSPVSSTLLDSSPSSPLPVTSLILPGLVKTTEVLDTSSEPKTSSSPNLSSTSVEIPATSEIMTDTEKIHPSSNTAVAKVRTSSSVHESHSSVLADSETTITIPSMGITSAVDDTTVFTSNPAFSETRRIPTEPTFSLTPGFRETSTSEETTSITETSAVLYGVPTSATTEVSMTEIMSSNRIHIPDSDQSTMSPDIITEVITRLSSSSMMSESTQMTITTQKSSPGATAQSTLTLATTTAPLARTHSTVPPRFLHSEMTTLMSRSPENPSWKSSLFVEKTSSSSSLLSLPVTTSPSVSSTLPQSIPSSSFSVTSLLTPGMVKTTDTSTEPGTSLSPNLSGTSVEILAASEVTTDTEKIHPSSSMAVTNVGTTSSGHELYSSVSIHSEPSKATYPVGTPSSMAETSISTSMPANFETTGFEAEPFSHLTSGFRKTNMSLDTSSVTPTNTPSSPGSTHLLQSSKTDFTSSAKTSSPDWPPASQYTEIPVDIITPFNASPSITESTGITSFPESRFTMSVTESTHHLSTDLLPSAETISTGTVMPSLSEAMTSFATTGVPRAISGSGSPFSRTESGPGDATLSTIAESLPSSTPVPFSSSTFTTTDSSTIPALHEITSSSATPYRVDTSLGTESSTTEGRLVMVSTLDTSSQPGRTSSSPILDTRMTESVELGTVTSAYQVPSLSTRLTRTDGIMEHITKIPNEAAHRGTIRPVKGPQTSTSPASPKGLHTGGTKRMETTTTALKTTTTALKTTSRATLTTSVYTPTLGTLTPLNASMQMASTIPTEMMITTPYVFPDVPETTSSLATSLGAETSTALPRTTPSVFNRESETTASLVSRSGAERSPVIQTLDVSSSEPDTTASWVIHPAETIPTVSKTTPNFFHSELDTVSSTATSHGADVSSAIPTNISPSELDALTPLVTISGTDTSTTFPTLTKSPHETETRTTWLTHPAETSSTIPRTIPNFSHHESDATPSIATSPGAETSSAIPIMTVSPGAEDLVTSQVTSSGTDRNMTIPTLTLSPGEPKTIASLVTHPEAQTSSAIPTSTISPAVSRLVTSMVTSLAAKTSTTNRALTNSPGEPATTVSLVTHPAQTSPTVPWTTSIFFHSKSDTTPSMTTSHGAESSSAVPTPTVSTEVPGVVTPLVTSSRAVISTTIPILTLSPGEPETTPSMATSHGEEASSAIPTPTVSPGVPGVVTSLVTSSRAVTSTTIPILTFSLGEPETTPSMATSHGTEAGSAVPTVLPEVPGMVTSLVASSRAVTSTTLPTLTLSPGEPETTPSMATSHGAEASSTVPTVSPEVPGVVTSLVTSSSGVNSTSIPTLILSPGELETTPSMATSHGAEASSAVPTPTVSPGVSGVVTPLVTSSRAVTSTTIPILTLSSSEPETTPSMATSHGVEASSAVLTVSPEVPGMVTSLVTSSRAVTSTTIPTLTISSDEPETTTSLVTHSEAKMISAIPTLAVSPTVQGLVTSLVTSSGSETSAFSNLTVASSQPETIDSWVAHPGTEASSVVPTLTVSTGEPFTNISLVTHPAESSSTLPRTTSRFSHSELDTMPSTVTSPEAESSSAISTTISPGIPGVLTSLVTSSGRDISATFPTVPESPHESEATASWVTHPAVTSTTVPRTTPNYSHSEPDTTPSIATSPGAEATSDFPTITVSPDVPDMVTSQVTSSGTDTSITIPTLTLSSGEPETTTSFITYSETHTSSAIPTLPVSPGASKMLTSLVISSGTDSTTTFPTLTETPYEPETTAIQLIHPAETNTMVPRTTPKFSHSKSDTTLPVAITSPGPEASSAVSTTTISPDMSDLVTSLVPSSGTDTSTTFPTLSETPYEPETTATWLTHPAETSTTVSGTIPNFSHRGSDTAPSMVTSPGVDTRSGVPTTTIPPSIPGVVTSQVTSSATDTSTAIPTLTPSPGEPETTASSATHPGTQTGFTVPIRTVPSSEPDTMASWVTHPPQTSTPVSRTTSSFSHSSPDATPVMATSPRTEASSAVLTTISPGAPEMVTSQITSSGAATSTTVPTLTHSPGMPETTALLSTHPRTETSKTFPASTVFPQVSETTASLTIRPGAETSTALPTQTTSSLFTLLVTGTSRVDLSPTASPGVSAKTAPLSTHPGTETSTMIPTSTLSLGLLETTGLLATSSSAETSTSTLTLTVSPAVSGLSSASITTDKPQTVTSWNTETSPSVTSVGPPEFSRTVTGTTMTLIPSEMPTPPKTSHGEGVSPTTILRTTMVEATNLATTGSSPTVAKTTTTFNTLAGSLFTPLTTPGMSTLASESVTSRTSYNHRSWISTTSSYNRRYWTPATSTPVTSTFSPGISTSSIPSSTAATVPFMVPFTLNFTITNLQYEEDMRHPGSRKFNATERELQGLLKPLFRNSSLEYLYSGCRLASLRPEKDSSATAVDAICTHRPDPEDLGLDRERLYWELSNLTNGIQELGPYTLDRNSLYVNGFTHRSSMPTTSTPGTSTVDVGTSGTPSSSPSPTTAGPLLMPFTLNFTITNLQYEEDMRRTGSRKFNTMESVLQGLLKPLFKNTSVGPLYSGCRLTLLRPEKDGAATGVDAICTHRLDPKSPGLNREQLYWELSKLTNDIEELGPYTLDRNSLYVNGFTHQSSVSTTSTPGTSTVDLRTSGTPSSLSSPTIMAAGPLLVPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTSSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTTGPLLVPFTLNFTITNLQYEENMGHPGSRKFNITESVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTITNLQYEEDMRRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPMTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGPYALDNDSLFVNGFTHRSSVSTTSTPGTPTVYLGASKTPASIFGPSAASHLLILFTLNFTITNLRYEENMW

SEQ ID NO: 41 - protein, 6 aa, synthetic construct
  REGION 1..6: note = Synthetic: Anti-MUC16 7135 CDRL1
QSISSY

SEQ ID NO: 42 - skipped

SEQ ID NO: 43 - protein, 10 aa, synthetic construct
  REGION 1..10: note = Synthetic: Anti-MUC16 7135 CDRL3
QQSYSTPPIT

SEQ ID NO: 44 - protein, 8 aa, synthetic construct
  REGION 1..8: note = Synthetic: Anti-MUC16 7135 CDRH1
GFTFSYHE

SEQ ID NO: 45 - protein, 8 aa, synthetic construct
  REGION 1..8: note = Synthetic: Anti-MUC16 7135 CDRH2
IGSRGTTK

SEQ ID NO: 46 - protein, 14 aa, synthetic construct
  REGION 1..14: note = Synthetic: Anti-MUC16 7135 CDRH3
ASEVGSILGYSMDV

SEQ ID NO: 47 - protein, 108 aa, synthetic construct
  REGION 1..108: note = Synthetic: Anti-MUC16 7135 variable light chain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 48 - protein, 121 aa, synthetic construct
  REGION 1..121: note = Synthetic: Anti-MUC16 7135 variable heavy chain
EVQLVESGGGLVQAGGSLRLSCAASGFTFSYHEMNWVRQAPGKGLEWVSYIGSRGTTKHYADPVKGRFTISRDNAKNSLYLQMNSLRAEDTAVYYCASEVGSILGYSMDVWGQGTTVTVSS

SEQ ID NO: 49 - protein, 467 aa, synthetic construct
  REGION 1..467: note = Synthetic: pBB4000 CAR a.a. sequence without signal peptide
EVQLVESGGGLVQAGGSLRLSCAASGFTFSYHEMNWVRQAPGKGLEWVSYIGSRGTTKHYADPVKGRFTISRDNAKNSLYLQMNSLRAEDTAVYYCASEVGSILGYSMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 488 aa, synthetic construct
  REGION 1..488: note = Synthetic: pBB4000 CAR a.a. sequence with signal peptide
MALPVTALLLPLALLLHAARPEVQLVESGGGLVQAGGSLRLSCAASGFTFSYHEMNWVRQAPGKGLEWVSYIGSRGTTKHYADPVKGRFTISRDNAKNSLYLQMNSLRAEDTAVYYCASEVGSILGYSMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 467 aa, synthetic construct
  REGION 1..467: note = Synthetic: pBB4001 CAR a.a. sequence without signal peptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQAGGSLRLSCAASGFTFSYHEMNWVRQAPGKGLEWVSYIGSRGTTKHYADPVKGRFTISRDNAKNSLYLQMNSLRAEDTAVYYCASEVGSILGYSMDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 488 aa, synthetic construct
  REGION 1..488: note = Synthetic: pBB4001 CAR a.a. sequence with signal peptide
MALPVTALLLPLALLLHAARPDIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQAGGSLRLSCAASGFTFSYHEMNWVRQAPGKGLEWVSYIGSRGTTKHYADPVKGRFTISRDNAKNSLYLQMNSLRAEDTAVYYCASEVGSILGYSMDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 6 aa, synthetic construct
  REGION 1..6: note = Synthetic: Anti-MUC16 7138 CDRL1
QSISSY

SEQ ID NO: 54 - skipped

SEQ ID NO: 55 - protein, 10 aa, synthetic construct
  REGION 1..10: note = Synthetic: Anti-MUC16 7138 CDRL3
QQSYSTPPIT

SEQ ID NO: 56 - protein, 8 aa, synthetic construct
  REGION 1..8: note = Synthetic: Anti-MUC16 7138 CDRH1
GYTFTSSD

SEQ ID NO: 57 - protein, 8 aa, synthetic construct
  REGION 1..8: note = Synthetic: Anti-MUC16 7138 CDRH2
MNPNYGYT

SEQ ID NO: 58 - protein, 18 aa, synthetic construct
  REGION 1..18: note = Synthetic: Anti-MUC16 7138 CDRH3
ARVTYCSSVTSCFRPFTY

SEQ ID NO: 59 - protein, 108 aa, synthetic construct; identical to SEQ ID NO: 47
  REGION 1..108: note = Synthetic: Anti-MUC16 7138 variable light chain

SEQ ID NO: 60 - protein, 125 aa, synthetic construct
  REGION 1..125: note = Synthetic: Anti-MUC16 7138 variable heavy chain
QVQLVQSGAEVKKPGASVKVSCKASGYTFTSSDINWVRQATGQGLEWVGWMNPNYGYTGSARKFQDRVTMTRNTSLSTAYLELHSLRSEDTAVYYCARVTYCSSVTSCFRPFTYWGQGTLVTVSA

SEQ ID NO: 61 - protein, 471 aa, synthetic construct
  REGION 1..471: note = Synthetic: pBB4002 CAR a.a. sequence without signal peptide
QVQLVQSGAEVKKPGASVKVSCKASGYTFTSSDINWVRQATGQGLEWVGWMNPNYGYTGSARKFQDRVTMTRNTSLSTAYLELHSLRSEDTAVYYCARVTYCSSVTSCFRPFTYWGQGTLVTVSAGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 492 aa, synthetic construct
  REGION 1..492: note = Synthetic: pBB4002 CAR a.a. sequence with signal peptide
MALPVTALLLPLALLLHAARPQVQLVQSGAEVKKPGASVKVSCKASGYTFTSSDINWVRQATGQGLEWVGWMNPNYGYTGSARKFQDRVTMTRNTSLSTAYLELHSLRSEDTAVYYCARVTYCSSVTSCFRPFTYWGQGTLVTVSAGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 471 aa, synthetic construct
  REGION 1..471: note = Synthetic: pBB4003 CAR a.a. sequence without signal peptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVQSGAEVKKPGASVKVSCKASGYTFTSSDINWVRQATGQGLEWVGWMNPNYGYTGSARKFQDRVTMTRNTSLSTAYLELHSLRSEDTAVYYCARVTYCSSVTSCFRPFTYWGQGTLVTVSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 492 aa, synthetic construct
  REGION 1..492: note = Synthetic: pBB4003 CAR a.a. sequence with signal peptide
MALPVTALLLPLALLLHAARPDIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVQSGAEVKKPGASVKVSCKASGYTFTSSDINWVRQATGQGLEWVGWMNPNYGYTGSARKFQDRVTMTRNTSLSTAYLELHSLRSEDTAVYYCARVTYCSSVTSCFRPFTYWGQGTLVTVSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 6 aa, synthetic construct
  REGION 1..6: note = Synthetic: Anti-MUC16 8755 CDRL1
QSINIY

SEQ ID NO: 66 - skipped

SEQ ID NO: 67 - protein, 10 aa, synthetic construct
  REGION 1..10: note = Synthetic: Anti-MUC16 8755 CDRL3
QHSYSTPPIT

SEQ ID NO: 68 - protein, 8 aa, synthetic construct
  REGION 1..8: note = Synthetic: Anti-MUC16 8755 CDRH1
GFTFNDYA

SEQ ID NO: 69 - protein, 8 aa, synthetic construct
  REGION 1..8: note = Synthetic: Anti-MUC16 8755 CDRH2
IDWNGGSI

SEQ ID NO: 70 - protein, 15 aa, synthetic construct
  REGION 1..15: note = Synthetic: Anti-MUC16 8755 CDRH3
AKDIGHWNYIGGMDV

SEQ ID NO: 71 - protein, 108 aa, synthetic construct
  REGION 1..108: note = Synthetic: Anti-MUC16 8755 variable light chain
DIQMTQSPSSLSASVGDRVTITCRASQSINIYLNWYQQKPGKAPKFLIYAASSLQTGVPSRFSGSGSGTDFTLTISSLEPEDFATYYCQHSYSTPPITFGQGTRLEIK

SEQ ID NO: 72 - protein, 122 aa, synthetic construct
  REGION 1..122: note = Synthetic: Anti-MUC16 8755 variable heavy chain
EVQLVESGGGLVQPGRSLRLSCAASGFTFNDYAMHWVRQSPGKGLEWVSGIDWNGGSIDYADSVKGRFTISRDTAKNSLYLQMNSLKVEDTALYYCAKDIGHWNYIGGMDVWGQGTTVTVSS

SEQ ID NO: 73 - protein, 468 aa, synthetic construct
  REGION 1..468: note = Synthetic: pBB4004 CAR a.a. sequence without signal peptide
EVQLVESGGGLVQPGRSLRLSCAASGFTFNDYAMHWVRQSPGKGLEWVSGIDWNGGSIDYADSVKGRFTISRDTAKNSLYLQMNSLKVEDTALYYCAKDIGHWNYIGGMDVWGQGTTVTVSSGGGGSGGGGSGGGGSDIQMTQSPSSLSASVGDRVTITCRASQSINIYLNWYQQKPGKAPKFLIYAASSLQTGVPSRFSGSGSGTDFTLTISSLEPEDFATYYCQHSYSTPPITFGQGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 489 aa, synthetic construct
  REGION 1..489: note = Synthetic: pBB4004 CAR a.a. sequence with signal peptide
MALPVTALLLPLALLLHAARPEVQLVESGGGLVQPGRSLRLSCAASGFTFNDYAMHWVRQSPGKGLEWVSGIDWNGGSIDYADSVKGRFTISRDTAKNSLYLQMNSLKVEDTALYYCAKDIGHWNYIGGMDVWGQGTTVTVSSGGGGSGGGGSGGGGSDIQMTQSPSSLSASVGDRVTITCRASQSINIYLNWYQQKPGKAPKFLIYAASSLQTGVPSRFSGSGSGTDFTLTISSLEPEDFATYYCQHSYSTPPITFGQGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 468 aa, synthetic construct
  REGION 1..468: note = Synthetic: pBB4005 CAR a.a. sequence without signal peptide
DIQMTQSPSSLSASVGDRVTITCRASQSINIYLNWYQQKPGKAPKFLIYAASSLQTGVPSRFSGSGSGTDFTLTISSLEPEDFATYYCQHSYSTPPITFGQGTRLEIKGGGGSGGGGSGGGGSEVQLVESGGGLVQPGRSLRLSCAASGFTFNDYAMHWVRQSPGKGLEWVSGIDWNGGSIDYADSVKGRFTISRDTAKNSLYLQMNSLKVEDTALYYCAKDIGHWNYIGGMDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 489 aa, synthetic construct
  REGION 1..489: note = Synthetic: pBB4005 CAR a.a. sequence with signal peptide
MALPVTALLLPLALLLHAARPDIQMTQSPSSLSASVGDRVTITCRASQSINIYLNWYQQKPGKAPKFLIYAASSLQTGVPSRFSGSGSGTDFTLTISSLEPEDFATYYCQHSYSTPPITFGQGTRLEIKGGGGSGGGGSGGGGSEVQLVESGGGLVQPGRSLRLSCAASGFTFNDYAMHWVRQSPGKGLEWVSGIDWNGGSIDYADSVKGRFTISRDTAKNSLYLQMNSLKVEDTALYYCAKDIGHWNYIGGMDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - protein, 6 aa, synthetic construct
  REGION 1..6: note = Synthetic: Anti-MUC16 8767 CDRL1
QSISTY

SEQ ID NO: 78 - skipped

SEQ ID NO: 79 - protein, 10 aa, synthetic construct
  REGION 1..10: note = Synthetic: Anti-MUC16 8767 CDRL3
QQSYSTPPIT

SEQ ID NO: 80 - protein, 8 aa, synthetic construct
  REGION 1..8: note = Synthetic: Anti-MUC16 8767 CDRH1
GFTFSNYY

SEQ ID NO: 81 - protein, 8 aa, synthetic construct
  REGION 1..8: note = Synthetic: Anti-MUC16 8767 CDRH2
ISGRGSTI

SEQ ID NO: 82 - protein, 10 aa, synthetic construct
  REGION 1..10: note = Synthetic: Anti-MUC16 8767 CDRH3
VKDRGGYSPY

SEQ ID NO: 83 - protein, 108 aa, synthetic construct
  REGION 1..108: note = Synthetic: Anti-MUC16 8767 variable light chain
DIQMTQSPSSLSASVGDRVTITCRASQSISTYLNWYQQKPGKAPKLLIYTASSLQSGVPSRFSGSGSGTDFTLTISSLQPEDFATYYCQQSYSTPPITFGQGTRLEIK

SEQ ID NO: 84 - protein, 117 aa, synthetic construct
  REGION 1..117: note = Synthetic: Anti-MUC16 8767 variable heavy chain
QVQLVESGGGLVKPGGSLRLSCAASGFTFSNYYMSWVRQAPGKGLEWISYISGRGSTIFYADSVKGRITISRDNAKNSLFLQMNSLRAEDTAVYFCVKDRGGYSPYWGQGTLVTVSS

SEQ ID NO: 85 - protein, 463 aa, synthetic construct
  REGION 1..463: note = Synthetic: pBB4006 CAR a.a. sequence without signal peptide
QVQLVESGGGLVKPGGSLRLSCAASGFTFSNYYMSWVRQAPGKGLEWISYISGRGSTIFYADSVKGRITISRDNAKNSLFLQMNSLRAEDTAVYFCVKDRGGYSPYWGQGTLVTVSSGGGGSGGGGSGGGGSDIQMTQSPSSLSASVGDRVTITCRASQSISTYLNWYQQKPGKAPKLLIYTASSLQSGVPSRFSGSGSGTDFTLTISSLQPEDFATYYCQQSYSTPPITFGQGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 484 aa, synthetic construct
  REGION 1..484: note = Synthetic: pBB4006 CAR a.a. sequence with signal peptide
MALPVTALLLPLALLLHAARPQVQLVESGGGLVKPGGSLRLSCAASGFTFSNYYMSWVRQAPGKGLEWISYISGRGSTIFYADSVKGRITISRDNAKNSLFLQMNSLRAEDTAVYFCVKDRGGYSPYWGQGTLVTVSSGGGGSGGGGSGGGGSDIQMTQSPSSLSASVGDRVTITCRASQSISTYLNWYQQKPGKAPKLLIYTASSLQSGVPSRFSGSGSGTDFTLTISSLQPEDFATYYCQQSYSTPPITFGQGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 463 aa, synthetic construct
  REGION 1..463: note = Synthetic: pBB4007 CAR a.a. sequence without signal peptide
DIQMTQSPSSLSASVGDRVTITCRASQSISTYLNWYQQKPGKAPKLLIYTASSLQSGVPSRFSGSGSGTDFTLTISSLQPEDFATYYCQQSYSTPPITFGQGTRLEIKGGGGSGGGGSGGGGSQVQLVESGGGLVKPGGSLRLSCAASGFTFSNYYMSWVRQAPGKGLEWISYISGRGSTIFYADSVKGRITISRDNAKNSLFLQMNSLRAEDTAVYFCVKDRGGYSPYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 484 aa, synthetic construct
  REGION 1..484: note = Synthetic: pBB4007 CAR a.a. sequence with signal peptide
MALPVTALLLPLALLLHAARPDIQMTQSPSSLSASVGDRVTITCRASQSISTYLNWYQQKPGKAPKLLIYTASSLQSGVPSRFSGSGSGTDFTLTISSLQPEDFATYYCQQSYSTPPITFGQGTRLEIKGGGGSGGGGSGGGGSQVQLVESGGGLVKPGGSLRLSCAASGFTFSNYYMSWVRQAPGKGLEWISYISGRGSTIFYADSVKGRITISRDNAKNSLFLQMNSLRAEDTAVYFCVKDRGGYSPYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 6 aa, synthetic construct
  REGION 1..6: note = Synthetic: Anti-MUC16 8794 CDRL1
QSINSY

SEQ ID NO: 90 - skipped

SEQ ID NO: 91 - protein, 10 aa, synthetic construct
  REGION 1..10: note = Synthetic: Anti-MUC16 8794 CDRL3
QQSYSSPPIT

SEQ ID NO: 92 - protein, 8 aa, synthetic construct
  REGION 1..8: note = Synthetic: Anti-MUC16 8794 CDRH1
GFTFRDYS

SEQ ID NO: 93 - protein, 8 aa, synthetic construct
  REGION 1..8: note = Synthetic: Anti-MUC16 8794 CDRH2
VTFFNSAI

SEQ ID NO: 94 - protein, 13 aa, synthetic construct
  REGION 1..13: note = Synthetic: Anti-MUC16 8794 CDRH3
AREREPIVGGFDY

SEQ ID NO: 95 - protein, 108 aa, synthetic construct
  REGION 1..108: note = Synthetic: Anti-MUC16 8794 variable light chain
DIQMTQSPSSLSASVGDRVTITCRASQSINSYLNWYQQKPGKAPKLLIYAASSLQGGVPSRFSGGGSGTDFTLTITSLQPEDFATFYCQQSYSSPPITFGQGTRLEIK

SEQ ID NO: 96 - protein, 120 aa, synthetic construct
  REGION 1..120: note = Synthetic: Anti-MUC16 8794 variable heavy chain
QVQLVESGGGLVKPGGSLRLSCAASGFTFRDYSMSWIRQAPGKGLEWVSYVTFFNSAIYYADSVKGRFTISRDNAKNSLYLQMNSLRAEDTAVYYCAREREPIVGGFDYWGQGTLVTVSS

SEQ ID NO: 97 - protein, 466 aa, synthetic construct
  REGION 1..466: note = Synthetic: pBB4008 CAR a.a. sequence without signal peptide
QVQLVESGGGLVKPGGSLRLSCAASGFTFRDYSMSWIRQAPGKGLEWVSYVTFFNSAIYYADSVKGRFTISRDNAKNSLYLQMNSLRAEDTAVYYCAREREPIVGGFDYWGQGTLVTVSSGGGGSGGGGSGGGGSDIQMTQSPSSLSASVGDRVTITCRASQSINSYLNWYQQKPGKAPKLLIYAASSLQGGVPSRFSGGGSGTDFTLTITSLQPEDFATFYCQQSYSSPPITFGQGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 487 aa, synthetic construct
  REGION 1..487: note = Synthetic: pBB4008 CAR a.a. sequence with signal peptide
MALPVTALLLPLALLLHAARPQVQLVESGGGLVKPGGSLRLSCAASGFTFRDYSMSWIRQAPGKGLEWVSYVTFFNSAIYYADSVKGRFTISRDNAKNSLYLQMNSLRAEDTAVYYCAREREPIVGGFDYWGQGTLVTVSSGGGGSGGGGSGGGGSDIQMTQSPSSLSASVGDRVTITCRASQSINSYLNWYQQKPGKAPKLLIYAASSLQGGVPSRFSGGGSGTDFTLTITSLQPEDFATFYCQQSYSSPPITFGQGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - protein, 466 aa, synthetic construct
  REGION 1..466: note = Synthetic: pBB4009 CAR a.a. sequence without signal peptide
DIQMTQSPSSLSASVGDRVTITCRASQSINSYLNWYQQKPGKAPKLLIYAASSLQGGVPSRFSGGGSGTDFTLTITSLQPEDFATFYCQQSYSSPPITFGQGTRLEIKGGGGSGGGGSGGGGSQVQLVESGGGLVKPGGSLRLSCAASGFTFRDYSMSWIRQAPGKGLEWVSYVTFFNSAIYYADSVKGRFTISRDNAKNSLYLQMNSLRAEDTAVYYCAREREPIVGGFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - protein, 487 aa, synthetic construct
  REGION 1..487: note = Synthetic: pBB4009 CAR a.a. sequence with signal peptide
MALPVTALLLPLALLLHAARPDIQMTQSPSSLSASVGDRVTITCRASQSINSYLNWYQQKPGKAPKLLIYAASSLQGGVPSRFSGGGSGTDFTLTITSLQPEDFATFYCQQSYSSPPITFGQGTRLEIKGGGGSGGGGSGGGGSQVQLVESGGGLVKPGGSLRLSCAASGFTFRDYSMSWIRQAPGKGLEWVSYVTFFNSAIYYADSVKGRFTISRDNAKNSLYLQMNSLRAEDTAVYYCAREREPIVGGFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 7 aa, synthetic construct
  REGION 1..7: note = Synthetic: Anti-MUC16 8799 CDRL1
QSITSSY

SEQ ID NO: 102 - skipped

SEQ ID NO: 103 - protein, 9 aa, synthetic construct
  REGION 1..9: note = Synthetic: Anti-MUC16 8799 CDRL3
QQYGSSPWT

SEQ ID NO: 104 - protein, 8 aa, synthetic construct
  REGION 1..8: note = Synthetic: Anti-MUC16 8799 CDRH1
GFAFGDHT

SEQ ID NO: 105 - protein, 10 aa, synthetic construct
  REGION 1..10: note = Synthetic: Anti-MUC16 8799 CDRH2
IRSRAYGGTT

SEQ ID NO: 106 - protein, 16 aa, synthetic construct
  REGION 1..16: note = Synthetic: Anti-MUC16 8799 CDRH3
TSGGYDSSLHYYYYYH

SEQ ID NO: 107 - protein, 108 aa, synthetic construct
  REGION 1..108: note = Synthetic: Anti-MUC16 8799 variable light chain
EIVLTQSPGTLSLSPGERATLSCRASQSITSSYLAWYQQRPGQAPRLLIYGASSRATGIPDRFSGSGSGTDFTLTISRLEPEDFAVYYCQQYGSSPWTFGQGTKVEIK

SEQ ID NO: 108 - protein, 129 aa, synthetic construct
  REGION 1..129: note = Synthetic: Anti-MUC16 8799 variable heavy chain
EVQLVESGGGLEQPGRSLRLSCTASGFAFGDHTMSWVRQAPGKGLEWVGFIRSRAYGGTTEYAASVKGRFTISRDDSKSIAYLQMDSLKTEDTAVYYCTSGGYDSSLHYYYYYHGMDVWGRGTTVTVSS

SEQ ID NO: 109 - protein, 475 aa, synthetic construct
  REGION 1..475: note = Synthetic: pBB4010 CAR a.a. sequence without signal peptide
EVQLVESGGGLEQPGRSLRLSCTASGFAFGDHTMSWVRQAPGKGLEWVGFIRSRAYGGTTEYAASVKGRFTISRDDSKSIAYLQMDSLKTEDTAVYYCTSGGYDSSLHYYYYYHGMDVWGRGTTVTVSSGGGGSGGGGSGGGGSEIVLTQSPGTLSLSPGERATLSCRASQSITSSYLAWYQQRPGQAPRLLIYGASSRATGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 110 - protein, 496 aa, synthetic construct
  REGION 1..496: note = Synthetic: pBB4010 CAR a.a. sequence with signal peptide
MALPVTALLLPLALLLHAARPEVQLVESGGGLEQPGRSLRLSCTASGFAFGDHTMSWVRQAPGKGLEWVGFIRSRAYGGTTEYAASVKGRFTISRDDSKSIAYLQMDSLKTEDTAVYYCTSGGYDSSLHYYYYYHGMDVWGRGTTVTVSSGGGGSGGGGSGGGGSEIVLTQSPGTLSLSPGERATLSCRASQSITSSYLAWYQQRPGQAPRLLIYGASSRATGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - protein, 475 aa, synthetic construct
  REGION 1..475: note = Synthetic: pBB4011 CAR a.a. sequence without signal peptide
EIVLTQSPGTLSLSPGERATLSCRASQSITSSYLAWYQQRPGQAPRLLIYGASSRATGIPDRFSGSGSGTDFTLTISRLEPEDFAVYYCQQYGSSPWTFGQGTKVEIKGGGGSGGGGSGGGGSEVQLVESGGGLEQPGRSLRLSCTASGFAFGDHTMSWVRQAPGKGLEWVGFIRSRAYGGTTEYAASVKGRFTISRDDSKSIAYLQMDSLKTEDTAVYYCTSGGYDSSLHYYYYYHGMDVWGRGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - protein, 496 aa, synthetic construct
  REGION 1..496: note = Synthetic: pBB4011 CAR a.a. sequence with signal peptide
MALPVTALLLPLALLLHAARPEIVLTQSPGTLSLSPGERATLSCRASQSITSSYLAWYQQRPGQAPRLLIYGASSRATGIPDRFSGSGSGTDFTLTISRLEPEDFAVYYCQQYGSSPWTFGQGTKVEIKGGGGSGGGGSGGGGSEVQLVESGGGLEQPGRSLRLSCTASGFAFGDHTMSWVRQAPGKGLEWVGFIRSRAYGGTTEYAASVKGRFTISRDDSKSIAYLQMDSLKTEDTAVYYCTSGGYDSSLHYYYYYHGMDVWGRGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 113 - protein, 7 aa, synthetic construct
  REGION 1..7: note = Synthetic: Anti-MUC16 8804 CDRL1
QTVSSSY

SEQ ID NO: 114 - skipped

SEQ ID NO: 115 - protein, 9 aa, synthetic construct
  REGION 1..9: note = Synthetic: Anti-MUC16 8804 CDRL3
QQYGSSPWT

SEQ ID NO: 116 - protein, 8 aa, synthetic construct
  REGION 1..8: note = Synthetic: Anti-MUC16 8804 CDRH1
GLTFGDYG

SEQ ID NO: 117 - protein, 10 aa, synthetic construct
  REGION 1..10: note = Synthetic: Anti-MUC16 8804 CDRH2
IRSKGYGGTT

SEQ ID NO: 118 - protein, 16 aa, synthetic construct
  REGION 1..16: note = Synthetic: Anti-MUC16 8804 CDRH3
TSGGYDSSVHYYYYYY

SEQ ID NO: 119 - protein, 108 aa, synthetic construct
  REGION 1..108: note = Synthetic: Anti-MUC16 8804 variable light chain
EIVLTQSPGTLSLSPGERATLSCRASQTVSSSYLAWYQQKPGQAPRLLIYGASSRATGIPDRFSGSGSGTDFTLTISRLEPEDFAVYYCQQYGSSPWTFGQGTKVEIK

SEQ ID NO: 120 - protein, 129 aa, synthetic construct
  REGION 1..129: note = Synthetic: Anti-MUC16 8804 variable heavy chain
EVQLVESGGGLVQPGRSLRLSCTGSGLTFGDYGMSWVRQAPGKGLEWVGFIRSKGYGGTTEYAASVKGRFTISRDDSKSTAYLQMNSLKTEDTAVYYCTSGGYDSSVHYYYYYYAMDVWGQGTTVTVSS

SEQ ID NO: 121 - protein, 475 aa, synthetic construct
  REGION 1..475: note = Synthetic: pBB4012 CAR a.a. sequence without signal peptide
EVQLVESGGGLVQPGRSLRLSCTGSGLTFGDYGMSWVRQAPGKGLEWVGFIRSKGYGGTTEYAASVKGRFTISRDDSKSTAYLQMNSLKTEDTAVYYCTSGGYDSSVHYYYYYYAMDVWGQGTTVTVSSGGGGSGGGGSGGGGSEIVLTQSPGTLSLSPGERATLSCRASQTVSSSYLAWYQQKPGQAPRLLIYGASSRATGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - protein, 496 aa, synthetic construct
  REGION 1..496: note = Synthetic: pBB4012 CAR a.a. sequence with signal peptide
MALPVTALLLPLALLLHAARPEVQLVESGGGLVQPGRSLRLSCTGSGLTFGDYGMSWVRQAPGKGLEWVGFIRSKGYGGTTEYAASVKGRFTISRDDSKSTAYLQMNSLKTEDTAVYYCTSGGYDSSVHYYYYYYAMDVWGQGTTVTVSSGGGGSGGGGSGGGGSEIVLTQSPGTLSLSPGERATLSCRASQTVSSSYLAWYQQKPGQAPRLLIYGASSRATGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 475 aa, synthetic construct
  REGION 1..475: note = Synthetic: pBB4013 CAR a.a. sequence without signal peptide
EIVLTQSPGTLSLSPGERATLSCRASQTVSSSYLAWYQQKPGQAPRLLIYGASSRATGIPDRFSGSGSGTDFTLTISRLEPEDFAVYYCQQYGSSPWTFGQGTKVEIKGGGGSGGGGSGGGGSEVQLVESGGGLVQPGRSLRLSCTGSGLTFGDYGMSWVRQAPGKGLEWVGFIRSKGYGGTTEYAASVKGRFTISRDDSKSTAYLQMNSLKTEDTAVYYCTSGGYDSSVHYYYYYYAMDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - protein, 496 aa, synthetic construct
  REGION 1..496: note = Synthetic: pBB4013 CAR a.a. sequence with signal peptide
MALPVTALLLPLALLLHAARPEIVLTQSPGTLSLSPGERATLSCRASQTVSSSYLAWYQQKPGQAPRLLIYGASSRATGIPDRFSGSGSGTDFTLTISRLEPEDFAVYYCQQYGSSPWTFGQGTKVEIKGGGGSGGGGSGGGGSEVQLVESGGGLVQPGRSLRLSCTGSGLTFGDYGMSWVRQAPGKGLEWVGFIRSKGYGGTTEYAASVKGRFTISRDDSKSTAYLQMNSLKTEDTAVYYCTSGGYDSSVHYYYYYYAMDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 467 aa, synthetic construct
  REGION 1..467: note = Synthetic: pBB4014 CAR a.a. sequence without signal peptide
QVQLVESGGGVVQPGRSLRLSCVASGFTFSNYGIHWVRQAPGKGLEWVAVISDDGSFKFYADSVKGRFTISRDNSKNTLYLQMNSLRVEDSAVYHCAKWQHNWNDGGFDYWGQGTLVTVSSGGGGSGGGGSGGGGSEIVLTQSPDTLSLSPGERATLSCRASQSLSSNYLAWYRQKPGQAPRLLIYGISSRATGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 488 aa, synthetic construct
  REGION 1..488: note = Synthetic: pBB4014 CAR a.a. sequence with signal peptide
MALPVTALLLPLALLLHAARPQVQLVESGGGVVQPGRSLRLSCVASGFTFSNYGIHWVRQAPGKGLEWVAVISDDGSFKFYADSVKGRFTISRDNSKNTLYLQMNSLRVEDSAVYHCAKWQHNWNDGGFDYWGQGTLVTVSSGGGGSGGGGSGGGGSEIVLTQSPDTLSLSPGERATLSCRASQSLSSNYLAWYRQKPGQAPRLLIYGISSRATGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 7 aa, synthetic construct
  REGION 1..7: note = Synthetic: Anti-MUC16 8810 CDRL1
QSVSNRY

SEQ ID NO: 128 - skipped

SEQ ID NO: 129 - protein, 9 aa, synthetic construct
  REGION 1..9: note = Synthetic: Anti-MUC16 8810 CDRL3
HQYGSSPWT

SEQ ID NO: 130 - protein, 8 aa, synthetic construct
  REGION 1..8: note = Synthetic: Anti-MUC16 8810 CDRH1
GFTFSSYA

SEQ ID NO: 131 - protein, 8 aa, synthetic construct
  REGION 1..8: note = Synthetic: Anti-MUC16 8810 CDRH2
ISYNGGTT

SEQ ID NO: 132 - protein, 6 aa, synthetic construct
  REGION 1..6: note = Synthetic: Anti-MUC16 8810 CDRH3
ARAAAP

SEQ ID NO: 133 - protein, 108 aa, synthetic construct
  REGION 1..108: note = Synthetic: Anti-MUC16 8810 variable light chain
EIVLTQSPDTLSLSPGERATLSCRASQSVSNRYLAWYQQKPGQAPRLLIYGASSRATGIPDRFSGSGSGTDFTLTISRLEPEDFAVYYCHQYGSSPWTFGQGTKVEIK

SEQ ID NO: 134 - protein, 113 aa, synthetic construct
  REGION 1..113: note = Synthetic: Anti-MUC16 8810 variable heavy chain
EVQLVESGGALVQPGGSLRLSCAASGFTFSSYAMHWVRQAPGKGLEFVSTISYNGGTTYYADSVKGRFTVSRDNSKNTVFLRMGSLRTEDMAVYYCARAAAPWGQGTLVTVSS

SEQ ID NO: 135 - protein, 459 aa, synthetic construct
  REGION 1..459: note = Synthetic: pBB4016 CAR a.a. sequence without signal peptide
EVQLVESGGALVQPGGSLRLSCAASGFTFSSYAMHWVRQAPGKGLEFVSTISYNGGTTYYADSVKGRFTVSRDNSKNTVFLRMGSLRTEDMAVYYCARAAAPWGQGTLVTVSSGGGGSGGGGSGGGGSEIVLTQSPDTLSLSPGERATLSCRASQSVSNRYLAWYQQKPGQAPRLLIYGASSRATGIPDRFSGSGSGTDFTLTISRLEPEDFAVYYCHQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - protein, 480 aa, synthetic construct
  REGION 1..480: note = Synthetic: pBB4016 CAR a.a. sequence with signal peptide
MALPVTALLLPLALLLHAARPEVQLVESGGALVQPGGSLRLSCAASGFTFSSYAMHWVRQAPGKGLEFVSTISYNGGTTYYADSVKGRFTVSRDNSKNTVFLRMGSLRTEDMAVYYCARAAAPWGQGTLVTVSSGGGGSGGGGSGGGGSEIVLTQSPDTLSLSPGERATLSCRASQSVSNRYLAWYQQKPGQAPRLLIYGASSRATGIPDRFSGSGSGTDFTLTISRLEPEDFAVYYCHQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 459 aa, synthetic construct
  REGION 1..459: note = Synthetic: pBB4017 CAR a.a. sequence without signal peptide
EIVLTQSPDTLSLSPGERATLSCRASQSVSNRYLAWYQQKPGQAPRLLIYGASSRATGIPDRFSGSGSGTDFTLTISRLEPEDFAVYYCHQYGSSPWTFGQGTKVEIKGGGGSGGGGSGGGGSEVQLVESGGALVQPGGSLRLSCAASGFTFSSYAMHWVRQAPGKGLEFVSTISYNGGTTYYADSVKGRFTVSRDNSKNTVFLRMGSLRTEDMAVYYCARAAAPWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 480 aa, synthetic construct
  REGION 1..480: note = Synthetic: pBB4017 CAR a.a. sequence with signal peptide
MALPVTALLLPLALLLHAARPEIVLTQSPDTLSLSPGERATLSCRASQSVSNRYLAWYQQKPGQAPRLLIYGASSRATGIPDRFSGSGSGTDFTLTISRLEPEDFAVYYCHQYGSSPWTFGQGTKVEIKGGGGSGGGGSGGGGSEVQLVESGGALVQPGGSLRLSCAASGFTFSSYAMHWVRQAPGKGLEFVSTISYNGGTTYYADSVKGRFTVSRDNSKNTVFLRMGSLRTEDMAVYYCARAAAPWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 7 aa, synthetic construct
  REGION 1..7: note = Synthetic: Anti-MUC16 8813 CDRL1
QSVSSSY

SEQ ID NO: 140 - skipped

SEQ ID NO: 141 - protein, 9 aa, synthetic construct
  REGION 1..9: note = Synthetic: Anti-MUC16 8813 CDRL3
QQYGSSPWT

SEQ ID NO: 142 - protein, 8 aa, synthetic construct
  REGION 1..8: note = Synthetic: Anti-MUC16 8813 CDRH1
GYTLTGYY

SEQ ID NO: 143 - protein, 8 aa, synthetic construct
  REGION 1..8: note = Synthetic: Anti-MUC16 8813 CDRH2
INPNNGGT

SEQ ID NO: 144 - protein, 13 aa, synthetic construct
  REGION 1..13: note = Synthetic: Anti-MUC16 8813 CDRH3
ARSLELLPDGMDV

SEQ ID NO: 145 - protein, 108 aa, synthetic construct
  REGION 1..108: note = Synthetic: Anti-MUC16 8813 variable light chain
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 146 - protein, 120 aa, synthetic construct
  REGION 1..120: note = Synthetic: Anti-MUC16 8813 variable heavy chain
QVQLVQSGAEVKKPGASVKVSCKAFGYTLTGYYIHWVRQAPGQGLEWMGWINPNNGGTNYAQKFQGRVTMTRDKSISTAYMELSRLRSDDTAVYYCARSLELLPDGMDVWGQGTTVTVSS

SEQ ID NO: 147 - protein, 466 aa, synthetic construct
  REGION 1..466: note = Synthetic: pBB4018 CAR a.a. sequence without signal peptide
QVQLVQSGAEVKKPGASVKVSCKAFGYTLTGYYIHWVRQAPGQGLEWMGWINPNNGGTNYAQKFQGRVTMTRDKSISTAYMELSRLRSDDTAVYYCARSLELLPDGMDVWGQGTTVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 148 - protein, 487 aa, synthetic construct
  REGION 1..487: note = Synthetic: pBB4018 CAR a.a. sequence with signal peptide
MALPVTALLLPLALLLHAARPQVQLVQSGAEVKKPGASVKVSCKAFGYTLTGYYIHWVRQAPGQGLEWMGWINPNNGGTNYAQKFQGRVTMTRDKSISTAYMELSRLRSDDTAVYYCARSLELLPDGMDVWGQGTTVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 149 - protein, 466 aa, synthetic construct
  REGION 1..466: note = Synthetic: pBB4019 CAR a.a. sequence without signal peptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSGGGGSGGGGSQVQLVQSGAEVKKPGASVKVSCKAFGYTLTGYYIHWVRQAPGQGLEWMGWINPNNGGTNYAQKFQGRVTMTRDKSISTAYMELSRLRSDDTAVYYCARSLELLPDGMDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 150 - protein, 487 aa, synthetic construct
  REGION 1..487: note = Synthetic: pBB4019 CAR a.a. sequence with signal peptide
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSGGGGSGGGGSQVQLVQSGAEVKKPGASVKVSCKAFGYTLTGYYIHWVRQAPGQGLEWMGWINPNNGGTNYAQKFQGRVTMTRDKSISTAYMELSRLRSDDTAVYYCARSLELLPDGMDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 151 - protein, 642 aa, Homo sapiens
  REGION 1..642: note = misc_feature - Human MUC16 ectodomain ( nub )
PGSRKFNTTERVLQGLLRPLFKNTSVGPLYSGCRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMQHLLSPLFQRSSLGARYTGCRVIALRSVKNGAETRVDLLCTYLQPLSGPGLPIKQVFHELSQQTHGITRLGPYSLDKDSLYLNGYNEPGPDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLP

SEQ ID NO: 152 - protein, 21 aa, synthetic construct
  REGION 1..21: note = Synthetic: CD8 signal peptide
MALPVTALLLPLALLLHAARP

SEQ ID NO: 153 - protein, 45 aa, synthetic construct
  REGION 1..45: note = Synthetic: CD8 hinge region
TTTPAPRPPTPAPTIASQPLSLRPEACRPAAGGAVHTRGLDFACD

SEQ ID NO: 154 - protein, 21 aa, synthetic construct
  REGION 1..21: note = Synthetic: CD8 transmembrane region
IYIWAPLAGTCGVLLLSLVIT

SEQ ID NO: 155 - protein, 42 aa, synthetic construct
  REGION 1..42: note = Synthetic: 4-1BB co-stimulatory region
KRGRKKLLYIFKQPFMRPVQTTQEEDGCSCRFPEEEEGGCEL

SEQ ID NO: 156 - protein, 112 aa, synthetic construct
  REGION 1..112: note = Synthetic: CD3 signaling region
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR